SEQUENCE LISTING (1497 sequences)

SEQ ID NO: 1 - protein, 7 aa, Mus musculus
  murine VH CDR1 antibody amino acid sequence
TSGMSVG

SEQ ID NO: 2 - protein, 16 aa, Mus musculus
  murine VH CDR2 antibody amino acid sequence
DIWWDDKKDYNPSLKS

SEQ ID NO: 3 - protein, 10 aa, Mus musculus
  murine VH CDR3 antibody amino acid sequence
SMITNWYFDV

SEQ ID NO: 4 - protein, 10 aa, Artificial Sequence
  Murine-derived VL CDR1 antibody amino acid sequence
KCQLSVGYMH

SEQ ID NO: 5 - protein, 7 aa, Mus musculus
  murine VL CDR2 antibody amino acid sequence
DTSKLAS

SEQ ID NO: 6 - protein, 9 aa, Mus musculus
  murine VL CDR3 antibody amino acid sequence
FQGSGYPFT

SEQ ID NO: 7 - protein, 120 aa, Artificial Sequence
  Murine-derived CDR sequences in a human framework - VH Domain
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 8 - protein, 106 aa, Artificial Sequence
  Murine-derived CDR sequences in a human framework - VL Domain
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIK

SEQ ID NO: 9 - protein, 120 aa, Artificial Sequence
  Murine-derived CDR sequences in a human framework - VH Domain
QVTLRESGPALVKPTQTLTLTCTFSGFSLSTAGMSVGWIRQPPGKALEWLADIWWDDKKDYNPSLKSRLTISKDTSKNQVVLKVTNMDPADTATYYCARSMITNFYFDVWGQGTTVTVSS

SEQ ID NO: 10 - protein, 7 aa, Artificial Sequence
  Murine-derived CDR sequence
TAGMSVG

SEQ ID NO: 11 - protein, 106 aa, Artificial Sequence [sequence omitted]

SEQ ID NO: 12 - protein, 10 aa, Artificial Sequence
  Murine-derived CDR sequence
SMITNFYFDV

SEQ ID NO: 13 - protein, 106 aa, Artificial Sequence
  Murine-derived CDR sequences in a human framework - VL Domain
DIQMTQSPSTLSASVGDRVTITCSASSSVGYMHWYQQKPGKAPKLLIYDTFKLASGVPSRFSGSGSGTEFTLTISSLQPDDFATYYCFQFSGYPFTFGGGTKVEIK

SEQ ID NO: 14 - protein, 10 aa, Artificial Sequence
  Murine-derived CDR sequence
SASSSVGYMH

SEQ ID NO: 15 - protein, 7 aa, Artificial Sequence
  Murine-derived CDR sequence
DTFKLAS

SEQ ID NO: 16 - protein, 9 aa, Artificial Sequence
  Murine-derived CDR sequences
FQFSGYPFT

SEQ ID NO: 17 - protein, 120 aa, Artificial Sequence
  Murine-derived CDR sequences in a human framework - VH Domain
QVTLRESGPALVKPTQTLTLTCTFSGFSLSTPGMSVGWIRQPPGKALEWLADIWWDDKKHYNPSLKDRLTISKDTSKNQVVLKVTNMDPADTATYYCARDMIFNFYFDVWGQGTTVTVSS

SEQ ID NO: 18 - protein, 7 aa, Artificial Sequence
  Murine-derived CDR sequences
TPGMSVG

SEQ ID NO: 19 - protein, 16 aa, Artificial Sequence
  Murine-derived CDR sequences
DIWWDDKKHYNPSLKD

SEQ ID NO: 20 - protein, 10 aa, Artificial Sequence
  Murine-derived CDR sequences
DMIFNFYFDV

SEQ ID NO: 21 - protein, 106 aa, Artificial Sequence
  Murine-derived CDR sequences in a human framework - VL Domain
DIQMTQSPSTLSASVGDRVTITCSLSSRVGYMHWYQQKPGKAPKLLIYDTFYLSSGVPSRFSGSGSGTEFTLTISSLQPDDFATYYCFQGSGYPFTFGGGTKVEIK

SEQ ID NO: 22 - protein, 10 aa, Artificial Sequence
  Murine-derived CDR sequences
SLSSRVGYMH

SEQ ID NO: 23 - protein, 7 aa, Artificial Sequence
  Murine-derived CDR sequences
DTFYLSS

SEQ ID NO: 24 - protein, 120 aa, Artificial Sequence
  Murine-derived CDR sequences in a human framework - VH Domain
QVTLRESGPALVKPTQTLTLTCTFSGFSLSTPGMSVGWIRQPPGKALEWLADIWWDGKKHYNPSLKDRLTISKDTSKNQVVLKVTNMDPADTATYYCARDMIFNFYFDVWGQGTTVTVSS

SEQ ID NO: 25 - protein, 16 aa, Artificial Sequence
  Murine-derived CDR sequences
DIWWDGKKHYNPSLKD

SEQ ID NO: 26 - protein, 106 aa, Artificial Sequence
  Murine-derived CDR sequences in a human framework- VL Domain
DIQMTQSPSTLSASVGDRVTITCSLSSRVGYMHWYQQKPGKAPKLLIYDTRGLPSGVPSRFSGSGSGTEFTLTISSLQPDDFATYYCFQGSGYPFTFGGGTKVEIK

SEQ ID NO: 27 - protein, 7 aa, Artificial Sequence
  Murine-derived CDR sequences
DTRGLPS

SEQ ID NO: 28 - protein, 120 aa, Artificial Sequence
  Murine-derived CDR sequences in a human framework- VH Domain
QVTLRESGPALVKPTQTLTLTCTFSGFSLSTPGMSVGWIRQPPGKALEWLADIWWDGKKHYNPSLKDRLTISKDTSKNQVVLKVTNMDPADTATYYCARDMIFNWYFDVWGQGTTVTVSS

SEQ ID NO: 29 - protein, 10 aa, Artificial Sequence
  Murine-derived CDR sequences
DMIFNWYFDV

SEQ ID NO: 30 - protein, 106 aa, Artificial Sequence
  Murine-derived CDR sequences in a human framework- VL Domain
DIQMTQSPSTLSASVGDRVTITCSPSSRVGYMHWYQQKPGKAPKLLIYDTMRLASGVPSRFSGSGSGTEFTLTISSLQPDDFATYYCFQGSGYPFTFGGGTKVEIK

SEQ ID NO: 31 - protein, 10 aa, Artificial Sequence
  Murine-derived CDR sequences
SPSSRVGYMH

SEQ ID NO: 32 - protein, 7 aa, Artificial Sequence
  Murine-derived CDR sequences
DTMRLAS

SEQ ID NO: 33 - protein, 120 aa, Artificial Sequence
  Murine-derived CDR sequences in a human framework- VH Domain
QVTLRESGPALVKPTQTLTLTCTFSGFSLSTAGMSVGWIRQPPGKALEWLADIWWDGKKHYNPSLKDRLTISKDTSKNQVVLKVTNMDPADTATYYCARDMIFNWYFDVWGQGTTVTVSS

SEQ ID NO: 34 - protein, 106 aa, Artificial Sequence
  Murine-derived CDR sequences in a human framework- VL Domain
DIQMTQSPSTLSASVGDRVTITCSLSSRVGYMHWYQQKPGKAPKLLIYDTFKLSSGVPSRFSGSGSGTEFTLTISSLQPDDFATYYCFQGSGYPFTFGGGTKVEIK

SEQ ID NO: 35 - protein, 7 aa, Artificial Sequence
  Murine-derived CDR sequences
DTFKLSS

SEQ ID NO: 36 - protein, 120 aa, Artificial Sequence
  Murine-derived CDR sequences in a human framework- VH Domain
QVTLRESGPALVKPTQTLTLTCTFSGFSLSTAGMSVGWIRQPPGKALEWLADIWWDGKKDYNPSLKDRLTISKDTSKNQVVLKVTNMDPADTATYYCARDMIFNFYFDVWGQGTTVTVSS

SEQ ID NO: 37 - protein, 16 aa, Artificial Sequence
  Murine-derived CDR sequences
DIWWDGKKDYNPSLKD

SEQ ID NO: 38 - protein, 106 aa, Artificial Sequence
  Murine-derived CDR sequences in a human framework- VL Domain
DIQMTQSPSTLSASVGDRVTITCSASSRVGYMHWYQQKPGKAPKLLIYDTFKLSSGVPSRFSGSGSGTEFTLTISSLQPDDFATYYCFQGSGYPFTFGGGTKVEIK

SEQ ID NO: 39 - protein, 10 aa, Artificial Sequence
  Murine-derived CDR sequences
SASSRVGYMH

SEQ ID NO: 40 - protein, 120 aa, Artificial Sequence
  Murine-derived CDR sequences in a human framework- VH Domain
QVTLRESGPALVKPTQTLTLTCTFSGFSLSTAGMSVGWIRQPPGKALEWLADIWWDGKKSYNPSLKDRLTISKDTSKNQVVLKVTNMDPADTATYYCARDMIFNFYFDVWGQGTTVTVSS

SEQ ID NO: 41 - protein, 16 aa, Artificial Sequence
  Murine-derived CDR sequences
DIWWDGKKSYNPSLKD

SEQ ID NO: 42 - protein, 106 aa, Artificial Sequence
  Murine-derived CDR sequences in a human framework- VL Domain
DIQMTQSPSTLSASVGDRVTITCSLSSRVGYMHWYQQKPGKAPKLLIYDTMYQSSGVPSRFSGSGSGTEFTLTISSLQPDDFATYYCFQGSGYPFTFGGGTKVEIK

SEQ ID NO: 43 - protein, 7 aa, Artificial Sequence
  Murine-derived CDR sequences
DTMYQSS

SEQ ID NO: 44 - protein, 120 aa, Artificial Sequence
  Murine-derived CDR sequences in a human framework- VH Domain
QVTLRESGPALVKPTQTLTLTCTFSGFSLSTAGMSVGWIRQPPGKALEWLADIWWDDKKSYNPSLKDRLTISKDTSKNQVVLKVTNMDPADTATYYCARDMIFNFYFDVWGQGTTVTVSS

SEQ ID NO: 45 - protein, 16 aa, Artificial Sequence
  Murine-derived CDR sequences
DIWWDDKKSYNPSLKD

SEQ ID NO: 46 - protein, 106 aa, Artificial Sequence
  Murine-derived CDR sequences in a human framework- VL Domain
DIQMTQSPSTLSASVGDRVTITCLPSSRVGYMHWYQQKPGKAPKLLIYDTMYQSSGVPSRFSGSGSGTEFTLTISSLQPDDFATYYCFQGSGYPFTFGGGTKVEIK

SEQ ID NO: 47 - protein, 10 aa, Artificial Sequence
  Murine-derived CDR sequences
LPSSRVGYMH

SEQ ID NO: 48 - protein, 120 aa, Artificial Sequence [sequence omitted]

SEQ ID NO: 49 - protein, 106 aa, Artificial Sequence
  Murine-derived CDR sequences in a human framework- VL Domain
DIQMTQSPSTLSASVGDRVTITCSASSRVGYMHWYQQKPGKAPKLLIYDTFFLDSGVPSRFSGSGSGTEFTLTISSLQPDDFATYYCFQGSGYPFTFGGGTKVEIK

SEQ ID NO: 50 - protein, 7 aa, Artificial Sequence
  Murine-derived CDR sequences
DTFFLDS

SEQ ID NO: 51 - protein, 120 aa, Artificial Sequence
  Murine-derived CDR sequences in a human framework- VH Domain
QVTLRESGPALVKPTQTLTLTCTFSGFSLSTAGMSVGWIRQPPGKALEWLADIWWDDKKSYNPSLKDRLTISKDTSKNQVVLKVTNMDPADTATYYCARDMIFNWYFDVWGQGTTVTVSS

SEQ ID NO: 52 - protein, 106 aa, Artificial Sequence
  Murine-derived CDR sequences in a human framework- VL Domain
DIQMTQSPSTLSASVGDRVTITCSPSSRVGYMHWYQQKPGKAPKLLIYDTRYQSSGVPSRFSGSGSGTEFTLTISSLQPDDFATYYCFQGSGYPFTFGGGTKVEIK

SEQ ID NO: 53 - protein, 7 aa, Artificial Sequence
  Murine-derived CDR sequences
DTRYQSS

SEQ ID NO: 54 - protein, 106 aa, Artificial Sequence
  Murine-derived CDR sequences in a human framework- VL Domain
DIQMTQSPSTLSASVGDRVTITCSASSSVGYMHWYQQKPGKAPKLLIYDTSKLASGVPSRFSGSGSGTEFTLTISSLQPDDFATYYCFQGSGYPFTFGGGTKVEIK

SEQ ID NO: 55 - protein, 120 aa, Artificial Sequence
  Murine-derived CDR sequences in a human framework- VH Domain
QVTLRESGPALVKPTQTLTLTCTFSGFSLSTAGMSVGWIRQPPGKALEWLADIWWDDKKDYNPSLKSRLTISKDTSKNQVVLKVTNMDPADTATYYCARDMIFNWYFDVWGQGTTVTVSS

SEQ ID NO: 56 - protein, 106 aa, Artificial Sequence
  Murine-derived CDR sequences in a human framework- VL Domain
DIQMTQSPSTLSASVGDRVTITCSASSSVGYMHWYQQKPGKAPKLLIYDTFKLASGVPSRFSGSGSGTEFTLTISSLQPDDFATYYCFQGSGYPFTFGGGTKVEIK

SEQ ID NO: 57 - protein, 7 aa, Artificial Sequence
  Murine-derived CDR sequences
DTYKQTS

SEQ ID NO: 58 - protein, 106 aa, Artificial Sequence
  Murine-derived CDR sequences in a human framework- VL Domain
DIQMTQSPSTLSASVGDRVTITCSASSSVGYMHWYQQKPGKAPKLLIYDTRYLSSGVPSRFSGSGSGTEFTLTISSLQPDDFATYYCFQGSGYPFTFGGGTKVEIK

SEQ ID NO: 59 - protein, 7 aa, Artificial Sequence
  Murine-derived CDR sequences
DTRYLSS

SEQ ID NO: 60 - protein, 106 aa, Artificial Sequence
  Murine-derived CDR sequences in a human framework- VL Domain
DIQMTQSPSTLSASVGDRVTITCSASSSVGYMHWYQQKPGKAPKLLIYDTFKLASGVPSRFSGSGSGTEFTLTISSLQPDDFATYYCFQGSFYPFTFGGGTKVEIK

SEQ ID NO: 61 - protein, 9 aa, Artificial Sequence
  Murine-derived CDR sequences
FQGSFYPFT

SEQ ID NO: 62 - protein, 106 aa, Artificial Sequence
  Murine-derived CDR sequences in a human framework- VL Domain
DIQMTQSPSTLSASVGDRVTITCSASSSVGYMHWYQQKPGKAPKLLIYDTFKLTSGVPSRFSGSGSGTEFTLTISSLQPDDFATYYCFQGSGYPFTFGGGTKVEIK

SEQ ID NO: 63 - protein, 7 aa, Artificial Sequence
  Murine-derived CDR sequences
DTFKLTS

SEQ ID NO: 64 - protein, 106 aa, Artificial Sequence
  Murine-derived CDR sequences in a human framework- VL Domain
DIQMTQSPSTLSASVGDRVTITCSASSRVGYMHWYQQKPGKAPKLLIYDTFKLASGVPSRFSGSGSGTEFTLTISSLQPDDFATYYCFQGSGYPFTFGGGTKVEIK

SEQ ID NO: 65 - protein, 106 aa, Artificial Sequence
  Murine-derived CDR sequences in a human framework- VL Domain
DIQMTQSPSTLSASVGDRVTITCSASSSVGYMHWYQQKPGKAPKLLIYDTFRLASGVPSRFSGSGSGTEFTLTISSLQPDDFATYYCFQGSGYPFTFGGGTKVEIK

SEQ ID NO: 66 - protein, 7 aa, Artificial Sequence
  Murine-derived CDR sequences
DTFRLAS

SEQ ID NO: 67 - protein, 120 aa, Artificial Sequence
  Murine-derived CDR sequences in a human framework- VH Domain
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNWYFDVWGQGTTVTVSS

SEQ ID NO: 68 - protein, 106 aa, Artificial Sequence
  Murine-derived CDR sequences in a human framework- VL Domain
DIQMTQSPSTLSASVGDRVTITCSPSSRVGYMHWYQQKPGKAPKLLIYDTYRHSSGVPSRFSGSGSGTEFTLTISSLQPDDFATYYCFQGSGYPFTFGGGTKVEIK

SEQ ID NO: 69 - protein, 7 aa, Artificial Sequence
  Murine-derived CDR sequences
DTYRHSS

SEQ ID NO: 70 - protein, 106 aa, Artificial Sequence
  Murine-derived CDR sequences in a human framework- VL Domain
DIQMTQSPSTLSASVGDRVTITCSASSSVGYMHWYQQKPGKAPKLLIYDTYKQTSGVPSRFSGSGSGTEFTLTISSLQPDDFATYYCFQGSGYPFTFGGGTKVEIK

SEQ ID NO: 71 - protein, 106 aa, Artificial Sequence
  Murine-derived CDR sequences in a human framework- VL Domain
DIQMTQSPSTLSASVGDRVTITCSLSSSVGYMHWYQQKPGKAPKLLIYDTFFHRSGVPSRFSGSGSGTEFTLTISSLQPDDFATYYCFQGSGYPFTFGGGTKVEIK

SEQ ID NO: 72 - protein, 10 aa, Artificial Sequence
  Murine-derived CDR sequences
SLSSSVGYMH

SEQ ID NO: 73 - protein, 7 aa, Artificial Sequence
  Murine-derived CDR sequences
DTFFHRS

SEQ ID NO: 74 - protein, 106 aa, Artificial Sequence
  Murine-derived CDR sequences in a human framework- VL Domain
DIQMTQSPSTLSASVGDRVTITCSASSRVGYMHWYQQKPGKAPKLLIYDTLLLDSGVPSRFSGSGSGTEFTLTISSLQPDDFATYYCFQGSGYPFTFGGGTKVEIK

SEQ ID NO: 75 - protein, 7 aa, Artificial Sequence
  Murine-derived CDR sequences
DTLLLDS

SEQ ID NO: 76 - protein, 106 aa, Artificial Sequence
  Murine-derived CDR sequences in a human framework- VL Domain
DIQMTQSPSTLSASVGDRVTITCSASSRVGYMHWYQQKPGKAPKLLIYDTSFLDSGVPSRFSGSGSGTEFTLTISSLQPDDFATYYCFQGSGYPFTFGGGTKVEIK

SEQ ID NO: 77 - protein, 7 aa, Artificial Sequence
  Murine-derived CDR sequences
DTSFLDS

SEQ ID NO: 78 - protein, 120 aa, Artificial Sequence
  Murine-derived CDR sequences in a human framework- VH Domain
QVTLRESGPALVKPTQTLTLTCTFSGFSLSTAGMSVGWIRQPPGKALEWLADIWWDDKKDYNPSLKSRLTISKDTSKNQVVLKVTNMDPADTATYYCARDMITNFYFDVWGQGTTVTVSS

SEQ ID NO: 79 - protein, 10 aa, Artificial Sequence
  Murine-derived CDR sequences
DMITNFYFDV

SEQ ID NO: 80 - protein, 10 aa, Artificial Sequence
  Murine-derived CDR sequences
KCQSSVGYMH

SEQ ID NO: 81 - protein, 7 aa, Artificial Sequence
  Murine-derived CDR sequences
DTSYLAS

SEQ ID NO: 82 - protein, 16 aa, Artificial Sequence
  Murine-derived CDR sequences
DIWWDDKKHYNPSLKS

SEQ ID NO: 83 - protein, 10 aa, Artificial Sequence
  Murine-derived CDR sequences
DMITNWYFDV

SEQ ID NO: 84 - protein, 10 aa, Artificial Sequence
  Murine-derived CDR sequences
KCQSRVGYMH

SEQ ID NO: 85 - protein, 7 aa, Artificial Sequence
  Murine-derived CDR sequences
DTSYLSS

SEQ ID NO: 86 - protein, 16 aa, Artificial Sequence
  Murine-derived CDR sequences
DIWWDDKKDYNPSLKD

SEQ ID NO: 87 - protein, 10 aa, Artificial Sequence
  Murine-derived CDR sequences
KCQLRVGYMH

SEQ ID NO: 88 - protein, 7 aa, Artificial Sequence
  Murine-derived CDR sequences
DTKKLSS

SEQ ID NO: 89 - protein, 10 aa, Artificial Sequence
  Murine-derived CDR sequences
KLQLSVGYMH

SEQ ID NO: 90 - protein, 7 aa, Artificial Sequence
  Murine-derived CDR sequences
DTFYLSS

SEQ ID NO: 91 - protein, 16 aa, Artificial Sequence
  Murine-derived CDR sequences
DIWWDDKKHYNPSLKS

SEQ ID NO: 92 - protein, 10 aa, Artificial Sequence
  Murine-derived CDR sequences
KLQSSVGYMH

SEQ ID NO: 93 - protein, 16 aa, Artificial Sequence
  Murine-derived CDR sequences
DIWWDDKKHYNPSLKS

SEQ ID NO: 94 - protein, 10 aa, Artificial Sequence
  Murine-derived CDR sequences
SMIFNWYFDV

SEQ ID NO: 95 - protein, 10 aa, Artificial Sequence
  Murine-derived CDR sequences
KLQSRVGYMH

SEQ ID NO: 96 - protein, 7 aa, Artificial Sequence
  Murine-derived CDR sequences
DTFKLSS

SEQ ID NO: 97 - protein, 10 aa, Artificial Sequence
  Murine-derived CDR sequences
SMIFNFYFDV

SEQ ID NO: 98 - protein, 10 aa, Artificial Sequence
  Murine-derived CDR sequences
KLQLRVGYMH

SEQ ID NO: 99 - protein, 7 aa, Artificial Sequence
  Murine-derived CDR sequences
DTFYLAS

SEQ ID NO: 100 - protein, 16 aa, Artificial Sequence
  Murine-derived CDR sequences
DIWWDGKKDYNPSLKS

SEQ ID NO: 101 - protein, 10 aa, Artificial Sequence
  Murine-derived CDR sequences
KLSLSVGYMH

SEQ ID NO: 102 - protein, 7 aa, Artificial Sequence
  Murine-derived CDR sequences
DTSKLPS

SEQ ID NO: 103 - protein, 16 aa, Artificial Sequence
  Murine-derived CDR sequences
DIWWDGKKDYNPSLKD

SEQ ID NO: 104 - protein, 10 aa, Artificial Sequence
  Murine-derived CDR sequences
KLSSSVGYMH

SEQ ID NO: 105 - protein, 7 aa, Artificial Sequence
  Murine-derived CDR sequences
DTSGLAS

SEQ ID NO: 106 - protein, 16 aa, Artificial Sequence
  Murine-derived CDR sequences
DIWWDGKKHYNPSLKS

SEQ ID NO: 107 - protein, 10 aa, Artificial Sequence
  Murine-derived CDR sequences
KLSSRVGYMH

SEQ ID NO: 108 - protein, 7 aa, Artificial Sequence
  Murine-derived CDR sequences
DTSGLPS

SEQ ID NO: 109 - protein, 16 aa, Artificial Sequence
  Murine-derived CDR sequences
DIWWDDKKSYNPSLKS

SEQ ID NO: 110 - protein, 10 aa, Artificial Sequence
  Murine-derived CDR sequences
KLSLRVGYMH

SEQ ID NO: 111 - protein, 16 aa, Artificial Sequence
  Murine-derived CDR sequences
DIWWDDKKSYNPSLKD

SEQ ID NO: 112 - protein, 10 aa, Artificial Sequence
  Murine-derived CDR sequences
KCSLSVGYMH

SEQ ID NO: 113 - protein, 7 aa, Artificial Sequence
  Murine-derived CDR sequences
DTRKLAS

SEQ ID NO: 114 - protein, 16 aa, Artificial Sequence
  Murine-derived CDR sequences
DIWWDGKKSYNPSLKS

SEQ ID NO: 115 - protein, 10 aa, Artificial Sequence
  Murine-derived CDR sequences
KCSSSVGYMH

SEQ ID NO: 116 - protein, 7 aa, Artificial Sequence
  Murine-derived CDR sequences
DTRGLAS

SEQ ID NO: 117 - protein, 10 aa, Artificial Sequence
  Murine-derived CDR sequences
KCSSRVGYMH

SEQ ID NO: 118 - protein, 7 aa, Artificial Sequence
  Murine-derived CDR sequences
DTRKLPS

SEQ ID NO: 119 - protein, 10 aa, Artificial Sequence
  Murine-derived CDR sequences
KCSLRVGYMH

SEQ ID NO: 120 - protein, 10 aa, Artificial Sequence
  Murine-derived CDR sequences
SLSLSVGYMH

SEQ ID NO: 121 - protein, 7 aa, Artificial Sequence
  Murine-derived CDR sequences
DTMKLAS

SEQ ID NO: 122 - protein, 10 aa, Artificial Sequence
  Murine-derived CDR sequences
SLSSSVGYMH

SEQ ID NO: 123 - protein, 7 aa, Artificial Sequence
  Murine-derived CDR sequences
DTSRLAS

SEQ ID NO: 124 - protein, 7 aa, Artificial Sequence
  Murine-derived CDR sequences
DTSLLAS

SEQ ID NO: 125 - protein, 10 aa, Artificial Sequence
  Murine-derived CDR sequences
SLSLRVGYMH

SEQ ID NO: 126 - protein, 7 aa, Artificial Sequence
  Murine-derived CDR sequences
DTSLLDS

SEQ ID NO: 127 - protein, 10 aa, Artificial Sequence
  Murine-derived CDR sequences
SCQLSVGYMH

SEQ ID NO: 128 - protein, 7 aa, Artificial Sequence
  Murine-derived CDR sequences
DTSKLDS

SEQ ID NO: 129 - protein, 10 aa, Artificial Sequence
  Murine-derived CDR sequences
SCQSSVGYMH

SEQ ID NO: 130 - protein, 10 aa, Artificial Sequence
  Murine-derived CDR sequences
SCQSRVGYMH

SEQ ID NO: 131 - protein, 7 aa, Artificial Sequence
  Murine-derived CDR sequences
DTLKLDS

SEQ ID NO: 132 - protein, 10 aa, Artificial Sequence
  Murine-derived CDR sequences
SCQLRVGYMH

SEQ ID NO: 133 - protein, 7 aa, Artificial Sequence
  Murine-derived CDR sequences
DTLLLAS

SEQ ID NO: 134 - protein, 10 aa, Artificial Sequence
  Murine-derived CDR sequences
SLQLSVGYMH

SEQ ID NO: 135 - protein, 7 aa, Artificial Sequence
  Murine-derived CDR sequences
DTLKLAS

SEQ ID NO: 136 - protein, 10 aa, Artificial Sequence
  Murine-derived CDR sequences
SLQSSVGYMH

SEQ ID NO: 137 - protein, 7 aa, Artificial Sequence
  Murine-derived CDR sequences
DTSKLSS

SEQ ID NO: 138 - protein, 10 aa, Artificial Sequence
  Murine-derived CDR sequences
SLQSRVGYMH

SEQ ID NO: 139 - protein, 7 aa, Artificial Sequence
  Murine-derived CDR sequences
DTSKQAS

SEQ ID NO: 140 - protein, 10 aa, Artificial Sequence
  Murine-derived CDR sequences
SLQLRVGYMH

SEQ ID NO: 141 - protein, 7 aa, Artificial Sequence
  Murine-derived CDR sequences
DTSKQSS

SEQ ID NO: 142 - protein, 10 aa, Artificial Sequence
  Murine-derived CDR sequences
SCSLSVGYMH

SEQ ID NO: 143 - protein, 7 aa, Artificial Sequence
  Murine-derived CDR sequences
DTSYLAS

SEQ ID NO: 144 - protein, 10 aa, Artificial Sequence
  Murine-derived CDR sequences
SCSSSVGYMH

SEQ ID NO: 145 - protein, 7 aa, Artificial Sequence
  Murine-derived CDR sequences
DTSYLSS

SEQ ID NO: 146 - protein, 10 aa, Artificial Sequence
  Murine-derived CDR sequences
SCSSRVGYMH

SEQ ID NO: 147 - protein, 7 aa, Artificial Sequence
  Murine-derived CDR sequences
DTSYQAS

SEQ ID NO: 148 - protein, 10 aa, Artificial Sequence
  Murine-derived CDR sequences
SCSLRVGYMH

SEQ ID NO: 149 - protein, 7 aa, Artificial Sequence
  Murine-derived CDR sequences
DTSYQSS

SEQ ID NO: 150 - protein, 10 aa, Artificial Sequence
  Murine-derived CDR sequences
KPSSRVGYMH

SEQ ID NO: 151 - protein, 7 aa, Artificial Sequence
  Murine-derived CDR sequences
DTMYQAS

SEQ ID NO: 152 - protein, 10 aa, Artificial Sequence
  Murine-derived CDR sequences
KPSLRVGYMH

SEQ ID NO: 153 - protein, 10 aa, Artificial Sequence
  Murine-derived CDR sequences
KPSSSVGYMH

SEQ ID NO: 154 - protein, 7 aa, Artificial Sequence
  Murine-derived CDR sequences
DTMKQAS

SEQ ID NO: 155 - protein, 10 aa, Artificial Sequence
  Murine-derived CDR sequences
KPSLSVGYMH

SEQ ID NO: 156 - protein, 7 aa, Artificial Sequence
  Murine-derived CDR sequences
DTMKQSS

SEQ ID NO: 157 - protein, 10 aa, Artificial Sequence
  Murine-derived CDR sequences
KPQSRVGYMH

SEQ ID NO: 158 - protein, 7 aa, Artificial Sequence
  Murine-derived CDR sequences
DTMYLAS

SEQ ID NO: 159 - protein, 10 aa, Artificial Sequence
  Murine-derived CDR sequences
KPQLRVGYMH

SEQ ID NO: 160 - protein, 7 aa, Artificial Sequence
  Murine-derived CDR sequences
DTMYLSS

SEQ ID NO: 161 - protein, 10 aa, Artificial Sequence
  Murine-derived CDR sequences
KPQSSVGYMH

SEQ ID NO: 162 - protein, 7 aa, Artificial Sequence
  Murine-derived CDR sequences
DTMKLAS

SEQ ID NO: 163 - protein, 10 aa, Artificial Sequence
  Murine-derived CDR sequences
KPQLSVGYMH

SEQ ID NO: 164 - protein, 7 aa, Artificial Sequence
  Murine-derived CDR sequences
DTMKLSS

SEQ ID NO: 165 - protein, 7 aa, Artificial Sequence
  Murine-derived CDR sequences
DTSKLSS

SEQ ID NO: 166 - protein, 10 aa, Artificial Sequence
  Murine-derived CDR sequences
SPSLRVGYMH

SEQ ID NO: 167 - protein, 7 aa, Artificial Sequence
  Murine-derived CDR sequences
DTRYQAS

SEQ ID NO: 168 - protein, 10 aa, Artificial Sequence
  Murine-derived CDR sequences
SPSSSVGYMH

SEQ ID NO: 169 - protein, 10 aa, Artificial Sequence
  Murine-derived CDR sequences
SPSLSVGYMH

SEQ ID NO: 170 - protein, 7 aa, Artificial Sequence
  Murine-derived CDR sequences
DTRYQAS

SEQ ID NO: 171 - protein, 10 aa, Artificial Sequence
  Murine-derived CDR sequences
SPQSRVGYMH

SEQ ID NO: 172 - protein, 7 aa, Artificial Sequence
  Murine-derived CDR sequences
DTRKQSS

SEQ ID NO: 173 - protein, 10 aa, Artificial Sequence
  Murine-derived CDR sequences
SPQLRVGYMH

SEQ ID NO: 174 - protein, 7 aa, Artificial Sequence
  Murine-derived CDR sequences
DTRKLAS

SEQ ID NO: 175 - protein, 7 aa, Artificial Sequence
  Murine-derived CDR sequences
DTRKLSS

SEQ ID NO: 176 - protein, 10 aa, Artificial Sequence
  Murine-derived CDR sequences
SPQSSVGYMH

SEQ ID NO: 177 - protein, 10 aa, Artificial Sequence
  Murine-derived CDR sequences
SPQLSVGYMH

SEQ ID NO: 178 - protein, 7 aa, Artificial Sequence
  Murine-derived CDR sequences
DTRYLAS

SEQ ID NO: 179 - protein, 10 aa, Artificial Sequence
  Murine-derived CDR sequences
KAQSRVGYMH

SEQ ID NO: 180 - protein, 10 aa, Artificial Sequence
  Murine-derived CDR sequences
KAQLRVGYMH

SEQ ID NO: 181 - protein, 10 aa, Artificial Sequence
  Murine-derived CDR sequences
KAQSSVGYMH

SEQ ID NO: 182 - protein, 10 aa, Artificial Sequence
  Murine-derived CDR sequences
KAQLSVGYMH

SEQ ID NO: 183 - protein, 10 aa, Artificial Sequence
  Murine-derived CDR sequences
KASSRVGYMH

SEQ ID NO: 184 - protein, 10 aa, Artificial Sequence
  Murine-derived CDR sequences
KASLRVGYMH

SEQ ID NO: 185 - protein, 10 aa, Artificial Sequence
  Murine-derived CDR sequences
KASSSVGYMH

SEQ ID NO: 186 - protein, 10 aa, Artificial Sequence
  Murine-derived CDR sequences
KASLSVGYMH

SEQ ID NO: 187 - protein, 10 aa, Artificial Sequence
  Murine-derived CDR sequences
SASLRVGYMH

SEQ ID NO: 188 - protein, 10 aa, Artificial Sequence
  Murine-derived CDR sequences
SASLSVGYMH

SEQ ID NO: 189 - protein, 10 aa, Artificial Sequence
  Murine-derived CDR sequences
SAQSRVGYMH

SEQ ID NO: 190 - protein, 10 aa, Artificial Sequence
  Murine-derived CDR sequences
SAQLRVGYMH

SEQ ID NO: 191 - protein, 10 aa, Artificial Sequence
  Murine-derived CDR sequences
SAQSSVGYMH

SEQ ID NO: 192 - protein, 10 aa, Artificial Sequence
  Murine-derived CDR sequences
LPSLSVGYMH

SEQ ID NO: 193 - protein, 10 aa, Artificial Sequence
  Murine-derived CDR sequences
LPSSSVGYMH

SEQ ID NO: 194 - protein, 10 aa, Artificial Sequence
  Murine-derived CDR sequences
LPSLRVGYMH

SEQ ID NO: 195 - protein, 10 aa, Artificial Sequence
  Murine-derived CDR sequences
LCSSRVGYMH

SEQ ID NO: 196 - protein, 10 aa, Artificial Sequence
  Murine-derived CDR sequences
LCSLSVGYMH

SEQ ID NO: 197 - protein, 10 aa, Artificial Sequence
  Murine-derived CDR sequences
LCSSSVGYMH

SEQ ID NO: 198 - protein, 10 aa, Artificial Sequence
  Murine-derived CDR sequences
LCSLRVGYMH

SEQ ID NO: 199 - protein, 10 aa, Artificial Sequence
  Murine-derived CDR sequences
LPQSRVGYMH

SEQ ID NO: 200 - protein, 10 aa, Artificial Sequence
  Murine-derived CDR sequences
LPQLSVGYMH

SEQ ID NO: 201 - protein, 10 aa, Artificial Sequence
  Murine-derived CDR sequences
LPQSSVGYMH

SEQ ID NO: 202 - protein, 10 aa, Artificial Sequence
  Murine-derived CDR sequences
LPQLRVGYMH

SEQ ID NO: 203 - protein, 10 aa, Artificial Sequence
  Murine-derived CDR sequences
LCQSRVGYMH

SEQ ID NO: 204 - protein, 10 aa, Artificial Sequence
  Murine-derived CDR sequences
LCQLSVGYMH

SEQ ID NO: 205 - protein, 10 aa, Artificial Sequence
  Murine-derived CDR sequences
LCQSSVGYMH

SEQ ID NO: 206 - protein, 10 aa, Artificial Sequence
  Murine-derived CDR sequences
LCQLRVGYMH

SEQ ID NO: 207 - protein, 10 aa, Artificial Sequence
  Murine-derived CDR sequences
SAQLSVGYMH

SEQ ID NO: 208 - protein, 450 aa, Artificial Sequence
  Murine-derived CDR sequences in a human framework- VH Chain
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 213 aa, Artificial Sequence
  Murine-derived CDR sequences in a human framework- VL Chain
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWDDKKDYNPSLKSRLTISKDTSKNQVVLKVTNMDPADTATYYCARSMITNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 213 aa, Artificial Sequence
  Murine-derived CDR sequences in a human framework- VL Chain
DIQMTQSPSTLSASVGDRVTITCSASSSVGYMHWYQQKPGKAPKLLIYDTFKLASGVPSRFSGSGSGTEFTLTISSLQPDDFATYYCFQFSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 212 - protein, 450 aa, Artificial Sequence
  Murine-derived CDR sequences in a human framework- VH Chain
QVTLRESGPALVKPTQTLTLTCTFSGFSLSTPGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 213 aa, Artificial Sequence
  Murine-derived CDR sequences in a human framework- VL Chain
DIQMTQSPSTLSASVGDRVTITCSLSSRVGYMHWYQQKPGKAPKLLIYDTFYLSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 450 aa, Artificial Sequence
  Murine-derived CDR sequences in a human framework- VH Chain
QVTLRESGPALVKPTQTLTLTCTFSGFSLSTPGMSVGWIRQPPGKALEWLADIWWDGKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 213 aa, Artificial Sequence
  Murine-derived CDR sequences in a human framework- VL Chain
DIQMTQSPSTLSASVGDRVTITCSLSSRVGYMHWYQQKPGKAPKLLIYDTRGLPSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 450 aa, Artificial Sequence
  Murine-derived CDR sequences in a human framework- VH Chain
QVTLRESGPALVKPTQTLTLTCTFSGFSLSTPGMSVGWIRQPPGKALEWLADIWWDGKKHYNPSLKDRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 213 aa, Artificial Sequence
  Murine-derived CDR sequences in a human framework- VL Chain
DIQMTQSPSTLSASVGDRVTITCSPSSRVGYMHWYQQKPGKAPKLLIYDTMRLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWDGKKHYNPSLKDRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 213 aa, Artificial Sequence
  Murine-derived CDR sequences in a human framework- VL Chain
DIQMTQSPSTLSASVGDRVTITCSLSSRVGYMHWYQQKPGKAPKLLIYDTFKLSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWDGKKDYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 213 aa, Artificial Sequence
  Murine-derived CDR sequences in a human framework- VL Chain
DIQMTQSPSTLSASVGDRVTITCSASSRVGYMHWYQQKPGKAPKLLIYDTFKLSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWDGKKSYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 213 aa, Artificial Sequence
  Murine-derived CDR sequences in a human framework- VL Chain
DIQMTQSPSTLSASVGDRVTITCSLSSRVGYMHWYQQKPGKAPKLLIYDTMYQSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 224 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWDDKKSYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - protein, 213 aa, Artificial Sequence
  Murine-derived CDR sequences in a human framework- VL Chain
DIQMTQSPSTLSASVGDRVTITCLPSSRVGYMHWYQQKPGKAPKLLIYDTMYQSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 213 aa, Artificial Sequence
  Murine-derived CDR sequences in a human framework- VL Chain
DIQMTQSPSTLSASVGDRVTITCSASSRVGYMHWYQQKPGKAPKLLIYDTFFLDSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWDDKKSYNPSLKDRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 213 aa, Artificial Sequence
  Murine-derived CDR sequences in a human framework- VL Chain
DIQMTQSPSTLSASVGDRVTITCSPSSRVGYMHWYQQKPGKAPKLLIYDTRYQSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 450 aa, Artificial Sequence
  Murine-derived CDR sequences in a human framework- VH Chain
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 213 aa, Artificial Sequence
  Murine-derived CDR sequences in a human framework- VL Chain
DIQMTQSPSTLSASVGDRVTITCSASSSVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWDDKKDYNPSLKSRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 213 aa, Artificial Sequence
  Murine-derived CDR sequences in a human framework- VL Chain
DIQMTQSPSTLSASVGDRVTITCSASSSVGYMHWYQQKPGKAPKLLIYDTFKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 234 - protein, 450 aa, Artificial Sequence; identical to SEQ ID NO: 232
  Murine-derived CDR sequences in a human framework- VH Chain

SEQ ID NO: 235 - protein, 213 aa, Artificial Sequence
  Murine-derived CDR sequences in a human framework- VL Chain
DIQMTQSPSTLSASVGDRVTITCSASSSVGYMHWYQQKPGKAPKLLIYDTYKQTSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 450 aa, Artificial Sequence; identical to SEQ ID NO: 232
  Murine-derived CDR sequences in a human framework- VH Chain

SEQ ID NO: 237 - protein, 213 aa, Artificial Sequence
  Murine-derived CDR sequences in a human framework- VL Chain
DIQMTQSPSTLSASVGDRVTITCSASSSVGYMHWYQQKPGKAPKLLIYDTRYLSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWDDKKDYNPSLKSRLTISKDTSKNQVVLKVTNMDPADTATYYCARDMITNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 213 aa, Artificial Sequence; identical to SEQ ID NO: 233
  Murine-derived CDR sequences in a human framework- VL Chain

SEQ ID NO: 240 - protein, 450 aa, Artificial Sequence; identical to SEQ ID NO: 210
  Murine-derived CDR sequences in a human framework- VH Chain

SEQ ID NO: 241 - protein, 213 aa, Artificial Sequence
  Murine-derived CDR sequences in a human framework- VH Chain
DIQMTQSPSTLSASVGDRVTITCSASSSVGYMHWYQQKPGKAPKLLIYDTFKLASGVPSRFSGSGSGTEFTLTISSLQPDDFATYYCFQGSFYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 450 aa, Artificial Sequence; identical to SEQ ID NO: 238
  Murine-derived CDR sequences in a human framework- VH Chain

SEQ ID NO: 243 - protein, 213 aa, Artificial Sequence
  Murine-derived CDR sequences in a human framework- VL Chain
DIQMTQSPSTLSASVGDRVTITCSASSSVGYMHWYQQKPGKAPKLLIYDTFKLTSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 450 aa, Artificial Sequence; identical to SEQ ID NO: 238
  Murine-derived CDR sequences in a human framework- VH Chain

SEQ ID NO: 245 - protein, 213 aa, Artificial Sequence
  Murine-derived CDR sequences in a human framework- VL Chain
DIQMTQSPSTLSASVGDRVTITCSASSRVGYMHWYQQKPGKAPKLLIYDTFKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 450 aa, Artificial Sequence; identical to SEQ ID NO: 238
  Murine-derived CDR sequences in a human framework- VH Chain

SEQ ID NO: 247 - protein, 213 aa, Artificial Sequence
  Murine-derived CDR sequences in a human framework- VL Chain
DIQMTQSPSTLSASVGDRVTITCSASSSVGYMHWYQQKPGKAPKLLIYDTFRLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 213 aa, Artificial Sequence
  Murine-derived CDR sequences in a human framework- VL Chain
DIQMTQSPSTLSASVGDRVTITCSPSSRVGYMHWYQQKPGKAPKLLIYDTYRHSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 450 aa, Artificial Sequence; identical to SEQ ID NO: 218
  Murine-derived CDR sequences in a human framework- VH Chain

SEQ ID NO: 251 - protein, 213 aa, Artificial Sequence
  Murine-derived CDR sequences in a human framework- VL Chain
DIQMTQSPSTLSASVGDRVTITCSLSSSVGYMHWYQQKPGKAPKLLIYDTFFHRSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 450 aa, Artificial Sequence; identical to SEQ ID NO: 226
  Murine-derived CDR sequences in a human framework- VH Chain

SEQ ID NO: 253 - protein, 213 aa, Artificial Sequence
  Murine-derived CDR sequences in a human framework- VL Chain
DIQMTQSPSTLSASVGDRVTITCSASSRVGYMHWYQQKPGKAPKLLIYDTLLLDSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 254 - protein, 450 aa, Artificial Sequence; identical to SEQ ID NO: 226
  Murine-derived CDR sequences in a human framework- VH Chain

SEQ ID NO: 255 - protein, 213 aa, Artificial Sequence
  Murine-derived CDR sequences in a human framework- VL Chain
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 256 - protein, 450 aa, Artificial Sequence; identical to SEQ ID NO: 226
  Murine-derived CDR sequences in a human framework- VH Chain

SEQ ID NO: 257 - protein, 213 aa, Artificial Sequence
  Murine-derived CDR sequences in a human framework- VL Chain
DIQMTQSPSTLSASVGDRVTITCSASSRVGYMHWYQQKPGKAPKLLIYDTSFLDSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - DNA, 26 bp, Artificial
  Description of Artificial Sequence Primer
agtgtcttaaccagcaaagtgttaga

SEQ ID NO: 259 - DNA, 26 bp, Artificial Sequence
  Description of Artificial Sequence Primer
tcattgacttgagatattgatgcatc

SEQ ID NO: 260 - protein, 15 aa, Artificial Sequence
  Linker for constructing humanized antibodies
GGGGSGGGGSGGGGS

SEQ ID NO: 261 - protein, 15 aa, Artificial Sequence
  Linker for constructing humanized antibodies
ESGRSGGGGSGGGGS

SEQ ID NO: 262 - protein, 14 aa, Artificial Sequence
  Linker for constructing humanized antibodies
EGKSSGSGSESKST

SEQ ID NO: 263 - protein, 15 aa, Artificial Sequence
  Linker for constructing humanized antibodies
EGKSSGSGSESKSTQ

SEQ ID NO: 264 - protein, 14 aa, Artificial Sequence
  Linker for constructing humanized antibodies
EGKSSGSGSESKVD

SEQ ID NO: 265 - protein, 14 aa, Artificial Sequence
  Linker for constructing humanized antibodies
GSTSGSGKSSEGKG

SEQ ID NO: 266 - protein, 18 aa, Artificial Sequence
  Linker for constructing humanized antibodies
KESGSVSSEQLAQFRSLD

SEQ ID NO: 267 - protein, 16 aa, Artificial Sequence
  Linker for constructing humanized antibodies
ESGSVSSEELAFRSLD

SEQ ID NO: 268 - protein, 4 aa, Homo sapiens
  intrabody
KDEL

SEQ ID NO: 269 - protein, 4 aa, Homo sapiens
  intrabody
DDEL

SEQ ID NO: 270 - protein, 4 aa, Homo sapiens
  intrabody
DEEL

SEQ ID NO: 271 - protein, 4 aa, Homo sapiens
  intrabody
QEDL

SEQ ID NO: 272 - protein, 4 aa, Homo sapiens
  intrabody
RDEL

SEQ ID NO: 273 - protein, 7 aa, Homo sapiens
  intrabody
PKKKRKV

SEQ ID NO: 274 - protein, 7 aa, Homo sapiens
  intrabody
PQKKIKS

SEQ ID NO: 275 - protein, 5 aa, Homo sapiens
  intrabody
QPKKP

SEQ ID NO: 276 - protein, 4 aa, Homo sapiens
  intrabody
RKKR

SEQ ID NO: 277 - protein, 5 aa, Homo sapiens
  intrabody
KKKRK

SEQ ID NO: 278 - protein, 12 aa, Homo sapiens
  intrabody
RKKRRQRRRAHQ

SEQ ID NO: 279 - protein, 16 aa, Homo sapiens
  intrabody
RQARRNRRRRWRERQR

SEQ ID NO: 280 - protein, 19 aa, Homo sapiens
  intrabody
MPLTRRRPAASQALAPPTP

SEQ ID NO: 281 - protein, 15 aa, Homo sapiens
  intrabody
MDDQRDLISNNEQLP

SEQ ID NO: 282 - protein, 32 aa, Homo sapiens
  intrabody
MLFNLRXXLNNAAFRHGHNFMVRNFRCGQPLX

SEQ ID NO: 283 - protein, 3 aa, Homo sapiens
  intrabody
AKL

SEQ ID NO: 284 - protein, 6 aa, Homo sapiens
  intrabody
SDYQRL

SEQ ID NO: 285 - protein, 8 aa, Homo sapiens
  intrabody
GCVCSSNP

SEQ ID NO: 286 - protein, 8 aa, Homo sapiens
  intrabody
GQTVTTPL

SEQ ID NO: 287 - protein, 8 aa, Homo sapiens
  intrabody
GQELSQHE

SEQ ID NO: 288 - protein, 8 aa, Homo sapiens
  intrabody
GNSPSYNP

SEQ ID NO: 289 - protein, 8 aa, Homo sapiens
  intrabody
GVSGSKGQ

SEQ ID NO: 290 - protein, 8 aa, Homo sapiens
  intrabody
GQTITTPL

SEQ ID NO: 291 - protein, 8 aa, Homo sapiens
  intrabody
GQTLTTPL

SEQ ID NO: 292 - protein, 8 aa, Homo sapiens
  intrabody
GQIFSRSA

SEQ ID NO: 293 - protein, 8 aa, Homo sapiens
  intrabody
GQIHGLSP

SEQ ID NO: 294 - protein, 8 aa, Homo sapiens
  intrabody
GARASVLS

SEQ ID NO: 295 - protein, 8 aa, Homo sapiens
  intrabody
GCTLSAEE

SEQ ID NO: 296 - protein, 16 aa, Homo sapiens
  intrabody
AAVALLPAVLLALLAP

SEQ ID NO: 297 - protein, 12 aa, Homo sapiens
  intrabody
AAVLLPVLLAAP

SEQ ID NO: 298 - protein, 15 aa, Homo sapiens
  intrabody
VTVLALGALAGVGVG

SEQ ID NO: 299 - DNA, 30 bp, Artificial Sequence
  Description of Artificial Sequence Primer
ccagcagtaccacttccttgccctgcgccg

SEQ ID NO: 300 - DNA, 30 bp, Artificial Sequence
  Description of Artificial Sequence Primer
gccgcgtcccgttccttcaccatgacgacc

SEQ ID NO: 301 - DNA, 31 bp, Artificial Sequence
  Description of Artificial Sequence Primer
ccagcagtaccgcttccttgccctgcggccg

SEQ ID NO: 302 - DNA, 30 bp, Artificial Sequence; identical to SEQ ID NO: 300
  Description of Artificial Sequence Primer

SEQ ID NO: 303 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWGDKGHYNPSLKDRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 304 - protein, 120 aa, Artificial Sequence
  Murine-derived CDR sequences in a human framework- VH Domain
QVTLRESGPALVKPTQTLTLTCTFSGFSLSTAGMSVGWIRQPPGKALEWLADIWWGDKGHYNPSLKDRLTISKDTSKNQVVLKVTNMDPADTATYYCARDMIFNWYFDVWGQGTTVTVSS

SEQ ID NO: 305 - protein, 16 aa, Artificial Sequence
  Murine-derived CDR sequences
DIWWGDKGHYNPSLKD

SEQ ID NO: 306 - protein, 213 aa, Artificial Sequence
  Murine-derived CDR sequences in a human framework- VL Chain
DIQMTQSPSTLSASVGDRVTITCSASSSVGYMHWYQQKPGKAPKLLIYDTFYLHSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 307 - protein, 106 aa, Artificial Sequence
  Murine-derived CDR sequences in a human framework- VL Domain
DIQMTQSPSTLSASVGDRVTITCSASSSVGYMHWYQQKPGKAPKLLIYDTFYLHSGVPSRFSGSGSGTEFTLTISSLQPDDFATYYCFQGSGYPFTFGGGTKVEIK

SEQ ID NO: 308 - protein, 7 aa, Artificial Sequence
  Murine-derived CDR sequences
DTFYLHS

SEQ ID NO: 309 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWDDKKSYNPSLKDRLTISKDTSKNQVVLKVTNMDPADTATYYCARDMITNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 120 aa, Artificial Sequence
  Murine-derived CDR sequences in a human framework- VH Domain
QVTLRESGPALVKPTQTLTLTCTFSGFSLSTAGMSVGWIRQPPGKALEWLADIWWDDKKSYNPSLKDRLTISKDTSKNQVVLKVTNMDPADTATYYCARDMITNWYFDVWGQGTTVTVSS

SEQ ID NO: 311 - protein, 10 aa, Artificial Sequence
  Murine-derived CDR sequences
DMITNWYFDV

SEQ ID NO: 312 - protein, 213 aa, Artificial Sequence
  Murine-derived CDR sequences in a human framework- VL Chain
DIQMTQSPSTLSASVGDRVTITCLLSSRVGYMHWYQQKPGKAPKLLIYDTYYQTSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 313 - protein, 106 aa, Artificial Sequence
  Murine-derived CDR sequences in a human framework- VL Domain
DIQMTQSPSTLSASVGDRVTITCLLSSRVGYMHWYQQKPGKAPKLLIYDTYYQTSGVPSRFSGSGSGTEFTLTISSLQPDDFATYYCFQGSGYPFTFGGGTKVEIK

SEQ ID NO: 314 - protein, 10 aa, Artificial Sequence
  Murine-derived CDR sequences
LLSSRVGYMH

SEQ ID NO: 315 - protein, 7 aa, Artificial Sequence
  Murine-derived CDR sequences
DTYYQTS

SEQ ID NO: 316 - protein, 450 aa, Artificial Sequence; identical to SEQ ID NO: 248
  Murine-derived CDR sequences in a human framework- VH Chain

SEQ ID NO: 317 - protein, 120 aa, Artificial Sequence; identical to SEQ ID NO: 67
  Murine-derived CDR sequences in a human framework- VH Domain

SEQ ID NO: 318 - protein, 213 aa, Artificial Sequence
  Murine-derived CDR sequences in a human framework- VL Chain
DIQMTQSPSTLSASVGDRVTITCLLSSRVGYMHWYQQKPGKAPKLLIYDTMYQASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 319 - protein, 106 aa, Artificial Sequence
  Murine-derived CDR sequences in a human framework- VL Domain
DIQMTQSPSTLSASVGDRVTITCLLSSRVGYMHWYQQKPGKAPKLLIYDTMYQASGVPSRFSGSGSGTEFTLTISSLQPDDFATYYCFQGSGYPFTFGGGTKVEIK

SEQ ID NO: 320 - protein, 10 aa, Artificial Sequence
  Murine-derived CDR sequences
LLSSRVGYMH

SEQ ID NO: 321 - protein, 7 aa, Artificial Sequence
  Murine-derived CDR sequences
DTMYQAS

SEQ ID NO: 322 - protein, 450 aa, Artificial Sequence; identical to SEQ ID NO: 248
  Murine-derived CDR sequences in a human framework- VH Chain

SEQ ID NO: 323 - protein, 120 aa, Artificial Sequence; identical to SEQ ID NO: 67
  Murine-derived CDR sequences in a human framework- VH Domain

SEQ ID NO: 324 - protein, 213 aa, Artificial Sequence
  Murine-derived CDR sequences in a human framework- VL Chain
DIQMTQSPSTLSASVGDRVTITCSLSSRVGYMHWYQQKPGKAPKLLIYDTYYLPSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 325 - protein, 106 aa, Artificial Sequence
  Murine-derived CDR sequences in a human framework- VL Domain
DIQMTQSPSTLSASVGDRVTITCSLSSRVGYMHWYQQKPGKAPKLLIYDTYYLPSGVPSRFSGSGSGTEFTLTISSLQPDDFATYYCFQGSGYPFTFGGGTKVEIK

SEQ ID NO: 326 - protein, 7 aa, Artificial Sequence
  Murine-derived CDR sequences
DTYYLPS

SEQ ID NO: 327 - protein, 450 aa, Artificial Sequence
  Murine-derived CDR sequences in a human framework- VH Chain
QVTLRESGPALVKPTQTLTLTCTFSGFSLSTAGMSVGWIRQPPGKALEWLADIWWDDKKDYNPSLKDRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 120 aa, Artificial Sequence
  Murine-derived CDR sequences in a human framework- VH Domain
QVTLRESGPALVKPTQTLTLTCTFSGFSLSTAGMSVGWIRQPPGKALEWLADIWWDDKKDYNPSLKDRLTISKDTSKNQVVLKVTNMDPADTATYYCARDMIFNWYFDVWGQGTTVTVSS

SEQ ID NO: 329 - protein, 16 aa, Artificial Sequence
  Murine-derived CDR sequences
DIWWDDKKDYNPSLKD

SEQ ID NO: 330 - protein, 213 aa, Artificial Sequence
  Murine-derived CDR sequences in a human framework- VL Chain
DIQMTQSPSTLSASVGDRVTITCSLSSRVGYMHWYQQKPGKAPKLLIYDTFRHTSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 331 - protein, 106 aa, Artificial Sequence
  Murine-derived CDR sequences in a human framework- VL Domain
DIQMTQSPSTLSASVGDRVTITCSLSSRVGYMHWYQQKPGKAPKLLIYDTFRHTSGVPSRFSGSGSGTEFTLTISSLQPDDFATYYCFQGSGYPFTFGGGTKVEIK

SEQ ID NO: 332 - protein, 7 aa, Artificial Sequence
  Murine-derived CDR sequences
DTFRHTS

SEQ ID NO: 333 - protein, 213 aa, Artificial Sequence
  Murine-derived CDR sequences in a human framework- VL Chain
DIQMTQSPSTLSASVGDRVTITCSPSSSVGYMHWYQQKPGKAPKLLIYDTYYLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 106 aa, Artificial Sequence
  Murine-derived CDR sequences in a human framework- VL Domain
DIQMTQSPSTLSASVGDRVTITCSPSSSVGYMHWYQQKPGKAPKLLIYDTYYLASGVPSRFSGSGSGTEFTLTISSLQPDDFATYYCFQGSGYPFTFGGGTKVEIK

SEQ ID NO: 335 - protein, 10 aa, Artificial Sequence
  Murine-derived CDR sequences
SPSSSVGYMH

SEQ ID NO: 336 - protein, 7 aa, Artificial Sequence
  Murine-derived CDR sequences
DTYYLAS

SEQ ID NO: 337 - protein, 365 aa, Homo sapiens
  Human FcRn
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 338 - protein, 365 aa, Murine
MGMPLPWALSLLLVLLPQTWGSETRPPLMYHLTAVSNPSTGLPSFWATGWLGPQQYLTYNSLRQEADPCGAWVWENQVSWYWEKETTDLKSKEQLFLEALKTLEKILNGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSSVPVVGIVLGLLLVVVAIAGGVLLWGRMRSGLPAPWLSLSGDDSGDLLPGGNLPPEAEPQGANAFPATS

SEQ ID NO: 339 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 340 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 341 - protein, 15 aa, Homo sapiens
EPKSCDKTHTCPPCP

SEQ ID NO: 342 - protein, 232 aa, Homo sapiens
  Human hinge Fc region
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 343 - protein, 120 aa, Artificial Sequence
  Murine-derived CDR sequences in a human framework- VH Domain
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGQGTTVTVSS

SEQ ID NO: 344 - protein, 7 aa, Artificial Sequence
  Murine-derived CDR sequences
VLHQDWL

SEQ ID NO: 345 - protein, 6 aa, Artificial Sequence
  Murine-derived CDR sequences
LMISRT

SEQ ID NO: 346 - protein, 9 aa, Artificial Sequence
  Murine-derived CDR sequences
MHEALHNHY

SEQ ID NO: 347 - protein, 5 aa, Artificial Sequence
  Murine-derived CDR sequences
GQPEN

SEQ ID NO: 348 - protein, 6 aa, Artificial Sequence
  Human-derived CDR sequence
LYITRE

SEQ ID NO: 349 - protein, 6 aa, Artificial Sequence
  Human-derived CDR sequence
LYISRT

SEQ ID NO: 350 - protein, 6 aa, Artificial Sequence
  Human-derived CDR sequence
LYISRS

SEQ ID NO: 351 - protein, 6 aa, Artificial Sequence
  Human-derived CDR sequence
LYISRR

SEQ ID NO: 352 - protein, 6 aa, Artificial Sequence
  Human-derived CDR sequence
LYISRQ

SEQ ID NO: 353 - protein, 6 aa, Artificial Sequence
  Human-derived CDR sequence
LWISRT

SEQ ID NO: 354 - protein, 6 aa, Artificial Sequence
  Human-derived CDR sequence
LYISLQ

SEQ ID NO: 355 - protein, 6 aa, Artificial Sequence
  Human-derived CDR sequence
LFISRD

SEQ ID NO: 356 - protein, 6 aa, Artificial Sequence
  Human-derived CDR sequence
LFISRT

SEQ ID NO: 357 - protein, 6 aa, Artificial Sequence
  Human-derived CDR sequence
LFISRR

SEQ ID NO: 358 - protein, 6 aa, Artificial Sequence
  Human-derived CDR sequence
LFITGA

SEQ ID NO: 359 - protein, 6 aa, Artificial Sequence
  Human-derived CDR sequence
LSISRE

SEQ ID NO: 360 - protein, 6 aa, Artificial Sequence
  Human-derived CDR sequence
RTISIS

SEQ ID NO: 361 - protein, 7 aa, Artificial Sequence
  Human-derived CDR sequence
TPHSDWL

SEQ ID NO: 362 - protein, 7 aa, Artificial Sequence
  Human-derived CDR sequence
IPHEDWL

SEQ ID NO: 363 - protein, 5 aa, Artificial Sequence
  Human-derived CDR sequence
RTREP

SEQ ID NO: 364 - protein, 5 aa, Artificial Sequence
  Human-derived CDR sequence
DPPES

SEQ ID NO: 365 - protein, 5 aa, Artificial Sequence
  Human-derived CDR sequence
SDPEP

SEQ ID NO: 366 - protein, 5 aa, Artificial Sequence
  Human-derived CDR sequence
TSHEN

SEQ ID NO: 367 - protein, 5 aa, Artificial Sequence
  Human-derived CDR sequence
SKSEN

SEQ ID NO: 368 - protein, 5 aa, Artificial Sequence
  Human-derived CDR sequence
HRSEN

SEQ ID NO: 369 - protein, 5 aa, Artificial Sequence
  Human-derived CDR sequence
KIREN

SEQ ID NO: 370 - protein, 5 aa, Artificial Sequence
  Human-derived CDR sequence
GITES

SEQ ID NO: 371 - protein, 5 aa, Artificial Sequence
  Human-derived CDR sequence
SMAEP

SEQ ID NO: 372 - protein, 9 aa, Artificial Sequence
  Human-derived CDR sequence
MHEALRYHH

SEQ ID NO: 373 - protein, 9 aa, Artificial Sequence
  Human-derived CDR sequence
MHEALHFHH

SEQ ID NO: 374 - protein, 9 aa, Artificial Sequence
  Human-derived CDR sequence
MHEALKFHH

SEQ ID NO: 375 - protein, 9 aa, Artificial Sequence
  Human-derived CDR sequence
MHEALSYHR

SEQ ID NO: 376 - protein, 9 aa, Artificial Sequence
  Human-derived CDR sequence
THEALHYHT

SEQ ID NO: 377 - protein, 9 aa, Artificial Sequence
  Human-derived CDR sequence
MHEALHYHY

SEQ ID NO: 378 - DNA, 54 bp, Artificial Sequence
  Human Degenerate oligos used to combine with TAAssDNA template to make Library 1
catgtgacctcaggsnnsnnsnngatsnnsnnggtgtccttgggttttgggggg

SEQ ID NO: 379 - DNA, 53 bp, Artificial Sequence
  Human Degenerate oligos used to combine with TAAssDNA template to make Library 2
gcacttgtactccttgccattsnnccasnnsnngtgsnnsnnggtgaggacgc

SEQ ID NO: 380 - DNA, 38 bp, Artificial Sequence
  Human Degenerate oligos used to combine with TAAssDNA template to make Library 3
ggtcttgtagttsnnctcsnnsnnsnnattgctctccc

SEQ ID NO: 381 - DNA, 53 bp, Artificial Sequence
  Human Degenerate oligos used to combine with TAAssDNA template to make Library 4
ggctcttctgcgtsnngtgsnnsnncagagcctcatgsnncacggagcatgag

SEQ ID NO: 382 - DNA, 45 bp, Artificial Sequence
  Human site-directed mutagenesis primer
gcatgtgacctcaggttcccgagtgatatagagggtgtccttggg

SEQ ID NO: 383 - DNA, 45 bp, Artificial Sequence
  Human site-directed mutagenesis primer
cccaaggacaccctctatatcactcgggaacctgaggtcacatgc

SEQ ID NO: 384 - protein, 16 aa, Artificial Sequence
  Murine-derived CDR sequences
DIWWDDKGDYNPSLKS

SEQ ID NO: 385 - protein, 16 aa, Artificial Sequence
  Murine-derived CDR sequences
DIWWDDKGDYNPSLKD

SEQ ID NO: 386 - protein, 16 aa, Artificial Sequence
  Murine-derived CDR sequences
DIWWDDKGHYNPSLKS

SEQ ID NO: 387 - protein, 16 aa, Artificial Sequence
  Murine-derived CDR sequences
DIWWDDKGHYNPSLKD

SEQ ID NO: 388 - protein, 16 aa, Artificial Sequence
  Murine-derived CDR sequences
DIWWDDKGSYNPSLKS

SEQ ID NO: 389 - protein, 16 aa, Artificial Sequence
  Murine-derived CDR sequences
DIWWDDKGSYNPSLKD

SEQ ID NO: 390 - protein, 16 aa, Artificial Sequence
  Murine-derived CDR sequences
DIWWDGKGDYNPSLKS

SEQ ID NO: 391 - protein, 16 aa, Artificial Sequence
  Murine-derived CDR sequences
DIWWDGKGDYNPSLKD

SEQ ID NO: 392 - protein, 16 aa, Artificial Sequence
  Murine-derived CDR sequences
DIWWDGKGHYNPSLKS

SEQ ID NO: 393 - protein, 16 aa, Artificial Sequence
  Murine-derived CDR sequences
DIWWDGKGHYNPSLKD

SEQ ID NO: 394 - protein, 16 aa, Artificial Sequence
  Murine-derived CDR sequences
DIWWDGKGSYNPSLKS

SEQ ID NO: 395 - protein, 16 aa, Artificial Sequence
  Murine-derived CDR sequences
DIWWDGKGSYNPSLKD

SEQ ID NO: 396 - protein, 10 aa, Artificial Sequence
  Murine-derived CDR sequences
KCQLFVGYMH

SEQ ID NO: 397 - protein, 10 aa, Artificial Sequence
  Murine-derived CDR sequences
KCQSFVGYMH

SEQ ID NO: 398 - protein, 10 aa, Artificial Sequence
  Murine-derived CDR sequences
KCQVSVGYMH

SEQ ID NO: 399 - protein, 10 aa, Artificial Sequence
  Murine-derived CDR sequences
KCQVRVGYMH

SEQ ID NO: 400 - protein, 10 aa, Artificial Sequence
  Murine-derived CDR sequences
KCQVFVGYMH

SEQ ID NO: 401 - protein, 10 aa, Artificial Sequence
  Murine-derived CDR sequences
KCSLFVGYMH

SEQ ID NO: 402 - protein, 10 aa, Artificial Sequence
  Murine-derived CDR sequences
KCSSFVGYMH

SEQ ID NO: 403 - protein, 10 aa, Artificial Sequence
  Murine-derived CDR sequences
KCSVSVGYMH

SEQ ID NO: 404 - protein, 10 aa, Artificial Sequence
  Murine-derived CDR sequences
KCSVRVGYMH

SEQ ID NO: 405 - protein, 10 aa, Artificial Sequence
  Murine-derived CDR sequences
KCSVFVGYMH

SEQ ID NO: 406 - protein, 10 aa, Artificial Sequence
  Murine-derived CDR sequences
KAQLFVGYMH

SEQ ID NO: 407 - protein, 10 aa, Artificial Sequence
  Murine-derived CDR sequences
KAQSFVGYMH

SEQ ID NO: 408 - protein, 10 aa, Artificial Sequence
  Murine-derived CDR sequences
KAQVSVGYMH

SEQ ID NO: 409 - protein, 10 aa, Artificial Sequence
  Murine-derived CDR sequences
KAQVRVGYMH

SEQ ID NO: 410 - protein, 10 aa, Artificial Sequence
  Murine-derived CDR sequences
KAQVFVGYMH

SEQ ID NO: 411 - protein, 10 aa, Artificial Sequence
  Murine-derived CDR sequences
KASLFVGYMH

SEQ ID NO: 412 - protein, 10 aa, Artificial Sequence
  Murine-derived CDR sequences
KASSFVGYMH

SEQ ID NO: 413 - protein, 10 aa, Artificial Sequence
  Murine-derived CDR sequences
KASVSVGYMH

SEQ ID NO: 414 - protein, 10 aa, Artificial Sequence
  Murine-derived CDR sequences
KASVRVGYMH

SEQ ID NO: 415 - protein, 10 aa, Artificial Sequence
  Murine-derived CDR sequences
KASVFVGYMH

SEQ ID NO: 416 - protein, 10 aa, Artificial Sequence
  Murine-derived CDR sequences
KLQLFVGYMH

SEQ ID NO: 417 - protein, 10 aa, Artificial Sequence
  Murine-derived CDR sequences
KLQSFVGYMH

SEQ ID NO: 418 - protein, 10 aa, Artificial Sequence
  Murine-derived CDR sequences
KLQVSVGYMH

SEQ ID NO: 419 - protein, 10 aa, Artificial Sequence
  Murine-derived CDR sequences
KLQVRVGYMH

SEQ ID NO: 420 - protein, 10 aa, Artificial Sequence
  Murine-derived CDR sequences
KLQVFVGYMH

SEQ ID NO: 421 - protein, 10 aa, Artificial Sequence
  Murine-derived CDR sequences
KLSLFVGYMH

SEQ ID NO: 422 - protein, 10 aa, Artificial Sequence
  Murine-derived CDR sequences
KLSSFVGYMH

SEQ ID NO: 423 - protein, 10 aa, Artificial Sequence
  Murine-derived CDR sequences
KLSVSVGYMH

SEQ ID NO: 424 - protein, 10 aa, Artificial Sequence
  Murine-derived CDR sequences
KLSVRVGYMH

SEQ ID NO: 425 - protein, 10 aa, Artificial Sequence
  Murine-derived CDR sequences
KLSVFVGYMH

SEQ ID NO: 426 - protein, 10 aa, Artificial Sequence
  Murine-derived CDR sequences
KPQLFVGYMH

SEQ ID NO: 427 - protein, 10 aa, Artificial Sequence
  Murine-derived CDR sequences
KPQSFVGYMH

SEQ ID NO: 428 - protein, 10 aa, Artificial Sequence
  Murine-derived CDR sequences
KPQVSVGYMH

SEQ ID NO: 429 - protein, 10 aa, Artificial Sequence
  Murine-derived CDR sequences
KPQVRVGYMH

SEQ ID NO: 430 - protein, 10 aa, Artificial Sequence
  Murine-derived CDR sequences
KPQVFVGYMH

SEQ ID NO: 431 - protein, 10 aa, Artificial Sequence
  Murine-derived CDR sequences
KPSLFVGYMH

SEQ ID NO: 432 - protein, 10 aa, Artificial Sequence
  Murine-derived CDR sequences
KPSSFVGYMH

SEQ ID NO: 433 - protein, 10 aa, Artificial Sequence
  Murine-derived CDR sequences
KPSVSVGYMH

SEQ ID NO: 434 - protein, 10 aa, Artificial Sequence
  Murine-derived CDR sequences
KPSVRVGYMH

SEQ ID NO: 435 - protein, 10 aa, Artificial Sequence
  Murine-derived CDR sequences
KPSVFVGYMH

SEQ ID NO: 436 - protein, 10 aa, Artificial Sequence
  Murine-derived CDR sequences
SCQLFVGYMH

SEQ ID NO: 437 - protein, 10 aa, Artificial Sequence
  Murine-derived CDR sequences
SCQSFVGYMH

SEQ ID NO: 438 - protein, 10 aa, Artificial Sequence
  Murine-derived CDR sequences
SCQVSVGYMH

SEQ ID NO: 439 - protein, 10 aa, Artificial Sequence
  Murine-derived CDR sequences
SCQVRVGYMH

SEQ ID NO: 440 - protein, 10 aa, Artificial Sequence
  Murine-derived CDR sequences
SCQVFVGYMH

SEQ ID NO: 441 - protein, 10 aa, Artificial Sequence
  Murine-derived CDR sequences
SCSLFVGYMH

SEQ ID NO: 442 - protein, 10 aa, Artificial Sequence
  Murine-derived CDR sequences
SCSSFVGYMH

SEQ ID NO: 443 - protein, 10 aa, Artificial Sequence
  Murine-derived CDR sequences
SCSVSVGYMH

SEQ ID NO: 444 - protein, 10 aa, Artificial Sequence
  Murine-derived CDR sequences
SCSVRVGYMH

SEQ ID NO: 445 - protein, 10 aa, Artificial Sequence
  Murine-derived CDR sequences
SCSVFVGYMH

SEQ ID NO: 446 - protein, 10 aa, Artificial Sequence
  Murine-derived CDR sequences
SAQLFVGYMH

SEQ ID NO: 447 - protein, 10 aa, Artificial Sequence
  Murine-derived CDR sequences
SAQSFVGYMH

SEQ ID NO: 448 - protein, 10 aa, Artificial Sequence
  Murine-derived CDR sequences
SAQVSVGYMH

SEQ ID NO: 449 - protein, 10 aa, Artificial Sequence
  Murine-derived CDR sequences
SAQVRVGYMH

SEQ ID NO: 450 - protein, 10 aa, Artificial Sequence
  Murine-derived CDR sequences
SAQVFVGYMH

SEQ ID NO: 451 - protein, 10 aa, Artificial Sequence
  Murine-derived CDR sequences
SASLFVGYMH

SEQ ID NO: 452 - protein, 10 aa, Artificial Sequence
  Murine-derived CDR sequences
SASSFVGYMH

SEQ ID NO: 453 - protein, 10 aa, Artificial Sequence
  Murine-derived CDR sequences
SASVSVGYMH

SEQ ID NO: 454 - protein, 10 aa, Artificial Sequence
  Murine-derived CDR sequences
SASVRVGYMH

SEQ ID NO: 455 - protein, 10 aa, Artificial Sequence
  Murine-derived CDR sequences
SASVFVGYMH

SEQ ID NO: 456 - protein, 10 aa, Artificial Sequence
  Murine-derived CDR sequences
SLQLFVGYMH

SEQ ID NO: 457 - protein, 10 aa, Artificial Sequence
  Murine-derived CDR sequences
SLQSFVGYMH

SEQ ID NO: 458 - protein, 10 aa, Artificial Sequence
  Murine-derived CDR sequences
SLQVSVGYMH

SEQ ID NO: 459 - protein, 10 aa, Artificial Sequence
  Murine-derived CDR sequences
SLQVRVGYMH

SEQ ID NO: 460 - protein, 10 aa, Artificial Sequence
  Murine-derived CDR sequences
SLQVFVGYMH

SEQ ID NO: 461 - protein, 10 aa, Artificial Sequence
  Murine-derived CDR sequences
SLSLFVGYMH

SEQ ID NO: 462 - protein, 10 aa, Artificial Sequence
  Murine-derived CDR sequences
SLSSFVGYMH

SEQ ID NO: 463 - protein, 10 aa, Artificial Sequence
  Murine-derived CDR sequences
SLSVSVGYMH

SEQ ID NO: 464 - protein, 10 aa, Artificial Sequence
  Murine-derived CDR sequences
SLSVRVGYMH

SEQ ID NO: 465 - protein, 10 aa, Artificial Sequence
  Murine-derived CDR sequences
SLSVFVGYMH

SEQ ID NO: 466 - protein, 10 aa, Artificial Sequence
  Murine-derived CDR sequences
SPQLFVGYMH

SEQ ID NO: 467 - protein, 10 aa, Artificial Sequence
  Murine-derived CDR sequences
SPQSFVGYMH

SEQ ID NO: 468 - protein, 10 aa, Artificial Sequence
  Murine-derived CDR sequences
SPQVSVGYMH

SEQ ID NO: 469 - protein, 10 aa, Artificial Sequence
  Murine-derived CDR sequences
SPQVRVGYMH

SEQ ID NO: 470 - protein, 10 aa, Artificial Sequence
  Murine-derived CDR sequences
SPQVFVGYMH

SEQ ID NO: 471 - protein, 10 aa, Artificial Sequence
  Murine-derived CDR sequences
SPSLFVGYMH

SEQ ID NO: 472 - protein, 10 aa, Artificial Sequence
  Murine-derived CDR sequences
SPSSFVGYMH

SEQ ID NO: 473 - protein, 10 aa, Artificial Sequence
  Murine-derived CDR sequences
SPSVSVGYMH

SEQ ID NO: 474 - protein, 10 aa, Artificial Sequence
  Murine-derived CDR sequences
SPSVRVGYMH

SEQ ID NO: 475 - protein, 10 aa, Artificial Sequence
  Murine-derived CDR sequences
SPSVFVGYMH

SEQ ID NO: 476 - protein, 10 aa, Artificial Sequence
  Murine-derived CDR sequences
LCQLFVGYMH

SEQ ID NO: 477 - protein, 10 aa, Artificial Sequence
  Murine-derived CDR sequences
LCQSFVGYMH

SEQ ID NO: 478 - protein, 10 aa, Artificial Sequence
  Murine-derived CDR sequences
LCQVSVGYMH

SEQ ID NO: 479 - protein, 10 aa, Artificial Sequence
  Murine-derived CDR sequences
LCQVRVGYMH

SEQ ID NO: 480 - protein, 10 aa, Artificial Sequence
  Murine-derived CDR sequences
LCQVFVGYMH

SEQ ID NO: 481 - protein, 10 aa, Artificial Sequence
  Murine-derived CDR sequences
LCSLFVGYMH

SEQ ID NO: 482 - protein, 10 aa, Artificial Sequence
  Murine-derived CDR sequences
LCSSFVGYMH

SEQ ID NO: 483 - protein, 10 aa, Artificial Sequence
  Murine-derived CDR sequences
LCSVSVGYMH

SEQ ID NO: 484 - protein, 10 aa, Artificial Sequence
  Murine-derived CDR sequences
LCSVRVGYMH

SEQ ID NO: 485 - protein, 10 aa, Artificial Sequence
  Murine-derived CDR sequences
LCSVFVGYMH

SEQ ID NO: 486 - protein, 10 aa, Artificial Sequence
  Murine-derived CDR sequences
LAQLSVGYMH

SEQ ID NO: 487 - protein, 10 aa, Artificial Sequence
  Murine-derived CDR sequences
LAQLRVGYMH

SEQ ID NO: 488 - protein, 10 aa, Artificial Sequence
  Murine-derived CDR sequences
LAQLFVGYMH

SEQ ID NO: 489 - protein, 10 aa, Artificial Sequence
  Murine-derived CDR sequences
LAQSSVGYMH

SEQ ID NO: 490 - protein, 10 aa, Artificial Sequence
  Murine-derived CDR sequences
LAQSRVGYMH

SEQ ID NO: 491 - protein, 10 aa, Artificial Sequence
  Murine-derived CDR sequences
LAQSFVGYMH

SEQ ID NO: 492 - protein, 10 aa, Artificial Sequence
  Murine-derived CDR sequences
LAQVSVGYMH

SEQ ID NO: 493 - protein, 10 aa, Artificial Sequence
  Murine-derived CDR sequences
LAQVRVGYMH

SEQ ID NO: 494 - protein, 10 aa, Artificial Sequence
  Murine-derived CDR sequences
LAQVFVGYMH

SEQ ID NO: 495 - protein, 10 aa, Artificial Sequence
  Murine-derived CDR sequences
LASLSVGYMH

SEQ ID NO: 496 - protein, 10 aa, Artificial Sequence
  Murine-derived CDR sequences
LASLRVGYMH

SEQ ID NO: 497 - protein, 10 aa, Artificial Sequence
  Murine-derived CDR sequences
LASLFVGYMH

SEQ ID NO: 498 - protein, 10 aa, Artificial Sequence
  Murine-derived CDR sequences
LASSSVGYMH

SEQ ID NO: 499 - protein, 10 aa, Artificial Sequence
  Murine-derived CDR sequences
LASSRVGYMH

SEQ ID NO: 500 - protein, 10 aa, Artificial Sequence
  Murine-derived CDR sequences
LASSFVGYMH

SEQ ID NO: 501 - protein, 10 aa, Artificial Sequence
  Murine-derived CDR sequences
LASVSVGYMH

SEQ ID NO: 502 - protein, 10 aa, Artificial Sequence
  Murine-derived CDR sequences
LASVRVGYMH

SEQ ID NO: 503 - protein, 10 aa, Artificial Sequence
  Murine-derived CDR sequences
LASVFVGYMH

SEQ ID NO: 504 - protein, 10 aa, Artificial Sequence
  Murine-derived CDR sequences
LLQLSVGYMH

SEQ ID NO: 505 - protein, 10 aa, Artificial Sequence
  Murine-derived CDR sequences
LLQLRVGYMH

SEQ ID NO: 506 - protein, 10 aa, Artificial Sequence
  Murine-derived CDR sequences
LLQLFVGYMH

SEQ ID NO: 507 - protein, 10 aa, Artificial Sequence
  Murine-derived CDR sequences
LLQSSVGYMH

SEQ ID NO: 508 - protein, 10 aa, Artificial Sequence
  Murine-derived CDR sequences
LLQSRVGYMH

SEQ ID NO: 509 - protein, 10 aa, Artificial Sequence
  Murine-derived CDR sequences
LLQSFVGYMH

SEQ ID NO: 510 - protein, 10 aa, Artificial Sequence
  Murine-derived CDR sequences
LLQVSVGYMH

SEQ ID NO: 511 - protein, 10 aa, Artificial Sequence
  Murine-derived CDR sequences
LLQVRVGYMH

SEQ ID NO: 512 - protein, 10 aa, Artificial Sequence
  Murine-derived CDR sequences
LLQVFVGYMH

SEQ ID NO: 513 - protein, 10 aa, Artificial Sequence
  Murine-derived CDR sequences
LLSLSVGYMH

SEQ ID NO: 514 - protein, 10 aa, Artificial Sequence
  Murine-derived CDR sequences
LLSLRVGYMH

SEQ ID NO: 515 - protein, 10 aa, Artificial Sequence
  Murine-derived CDR sequences
LLSLFVGYMH

SEQ ID NO: 516 - protein, 10 aa, Artificial Sequence
  Murine-derived CDR sequences
LLSSSVGYMH

SEQ ID NO: 517 - protein, 10 aa, Artificial Sequence
  Murine-derived CDR sequences
LLSSRVGYMH

SEQ ID NO: 518 - protein, 10 aa, Artificial Sequence
  Murine-derived CDR sequences
LLSSFVGYMH

SEQ ID NO: 519 - protein, 10 aa, Artificial Sequence
  Murine-derived CDR sequences
LLSVSVGYMH

SEQ ID NO: 520 - protein, 10 aa, Artificial Sequence
  Murine-derived CDR sequences
LLSVRVGYMH

SEQ ID NO: 521 - protein, 10 aa, Artificial Sequence
  Murine-derived CDR sequences
LLSVFVGYMH

SEQ ID NO: 522 - protein, 10 aa, Artificial Sequence
  Murine-derived CDR sequences
LPQLFVGYMH

SEQ ID NO: 523 - protein, 10 aa, Artificial Sequence
  Murine-derived CDR sequences
LPQSFVGYMH

SEQ ID NO: 524 - protein, 10 aa, Artificial Sequence
  Murine-derived CDR sequences
LPQVSVGYMH

SEQ ID NO: 525 - protein, 10 aa, Artificial Sequence
  Murine-derived CDR sequences
LPQVRVGYMH

SEQ ID NO: 526 - protein, 10 aa, Artificial Sequence
  Murine-derived CDR sequences
LPQVFVGYMH

SEQ ID NO: 527 - protein, 10 aa, Artificial Sequence
  Murine-derived CDR sequences
LPSLFVGYMH

SEQ ID NO: 528 - protein, 10 aa, Artificial Sequence
  Murine-derived CDR sequences
LPSSFVGYMH

SEQ ID NO: 529 - protein, 10 aa, Artificial Sequence
  Murine-derived CDR sequences
LPSVSVGYMH

SEQ ID NO: 530 - protein, 10 aa, Artificial Sequence
  Murine-derived CDR sequences
LPSVRVGYMH

SEQ ID NO: 531 - protein, 10 aa, Artificial Sequence
  Murine-derived CDR sequences
LPSVFVGYMH

SEQ ID NO: 532 - protein, 7 aa, Artificial Sequence
  Murine-derived CDR sequences
DTSKLKS

SEQ ID NO: 533 - protein, 7 aa, Artificial Sequence
  Murine-derived CDR sequences
DTSKLRS

SEQ ID NO: 534 - protein, 7 aa, Artificial Sequence
  Murine-derived CDR sequences
DTSKLHS

SEQ ID NO: 535 - protein, 7 aa, Artificial Sequence
  Murine-derived CDR sequences
DTSKLTS

SEQ ID NO: 536 - protein, 7 aa, Artificial Sequence
  Murine-derived CDR sequences
DTSKHAS

SEQ ID NO: 537 - protein, 7 aa, Artificial Sequence
  Murine-derived CDR sequences
DTSKHSS

SEQ ID NO: 538 - protein, 7 aa, Artificial Sequence
  Murine-derived CDR sequences
DTSKHKS

SEQ ID NO: 539 - protein, 7 aa, Artificial Sequence
  Murine-derived CDR sequences
DTSKHRS

SEQ ID NO: 540 - protein, 7 aa, Artificial Sequence
  Murine-derived CDR sequences
DTSKHHS

SEQ ID NO: 541 - protein, 7 aa, Artificial Sequence
  Murine-derived CDR sequences
DTSKHPS

SEQ ID NO: 542 - protein, 7 aa, Artificial Sequence
  Murine-derived CDR sequences
DTSKHTS

SEQ ID NO: 543 - protein, 7 aa, Artificial Sequence
  Murine-derived CDR sequences
DTSKHDS

SEQ ID NO: 544 - protein, 7 aa, Artificial Sequence
  Murine-derived CDR sequences
DTSKQKS

SEQ ID NO: 545 - protein, 7 aa, Artificial Sequence
  Murine-derived CDR sequences
DTSKQRS

SEQ ID NO: 546 - protein, 7 aa, Artificial Sequence
  Murine-derived CDR sequences
DTSKQHS

SEQ ID NO: 547 - protein, 7 aa, Artificial Sequence
  Murine-derived CDR sequences
DTSKQPS

SEQ ID NO: 548 - protein, 7 aa, Artificial Sequence
  Murine-derived CDR sequences
DTSKQTS

SEQ ID NO: 549 - protein, 7 aa, Artificial Sequence
  Murine-derived CDR sequences
DTSKQDS

SEQ ID NO: 550 - protein, 7 aa, Artificial Sequence
  Murine-derived CDR sequences
DTSGLSS

SEQ ID NO: 551 - protein, 7 aa, Artificial Sequence
  Murine-derived CDR sequences
DTSGLKS

SEQ ID NO: 552 - protein, 7 aa, Artificial Sequence
  Murine-derived CDR sequences
DTSGLRS

SEQ ID NO: 553 - protein, 7 aa, Artificial Sequence
  Murine-derived CDR sequences
DTSGLHS

SEQ ID NO: 554 - protein, 7 aa, Artificial Sequence
  Murine-derived CDR sequences
DTSGLTS

SEQ ID NO: 555 - protein, 7 aa, Artificial Sequence
  Murine-derived CDR sequences
DTSGLDS

SEQ ID NO: 556 - protein, 7 aa, Artificial Sequence
  Murine-derived CDR sequences
DTSGHAS

SEQ ID NO: 557 - protein, 7 aa, Artificial Sequence
  Murine-derived CDR sequences
DTSGHSS

SEQ ID NO: 558 - protein, 7 aa, Artificial Sequence
  Murine-derived CDR sequences
DTSGHKS

SEQ ID NO: 559 - protein, 7 aa, Artificial Sequence
  Murine-derived CDR sequences
DTSGHRS

SEQ ID NO: 560 - protein, 7 aa, Artificial Sequence
  Murine-derived CDR sequences
DTSGHHS

SEQ ID NO: 561 - protein, 7 aa, Artificial Sequence
  Murine-derived CDR sequences
DTSGHPS

SEQ ID NO: 562 - protein, 7 aa, Artificial Sequence
  Murine-derived CDR sequences
DTSGHTS

SEQ ID NO: 563 - protein, 7 aa, Artificial Sequence
  Murine-derived CDR sequences
DTSGHDS

SEQ ID NO: 564 - protein, 7 aa, Artificial Sequence
  Murine-derived CDR sequences
DTSGQAS

SEQ ID NO: 565 - protein, 7 aa, Artificial Sequence
  Murine-derived CDR sequences
DTSGQSS

SEQ ID NO: 566 - protein, 7 aa, Artificial Sequence
  Murine-derived CDR sequences
DTSGQKS

SEQ ID NO: 567 - protein, 7 aa, Artificial Sequence
  Murine-derived CDR sequences
DTSGQRS

SEQ ID NO: 568 - protein, 7 aa, Artificial Sequence
  Murine-derived CDR sequences
DTSGQHS

SEQ ID NO: 569 - protein, 7 aa, Artificial Sequence
  Murine-derived CDR sequences
DTSGQPS

SEQ ID NO: 570 - protein, 7 aa, Artificial Sequence
  Murine-derived CDR sequences
DTSGQTS

SEQ ID NO: 571 - protein, 7 aa, Artificial Sequence
  Murine-derived CDR sequences
DTSGQDS

SEQ ID NO: 572 - protein, 7 aa, Artificial Sequence
  Murine-derived CDR sequences
DTSRLSS

SEQ ID NO: 573 - protein, 7 aa, Artificial Sequence
  Murine-derived CDR sequences
DTSRLKS

SEQ ID NO: 574 - protein, 7 aa, Artificial Sequence
  Murine-derived CDR sequences
DTSRLRS

SEQ ID NO: 575 - protein, 7 aa, Artificial Sequence
  Murine-derived CDR sequences
DTSRLHS

SEQ ID NO: 576 - protein, 7 aa, Artificial Sequence
  Murine-derived CDR sequences
DTSRLPS

SEQ ID NO: 577 - protein, 7 aa, Artificial Sequence
  Murine-derived CDR sequences
DTSRLTS

SEQ ID NO: 578 - protein, 7 aa, Artificial Sequence
  Murine-derived CDR sequences
DTSRLDS

SEQ ID NO: 579 - protein, 7 aa, Artificial Sequence
  Murine-derived CDR sequences
DTSRHAS

SEQ ID NO: 580 - protein, 7 aa, Artificial Sequence
  Murine-derived CDR sequences
DTSRHSS

SEQ ID NO: 581 - protein, 7 aa, Artificial Sequence
  Murine-derived CDR sequences
DTSRHKS

SEQ ID NO: 582 - protein, 7 aa, Artificial Sequence
  Murine-derived CDR sequences
DTSRHRS

SEQ ID NO: 583 - protein, 7 aa, Artificial Sequence
  Murine-derived CDR sequences
DTSRHHS

SEQ ID NO: 584 - protein, 7 aa, Artificial Sequence
  Murine-derived CDR sequences
DTSRHPS

SEQ ID NO: 585 - protein, 7 aa, Artificial Sequence
  Murine-derived CDR sequences
DTSRHTS

SEQ ID NO: 586 - protein, 7 aa, Artificial Sequence
  Murine-derived CDR sequences
DTSRHDS

SEQ ID NO: 587 - protein, 7 aa, Artificial Sequence
  Murine-derived CDR sequences
DTSRQAS

SEQ ID NO: 588 - protein, 7 aa, Artificial Sequence
  Murine-derived CDR sequences
DTSRQSS

SEQ ID NO: 589 - protein, 7 aa, Artificial Sequence
  Murine-derived CDR sequences
DTSRQKS

SEQ ID NO: 590 - protein, 7 aa, Artificial Sequence
  Murine-derived CDR sequences
DTSRQRS

SEQ ID NO: 591 - protein, 7 aa, Artificial Sequence
  Murine-derived CDR sequences
DTSRQHS

SEQ ID NO: 592 - protein, 7 aa, Artificial Sequence
  Murine-derived CDR sequences
DTSRQPS

SEQ ID NO: 593 - protein, 7 aa, Artificial Sequence
  Murine-derived CDR sequences
DTSRQTS

SEQ ID NO: 594 - protein, 7 aa, Artificial Sequence
  Murine-derived CDR sequences
DTSRQDS

SEQ ID NO: 595 - protein, 7 aa, Artificial Sequence
  Murine-derived CDR sequences
DTSYLKS

SEQ ID NO: 596 - protein, 7 aa, Artificial Sequence
  Murine-derived CDR sequences
DTSYLRS

SEQ ID NO: 597 - protein, 7 aa, Artificial Sequence
  Murine-derived CDR sequences
DTSYLHS

SEQ ID NO: 598 - protein, 7 aa, Artificial Sequence
  Murine-derived CDR sequences
DTSYLPS

SEQ ID NO: 599 - protein, 7 aa, Artificial Sequence
  Murine-derived CDR sequences
DTSYLTS

SEQ ID NO: 600 - protein, 7 aa, Artificial Sequence
  Murine-derived CDR sequences
DTSYLDS

SEQ ID NO: 601 - protein, 7 aa, Artificial Sequence
  Murine-derived CDR sequences
DTSYHAS

SEQ ID NO: 602 - protein, 7 aa, Artificial Sequence
  Murine-derived CDR sequences
DTSYHSS

SEQ ID NO: 603 - protein, 7 aa, Artificial Sequence
  Murine-derived CDR sequences
DTSYHKS

SEQ ID NO: 604 - protein, 7 aa, Artificial Sequence
  Murine-derived CDR sequences
DTSYHRS

SEQ ID NO: 605 - protein, 7 aa, Artificial Sequence
  Murine-derived CDR sequences
DTSYHHS

SEQ ID NO: 606 - protein, 7 aa, Artificial Sequence
  Murine-derived CDR sequences
DTSYHPS

SEQ ID NO: 607 - protein, 7 aa, Artificial Sequence
  Murine-derived CDR sequences
DTSYHTS

SEQ ID NO: 608 - protein, 7 aa, Artificial Sequence
  Murine-derived CDR sequences
DTSYHDS

SEQ ID NO: 609 - protein, 7 aa, Artificial Sequence
  Murine-derived CDR sequences
DTSYQKS

SEQ ID NO: 610 - protein, 7 aa, Artificial Sequence
  Murine-derived CDR sequences
DTSYQRS

SEQ ID NO: 611 - protein, 7 aa, Artificial Sequence
  Murine-derived CDR sequences
DTSYQHS

SEQ ID NO: 612 - protein, 7 aa, Artificial Sequence
  Murine-derived CDR sequences
DTSYQPS

SEQ ID NO: 613 - protein, 7 aa, Artificial Sequence
  Murine-derived CDR sequences
DTSYQTS

SEQ ID NO: 614 - protein, 7 aa, Artificial Sequence
  Murine-derived CDR sequences
DTSYQDS

SEQ ID NO: 615 - protein, 7 aa, Artificial Sequence
  Murine-derived CDR sequences
DTSFLAS

SEQ ID NO: 616 - protein, 7 aa, Artificial Sequence
  Murine-derived CDR sequences
DTSFLSS

SEQ ID NO: 617 - protein, 7 aa, Artificial Sequence
  Murine-derived CDR sequences
DTSFLKS

SEQ ID NO: 618 - protein, 7 aa, Artificial Sequence
  Murine-derived CDR sequences
DTSFLRS

SEQ ID NO: 619 - protein, 7 aa, Artificial Sequence
  Murine-derived CDR sequences
DTSFLHS

SEQ ID NO: 620 - protein, 7 aa, Artificial Sequence
  Murine-derived CDR sequences
DTSFLPS

SEQ ID NO: 621 - protein, 7 aa, Artificial Sequence
  Murine-derived CDR sequences
DTSFLTS

SEQ ID NO: 622 - protein, 7 aa, Artificial Sequence
  Murine-derived CDR sequences
DTSFHAS

SEQ ID NO: 623 - protein, 7 aa, Artificial Sequence
  Murine-derived CDR sequences
DTSFHSS

SEQ ID NO: 624 - protein, 7 aa, Artificial Sequence
  Murine-derived CDR sequences
DTSFHKS

SEQ ID NO: 625 - protein, 7 aa, Artificial Sequence
  Murine-derived CDR sequences
DTSFHRS

SEQ ID NO: 626 - protein, 7 aa, Artificial Sequence
  Murine-derived CDR sequences
DTSFHHS

SEQ ID NO: 627 - protein, 7 aa, Artificial Sequence
  Murine-derived CDR sequences
DTSFHPS

SEQ ID NO: 628 - protein, 7 aa, Artificial Sequence
  Murine-derived CDR sequences
DTSFHTS

SEQ ID NO: 629 - protein, 7 aa, Artificial Sequence
  Murine-derived CDR sequences
DTSFHDS

SEQ ID NO: 630 - protein, 7 aa, Artificial Sequence
  Murine-derived CDR sequences
DTSFQAS

SEQ ID NO: 631 - protein, 7 aa, Artificial Sequence
  Murine-derived CDR sequences
DTSFQSS

SEQ ID NO: 632 - protein, 7 aa, Artificial Sequence
  Murine-derived CDR sequences
DTSFQKS

SEQ ID NO: 633 - protein, 7 aa, Artificial Sequence
  Murine-derived CDR sequences
DTSFQRS

SEQ ID NO: 634 - protein, 7 aa, Artificial Sequence
  Murine-derived CDR sequences
DTSFQHS

SEQ ID NO: 635 - protein, 7 aa, Artificial Sequence
  Murine-derived CDR sequences
DTSFQPS

SEQ ID NO: 636 - protein, 7 aa, Artificial Sequence
  Murine-derived CDR sequences
DTSFQTS

SEQ ID NO: 637 - protein, 7 aa, Artificial Sequence
  Murine-derived CDR sequences
DTSFQDS

SEQ ID NO: 638 - protein, 7 aa, Artificial Sequence
  Murine-derived CDR sequences
DTSLLSS

SEQ ID NO: 639 - protein, 7 aa, Artificial Sequence
  Murine-derived CDR sequences
DTSLLKS

SEQ ID NO: 640 - protein, 7 aa, Artificial Sequence
  Murine-derived CDR sequences
DTSLLRS

SEQ ID NO: 641 - protein, 7 aa, Artificial Sequence
  Murine-derived CDR sequences
DTSLLHS

SEQ ID NO: 642 - protein, 7 aa, Artificial Sequence
  Murine-derived CDR sequences
DTSLLPS

SEQ ID NO: 643 - protein, 7 aa, Artificial Sequence
  Murine-derived CDR sequences
DTSLLTS

SEQ ID NO: 644 - protein, 7 aa, Artificial Sequence
  Murine-derived CDR sequences
DTSLHAS

SEQ ID NO: 645 - protein, 7 aa, Artificial Sequence
  Murine-derived CDR sequences
DTSLHSS

SEQ ID NO: 646 - protein, 7 aa, Artificial Sequence
  Murine-derived CDR sequences
DTSLHKS

SEQ ID NO: 647 - protein, 7 aa, Artificial Sequence
  Murine-derived CDR sequences
DTSLHRS

SEQ ID NO: 648 - protein, 7 aa, Artificial Sequence
  Murine-derived CDR sequences
DTSLHHS

SEQ ID NO: 649 - protein, 7 aa, Artificial Sequence
  Murine-derived CDR sequences
DTSLHPS

SEQ ID NO: 650 - protein, 7 aa, Artificial Sequence
  Murine-derived CDR sequences
DTSLHTS

SEQ ID NO: 651 - protein, 7 aa, Artificial Sequence
  Murine-derived CDR sequences
DTSLHDS

SEQ ID NO: 652 - protein, 7 aa, Artificial Sequence
  Murine-derived CDR sequences
DTSLQAS

SEQ ID NO: 653 - protein, 7 aa, Artificial Sequence
  Murine-derived CDR sequences
DTSLQSS

SEQ ID NO: 654 - protein, 7 aa, Artificial Sequence
  Murine-derived CDR sequences
DTSLQKS

SEQ ID NO: 655 - protein, 7 aa, Artificial Sequence
  Murine-derived CDR sequences
DTSLQRS

SEQ ID NO: 656 - protein, 7 aa, Artificial Sequence
  Murine-derived CDR sequences
DTSLQHS

SEQ ID NO: 657 - protein, 7 aa, Artificial Sequence
  Murine-derived CDR sequences
DTSLQPS

SEQ ID NO: 658 - protein, 7 aa, Artificial Sequence
  Murine-derived CDR sequences
DTSLQTS

SEQ ID NO: 659 - protein, 7 aa, Artificial Sequence
  Murine-derived CDR sequences
DTSLQDS

SEQ ID NO: 660 - protein, 7 aa, Artificial Sequence
  Murine-derived CDR sequences
DTFKLKS

SEQ ID NO: 661 - protein, 7 aa, Artificial Sequence
  Murine-derived CDR sequences
DTFKLRS

SEQ ID NO: 662 - protein, 7 aa, Artificial Sequence
  Murine-derived CDR sequences
DTFKLHS

SEQ ID NO: 663 - protein, 7 aa, Artificial Sequence
  Murine-derived CDR sequences
DTFKLPS

SEQ ID NO: 664 - protein, 7 aa, Artificial Sequence
  Murine-derived CDR sequences
DTFKLTS

SEQ ID NO: 665 - protein, 7 aa, Artificial Sequence
  Murine-derived CDR sequences
DTFKLDS

SEQ ID NO: 666 - protein, 7 aa, Artificial Sequence
  Murine-derived CDR sequences
DTFKHAS

SEQ ID NO: 667 - protein, 7 aa, Artificial Sequence
  Murine-derived CDR sequences
DTFKHSS

SEQ ID NO: 668 - protein, 7 aa, Artificial Sequence
  Murine-derived CDR sequences
DTFKHKS

SEQ ID NO: 669 - protein, 7 aa, Artificial Sequence
  Murine-derived CDR sequences
DTFKHRS

SEQ ID NO: 670 - protein, 7 aa, Artificial Sequence
  Murine-derived CDR sequences
DTFKHHS

SEQ ID NO: 671 - protein, 7 aa, Artificial Sequence
  Murine-derived CDR sequences
DTFKHPS

SEQ ID NO: 672 - protein, 7 aa, Artificial Sequence
  Murine-derived CDR sequences
DTFKHTS

SEQ ID NO: 673 - protein, 7 aa, Artificial Sequence
  Murine-derived CDR sequences
DTFKHDS

SEQ ID NO: 674 - protein, 7 aa, Artificial Sequence
  Murine-derived CDR sequences
DTFKQAS

SEQ ID NO: 675 - protein, 7 aa, Artificial Sequence
  Murine-derived CDR sequences
DTFKQSS

SEQ ID NO: 676 - protein, 7 aa, Artificial Sequence
  Murine-derived CDR sequences
DTFKQKS

SEQ ID NO: 677 - protein, 7 aa, Artificial Sequence
  Murine-derived CDR sequences
DTFKQRS

SEQ ID NO: 678 - protein, 7 aa, Artificial Sequence
  Murine-derived CDR sequences
DTFKQHS

SEQ ID NO: 679 - protein, 7 aa, Artificial Sequence
  Murine-derived CDR sequences
DTFKQPS

SEQ ID NO: 680 - protein, 7 aa, Artificial Sequence
  Murine-derived CDR sequences
DTFKQTS

SEQ ID NO: 681 - protein, 7 aa, Artificial Sequence
  Murine-derived CDR sequences
DTFKQDS

SEQ ID NO: 682 - protein, 7 aa, Artificial Sequence
  Murine-derived CDR sequences
DTFGLAS

SEQ ID NO: 683 - protein, 7 aa, Artificial Sequence
  Murine-derived CDR sequences
DTFGLSS

SEQ ID NO: 684 - protein, 7 aa, Artificial Sequence
  Murine-derived CDR sequences
DTFGLKS

SEQ ID NO: 685 - protein, 7 aa, Artificial Sequence
  Murine-derived CDR sequences
DTFGLRS

SEQ ID NO: 686 - protein, 7 aa, Artificial Sequence
  Murine-derived CDR sequences
DTFGLHS

SEQ ID NO: 687 - protein, 7 aa, Artificial Sequence
  Murine-derived CDR sequences
DTFGLPS

SEQ ID NO: 688 - protein, 7 aa, Artificial Sequence
  Murine-derived CDR sequences
DTFGLTS

SEQ ID NO: 689 - protein, 7 aa, Artificial Sequence
  Murine-derived CDR sequences
DTFGLDS

SEQ ID NO: 690 - protein, 7 aa, Artificial Sequence
  Murine-derived CDR sequences
DTFGHAS

SEQ ID NO: 691 - protein, 7 aa, Artificial Sequence
  Murine-derived CDR sequences
DTFGHSS

SEQ ID NO: 692 - protein, 7 aa, Artificial Sequence
  Murine-derived CDR sequences
DTFGHKS

SEQ ID NO: 693 - protein, 7 aa, Artificial Sequence
  Murine-derived CDR sequences
DTFGHRS

SEQ ID NO: 694 - protein, 7 aa, Artificial Sequence
  Murine-derived CDR sequences
DTFGHHS

SEQ ID NO: 695 - protein, 7 aa, Artificial Sequence
  Murine-derived CDR sequences
DTFGHPS

SEQ ID NO: 696 - protein, 7 aa, Artificial Sequence
  Murine-derived CDR sequences
DTFGHTS

SEQ ID NO: 697 - protein, 7 aa, Artificial Sequence
  Murine-derived CDR sequences
DTFGHDS

SEQ ID NO: 698 - protein, 7 aa, Artificial Sequence
  Murine-derived CDR sequences
DTFGQAS

SEQ ID NO: 699 - protein, 7 aa, Artificial Sequence
  Murine-derived CDR sequences
DTFGQSS

SEQ ID NO: 700 - protein, 7 aa, Artificial Sequence
  Murine-derived CDR sequences
DTFGQKS

SEQ ID NO: 701 - protein, 7 aa, Artificial Sequence
  Murine-derived CDR sequences
DTFGQRS

SEQ ID NO: 702 - protein, 7 aa, Artificial Sequence
  Murine-derived CDR sequences
DTFGQHS

SEQ ID NO: 703 - protein, 7 aa, Artificial Sequence
  Murine-derived CDR sequences
DTFGQPS

SEQ ID NO: 704 - protein, 7 aa, Artificial Sequence
  Murine-derived CDR sequences
DTFGQTS

SEQ ID NO: 705 - protein, 7 aa, Artificial Sequence
  Murine-derived CDR sequences
DTFGQDS

SEQ ID NO: 706 - protein, 7 aa, Artificial Sequence
  Murine-derived CDR sequences
DTFRLAS

SEQ ID NO: 707 - protein, 7 aa, Artificial Sequence
  Murine-derived CDR sequences
DTFRLSS

SEQ ID NO: 708 - protein, 7 aa, Artificial Sequence
  Murine-derived CDR sequences
DTFRLKS

SEQ ID NO: 709 - protein, 7 aa, Artificial Sequence
  Murine-derived CDR sequences
DTFRLRS

SEQ ID NO: 710 - protein, 7 aa, Artificial Sequence
  Murine-derived CDR sequences
DTFRLHS

SEQ ID NO: 711 - protein, 7 aa, Artificial Sequence
  Murine-derived CDR sequences
DTFRLPS

SEQ ID NO: 712 - protein, 7 aa, Artificial Sequence
  Murine-derived CDR sequences
DTFRLTS

SEQ ID NO: 713 - protein, 7 aa, Artificial Sequence
  Murine-derived CDR sequences
DTFRLDS

SEQ ID NO: 714 - protein, 7 aa, Artificial Sequence
  Murine-derived CDR sequences
DTFRHAS

SEQ ID NO: 715 - protein, 7 aa, Artificial Sequence
  Murine-derived CDR sequences
DTFRHSS

SEQ ID NO: 716 - protein, 7 aa, Artificial Sequence
  Murine-derived CDR sequences
DTFRHKS

SEQ ID NO: 717 - protein, 7 aa, Artificial Sequence
  Murine-derived CDR sequences
DTFRHRS

SEQ ID NO: 718 - protein, 7 aa, Artificial Sequence
  Murine-derived CDR sequences
DTFRHHS

SEQ ID NO: 719 - protein, 7 aa, Artificial Sequence
  Murine-derived CDR sequences
DTFRHPS

SEQ ID NO: 720 - protein, 7 aa, Artificial Sequence
  Murine-derived CDR sequences
DTFRHTS

SEQ ID NO: 721 - protein, 7 aa, Artificial Sequence
  Murine-derived CDR sequences
DTFRHDS

SEQ ID NO: 722 - protein, 7 aa, Artificial Sequence
  Murine-derived CDR sequences
DTFRQAS

SEQ ID NO: 723 - protein, 7 aa, Artificial Sequence
  Murine-derived CDR sequences
DTFRQSS

SEQ ID NO: 724 - protein, 7 aa, Artificial Sequence
  Murine-derived CDR sequences
DTFRQKS

SEQ ID NO: 725 - protein, 7 aa, Artificial Sequence
  Murine-derived CDR sequences
DTFRQRS

SEQ ID NO: 726 - protein, 7 aa, Artificial Sequence
  Murine-derived CDR sequences
DTFRQHS

SEQ ID NO: 727 - protein, 7 aa, Artificial Sequence
  Murine-derived CDR sequences
DTFRQPS

SEQ ID NO: 728 - protein, 7 aa, Artificial Sequence
  Murine-derived CDR sequences
DTFRQTS

SEQ ID NO: 729 - protein, 7 aa, Artificial Sequence
  Murine-derived CDR sequences
DTFRQDS

SEQ ID NO: 730 - protein, 7 aa, Artificial Sequence
  Murine-derived CDR sequences
DTFYLKS

SEQ ID NO: 731 - protein, 7 aa, Artificial Sequence
  Murine-derived CDR sequences
DTFYLRS

SEQ ID NO: 732 - protein, 7 aa, Artificial Sequence
  Murine-derived CDR sequences
DTFYLHS

SEQ ID NO: 733 - protein, 7 aa, Artificial Sequence
  Murine-derived CDR sequences
DTFYLPS

SEQ ID NO: 734 - protein, 7 aa, Artificial Sequence
  Murine-derived CDR sequences
DTFYLTS

SEQ ID NO: 735 - protein, 7 aa, Artificial Sequence
  Murine-derived CDR sequences
DTFYLDS

SEQ ID NO: 736 - protein, 7 aa, Artificial Sequence
  Murine-derived CDR sequences
DTFYHAS

SEQ ID NO: 737 - protein, 7 aa, Artificial Sequence
  Murine-derived CDR sequences
DTFYHSS

SEQ ID NO: 738 - protein, 7 aa, Artificial Sequence
  Murine-derived CDR sequences
DTFYHKS

SEQ ID NO: 739 - protein, 7 aa, Artificial Sequence
  Murine-derived CDR sequences
DTFYHRS

SEQ ID NO: 740 - protein, 7 aa, Artificial Sequence
  Murine-derived CDR sequences
DTFYHHS

SEQ ID NO: 741 - protein, 7 aa, Artificial Sequence
  Murine-derived CDR sequences
DTFYHPS

SEQ ID NO: 742 - protein, 7 aa, Artificial Sequence
  Murine-derived CDR sequences
DTFYHTS

SEQ ID NO: 743 - protein, 7 aa, Artificial Sequence
  Murine-derived CDR sequences
DTFYHDS

SEQ ID NO: 744 - protein, 7 aa, Artificial Sequence
  Murine-derived CDR sequences
DTFYQAS

SEQ ID NO: 745 - protein, 7 aa, Artificial Sequence
  Murine-derived CDR sequences
DTFYQSS

SEQ ID NO: 746 - protein, 7 aa, Artificial Sequence
  Murine-derived CDR sequences
DTFYQKS

SEQ ID NO: 747 - protein, 7 aa, Artificial Sequence
  Murine-derived CDR sequences
DTFYQRS

SEQ ID NO: 748 - protein, 7 aa, Artificial Sequence
  Murine-derived CDR sequences
DTFYQHS

SEQ ID NO: 749 - protein, 7 aa, Artificial Sequence
  Murine-derived CDR sequences
DTFYQPS

SEQ ID NO: 750 - protein, 7 aa, Artificial Sequence
  Murine-derived CDR sequences
DTFYQTS

SEQ ID NO: 751 - protein, 7 aa, Artificial Sequence
  Murine-derived CDR sequences
DTFYQDS

SEQ ID NO: 752 - protein, 7 aa, Artificial Sequence
  Murine-derived CDR sequences
DTFFLAS

SEQ ID NO: 753 - protein, 7 aa, Artificial Sequence
  Murine-derived CDR sequences
DTFFLSS

SEQ ID NO: 754 - protein, 7 aa, Artificial Sequence
  Murine-derived CDR sequences
DTFFLKS

SEQ ID NO: 755 - protein, 7 aa, Artificial Sequence
  Murine-derived CDR sequences
DTFFLRS

SEQ ID NO: 756 - protein, 7 aa, Artificial Sequence
  Murine-derived CDR sequences
DTFFLHS

SEQ ID NO: 757 - protein, 7 aa, Artificial Sequence
  Murine-derived CDR sequences
DTFFLPS

SEQ ID NO: 758 - protein, 7 aa, Artificial Sequence
  Murine-derived CDR sequences
DTFFLTS

SEQ ID NO: 759 - protein, 7 aa, Artificial Sequence
  Murine-derived CDR sequences
DTFFHAS

SEQ ID NO: 760 - protein, 7 aa, Artificial Sequence
  Murine-derived CDR sequences
DTFFHSS

SEQ ID NO: 761 - protein, 7 aa, Artificial Sequence
  Murine-derived CDR sequences
DTFFHKS

SEQ ID NO: 762 - protein, 7 aa, Artificial Sequence
  Murine-derived CDR sequences
DTFFHRS

SEQ ID NO: 763 - protein, 7 aa, Artificial Sequence
  Murine-derived CDR sequences
DTFFHHS

SEQ ID NO: 764 - protein, 7 aa, Artificial Sequence
  Murine-derived CDR sequences
DTFFHPS

SEQ ID NO: 765 - protein, 7 aa, Artificial Sequence
  Murine-derived CDR sequences
DTFFHTS

SEQ ID NO: 766 - protein, 7 aa, Artificial Sequence
  Murine-derived CDR sequences
DTFFHDS

SEQ ID NO: 767 - protein, 7 aa, Artificial Sequence
  Murine-derived CDR sequences
DTFFQAS

SEQ ID NO: 768 - protein, 7 aa, Artificial Sequence
  Murine-derived CDR sequences
DTFFQSS

SEQ ID NO: 769 - protein, 7 aa, Artificial Sequence
  Murine-derived CDR sequences
DTFFQKS

SEQ ID NO: 770 - protein, 7 aa, Artificial Sequence
  Murine-derived CDR sequences
DTFFQRS

SEQ ID NO: 771 - protein, 7 aa, Artificial Sequence
  Murine-derived CDR sequences
DTFFQHS

SEQ ID NO: 772 - protein, 7 aa, Artificial Sequence
  Murine-derived CDR sequences
DTFFQPS

SEQ ID NO: 773 - protein, 7 aa, Artificial Sequence
  Murine-derived CDR sequences
DTFFQTS

SEQ ID NO: 774 - protein, 7 aa, Artificial Sequence
  Murine-derived CDR sequences
DTFFQDS

SEQ ID NO: 775 - protein, 7 aa, Artificial Sequence
  Murine-derived CDR sequences
DTFLLAS

SEQ ID NO: 776 - protein, 7 aa, Artificial Sequence
  Murine-derived CDR sequences
DTFLLSS

SEQ ID NO: 777 - protein, 7 aa, Artificial Sequence
  Murine-derived CDR sequences
DTFLLKS

SEQ ID NO: 778 - protein, 7 aa, Artificial Sequence
  Murine-derived CDR sequences
DTFLLRS

SEQ ID NO: 779 - protein, 7 aa, Artificial Sequence
  Murine-derived CDR sequences
DTFLLHS

SEQ ID NO: 780 - protein, 7 aa, Artificial Sequence
  Murine-derived CDR sequences
DTFLLPS

SEQ ID NO: 781 - protein, 7 aa, Artificial Sequence
  Murine-derived CDR sequences
DTFLLTS

SEQ ID NO: 782 - protein, 7 aa, Artificial Sequence
  Murine-derived CDR sequences
DTFLLDS

SEQ ID NO: 783 - protein, 7 aa, Artificial Sequence
  Murine-derived CDR sequences
DTFLHAS

SEQ ID NO: 784 - protein, 7 aa, Artificial Sequence
  Murine-derived CDR sequences
DTFLHSS

SEQ ID NO: 785 - protein, 7 aa, Artificial Sequence
  Murine-derived CDR sequences
DTFLHKS

SEQ ID NO: 786 - protein, 7 aa, Artificial Sequence
  Murine-derived CDR sequences
DTFLHRS

SEQ ID NO: 787 - protein, 7 aa, Artificial Sequence
  Murine-derived CDR sequences
DTFLHHS

SEQ ID NO: 788 - protein, 7 aa, Artificial Sequence
  Murine-derived CDR sequences
DTFLHPS

SEQ ID NO: 789 - protein, 7 aa, Artificial Sequence
  Murine-derived CDR sequences
DTFLHTS

SEQ ID NO: 790 - protein, 7 aa, Artificial Sequence
  Murine-derived CDR sequences
DTFLHDS

SEQ ID NO: 791 - protein, 7 aa, Artificial Sequence
  Murine-derived CDR sequences
DTFLQAS

SEQ ID NO: 792 - protein, 7 aa, Artificial Sequence
  Murine-derived CDR sequences
DTFLQSS

SEQ ID NO: 793 - protein, 7 aa, Artificial Sequence
  Murine-derived CDR sequences
DTFLQKS

SEQ ID NO: 794 - protein, 7 aa, Artificial Sequence
  Murine-derived CDR sequences
DTFLQRS

SEQ ID NO: 795 - protein, 7 aa, Artificial Sequence
  Murine-derived CDR sequences
DTFLQHS

SEQ ID NO: 796 - protein, 7 aa, Artificial Sequence
  Murine-derived CDR sequences
DTFLQPS

SEQ ID NO: 797 - protein, 7 aa, Artificial Sequence
  Murine-derived CDR sequences
DTFLQTS

SEQ ID NO: 798 - protein, 7 aa, Artificial Sequence
  Murine-derived CDR sequences
DTFLQDS

SEQ ID NO: 799 - protein, 7 aa, Artificial Sequence
  Murine-derived CDR sequences
DTYKLAS

SEQ ID NO: 800 - protein, 7 aa, Artificial Sequence
  Murine-derived CDR sequences
DTYKLSS

SEQ ID NO: 801 - protein, 7 aa, Artificial Sequence
  Murine-derived CDR sequences
DTYKLKS

SEQ ID NO: 802 - protein, 7 aa, Artificial Sequence
  Murine-derived CDR sequences
DTYKLRS

SEQ ID NO: 803 - protein, 7 aa, Artificial Sequence
  Murine-derived CDR sequences
DTYKLHS

SEQ ID NO: 804 - protein, 7 aa, Artificial Sequence
  Murine-derived CDR sequences
DTYKLPS

SEQ ID NO: 805 - protein, 7 aa, Artificial Sequence
  Murine-derived CDR sequences
DTYKLTS

SEQ ID NO: 806 - protein, 7 aa, Artificial Sequence
  Murine-derived CDR sequences
DTYKLDS

SEQ ID NO: 807 - protein, 7 aa, Artificial Sequence
  Murine-derived CDR sequences
DTYKHAS

SEQ ID NO: 808 - protein, 7 aa, Artificial Sequence
  Murine-derived CDR sequences
DTYKHSS

SEQ ID NO: 809 - protein, 7 aa, Artificial Sequence
  Murine-derived CDR sequences
DTYKHKS

SEQ ID NO: 810 - protein, 7 aa, Artificial Sequence
  Murine-derived CDR sequences
DTYKHRS

SEQ ID NO: 811 - protein, 7 aa, Artificial Sequence
  Murine-derived CDR sequences
DTYKHHS

SEQ ID NO: 812 - protein, 7 aa, Artificial Sequence
  Murine-derived CDR sequences
DTYKHPS

SEQ ID NO: 813 - protein, 7 aa, Artificial Sequence
  Murine-derived CDR sequences
DTYKHTS

SEQ ID NO: 814 - protein, 7 aa, Artificial Sequence
  Murine-derived CDR sequences
DTYKHDS

SEQ ID NO: 815 - protein, 7 aa, Artificial Sequence
  Murine-derived CDR sequences
DTYKQAS

SEQ ID NO: 816 - protein, 7 aa, Artificial Sequence
  Murine-derived CDR sequences
DTYKQSS

SEQ ID NO: 817 - protein, 7 aa, Artificial Sequence
  Murine-derived CDR sequences
DTYKQKS

SEQ ID NO: 818 - protein, 7 aa, Artificial Sequence
  Murine-derived CDR sequences
DTYKQRS

SEQ ID NO: 819 - protein, 7 aa, Artificial Sequence
  Murine-derived CDR sequences
DTYKQHS

SEQ ID NO: 820 - protein, 7 aa, Artificial Sequence
  Murine-derived CDR sequences
DTYKQPS

SEQ ID NO: 821 - protein, 7 aa, Artificial Sequence
  Murine-derived CDR sequences
DTYKQTS

SEQ ID NO: 822 - protein, 7 aa, Artificial Sequence
  Murine-derived CDR sequences
DTYKQDS

SEQ ID NO: 823 - protein, 7 aa, Artificial Sequence
  Murine-derived CDR sequences
DTYGLAS

SEQ ID NO: 824 - protein, 7 aa, Artificial Sequence
  Murine-derived CDR sequences
DTYGLSS

SEQ ID NO: 825 - protein, 7 aa, Artificial Sequence
  Murine-derived CDR sequences
DTYGLKS

SEQ ID NO: 826 - protein, 7 aa, Artificial Sequence
  Murine-derived CDR sequences
DTYGLRS

SEQ ID NO: 827 - protein, 7 aa, Artificial Sequence
  Murine-derived CDR sequences
DTYGLHS

SEQ ID NO: 828 - protein, 7 aa, Artificial Sequence
  Murine-derived CDR sequences
DTYGLPS

SEQ ID NO: 829 - protein, 7 aa, Artificial Sequence
  Murine-derived CDR sequences
DTYGLTS

SEQ ID NO: 830 - protein, 7 aa, Artificial Sequence
  Murine-derived CDR sequences
DTYGLDS

SEQ ID NO: 831 - protein, 7 aa, Artificial Sequence
  Murine-derived CDR sequences
DTYGHAS

SEQ ID NO: 832 - protein, 7 aa, Artificial Sequence
  Murine-derived CDR sequences
DTYGHSS

SEQ ID NO: 833 - protein, 7 aa, Artificial Sequence
  Murine-derived CDR sequences
DTYGHKS

SEQ ID NO: 834 - protein, 7 aa, Artificial Sequence
  Murine-derived CDR sequences
DTYGHRS

SEQ ID NO: 835 - protein, 7 aa, Artificial Sequence
  Murine-derived CDR sequences
DTYGHHS

SEQ ID NO: 836 - protein, 7 aa, Artificial Sequence
  Murine-derived CDR sequences
DTYGHPS

SEQ ID NO: 837 - protein, 7 aa, Artificial Sequence
  Murine-derived CDR sequences
DTYGHTS

SEQ ID NO: 838 - protein, 7 aa, Artificial Sequence
  Murine-derived CDR sequences
DTYGHDS

SEQ ID NO: 839 - protein, 7 aa, Artificial Sequence
  Murine-derived CDR sequences
DTYGQAS

SEQ ID NO: 840 - protein, 7 aa, Artificial Sequence
  Murine-derived CDR sequences
DTYGQSS

SEQ ID NO: 841 - protein, 7 aa, Artificial Sequence
  Murine-derived CDR sequences
DTYGQKS

SEQ ID NO: 842 - protein, 7 aa, Artificial Sequence
  Murine-derived CDR sequences
DTYGQRS

SEQ ID NO: 843 - protein, 7 aa, Artificial Sequence
  Murine-derived CDR sequences
DTYGQHS

SEQ ID NO: 844 - protein, 7 aa, Artificial Sequence
  Murine-derived CDR sequences
DTYGQPS

SEQ ID NO: 845 - protein, 7 aa, Artificial Sequence
  Murine-derived CDR sequences
DTYGQTS

SEQ ID NO: 846 - protein, 7 aa, Artificial Sequence
  Murine-derived CDR sequences
DTYGQDS

SEQ ID NO: 847 - protein, 7 aa, Artificial Sequence
  Murine-derived CDR sequences
DTYRLAS

SEQ ID NO: 848 - protein, 7 aa, Artificial Sequence
  Murine-derived CDR sequences
DTYRLSS

SEQ ID NO: 849 - protein, 7 aa, Artificial Sequence
  Murine-derived CDR sequences
DTYRLKS

SEQ ID NO: 850 - protein, 7 aa, Artificial Sequence
  Murine-derived CDR sequences
DTYRLRS

SEQ ID NO: 851 - protein, 7 aa, Artificial Sequence
  Murine-derived CDR sequences
DTYRLHS

SEQ ID NO: 852 - protein, 7 aa, Artificial Sequence
  Murine-derived CDR sequences
DTYRLPS

SEQ ID NO: 853 - protein, 7 aa, Artificial Sequence
  Murine-derived CDR sequences
DTYRLTS

SEQ ID NO: 854 - protein, 7 aa, Artificial Sequence
  Murine-derived CDR sequences
DTYRLDS

SEQ ID NO: 855 - protein, 7 aa, Artificial Sequence
  Murine-derived CDR sequences
DTYRHAS

SEQ ID NO: 856 - protein, 7 aa, Artificial Sequence
  Murine-derived CDR sequences
DTYRHSS

SEQ ID NO: 857 - protein, 7 aa, Artificial Sequence
  Murine-derived CDR sequences
DTYRHKS

SEQ ID NO: 858 - protein, 7 aa, Artificial Sequence
  Murine-derived CDR sequences
DTYRHRS

SEQ ID NO: 859 - protein, 7 aa, Artificial Sequence
  Murine-derived CDR sequences
DTYRHHS

SEQ ID NO: 860 - protein, 7 aa, Artificial Sequence
  Murine-derived CDR sequences
DTYRHPS

SEQ ID NO: 861 - protein, 7 aa, Artificial Sequence
  Murine-derived CDR sequences
DTYRHTS

SEQ ID NO: 862 - protein, 7 aa, Artificial Sequence
  Murine-derived CDR sequences
DTYRHDS

SEQ ID NO: 863 - protein, 7 aa, Artificial Sequence
  Murine-derived CDR sequences
DTYRQAS

SEQ ID NO: 864 - protein, 7 aa, Artificial Sequence
  Murine-derived CDR sequences
DTYRQSS

SEQ ID NO: 865 - protein, 7 aa, Artificial Sequence
  Murine-derived CDR sequences
DTYRQKS

SEQ ID NO: 866 - protein, 7 aa, Artificial Sequence
  Murine-derived CDR sequences
DTYRQRS

SEQ ID NO: 867 - protein, 7 aa, Artificial Sequence
  Murine-derived CDR sequences
DTYRQHS

SEQ ID NO: 868 - protein, 7 aa, Artificial Sequence
  Murine-derived CDR sequences
DTYRQPS

SEQ ID NO: 869 - protein, 7 aa, Artificial Sequence
  Murine-derived CDR sequences
DTYRQTS

SEQ ID NO: 870 - protein, 7 aa, Artificial Sequence
  Murine-derived CDR sequences
DTYRQDS

SEQ ID NO: 871 - protein, 7 aa, Artificial Sequence
  Murine-derived CDR sequences
DTYYLAS

SEQ ID NO: 872 - protein, 7 aa, Artificial Sequence
  Murine-derived CDR sequences
DTYYLSS

SEQ ID NO: 873 - protein, 7 aa, Artificial Sequence
  Murine-derived CDR sequences
DTYYLKS

SEQ ID NO: 874 - protein, 7 aa, Artificial Sequence
  Murine-derived CDR sequences
DTYYLRS

SEQ ID NO: 875 - protein, 7 aa, Artificial Sequence
  Murine-derived CDR sequences
DTYYLHS

SEQ ID NO: 876 - protein, 7 aa, Artificial Sequence
  Murine-derived CDR sequences
DTYYLPS

SEQ ID NO: 877 - protein, 7 aa, Artificial Sequence
  Murine-derived CDR sequences
DTYYLTS

SEQ ID NO: 878 - protein, 7 aa, Artificial Sequence
  Murine-derived CDR sequences
DTYYLDS

SEQ ID NO: 879 - protein, 7 aa, Artificial Sequence
  Murine-derived CDR sequences
DTYYHAS

SEQ ID NO: 880 - protein, 7 aa, Artificial Sequence
  Murine-derived CDR sequences
DTYYHSS

SEQ ID NO: 881 - protein, 7 aa, Artificial Sequence
  Murine-derived CDR sequences
DTYYHKS

SEQ ID NO: 882 - protein, 7 aa, Artificial Sequence
  Murine-derived CDR sequences
DTYYHRS

SEQ ID NO: 883 - protein, 7 aa, Artificial Sequence
  Murine-derived CDR sequences
DTYYHHS

SEQ ID NO: 884 - protein, 7 aa, Artificial Sequence
  Murine-derived CDR sequences
DTYYHPS

SEQ ID NO: 885 - protein, 7 aa, Artificial Sequence
  Murine-derived CDR sequences
DTYYHTS

SEQ ID NO: 886 - protein, 7 aa, Artificial Sequence
  Murine-derived CDR sequences
DTYYHDS

SEQ ID NO: 887 - protein, 7 aa, Artificial Sequence
  Murine-derived CDR sequences
DTYYQAS

SEQ ID NO: 888 - protein, 7 aa, Artificial Sequence
  Murine-derived CDR sequences
DTYYQSS

SEQ ID NO: 889 - protein, 7 aa, Artificial Sequence
  Murine-derived CDR sequences
DTYYQKS

SEQ ID NO: 890 - protein, 7 aa, Artificial Sequence
  Murine-derived CDR sequences
DTYYQRS

SEQ ID NO: 891 - protein, 7 aa, Artificial Sequence
  Murine-derived CDR sequences
DTYYQHS

SEQ ID NO: 892 - protein, 7 aa, Artificial Sequence
  Murine-derived CDR sequences
DTYYQPS

SEQ ID NO: 893 - protein, 7 aa, Artificial Sequence
  Murine-derived CDR sequences
DTYYQTS

SEQ ID NO: 894 - protein, 7 aa, Artificial Sequence
  Murine-derived CDR sequences
DTYYQDS

SEQ ID NO: 895 - protein, 7 aa, Artificial Sequence
  Murine-derived CDR sequences
DTYFLAS

SEQ ID NO: 896 - protein, 7 aa, Artificial Sequence
  Murine-derived CDR sequences
DTYFLSS

SEQ ID NO: 897 - protein, 7 aa, Artificial Sequence
  Murine-derived CDR sequences
DTYFLKS

SEQ ID NO: 898 - protein, 7 aa, Artificial Sequence
  Murine-derived CDR sequences
DTYFLRS

SEQ ID NO: 899 - protein, 7 aa, Artificial Sequence
  Murine-derived CDR sequences
DTYFLHS

SEQ ID NO: 900 - protein, 7 aa, Artificial Sequence
  Murine-derived CDR sequences
DTYFLPS

SEQ ID NO: 901 - protein, 7 aa, Artificial Sequence
  Murine-derived CDR sequences
DTYFLTS

SEQ ID NO: 902 - protein, 7 aa, Artificial Sequence
  Murine-derived CDR sequences
DTYFLDS

SEQ ID NO: 903 - protein, 7 aa, Artificial Sequence
  Murine-derived CDR sequences
DTYFHAS

SEQ ID NO: 904 - protein, 7 aa, Artificial Sequence
  Murine-derived CDR sequences
DTYFHSS

SEQ ID NO: 905 - protein, 7 aa, Artificial Sequence
  Murine-derived CDR sequences
DTYFHKS

SEQ ID NO: 906 - protein, 7 aa, Artificial Sequence
  Murine-derived CDR sequences
DTYFHRS

SEQ ID NO: 907 - protein, 7 aa, Artificial Sequence
  Murine-derived CDR sequences
DTYFHHS

SEQ ID NO: 908 - protein, 7 aa, Artificial Sequence
  Murine-derived CDR sequences
DTYFHPS

SEQ ID NO: 909 - protein, 7 aa, Artificial Sequence
  Murine-derived CDR sequences
DTYFHTS

SEQ ID NO: 910 - protein, 7 aa, Artificial Sequence
  Murine-derived CDR sequences
DTYFHDS

SEQ ID NO: 911 - protein, 7 aa, Artificial Sequence
  Murine-derived CDR sequences
DTYFQAS

SEQ ID NO: 912 - protein, 7 aa, Artificial Sequence
  Murine-derived CDR sequences
DTYFQSS

SEQ ID NO: 913 - protein, 7 aa, Artificial Sequence
  Murine-derived CDR sequences
DTYFQKS

SEQ ID NO: 914 - protein, 7 aa, Artificial Sequence
  Murine-derived CDR sequences
DTYFQRS

SEQ ID NO: 915 - protein, 7 aa, Artificial Sequence
  Murine-derived CDR sequences
DTYFQHS

SEQ ID NO: 916 - protein, 7 aa, Artificial Sequence
  Murine-derived CDR sequences
DTYFQPS

SEQ ID NO: 917 - protein, 7 aa, Artificial Sequence
  Murine-derived CDR sequences
DTYFQTS

SEQ ID NO: 918 - protein, 7 aa, Artificial Sequence
  Murine-derived CDR sequences
DTYFQDS

SEQ ID NO: 919 - protein, 7 aa, Artificial Sequence
  Murine-derived CDR sequences
DTYLLAS

SEQ ID NO: 920 - protein, 7 aa, Artificial Sequence
  Murine-derived CDR sequences
DTYLLSS

SEQ ID NO: 921 - protein, 7 aa, Artificial Sequence
  Murine-derived CDR sequences
DTYLLKS

SEQ ID NO: 922 - protein, 7 aa, Artificial Sequence
  Murine-derived CDR sequences
DTYLLRS

SEQ ID NO: 923 - protein, 7 aa, Artificial Sequence
  Murine-derived CDR sequences
DTYLLHS

SEQ ID NO: 924 - protein, 7 aa, Artificial Sequence
  Murine-derived CDR sequences
DTYLLPS

SEQ ID NO: 925 - protein, 7 aa, Artificial Sequence
  Murine-derived CDR sequences
DTYLLTS

SEQ ID NO: 926 - protein, 7 aa, Artificial Sequence
  Murine-derived CDR sequences
DTYLLDS

SEQ ID NO: 927 - protein, 7 aa, Artificial Sequence
  Murine-derived CDR sequences
DTYLHAS

SEQ ID NO: 928 - protein, 7 aa, Artificial Sequence
  Murine-derived CDR sequences
DTYLHSS

SEQ ID NO: 929 - protein, 7 aa, Artificial Sequence
  Murine-derived CDR sequences
DTYLHKS

SEQ ID NO: 930 - protein, 7 aa, Artificial Sequence
  Murine-derived CDR sequences
DTYLHRS

SEQ ID NO: 931 - protein, 7 aa, Artificial Sequence
  Murine-derived CDR sequences
DTYLHHS

SEQ ID NO: 932 - protein, 7 aa, Artificial Sequence
  Murine-derived CDR sequences
DTYLHPS

SEQ ID NO: 933 - protein, 7 aa, Artificial Sequence
  Murine-derived CDR sequences
DTYLHTS

SEQ ID NO: 934 - protein, 7 aa, Artificial Sequence
  Murine-derived CDR sequences
DTYLHDS

SEQ ID NO: 935 - protein, 7 aa, Artificial Sequence
  Murine-derived CDR sequences
DTYLQAS

SEQ ID NO: 936 - protein, 7 aa, Artificial Sequence
  Murine-derived CDR sequences
DTYLQSS

SEQ ID NO: 937 - protein, 7 aa, Artificial Sequence
  Murine-derived CDR sequences
DTYLQKS

SEQ ID NO: 938 - protein, 7 aa, Artificial Sequence
  Murine-derived CDR sequences
DTYLQRS

SEQ ID NO: 939 - protein, 7 aa, Artificial Sequence
  Murine-derived CDR sequences
DTYLQHS

SEQ ID NO: 940 - protein, 7 aa, Artificial Sequence
  Murine-derived CDR sequences
DTYLQPS

SEQ ID NO: 941 - protein, 7 aa, Artificial Sequence
  Murine-derived CDR sequences
DTYLQTS

SEQ ID NO: 942 - protein, 7 aa, Artificial Sequence
  Murine-derived CDR sequences
DTYLQDS

SEQ ID NO: 943 - protein, 7 aa, Artificial Sequence
  Murine-derived CDR sequences
DTRKLKS

SEQ ID NO: 944 - protein, 7 aa, Artificial Sequence
  Murine-derived CDR sequences
DTRKLRS

SEQ ID NO: 945 - protein, 7 aa, Artificial Sequence
  Murine-derived CDR sequences
DTRKLHS

SEQ ID NO: 946 - protein, 7 aa, Artificial Sequence
  Murine-derived CDR sequences
DTRKLTS

SEQ ID NO: 947 - protein, 7 aa, Artificial Sequence
  Murine-derived CDR sequences
DTRKLDS

SEQ ID NO: 948 - protein, 7 aa, Artificial Sequence
  Murine-derived CDR sequences
DTRKHAS

SEQ ID NO: 949 - protein, 7 aa, Artificial Sequence
  Murine-derived CDR sequences
DTRKHSS

SEQ ID NO: 950 - protein, 7 aa, Artificial Sequence
  Murine-derived CDR sequences
DTRKHKS

SEQ ID NO: 951 - protein, 7 aa, Artificial Sequence
  Murine-derived CDR sequences
DTRKHRS

SEQ ID NO: 952 - protein, 7 aa, Artificial Sequence
  Murine-derived CDR sequences
DTRKHHS

SEQ ID NO: 953 - protein, 7 aa, Artificial Sequence
  Murine-derived CDR sequences
DTRKHPS

SEQ ID NO: 954 - protein, 7 aa, Artificial Sequence
  Murine-derived CDR sequences
DTRKHTS

SEQ ID NO: 955 - protein, 7 aa, Artificial Sequence
  Murine-derived CDR sequences
DTRKHDS

SEQ ID NO: 956 - protein, 7 aa, Artificial Sequence
  Murine-derived CDR sequences
DTRKQKS

SEQ ID NO: 957 - protein, 7 aa, Artificial Sequence
  Murine-derived CDR sequences
DTRKQRS

SEQ ID NO: 958 - protein, 7 aa, Artificial Sequence
  Murine-derived CDR sequences
DTRKQHS

SEQ ID NO: 959 - protein, 7 aa, Artificial Sequence
  Murine-derived CDR sequences
DTRKQPS

SEQ ID NO: 960 - protein, 7 aa, Artificial Sequence
  Murine-derived CDR sequences
DTRKQTS

SEQ ID NO: 961 - protein, 7 aa, Artificial Sequence
  Murine-derived CDR sequences
DTRKQDS

SEQ ID NO: 962 - protein, 7 aa, Artificial Sequence
  Murine-derived CDR sequences
DTRGLSS

SEQ ID NO: 963 - protein, 7 aa, Artificial Sequence
  Murine-derived CDR sequences
DTRGLKS

SEQ ID NO: 964 - protein, 7 aa, Artificial Sequence
  Murine-derived CDR sequences
DTRGLRS

SEQ ID NO: 965 - protein, 7 aa, Artificial Sequence
  Murine-derived CDR sequences
DTRGLHS

SEQ ID NO: 966 - protein, 7 aa, Artificial Sequence
  Murine-derived CDR sequences
DTRGLTS

SEQ ID NO: 967 - protein, 7 aa, Artificial Sequence
  Murine-derived CDR sequences
DTRGLDS

SEQ ID NO: 968 - protein, 7 aa, Artificial Sequence
  Murine-derived CDR sequences
DTRGHAS

SEQ ID NO: 969 - protein, 7 aa, Artificial Sequence
  Murine-derived CDR sequences
DTRGHSS

SEQ ID NO: 970 - protein, 7 aa, Artificial Sequence
  Murine-derived CDR sequences
DTRGHKS

SEQ ID NO: 971 - protein, 7 aa, Artificial Sequence
  Murine-derived CDR sequences
DTRGHRS

SEQ ID NO: 972 - protein, 7 aa, Artificial Sequence
  Murine-derived CDR sequences
DTRGHHS

SEQ ID NO: 973 - protein, 7 aa, Artificial Sequence
  Murine-derived CDR sequences
DTRGHPS

SEQ ID NO: 974 - protein, 7 aa, Artificial Sequence
  Murine-derived CDR sequences
DTRGHTS

SEQ ID NO: 975 - protein, 7 aa, Artificial Sequence
  Murine-derived CDR sequences
DTRGHDS

SEQ ID NO: 976 - protein, 7 aa, Artificial Sequence
  Murine-derived CDR sequences
DTRGQAS

SEQ ID NO: 977 - protein, 7 aa, Artificial Sequence
  Murine-derived CDR sequences
DTRGQSS

SEQ ID NO: 978 - protein, 7 aa, Artificial Sequence
  Murine-derived CDR sequences
DTRGQKS

SEQ ID NO: 979 - protein, 7 aa, Artificial Sequence
  Murine-derived CDR sequences
DTRGQRS

SEQ ID NO: 980 - protein, 7 aa, Artificial Sequence
  Murine-derived CDR sequences
DTRGQHS

SEQ ID NO: 981 - protein, 7 aa, Artificial Sequence
  Murine-derived CDR sequences
DTRGQPS

SEQ ID NO: 982 - protein, 7 aa, Artificial Sequence
  Murine-derived CDR sequences
DTRGQTS

SEQ ID NO: 983 - protein, 7 aa, Artificial Sequence
  Murine-derived CDR sequences
DTRGQDS

SEQ ID NO: 984 - protein, 7 aa, Artificial Sequence
  Murine-derived CDR sequences
DTRRLAS

SEQ ID NO: 985 - protein, 7 aa, Artificial Sequence
  Murine-derived CDR sequences
DTRRLSS

SEQ ID NO: 986 - protein, 7 aa, Artificial Sequence
  Murine-derived CDR sequences
DTRRLKS

SEQ ID NO: 987 - protein, 7 aa, Artificial Sequence
  Murine-derived CDR sequences
DTRRLRS

SEQ ID NO: 988 - protein, 7 aa, Artificial Sequence
  Murine-derived CDR sequences
DTRRLHS

SEQ ID NO: 989 - protein, 7 aa, Artificial Sequence
  Murine-derived CDR sequences
DTRRLPS

SEQ ID NO: 990 - protein, 7 aa, Artificial Sequence
  Murine-derived CDR sequences
DTRRLTS

SEQ ID NO: 991 - protein, 7 aa, Artificial Sequence
  Murine-derived CDR sequences
DTRRLDS

SEQ ID NO: 992 - protein, 7 aa, Artificial Sequence
  Murine-derived CDR sequences
DTRRHAS

SEQ ID NO: 993 - protein, 7 aa, Artificial Sequence
  Murine-derived CDR sequences
DTRRHSS

SEQ ID NO: 994 - protein, 7 aa, Artificial Sequence
  Murine-derived CDR sequences
DTRRHKS

SEQ ID NO: 995 - protein, 7 aa, Artificial Sequence
  Murine-derived CDR sequences
DTRRHRS

SEQ ID NO: 996 - protein, 7 aa, Artificial Sequence
  Murine-derived CDR sequences
DTRRHHS

SEQ ID NO: 997 - protein, 7 aa, Artificial Sequence
  Murine-derived CDR sequences
DTRRHPS

SEQ ID NO: 998 - protein, 7 aa, Artificial Sequence
  Murine-derived CDR sequences
DTRRHTS

SEQ ID NO: 999 - protein, 7 aa, Artificial Sequence
  Murine-derived CDR sequences
DTRRHDS

SEQ ID NO: 1000 - protein, 7 aa, Artificial Sequence
  Murine-derived CDR sequences
DTRRQAS

SEQ ID NO: 1001 - protein, 7 aa, Artificial Sequence
  Murine-derived CDR sequences
DTRRQSS

SEQ ID NO: 1002 - protein, 7 aa, Artificial Sequence
  Murine-derived CDR sequences
DTRRQKS

SEQ ID NO: 1003 - protein, 7 aa, Artificial Sequence
  Murine-derived CDR sequences
DTRRQRS

SEQ ID NO: 1004 - protein, 7 aa, Artificial Sequence
  Murine-derived CDR sequences
DTRRQHS

SEQ ID NO: 1005 - protein, 7 aa, Artificial Sequence
  Murine-derived CDR sequences
DTRRQPS

SEQ ID NO: 1006 - protein, 7 aa, Artificial Sequence
  Murine-derived CDR sequences
DTRRQTS

SEQ ID NO: 1007 - protein, 7 aa, Artificial Sequence
  Murine-derived CDR sequences
DTRRQDS

SEQ ID NO: 1008 - protein, 7 aa, Artificial Sequence
  Murine-derived CDR sequences
DTRYLKS

SEQ ID NO: 1009 - protein, 7 aa, Artificial Sequence
  Murine-derived CDR sequences
DTRYLRS

SEQ ID NO: 1010 - protein, 7 aa, Artificial Sequence
  Murine-derived CDR sequences
DTRYLHS

SEQ ID NO: 1011 - protein, 7 aa, Artificial Sequence
  Murine-derived CDR sequences
DTRYLPS

SEQ ID NO: 1012 - protein, 7 aa, Artificial Sequence
  Murine-derived CDR sequences
DTRYLTS

SEQ ID NO: 1013 - protein, 7 aa, Artificial Sequence
  Murine-derived CDR sequences
DTRYLDS

SEQ ID NO: 1014 - protein, 7 aa, Artificial Sequence
  Murine-derived CDR sequences
DTRYHAS

SEQ ID NO: 1015 - protein, 7 aa, Artificial Sequence
  Murine-derived CDR sequences
DTRYHSS

SEQ ID NO: 1016 - protein, 7 aa, Artificial Sequence
  Murine-derived CDR sequences
DTRYHKS

SEQ ID NO: 1017 - protein, 7 aa, Artificial Sequence
  Murine-derived CDR sequences
DTRYHRS

SEQ ID NO: 1018 - protein, 7 aa, Artificial Sequence
  Murine-derived CDR sequences
DTRYHHS

SEQ ID NO: 1019 - protein, 7 aa, Artificial Sequence
  Murine-derived CDR sequences
DTRYHPS

SEQ ID NO: 1020 - protein, 7 aa, Artificial Sequence
  Murine-derived CDR sequences
DTRYHTS

SEQ ID NO: 1021 - protein, 7 aa, Artificial Sequence
  Murine-derived CDR sequences
DTRYHDS

SEQ ID NO: 1022 - protein, 7 aa, Artificial Sequence
  Murine-derived CDR sequences
DTRYQKS

SEQ ID NO: 1023 - protein, 7 aa, Artificial Sequence
  Murine-derived CDR sequences
DTRYQRS

SEQ ID NO: 1024 - protein, 7 aa, Artificial Sequence
  Murine-derived CDR sequences
DTRYQHS

SEQ ID NO: 1025 - protein, 7 aa, Artificial Sequence
  Murine-derived CDR sequences
DTRYQPS

SEQ ID NO: 1026 - protein, 7 aa, Artificial Sequence
  Murine-derived CDR sequences
DTRYQTS

SEQ ID NO: 1027 - protein, 7 aa, Artificial Sequence
  Murine-derived CDR sequences
DTRYQDS

SEQ ID NO: 1028 - protein, 7 aa, Artificial Sequence
  Murine-derived CDR sequences
DTRFLAS

SEQ ID NO: 1029 - protein, 7 aa, Artificial Sequence
  Murine-derived CDR sequences
DTRFLSS

SEQ ID NO: 1030 - protein, 7 aa, Artificial Sequence
  Murine-derived CDR sequences
DTRFLKS

SEQ ID NO: 1031 - protein, 7 aa, Artificial Sequence
  Murine-derived CDR sequences
DTRFLRS

SEQ ID NO: 1032 - protein, 7 aa, Artificial Sequence
  Murine-derived CDR sequences
DTRFLHS

SEQ ID NO: 1033 - protein, 7 aa, Artificial Sequence
  Murine-derived CDR sequences
DTRFLPS

SEQ ID NO: 1034 - protein, 7 aa, Artificial Sequence
  Murine-derived CDR sequences
DTRFLTS

SEQ ID NO: 1035 - protein, 7 aa, Artificial Sequence
  Murine-derived CDR sequences
DTRFLDS

SEQ ID NO: 1036 - protein, 7 aa, Artificial Sequence
  Murine-derived CDR sequences
DTRFHAS

SEQ ID NO: 1037 - protein, 7 aa, Artificial Sequence
  Murine-derived CDR sequences
DTRFHSS

SEQ ID NO: 1038 - protein, 7 aa, Artificial Sequence
  Murine-derived CDR sequences
DTRFHKS

SEQ ID NO: 1039 - protein, 7 aa, Artificial Sequence
  Murine-derived CDR sequences
DTRFHRS

SEQ ID NO: 1040 - protein, 7 aa, Artificial Sequence
  Murine-derived CDR sequences
DTRFHHS

SEQ ID NO: 1041 - protein, 7 aa, Artificial Sequence
  Murine-derived CDR sequences
DTRFHPS

SEQ ID NO: 1042 - protein, 7 aa, Artificial Sequence
  Murine-derived CDR sequences
DTRFHTS

SEQ ID NO: 1043 - protein, 7 aa, Artificial Sequence
  Murine-derived CDR sequences
DTRFHDS

SEQ ID NO: 1044 - protein, 7 aa, Artificial Sequence
  Murine-derived CDR sequences
DTRFQAS

SEQ ID NO: 1045 - protein, 7 aa, Artificial Sequence
  Murine-derived CDR sequences
DTRFQSS

SEQ ID NO: 1046 - protein, 7 aa, Artificial Sequence
  Murine-derived CDR sequences
DTRFQKS

SEQ ID NO: 1047 - protein, 7 aa, Artificial Sequence
  Murine-derived CDR sequences
DTRFQRS

SEQ ID NO: 1048 - protein, 7 aa, Artificial Sequence
  Murine-derived CDR sequences
DTRFQHS

SEQ ID NO: 1049 - protein, 7 aa, Artificial Sequence
  Murine-derived CDR sequences
DTRFQPS

SEQ ID NO: 1050 - protein, 7 aa, Artificial Sequence
  Murine-derived CDR sequences
DTRFQTS

SEQ ID NO: 1051 - protein, 7 aa, Artificial Sequence
  Murine-derived CDR sequences
DTRFQDS

SEQ ID NO: 1052 - protein, 7 aa, Artificial Sequence
  Murine-derived CDR sequences
DTRLLAS

SEQ ID NO: 1053 - protein, 7 aa, Artificial Sequence
  Murine-derived CDR sequences
DTRLLSS

SEQ ID NO: 1054 - protein, 7 aa, Artificial Sequence
  Murine-derived CDR sequences
DTRLLKS

SEQ ID NO: 1055 - protein, 7 aa, Artificial Sequence
  Murine-derived CDR sequences
DTRLLRS

SEQ ID NO: 1056 - protein, 7 aa, Artificial Sequence
  Murine-derived CDR sequences
DTRLLHS

SEQ ID NO: 1057 - protein, 7 aa, Artificial Sequence
  Murine-derived CDR sequences
DTRLLPS

SEQ ID NO: 1058 - protein, 7 aa, Artificial Sequence
  Murine-derived CDR sequences
DTRLLTS

SEQ ID NO: 1059 - protein, 7 aa, Artificial Sequence
  Murine-derived CDR sequences
DTRLLDS

SEQ ID NO: 1060 - protein, 7 aa, Artificial Sequence
  Murine-derived CDR sequences
DTRLHAS

SEQ ID NO: 1061 - protein, 7 aa, Artificial Sequence
  Murine-derived CDR sequences
DTRLHSS

SEQ ID NO: 1062 - protein, 7 aa, Artificial Sequence
  Murine-derived CDR sequences
DTRLHKS

SEQ ID NO: 1063 - protein, 7 aa, Artificial Sequence
  Murine-derived CDR sequences
DTRLHRS

SEQ ID NO: 1064 - protein, 7 aa, Artificial Sequence
  Murine-derived CDR sequences
DTRLHHS

SEQ ID NO: 1065 - protein, 7 aa, Artificial Sequence
  Murine-derived CDR sequences
DTRLHPS

SEQ ID NO: 1066 - protein, 7 aa, Artificial Sequence
  Murine-derived CDR sequences
DTRLHTS

SEQ ID NO: 1067 - protein, 7 aa, Artificial Sequence
  Murine-derived CDR sequences
DTRLHDS

SEQ ID NO: 1068 - protein, 7 aa, Artificial Sequence
  Murine-derived CDR sequences
DTRLQAS

SEQ ID NO: 1069 - protein, 7 aa, Artificial Sequence
  Murine-derived CDR sequences
DTRLQSS

SEQ ID NO: 1070 - protein, 7 aa, Artificial Sequence
  Murine-derived CDR sequences
DTRLQKS

SEQ ID NO: 1071 - protein, 7 aa, Artificial Sequence
  Murine-derived CDR sequences
DTRLQRS

SEQ ID NO: 1072 - protein, 7 aa, Artificial Sequence
  Murine-derived CDR sequences
DTRLQHS

SEQ ID NO: 1073 - protein, 7 aa, Artificial Sequence
  Murine-derived CDR sequences
DTRLQPS

SEQ ID NO: 1074 - protein, 7 aa, Artificial Sequence
  Murine-derived CDR sequences
DTRLQTS

SEQ ID NO: 1075 - protein, 7 aa, Artificial Sequence
  Murine-derived CDR sequences
DTRLQDS

SEQ ID NO: 1076 - protein, 7 aa, Artificial Sequence
  Murine-derived CDR sequences
DTMKLKS

SEQ ID NO: 1077 - protein, 7 aa, Artificial Sequence
  Murine-derived CDR sequences
DTMKLRS

SEQ ID NO: 1078 - protein, 7 aa, Artificial Sequence
  Murine-derived CDR sequences
DTMKLHS

SEQ ID NO: 1079 - protein, 7 aa, Artificial Sequence
  Murine-derived CDR sequences
DTMKLPS

SEQ ID NO: 1080 - protein, 7 aa, Artificial Sequence
  Murine-derived CDR sequences
DTMKLTS

SEQ ID NO: 1081 - protein, 7 aa, Artificial Sequence
  Murine-derived CDR sequences
DTMKLDS

SEQ ID NO: 1082 - protein, 7 aa, Artificial Sequence
  Murine-derived CDR sequences
DTMKHAS

SEQ ID NO: 1083 - protein, 7 aa, Artificial Sequence
  Murine-derived CDR sequences
DTMKHSS

SEQ ID NO: 1084 - protein, 7 aa, Artificial Sequence
  Murine-derived CDR sequences
DTMKHKS

SEQ ID NO: 1085 - protein, 7 aa, Artificial Sequence
  Murine-derived CDR sequences
DTMKHRS

SEQ ID NO: 1086 - protein, 7 aa, Artificial Sequence
  Murine-derived CDR sequences
DTMKHHS

SEQ ID NO: 1087 - protein, 7 aa, Artificial Sequence
  Murine-derived CDR sequences
DTMKHPS

SEQ ID NO: 1088 - protein, 7 aa, Artificial Sequence
  Murine-derived CDR sequences
DTMKHTS

SEQ ID NO: 1089 - protein, 7 aa, Artificial Sequence
  Murine-derived CDR sequences
DTMKHDS

SEQ ID NO: 1090 - protein, 7 aa, Artificial Sequence
  Murine-derived CDR sequences
DTMKQKS

SEQ ID NO: 1091 - protein, 7 aa, Artificial Sequence
  Murine-derived CDR sequences
DTMKQRS

SEQ ID NO: 1092 - protein, 7 aa, Artificial Sequence
  Murine-derived CDR sequences
DTMKQHS

SEQ ID NO: 1093 - protein, 7 aa, Artificial Sequence
  Murine-derived CDR sequences
DTMKQPS

SEQ ID NO: 1094 - protein, 7 aa, Artificial Sequence
  Murine-derived CDR sequences
DTMKQTS

SEQ ID NO: 1095 - protein, 7 aa, Artificial Sequence
  Murine-derived CDR sequences
DTMKQDS

SEQ ID NO: 1096 - protein, 7 aa, Artificial Sequence
  Murine-derived CDR sequences
DTMGLAS

SEQ ID NO: 1097 - protein, 7 aa, Artificial Sequence
  Murine-derived CDR sequences
DTMGLSS

SEQ ID NO: 1098 - protein, 7 aa, Artificial Sequence
  Murine-derived CDR sequences
DTMGLKS

SEQ ID NO: 1099 - protein, 7 aa, Artificial Sequence
  Murine-derived CDR sequences
DTMGLRS

SEQ ID NO: 1100 - protein, 7 aa, Artificial Sequence
  Murine-derived CDR sequences
DTMGLHS

SEQ ID NO: 1101 - protein, 7 aa, Artificial Sequence
  Murine-derived CDR sequences
DTMGLPS

SEQ ID NO: 1102 - protein, 7 aa, Artificial Sequence
  Murine-derived CDR sequences
DTMGLTS

SEQ ID NO: 1103 - protein, 7 aa, Artificial Sequence
  Murine-derived CDR sequences
DTMGLDS

SEQ ID NO: 1104 - protein, 7 aa, Artificial Sequence
  Murine-derived CDR sequences
DTMGHAS

SEQ ID NO: 1105 - protein, 7 aa, Artificial Sequence
  Murine-derived CDR sequences
DTMGHSS

SEQ ID NO: 1106 - protein, 7 aa, Artificial Sequence
  Murine-derived CDR sequences
DTMGHKS

SEQ ID NO: 1107 - protein, 7 aa, Artificial Sequence
  Murine-derived CDR sequences
DTMGHRS

SEQ ID NO: 1108 - protein, 7 aa, Artificial Sequence
  Murine-derived CDR sequences
DTMGHHS

SEQ ID NO: 1109 - protein, 7 aa, Artificial Sequence
  Murine-derived CDR sequences
DTMGHPS

SEQ ID NO: 1110 - protein, 7 aa, Artificial Sequence
  Murine-derived CDR sequences
DTMGHTS

SEQ ID NO: 1111 - protein, 7 aa, Artificial Sequence
  Murine-derived CDR sequences
DTMGHDS

SEQ ID NO: 1112 - protein, 7 aa, Artificial Sequence
  Murine-derived CDR sequences
DTMGQAS

SEQ ID NO: 1113 - protein, 7 aa, Artificial Sequence
  Murine-derived CDR sequences
DTMGQSS

SEQ ID NO: 1114 - protein, 7 aa, Artificial Sequence
  Murine-derived CDR sequences
DTMGQKS

SEQ ID NO: 1115 - protein, 7 aa, Artificial Sequence
  Murine-derived CDR sequences
DTMGQRS

SEQ ID NO: 1116 - protein, 7 aa, Artificial Sequence
  Murine-derived CDR sequences
DTMGQHS

SEQ ID NO: 1117 - protein, 7 aa, Artificial Sequence
  Murine-derived CDR sequences
DTMGQPS

SEQ ID NO: 1118 - protein, 7 aa, Artificial Sequence
  Murine-derived CDR sequences
DTMGQTS

SEQ ID NO: 1119 - protein, 7 aa, Artificial Sequence
  Murine-derived CDR sequences
DTMGQDS

SEQ ID NO: 1120 - protein, 7 aa, Artificial Sequence
  Murine-derived CDR sequences
DTMRLSS

SEQ ID NO: 1121 - protein, 7 aa, Artificial Sequence
  Murine-derived CDR sequences
DTMRLKS

SEQ ID NO: 1122 - protein, 7 aa, Artificial Sequence
  Murine-derived CDR sequences
DTMRLRS

SEQ ID NO: 1123 - protein, 7 aa, Artificial Sequence
  Murine-derived CDR sequences
DTMRLHS

SEQ ID NO: 1124 - protein, 7 aa, Artificial Sequence
  Murine-derived CDR sequences
DTMRLPS

SEQ ID NO: 1125 - protein, 7 aa, Artificial Sequence
  Murine-derived CDR sequences
DTMRLTS

SEQ ID NO: 1126 - protein, 7 aa, Artificial Sequence
  Murine-derived CDR sequences
DTMRLDS

SEQ ID NO: 1127 - protein, 7 aa, Artificial Sequence
  Murine-derived CDR sequences
DTMRHAS

SEQ ID NO: 1128 - protein, 7 aa, Artificial Sequence
  Murine-derived CDR sequences
DTMRHSS

SEQ ID NO: 1129 - protein, 7 aa, Artificial Sequence
  Murine-derived CDR sequences
DTMRHKS

SEQ ID NO: 1130 - protein, 7 aa, Artificial Sequence
  Murine-derived CDR sequences
DTMRHRS

SEQ ID NO: 1131 - protein, 7 aa, Artificial Sequence
  Murine-derived CDR sequences
DTMRHHS

SEQ ID NO: 1132 - protein, 7 aa, Artificial Sequence
  Murine-derived CDR sequences
DTMRHPS

SEQ ID NO: 1133 - protein, 7 aa, Artificial Sequence
  Murine-derived CDR sequences
DTMRHTS

SEQ ID NO: 1134 - protein, 7 aa, Artificial Sequence
  Murine-derived CDR sequences
DTMRHDS

SEQ ID NO: 1135 - protein, 7 aa, Artificial Sequence
  Murine-derived CDR sequences
DTMRQAS

SEQ ID NO: 1136 - protein, 7 aa, Artificial Sequence
  Murine-derived CDR sequences
DTMRQSS

SEQ ID NO: 1137 - protein, 7 aa, Artificial Sequence
  Murine-derived CDR sequences
DTMRQKS

SEQ ID NO: 1138 - protein, 7 aa, Artificial Sequence
  Murine-derived CDR sequences
DTMRQRS

SEQ ID NO: 1139 - protein, 7 aa, Artificial Sequence
  Murine-derived CDR sequences
DTMRQHS

SEQ ID NO: 1140 - protein, 7 aa, Artificial Sequence
  Murine-derived CDR sequences
DTMRQPS

SEQ ID NO: 1141 - protein, 7 aa, Artificial Sequence
  Murine-derived CDR sequences
DTMRQTS

SEQ ID NO: 1142 - protein, 7 aa, Artificial Sequence
  Murine-derived CDR sequences
DTMRQDS

SEQ ID NO: 1143 - protein, 7 aa, Artificial Sequence
  Murine-derived CDR sequences
DTMYLKS

SEQ ID NO: 1144 - protein, 7 aa, Artificial Sequence
  Murine-derived CDR sequences
DTMYLRS

SEQ ID NO: 1145 - protein, 7 aa, Artificial Sequence
  Murine-derived CDR sequences
DTMYLHS

SEQ ID NO: 1146 - protein, 7 aa, Artificial Sequence
  Murine-derived CDR sequences
DTMYLPS

SEQ ID NO: 1147 - protein, 7 aa, Artificial Sequence
  Murine-derived CDR sequences
DTMYLTS

SEQ ID NO: 1148 - protein, 7 aa, Artificial Sequence
  Murine-derived CDR sequences
DTMYLDS

SEQ ID NO: 1149 - protein, 7 aa, Artificial Sequence
  Murine-derived CDR sequences
DTMYHAS

SEQ ID NO: 1150 - protein, 7 aa, Artificial Sequence
  Murine-derived CDR sequences
DTMYHSS

SEQ ID NO: 1151 - protein, 7 aa, Artificial Sequence
  Murine-derived CDR sequences
DTMYHKS

SEQ ID NO: 1152 - protein, 7 aa, Artificial Sequence
  Murine-derived CDR sequences
DTMYHRS

SEQ ID NO: 1153 - protein, 7 aa, Artificial Sequence
  Murine-derived CDR sequences
DTMYHHS

SEQ ID NO: 1154 - protein, 7 aa, Artificial Sequence
  Murine-derived CDR sequences
DTMYHPS

SEQ ID NO: 1155 - protein, 7 aa, Artificial Sequence
  Murine-derived CDR sequences
DTMYHTS

SEQ ID NO: 1156 - protein, 7 aa, Artificial Sequence
  Murine-derived CDR sequences
DTMYHDS

SEQ ID NO: 1157 - protein, 7 aa, Artificial Sequence
  Murine-derived CDR sequences
DTMYQKS

SEQ ID NO: 1158 - protein, 7 aa, Artificial Sequence
  Murine-derived CDR sequences
DTMYQRS

SEQ ID NO: 1159 - protein, 7 aa, Artificial Sequence
  Murine-derived CDR sequences
DTMYQHS

SEQ ID NO: 1160 - protein, 7 aa, Artificial Sequence
  Murine-derived CDR sequences
DTMYQPS

SEQ ID NO: 1161 - protein, 7 aa, Artificial Sequence
  Murine-derived CDR sequences
DTMYQTS

SEQ ID NO: 1162 - protein, 7 aa, Artificial Sequence
  Murine-derived CDR sequences
DTMYQDS

SEQ ID NO: 1163 - protein, 7 aa, Artificial Sequence
  Murine-derived CDR sequences
DTMFLAS

SEQ ID NO: 1164 - protein, 7 aa, Artificial Sequence
  Murine-derived CDR sequences
DTMFLSS

SEQ ID NO: 1165 - protein, 7 aa, Artificial Sequence
  Murine-derived CDR sequences
DTMFLKS

SEQ ID NO: 1166 - protein, 7 aa, Artificial Sequence
  Murine-derived CDR sequences
DTMFLRS

SEQ ID NO: 1167 - protein, 7 aa, Artificial Sequence
  Murine-derived CDR sequences
DTMFLHS

SEQ ID NO: 1168 - protein, 7 aa, Artificial Sequence
  Murine-derived CDR sequences
DTMFLPS

SEQ ID NO: 1169 - protein, 7 aa, Artificial Sequence
  Murine-derived CDR sequences
DTMFLTS

SEQ ID NO: 1170 - protein, 7 aa, Artificial Sequence
  Murine-derived CDR sequences
DTMFLDS

SEQ ID NO: 1171 - protein, 7 aa, Artificial Sequence
  Murine-derived CDR sequences
DTMFHAS

SEQ ID NO: 1172 - protein, 7 aa, Artificial Sequence
  Murine-derived CDR sequences
DTMFHSS

SEQ ID NO: 1173 - protein, 7 aa, Artificial Sequence
  Murine-derived CDR sequences
DTMFHKS

SEQ ID NO: 1174 - protein, 7 aa, Artificial Sequence
  Murine-derived CDR sequences
DTMFHRS

SEQ ID NO: 1175 - protein, 7 aa, Artificial Sequence
  Murine-derived CDR sequences
DTMFHHS

SEQ ID NO: 1176 - protein, 7 aa, Artificial Sequence
  Murine-derived CDR sequences
DTMFHPS

SEQ ID NO: 1177 - protein, 7 aa, Artificial Sequence
  Murine-derived CDR sequences
DTMFHTS

SEQ ID NO: 1178 - protein, 7 aa, Artificial Sequence
  Murine-derived CDR sequences
DTMFHDS

SEQ ID NO: 1179 - protein, 7 aa, Artificial Sequence
  Murine-derived CDR sequences
DTMFQAS

SEQ ID NO: 1180 - protein, 7 aa, Artificial Sequence
  Murine-derived CDR sequences
DTMFQSS

SEQ ID NO: 1181 - protein, 7 aa, Artificial Sequence
  Murine-derived CDR sequences
DTMFQKS

SEQ ID NO: 1182 - protein, 7 aa, Artificial Sequence
  Murine-derived CDR sequences
DTMFQRS

SEQ ID NO: 1183 - protein, 7 aa, Artificial Sequence
  Murine-derived CDR sequences
DTMFQHS

SEQ ID NO: 1184 - protein, 7 aa, Artificial Sequence
  Murine-derived CDR sequences
DTMFQPS

SEQ ID NO: 1185 - protein, 7 aa, Artificial Sequence
  Murine-derived CDR sequences
DTMFQTS

SEQ ID NO: 1186 - protein, 7 aa, Artificial Sequence
  Murine-derived CDR sequences
DTMFQDS

SEQ ID NO: 1187 - protein, 7 aa, Artificial Sequence
  Murine-derived CDR sequences
DTMLLAS

SEQ ID NO: 1188 - protein, 7 aa, Artificial Sequence
  Murine-derived CDR sequences
DTMLLSS

SEQ ID NO: 1189 - protein, 7 aa, Artificial Sequence
  Murine-derived CDR sequences
DTMLLKS

SEQ ID NO: 1190 - protein, 7 aa, Artificial Sequence
  Murine-derived CDR sequences
DTMLLRS

SEQ ID NO: 1191 - protein, 7 aa, Artificial Sequence
  Murine-derived CDR sequences
DTMLLHS

SEQ ID NO: 1192 - protein, 7 aa, Artificial Sequence
  Murine-derived CDR sequences
DTMLLPS

SEQ ID NO: 1193 - protein, 7 aa, Artificial Sequence
  Murine-derived CDR sequences
DTMLLTS

SEQ ID NO: 1194 - protein, 7 aa, Artificial Sequence
  Murine-derived CDR sequences
DTMLLDS

SEQ ID NO: 1195 - protein, 7 aa, Artificial Sequence
  Murine-derived CDR sequences
DTMLHAS

SEQ ID NO: 1196 - protein, 7 aa, Artificial Sequence
  Murine-derived CDR sequences
DTMLHSS

SEQ ID NO: 1197 - protein, 7 aa, Artificial Sequence
  Murine-derived CDR sequences
DTMLHKS

SEQ ID NO: 1198 - protein, 7 aa, Artificial Sequence
  Murine-derived CDR sequences
DTMLHRS

SEQ ID NO: 1199 - protein, 7 aa, Artificial Sequence
  Murine-derived CDR sequences
DTMLHHS

SEQ ID NO: 1200 - protein, 7 aa, Artificial Sequence
  Murine-derived CDR sequences
DTMLHPS

SEQ ID NO: 1201 - protein, 7 aa, Artificial Sequence
  Murine-derived CDR sequences
DTMLHTS

SEQ ID NO: 1202 - protein, 7 aa, Artificial Sequence
  Murine-derived CDR sequences
DTMLHDS

SEQ ID NO: 1203 - protein, 7 aa, Artificial Sequence
  Murine-derived CDR sequences
DTMLQAS

SEQ ID NO: 1204 - protein, 7 aa, Artificial Sequence
  Murine-derived CDR sequences
DTMLQSS

SEQ ID NO: 1205 - protein, 7 aa, Artificial Sequence
  Murine-derived CDR sequences
DTMLQKS

SEQ ID NO: 1206 - protein, 7 aa, Artificial Sequence
  Murine-derived CDR sequences
DTMLQRS

SEQ ID NO: 1207 - protein, 7 aa, Artificial Sequence
  Murine-derived CDR sequences
DTMLQHS

SEQ ID NO: 1208 - protein, 7 aa, Artificial Sequence
  Murine-derived CDR sequences
DTMLQPS

SEQ ID NO: 1209 - protein, 7 aa, Artificial Sequence
  Murine-derived CDR sequences
DTMLQTS

SEQ ID NO: 1210 - protein, 7 aa, Artificial Sequence
  Murine-derived CDR sequences
DTMLQDS

SEQ ID NO: 1211 - protein, 7 aa, Artificial Sequence
  Murine-derived CDR sequences
DTKKLAS

SEQ ID NO: 1212 - protein, 7 aa, Artificial Sequence
  Murine-derived CDR sequences
DTKKLSS

SEQ ID NO: 1213 - protein, 7 aa, Artificial Sequence
  Murine-derived CDR sequences
DTKKLKS

SEQ ID NO: 1214 - protein, 7 aa, Artificial Sequence
  Murine-derived CDR sequences
DTKKLRS

SEQ ID NO: 1215 - protein, 7 aa, Artificial Sequence
  Murine-derived CDR sequences
DTKKLHS

SEQ ID NO: 1216 - protein, 7 aa, Artificial Sequence
  Murine-derived CDR sequences
DTKKLPS

SEQ ID NO: 1217 - protein, 7 aa, Artificial Sequence
  Murine-derived CDR sequences
DTKKLTS

SEQ ID NO: 1218 - protein, 7 aa, Artificial Sequence
  Murine-derived CDR sequences
DTKKLDS

SEQ ID NO: 1219 - protein, 7 aa, Artificial Sequence
  Murine-derived CDR sequences
DTKKHAS

SEQ ID NO: 1220 - protein, 7 aa, Artificial Sequence
  Murine-derived CDR sequences
DTKKHSS

SEQ ID NO: 1221 - protein, 7 aa, Artificial Sequence
  Murine-derived CDR sequences
DTKKHKS

SEQ ID NO: 1222 - protein, 7 aa, Artificial Sequence
  Murine-derived CDR sequences
DTKKHRS

SEQ ID NO: 1223 - protein, 7 aa, Artificial Sequence
  Murine-derived CDR sequences
DTKKHHS

SEQ ID NO: 1224 - protein, 7 aa, Artificial Sequence
  Murine-derived CDR sequences
DTKKHPS

SEQ ID NO: 1225 - protein, 7 aa, Artificial Sequence
  Murine-derived CDR sequences
DTKKHTS

SEQ ID NO: 1226 - protein, 7 aa, Artificial Sequence
  Murine-derived CDR sequences
DTKKHDS

SEQ ID NO: 1227 - protein, 7 aa, Artificial Sequence
  Murine-derived CDR sequences
DTKKQAS

SEQ ID NO: 1228 - protein, 7 aa, Artificial Sequence
  Murine-derived CDR sequences
DTKKQSS

SEQ ID NO: 1229 - protein, 7 aa, Artificial Sequence
  Murine-derived CDR sequences
DTKKQKS

SEQ ID NO: 1230 - protein, 7 aa, Artificial Sequence
  Murine-derived CDR sequences
DTKKQRS

SEQ ID NO: 1231 - protein, 7 aa, Artificial Sequence
  Murine-derived CDR sequences
DTKKQHS

SEQ ID NO: 1232 - protein, 7 aa, Artificial Sequence
  Murine-derived CDR sequences
DTKKQPS

SEQ ID NO: 1233 - protein, 7 aa, Artificial Sequence
  Murine-derived CDR sequences
DTKKQTS

SEQ ID NO: 1234 - protein, 7 aa, Artificial Sequence
  Murine-derived CDR sequences
DTKKQDS

SEQ ID NO: 1235 - protein, 7 aa, Artificial Sequence
  Murine-derived CDR sequences
DTKGLAS

SEQ ID NO: 1236 - protein, 7 aa, Artificial Sequence
  Murine-derived CDR sequences
DTKGLSS

SEQ ID NO: 1237 - protein, 7 aa, Artificial Sequence
  Murine-derived CDR sequences
DTKGLKS

SEQ ID NO: 1238 - protein, 7 aa, Artificial Sequence
  Murine-derived CDR sequences
DTKGLRS

SEQ ID NO: 1239 - protein, 7 aa, Artificial Sequence
  Murine-derived CDR sequences
DTKGLHS

SEQ ID NO: 1240 - protein, 7 aa, Artificial Sequence
  Murine-derived CDR sequences
DTKGLPS

SEQ ID NO: 1241 - protein, 7 aa, Artificial Sequence
  Murine-derived CDR sequences
DTKGLTS

SEQ ID NO: 1242 - protein, 7 aa, Artificial Sequence
  Murine-derived CDR sequences
DTKGLDS

SEQ ID NO: 1243 - protein, 7 aa, Artificial Sequence
  Murine-derived CDR sequences
DTKGHAS

SEQ ID NO: 1244 - protein, 7 aa, Artificial Sequence
  Murine-derived CDR sequences
DTKGHSS

SEQ ID NO: 1245 - protein, 7 aa, Artificial Sequence
  Murine-derived CDR sequences
DTKGHKS

SEQ ID NO: 1246 - protein, 7 aa, Artificial Sequence
  Murine-derived CDR sequences
DTKGHRS

SEQ ID NO: 1247 - protein, 7 aa, Artificial Sequence
  Murine-derived CDR sequences
DTKGHHS

SEQ ID NO: 1248 - protein, 7 aa, Artificial Sequence
  Murine-derived CDR sequences
DTKGHPS

SEQ ID NO: 1249 - protein, 7 aa, Artificial Sequence
  Murine-derived CDR sequences
DTKGHTS

SEQ ID NO: 1250 - protein, 7 aa, Artificial Sequence
  Murine-derived CDR sequences
DTKGHDS

SEQ ID NO: 1251 - protein, 7 aa, Artificial Sequence
  Murine-derived CDR sequences
DTKGQAS

SEQ ID NO: 1252 - protein, 7 aa, Artificial Sequence
  Murine-derived CDR sequences
DTKGQSS

SEQ ID NO: 1253 - protein, 7 aa, Artificial Sequence
  Murine-derived CDR sequences
DTKGQKS

SEQ ID NO: 1254 - protein, 7 aa, Artificial Sequence
  Murine-derived CDR sequences
DTKGQRS

SEQ ID NO: 1255 - protein, 7 aa, Artificial Sequence
  Murine-derived CDR sequences
DTKGQHS

SEQ ID NO: 1256 - protein, 7 aa, Artificial Sequence
  Murine-derived CDR sequences
DTKGQPS

SEQ ID NO: 1257 - protein, 7 aa, Artificial Sequence
  Murine-derived CDR sequences
DTKGQTS

SEQ ID NO: 1258 - protein, 7 aa, Artificial Sequence
  Murine-derived CDR sequences
DTKGQDS

SEQ ID NO: 1259 - protein, 7 aa, Artificial Sequence
  Murine-derived CDR sequences
DTKRLAS

SEQ ID NO: 1260 - protein, 7 aa, Artificial Sequence
  Murine-derived CDR sequences
DTKRLSS

SEQ ID NO: 1261 - protein, 7 aa, Artificial Sequence
  Murine-derived CDR sequences
DTKRLKS

SEQ ID NO: 1262 - protein, 7 aa, Artificial Sequence
  Murine-derived CDR sequences
DTKRLRS

SEQ ID NO: 1263 - protein, 7 aa, Artificial Sequence
  Murine-derived CDR sequences
DTKRLHS

SEQ ID NO: 1264 - protein, 7 aa, Artificial Sequence
  Murine-derived CDR sequences
DTKRLPS

SEQ ID NO: 1265 - protein, 7 aa, Artificial Sequence
  Murine-derived CDR sequences
DTKRLTS

SEQ ID NO: 1266 - protein, 7 aa, Artificial Sequence
  Murine-derived CDR sequences
DTKRLDS

SEQ ID NO: 1267 - protein, 7 aa, Artificial Sequence
  Murine-derived CDR sequences
DTKRHAS

SEQ ID NO: 1268 - protein, 7 aa, Artificial Sequence
  Murine-derived CDR sequences
DTKRHSS

SEQ ID NO: 1269 - protein, 7 aa, Artificial Sequence
  Murine-derived CDR sequences
DTKRHKS

SEQ ID NO: 1270 - protein, 7 aa, Artificial Sequence
  Murine-derived CDR sequences
DTKRHRS

SEQ ID NO: 1271 - protein, 7 aa, Artificial Sequence
  Murine-derived CDR sequences
DTKRHHS

SEQ ID NO: 1272 - protein, 7 aa, Artificial Sequence
  Murine-derived CDR sequences
DTKRHPS

SEQ ID NO: 1273 - protein, 7 aa, Artificial Sequence
  Murine-derived CDR sequences
DTKRHTS

SEQ ID NO: 1274 - protein, 7 aa, Artificial Sequence
  Murine-derived CDR sequences
DTKRHDS

SEQ ID NO: 1275 - protein, 7 aa, Artificial Sequence
  Murine-derived CDR sequences
DTKRQAS

SEQ ID NO: 1276 - protein, 7 aa, Artificial Sequence
  Murine-derived CDR sequences
DTKRQSS

SEQ ID NO: 1277 - protein, 7 aa, Artificial Sequence
  Murine-derived CDR sequences
DTKRQKS

SEQ ID NO: 1278 - protein, 7 aa, Artificial Sequence
  Murine-derived CDR sequences
DTKRQRS

SEQ ID NO: 1279 - protein, 7 aa, Artificial Sequence
  Murine-derived CDR sequences
DTKRQHS

SEQ ID NO: 1280 - protein, 7 aa, Artificial Sequence
  Murine-derived CDR sequences
DTKRQPS

SEQ ID NO: 1281 - protein, 7 aa, Artificial Sequence
  Murine-derived CDR sequences
DTKRQTS

SEQ ID NO: 1282 - protein, 7 aa, Artificial Sequence
  Murine-derived CDR sequences
DTKRQDS

SEQ ID NO: 1283 - protein, 7 aa, Artificial Sequence
  Murine-derived CDR sequences
DTKYLAS

SEQ ID NO: 1284 - protein, 7 aa, Artificial Sequence
  Murine-derived CDR sequences
DTKYLSS

SEQ ID NO: 1285 - protein, 7 aa, Artificial Sequence
  Murine-derived CDR sequences
DTKYLKS

SEQ ID NO: 1286 - protein, 7 aa, Artificial Sequence
  Murine-derived CDR sequences
DTKYLRS

SEQ ID NO: 1287 - protein, 7 aa, Artificial Sequence
  Murine-derived CDR sequences
DTKYLHS

SEQ ID NO: 1288 - protein, 7 aa, Artificial Sequence
  Murine-derived CDR sequences
DTKYLPS

SEQ ID NO: 1289 - protein, 7 aa, Artificial Sequence
  Murine-derived CDR sequences
DTKYLTS

SEQ ID NO: 1290 - protein, 7 aa, Artificial Sequence
  Murine-derived CDR sequences
DTKYLDS

SEQ ID NO: 1291 - protein, 7 aa, Artificial Sequence
  Murine-derived CDR sequences
DTKYHAS

SEQ ID NO: 1292 - protein, 7 aa, Artificial Sequence
  Murine-derived CDR sequences
DTKYHSS

SEQ ID NO: 1293 - protein, 7 aa, Artificial Sequence
  Murine-derived CDR sequences
DTKYHKS

SEQ ID NO: 1294 - protein, 7 aa, Artificial Sequence
  Murine-derived CDR sequences
DTKYHRS

SEQ ID NO: 1295 - protein, 7 aa, Artificial Sequence
  Murine-derived CDR sequences
DTKYHHS

SEQ ID NO: 1296 - protein, 7 aa, Artificial Sequence
  Murine-derived CDR sequences
DTKYHPS

SEQ ID NO: 1297 - protein, 7 aa, Artificial Sequence
  Murine-derived CDR sequences
DTKYHTS

SEQ ID NO: 1298 - protein, 7 aa, Artificial Sequence
  Murine-derived CDR sequences
DTKYHDS

SEQ ID NO: 1299 - protein, 7 aa, Artificial Sequence
  Murine-derived CDR sequences
DTKYQAS

SEQ ID NO: 1300 - protein, 7 aa, Artificial Sequence
  Murine-derived CDR sequences
DTKYQSS

SEQ ID NO: 1301 - protein, 7 aa, Artificial Sequence
  Murine-derived CDR sequences
DTKYQKS

SEQ ID NO: 1302 - protein, 7 aa, Artificial Sequence
  Murine-derived CDR sequences
DTKYQRS

SEQ ID NO: 1303 - protein, 7 aa, Artificial Sequence
  Murine-derived CDR sequences
DTKYQHS

SEQ ID NO: 1304 - protein, 7 aa, Artificial Sequence
  Murine-derived CDR sequences
DTKYQPS

SEQ ID NO: 1305 - protein, 7 aa, Artificial Sequence
  Murine-derived CDR sequences
DTKYQTS

SEQ ID NO: 1306 - protein, 7 aa, Artificial Sequence
  Murine-derived CDR sequences
DTKYQDS

SEQ ID NO: 1307 - protein, 7 aa, Artificial Sequence
  Murine-derived CDR sequences
DTKFLAS

SEQ ID NO: 1308 - protein, 7 aa, Artificial Sequence
  Murine-derived CDR sequences
DTKFLSS

SEQ ID NO: 1309 - protein, 7 aa, Artificial Sequence
  Murine-derived CDR sequences
DTKFLKS

SEQ ID NO: 1310 - protein, 7 aa, Artificial Sequence
  Murine-derived CDR sequences
DTKFLRS

SEQ ID NO: 1311 - protein, 7 aa, Artificial Sequence
  Murine-derived CDR sequences
DTKFLHS

SEQ ID NO: 1312 - protein, 7 aa, Artificial Sequence
  Murine-derived CDR sequences
DTKFLPS

SEQ ID NO: 1313 - protein, 7 aa, Artificial Sequence
  Murine-derived CDR sequences
DTKFLTS

SEQ ID NO: 1314 - protein, 7 aa, Artificial Sequence
  Murine-derived CDR sequences
DTKFLDS

SEQ ID NO: 1315 - protein, 7 aa, Artificial Sequence
  Murine-derived CDR sequences
DTKFHAS

SEQ ID NO: 1316 - protein, 7 aa, Artificial Sequence
  Murine-derived CDR sequences
DTKFHSS

SEQ ID NO: 1317 - protein, 7 aa, Artificial Sequence
  Murine-derived CDR sequences
DTKFHKS

SEQ ID NO: 1318 - protein, 7 aa, Artificial Sequence
  Murine-derived CDR sequences
DTKFHRS

SEQ ID NO: 1319 - protein, 7 aa, Artificial Sequence
  Murine-derived CDR sequences
DTKFHHS

SEQ ID NO: 1320 - protein, 7 aa, Artificial Sequence
  Murine-derived CDR sequences
DTKFHPS

SEQ ID NO: 1321 - protein, 7 aa, Artificial Sequence
  Murine-derived CDR sequences
DTKFHTS

SEQ ID NO: 1322 - protein, 7 aa, Artificial Sequence
  Murine-derived CDR sequences
DTKFHDS

SEQ ID NO: 1323 - protein, 7 aa, Artificial Sequence
  Murine-derived CDR sequences
DTKFQAS

SEQ ID NO: 1324 - protein, 7 aa, Artificial Sequence
  Murine-derived CDR sequences
DTKFQSS

SEQ ID NO: 1325 - protein, 7 aa, Artificial Sequence
  Murine-derived CDR sequences
DTKFQKS

SEQ ID NO: 1326 - protein, 7 aa, Artificial Sequence
  Murine-derived CDR sequences
DTKFQRS

SEQ ID NO: 1327 - protein, 7 aa, Artificial Sequence
  Murine-derived CDR sequences
DTKFQHS

SEQ ID NO: 1328 - protein, 7 aa, Artificial Sequence
  Murine-derived CDR sequences
DTKFQPS

SEQ ID NO: 1329 - protein, 7 aa, Artificial Sequence
  Murine-derived CDR sequences
DTKFQTS

SEQ ID NO: 1330 - protein, 7 aa, Artificial Sequence
  Murine-derived CDR sequences
DTKFQDS

SEQ ID NO: 1331 - protein, 7 aa, Artificial Sequence
  Murine-derived CDR sequences
DTKLLAS

SEQ ID NO: 1332 - protein, 7 aa, Artificial Sequence
  Murine-derived CDR sequences
DTKLLSS

SEQ ID NO: 1333 - protein, 7 aa, Artificial Sequence
  Murine-derived CDR sequences
DTKLLKS

SEQ ID NO: 1334 - protein, 7 aa, Artificial Sequence
  Murine-derived CDR sequences
DTKLLRS

SEQ ID NO: 1335 - protein, 7 aa, Artificial Sequence
  Murine-derived CDR sequences
DTKLLHS

SEQ ID NO: 1336 - protein, 7 aa, Artificial Sequence
  Murine-derived CDR sequences
DTKLLPS

SEQ ID NO: 1337 - protein, 7 aa, Artificial Sequence
  Murine-derived CDR sequences
DTKLLTS

SEQ ID NO: 1338 - protein, 7 aa, Artificial Sequence
  Murine-derived CDR sequences
DTKLLDS

SEQ ID NO: 1339 - protein, 7 aa, Artificial Sequence
  Murine-derived CDR sequences
DTKLHAS

SEQ ID NO: 1340 - protein, 7 aa, Artificial Sequence
  Murine-derived CDR sequences
DTKLHSS

SEQ ID NO: 1341 - protein, 7 aa, Artificial Sequence
  Murine-derived CDR sequences
DTKLHKS

SEQ ID NO: 1342 - protein, 7 aa, Artificial Sequence
  Murine-derived CDR sequences
DTKLHRS

SEQ ID NO: 1343 - protein, 7 aa, Artificial Sequence
  Murine-derived CDR sequences
DTKLHHS

SEQ ID NO: 1344 - protein, 7 aa, Artificial Sequence
  Murine-derived CDR sequences
DTKLHPS

SEQ ID NO: 1345 - protein, 7 aa, Artificial Sequence
  Murine-derived CDR sequences
DTKLHTS

SEQ ID NO: 1346 - protein, 7 aa, Artificial Sequence
  Murine-derived CDR sequences
DTKLHDS

SEQ ID NO: 1347 - protein, 7 aa, Artificial Sequence
  Murine-derived CDR sequences
DTKLQAS

SEQ ID NO: 1348 - protein, 7 aa, Artificial Sequence
  Murine-derived CDR sequences
DTKLQSS

SEQ ID NO: 1349 - protein, 7 aa, Artificial Sequence
  Murine-derived CDR sequences
DTKLQKS

SEQ ID NO: 1350 - protein, 7 aa, Artificial Sequence
  Murine-derived CDR sequences
DTKLQRS

SEQ ID NO: 1351 - protein, 7 aa, Artificial Sequence
  Murine-derived CDR sequences
DTKLQHS

SEQ ID NO: 1352 - protein, 7 aa, Artificial Sequence
  Murine-derived CDR sequences
DTKLQPS

SEQ ID NO: 1353 - protein, 7 aa, Artificial Sequence
  Murine-derived CDR sequences
DTKLQTS

SEQ ID NO: 1354 - protein, 7 aa, Artificial Sequence
  Murine-derived CDR sequences
DTKLQDS

SEQ ID NO: 1355 - protein, 7 aa, Artificial Sequence
  Murine-derived CDR sequences
DTLKLSS

SEQ ID NO: 1356 - protein, 7 aa, Artificial Sequence
  Murine-derived CDR sequences
DTLKLKS

SEQ ID NO: 1357 - protein, 7 aa, Artificial Sequence
  Murine-derived CDR sequences
DTLKLRS

SEQ ID NO: 1358 - protein, 7 aa, Artificial Sequence
  Murine-derived CDR sequences
DTLKLHS

SEQ ID NO: 1359 - protein, 7 aa, Artificial Sequence
  Murine-derived CDR sequences
DTLKLPS

SEQ ID NO: 1360 - protein, 7 aa, Artificial Sequence
  Murine-derived CDR sequences
DTLKLTS

SEQ ID NO: 1361 - protein, 7 aa, Artificial Sequence
  Murine-derived CDR sequences
DTLKHAS

SEQ ID NO: 1362 - protein, 7 aa, Artificial Sequence
  Murine-derived CDR sequences
DTLKHSS

SEQ ID NO: 1363 - protein, 7 aa, Artificial Sequence
  Murine-derived CDR sequences
DTLKHKS

SEQ ID NO: 1364 - protein, 7 aa, Artificial Sequence
  Murine-derived CDR sequences
DTLKHRS

SEQ ID NO: 1365 - protein, 7 aa, Artificial Sequence
  Murine-derived CDR sequences
DTLKHHS

SEQ ID NO: 1366 - protein, 7 aa, Artificial Sequence
  Murine-derived CDR sequences
DTLKHPS

SEQ ID NO: 1367 - protein, 7 aa, Artificial Sequence
  Murine-derived CDR sequences
DTLKHTS

SEQ ID NO: 1368 - protein, 7 aa, Artificial Sequence
  Murine-derived CDR sequences
DTLKHDS

SEQ ID NO: 1369 - protein, 7 aa, Artificial Sequence
  Murine-derived CDR sequences
DTLKQAS

SEQ ID NO: 1370 - protein, 7 aa, Artificial Sequence
  Murine-derived CDR sequences
DTLKQSS

SEQ ID NO: 1371 - protein, 7 aa, Artificial Sequence
  Murine-derived CDR sequences
DTLKQKS

SEQ ID NO: 1372 - protein, 7 aa, Artificial Sequence
  Murine-derived CDR sequences
DTLKQRS

SEQ ID NO: 1373 - protein, 7 aa, Artificial Sequence
  Murine-derived CDR sequences
DTLKQHS

SEQ ID NO: 1374 - protein, 7 aa, Artificial Sequence
  Murine-derived CDR sequences
DTLKQPS

SEQ ID NO: 1375 - protein, 7 aa, Artificial Sequence
  Murine-derived CDR sequences
DTLKQTS

SEQ ID NO: 1376 - protein, 7 aa, Artificial Sequence
  Murine-derived CDR sequences
DTLKQDS

SEQ ID NO: 1377 - protein, 7 aa, Artificial Sequence
  Murine-derived CDR sequences
DTLGLAS

SEQ ID NO: 1378 - protein, 7 aa, Artificial Sequence
  Murine-derived CDR sequences
DTLGLSS

SEQ ID NO: 1379 - protein, 7 aa, Artificial Sequence
  Murine-derived CDR sequences
DTLGLKS

SEQ ID NO: 1380 - protein, 7 aa, Artificial Sequence
  Murine-derived CDR sequences
DTLGLRS

SEQ ID NO: 1381 - protein, 7 aa, Artificial Sequence
  Murine-derived CDR sequences
DTLGLHS

SEQ ID NO: 1382 - protein, 7 aa, Artificial Sequence
  Murine-derived CDR sequences
DTLGLPS

SEQ ID NO: 1383 - protein, 7 aa, Artificial Sequence
  Murine-derived CDR sequences
DTLGLTS

SEQ ID NO: 1384 - protein, 7 aa, Artificial Sequence
  Murine-derived CDR sequences
DTLGLDS

SEQ ID NO: 1385 - protein, 7 aa, Artificial Sequence
  Murine-derived CDR sequences
DTLGHAS

SEQ ID NO: 1386 - protein, 7 aa, Artificial Sequence
  Murine-derived CDR sequences
DTLGHSS

SEQ ID NO: 1387 - protein, 7 aa, Artificial Sequence
  Murine-derived CDR sequences
DTLGHKS

SEQ ID NO: 1388 - protein, 7 aa, Artificial Sequence
  Murine-derived CDR sequences
DTLGHRS

SEQ ID NO: 1389 - protein, 7 aa, Artificial Sequence
  Murine-derived CDR sequences
DTLGHHS

SEQ ID NO: 1390 - protein, 7 aa, Artificial Sequence
  Murine-derived CDR sequences
DTLGHPS

SEQ ID NO: 1391 - protein, 7 aa, Artificial Sequence
  Murine-derived CDR sequences
DTLGHTS

SEQ ID NO: 1392 - protein, 7 aa, Artificial Sequence
  Murine-derived CDR sequences
DTLGHDS

SEQ ID NO: 1393 - protein, 7 aa, Artificial Sequence
  Murine-derived CDR sequences
DTLGQAS

SEQ ID NO: 1394 - protein, 7 aa, Artificial Sequence
  Murine-derived CDR sequences
DTLGQSS

SEQ ID NO: 1395 - protein, 7 aa, Artificial Sequence
  Murine-derived CDR sequences
DTLGQKS

SEQ ID NO: 1396 - protein, 7 aa, Artificial Sequence
  Murine-derived CDR sequences
DTLGQRS

SEQ ID NO: 1397 - protein, 7 aa, Artificial Sequence
  Murine-derived CDR sequences
DTLGQHS

SEQ ID NO: 1398 - protein, 7 aa, Artificial Sequence
  Murine-derived CDR sequences
DTLGQPS

SEQ ID NO: 1399 - protein, 7 aa, Artificial Sequence
  Murine-derived CDR sequences
DTLGQTS

SEQ ID NO: 1400 - protein, 7 aa, Artificial Sequence
  Murine-derived CDR sequences
DTLGQDS

SEQ ID NO: 1401 - protein, 7 aa, Artificial Sequence
  Murine-derived CDR sequences
DTLRLAS

SEQ ID NO: 1402 - protein, 7 aa, Artificial Sequence
  Murine-derived CDR sequences
DTLRLSS

SEQ ID NO: 1403 - protein, 7 aa, Artificial Sequence
  Murine-derived CDR sequences
DTLRLKS

SEQ ID NO: 1404 - protein, 7 aa, Artificial Sequence
  Murine-derived CDR sequences
DTLRLRS

SEQ ID NO: 1405 - protein, 7 aa, Artificial Sequence
  Murine-derived CDR sequences
DTLRLHS

SEQ ID NO: 1406 - protein, 7 aa, Artificial Sequence
  Murine-derived CDR sequences
DTLRLPS

SEQ ID NO: 1407 - protein, 7 aa, Artificial Sequence
  Murine-derived CDR sequences
DTLRLTS

SEQ ID NO: 1408 - protein, 7 aa, Artificial Sequence
  Murine-derived CDR sequences
DTLRLDS

SEQ ID NO: 1409 - protein, 7 aa, Artificial Sequence
  Murine-derived CDR sequences
DTLRHAS

SEQ ID NO: 1410 - protein, 7 aa, Artificial Sequence
  Murine-derived CDR sequences
DTLRHSS

SEQ ID NO: 1411 - protein, 7 aa, Artificial Sequence
  Murine-derived CDR sequences
DTLRHKS

SEQ ID NO: 1412 - protein, 7 aa, Artificial Sequence
  Murine-derived CDR sequences
DTLRHRS

SEQ ID NO: 1413 - protein, 7 aa, Artificial Sequence
  Murine-derived CDR sequences
DTLRHHS

SEQ ID NO: 1414 - protein, 7 aa, Artificial Sequence
  Murine-derived CDR sequences
DTLRHPS

SEQ ID NO: 1415 - protein, 7 aa, Artificial Sequence
  Murine-derived CDR sequences
DTLRHTS

SEQ ID NO: 1416 - protein, 7 aa, Artificial Sequence
  Murine-derived CDR sequences
DTLRHDS

SEQ ID NO: 1417 - protein, 7 aa, Artificial Sequence
  Murine-derived CDR sequences
DTLRQAS

SEQ ID NO: 1418 - protein, 7 aa, Artificial Sequence
  Murine-derived CDR sequences
DTLRQSS

SEQ ID NO: 1419 - protein, 7 aa, Artificial Sequence
  Murine-derived CDR sequences
DTLRQKS

SEQ ID NO: 1420 - protein, 7 aa, Artificial Sequence
  Murine-derived CDR sequences
DTLRQRS

SEQ ID NO: 1421 - protein, 7 aa, Artificial Sequence
  Murine-derived CDR sequences
DTLRQHS

SEQ ID NO: 1422 - protein, 7 aa, Artificial Sequence
  Murine-derived CDR sequences
DTLRQPS

SEQ ID NO: 1423 - protein, 7 aa, Artificial Sequence
  Murine-derived CDR sequences
DTLRQTS

SEQ ID NO: 1424 - protein, 7 aa, Artificial Sequence
  Murine-derived CDR sequences
DTLRQDS

SEQ ID NO: 1425 - protein, 7 aa, Artificial Sequence
  Murine-derived CDR sequences
DTLYLAS

SEQ ID NO: 1426 - protein, 7 aa, Artificial Sequence
  Murine-derived CDR sequences
DTLYLSS

SEQ ID NO: 1427 - protein, 7 aa, Artificial Sequence
  Murine-derived CDR sequences
DTLYLKS

SEQ ID NO: 1428 - protein, 7 aa, Artificial Sequence
  Murine-derived CDR sequences
DTLYLRS

SEQ ID NO: 1429 - protein, 7 aa, Artificial Sequence
  Murine-derived CDR sequences
DTLYLHS

SEQ ID NO: 1430 - protein, 7 aa, Artificial Sequence
  Murine-derived CDR sequences
DTLYLPS

SEQ ID NO: 1431 - protein, 7 aa, Artificial Sequence
  Murine-derived CDR sequences
DTLYLTS

SEQ ID NO: 1432 - protein, 7 aa, Artificial Sequence
  Murine-derived CDR sequences
DTLYLDS

SEQ ID NO: 1433 - protein, 7 aa, Artificial Sequence
  Murine-derived CDR sequences
DTLYHAS

SEQ ID NO: 1434 - protein, 7 aa, Artificial Sequence
  Murine-derived CDR sequences
DTLYHSS

SEQ ID NO: 1435 - protein, 7 aa, Artificial Sequence
  Murine-derived CDR sequences
DTLYHKS

SEQ ID NO: 1436 - protein, 7 aa, Artificial Sequence
  Murine-derived CDR sequences
DTLYHRS

SEQ ID NO: 1437 - protein, 7 aa, Artificial Sequence
  Murine-derived CDR sequences
DTLYHHS

SEQ ID NO: 1438 - protein, 7 aa, Artificial Sequence
  Murine-derived CDR sequences
DTLYHPS

SEQ ID NO: 1439 - protein, 7 aa, Artificial Sequence
  Murine-derived CDR sequences
DTLYHTS

SEQ ID NO: 1440 - protein, 7 aa, Artificial Sequence
  Murine-derived CDR sequences
DTLYHDS

SEQ ID NO: 1441 - protein, 7 aa, Artificial Sequence
  Murine-derived CDR sequences
DTLYQAS

SEQ ID NO: 1442 - protein, 7 aa, Artificial Sequence
  Murine-derived CDR sequences
DTLYQSS

SEQ ID NO: 1443 - protein, 7 aa, Artificial Sequence
  Murine-derived CDR sequences
DTLYQKS

SEQ ID NO: 1444 - protein, 7 aa, Artificial Sequence
  Murine-derived CDR sequences
DTLYQRS

SEQ ID NO: 1445 - protein, 7 aa, Artificial Sequence
  Murine-derived CDR sequences
DTLYQHS

SEQ ID NO: 1446 - protein, 7 aa, Artificial Sequence
  Murine-derived CDR sequences
DTLYQPS

SEQ ID NO: 1447 - protein, 7 aa, Artificial Sequence
  Murine-derived CDR sequences
DTLYQTS

SEQ ID NO: 1448 - protein, 7 aa, Artificial Sequence
  Murine-derived CDR sequences
DTLYQDS

SEQ ID NO: 1449 - protein, 7 aa, Artificial Sequence
  Murine-derived CDR sequences
DTLFLAS

SEQ ID NO: 1450 - protein, 7 aa, Artificial Sequence
  Murine-derived CDR sequences
DTLFLSS

SEQ ID NO: 1451 - protein, 7 aa, Artificial Sequence
  Murine-derived CDR sequences
DTLFLKS

SEQ ID NO: 1452 - protein, 7 aa, Artificial Sequence
  Murine-derived CDR sequences
DTLFLRS

SEQ ID NO: 1453 - protein, 7 aa, Artificial Sequence
  Murine-derived CDR sequences
DTLFLHS

SEQ ID NO: 1454 - protein, 7 aa, Artificial Sequence
  Murine-derived CDR sequences
DTLFLPS

SEQ ID NO: 1455 - protein, 7 aa, Artificial Sequence
  Murine-derived CDR sequences
DTLFLTS

SEQ ID NO: 1456 - protein, 7 aa, Artificial Sequence
  Murine-derived CDR sequences
DTLFLDS

SEQ ID NO: 1457 - protein, 7 aa, Artificial Sequence
  Murine-derived CDR sequences
DTLFHAS

SEQ ID NO: 1458 - protein, 7 aa, Artificial Sequence
  Murine-derived CDR sequences
DTLFHSS

SEQ ID NO: 1459 - protein, 7 aa, Artificial Sequence
  Murine-derived CDR sequences
DTLFHKS

SEQ ID NO: 1460 - protein, 7 aa, Artificial Sequence
  Murine-derived CDR sequences
DTLFHRS

SEQ ID NO: 1461 - protein, 7 aa, Artificial Sequence
  Murine-derived CDR sequences
DTLFHHS

SEQ ID NO: 1462 - protein, 7 aa, Artificial Sequence
  Murine-derived CDR sequences
DTLFHPS

SEQ ID NO: 1463 - protein, 7 aa, Artificial Sequence
  Murine-derived CDR sequences
DTLFHTS

SEQ ID NO: 1464 - protein, 7 aa, Artificial Sequence
  Murine-derived CDR sequences
DTLFHDS

SEQ ID NO: 1465 - protein, 7 aa, Artificial Sequence
  Murine-derived CDR sequences
DTLFQAS

SEQ ID NO: 1466 - protein, 7 aa, Artificial Sequence
  Murine-derived CDR sequences
DTLFQSS

SEQ ID NO: 1467 - protein, 7 aa, Artificial Sequence
  Murine-derived CDR sequences
DTLFQKS

SEQ ID NO: 1468 - protein, 7 aa, Artificial Sequence
  Murine-derived CDR sequences
DTLFQRS

SEQ ID NO: 1469 - protein, 7 aa, Artificial Sequence
  Murine-derived CDR sequences
DTLFQHS

SEQ ID NO: 1470 - protein, 7 aa, Artificial Sequence
  Murine-derived CDR sequences
DTLFQPS

SEQ ID NO: 1471 - protein, 7 aa, Artificial Sequence
  Murine-derived CDR sequences
DTLFQTS

SEQ ID NO: 1472 - protein, 7 aa, Artificial Sequence
  Murine-derived CDR sequences
DTLFQDS

SEQ ID NO: 1473 - protein, 7 aa, Artificial Sequence
  Murine-derived CDR sequences
DTLLLSS

SEQ ID NO: 1474 - protein, 7 aa, Artificial Sequence
  Murine-derived CDR sequences
DTLLLKS

SEQ ID NO: 1475 - protein, 7 aa, Artificial Sequence
  Murine-derived CDR sequences
DTLLLRS

SEQ ID NO: 1476 - protein, 7 aa, Artificial Sequence
  Murine-derived CDR sequences
DTLLLHS

SEQ ID NO: 1477 - protein, 7 aa, Artificial Sequence
  Murine-derived CDR sequences
DTLLLPS

SEQ ID NO: 1478 - protein, 7 aa, Artificial Sequence
  Murine-derived CDR sequences
DTLLLTS

SEQ ID NO: 1479 - protein, 7 aa, Artificial Sequence
  Murine-derived CDR sequences
DTLLHAS

SEQ ID NO: 1480 - protein, 7 aa, Artificial Sequence
  Murine-derived CDR sequences
DTLLHSS

SEQ ID NO: 1481 - protein, 7 aa, Artificial Sequence
  Murine-derived CDR sequences
DTLLHKS

SEQ ID NO: 1482 - protein, 7 aa, Artificial Sequence
  Murine-derived CDR sequences
DTLLHRS

SEQ ID NO: 1483 - protein, 7 aa, Artificial Sequence
  Murine-derived CDR sequences
DTLLHHS

SEQ ID NO: 1484 - protein, 7 aa, Artificial Sequence
  Murine-derived CDR sequences
DTLLHPS

SEQ ID NO: 1485 - protein, 7 aa, Artificial Sequence
  Murine-derived CDR sequences
DTLLHTS

SEQ ID NO: 1486 - protein, 7 aa, Artificial Sequence
  Murine-derived CDR sequences
DTLLHDS

SEQ ID NO: 1487 - protein, 7 aa, Artificial Sequence
  Murine-derived CDR sequences
DTLLQAS

SEQ ID NO: 1488 - protein, 7 aa, Artificial Sequence
  Murine-derived CDR sequences
DTLLQSS

SEQ ID NO: 1489 - protein, 7 aa, Artificial Sequence
  Murine-derived CDR sequences
DTLLQKS

SEQ ID NO: 1490 - protein, 7 aa, Artificial Sequence
  Murine-derived CDR sequences
DTLLQRS

SEQ ID NO: 1491 - protein, 7 aa, Artificial Sequence
  Murine-derived CDR sequences
DTLLQHS

SEQ ID NO: 1492 - protein, 7 aa, Artificial Sequence
  Murine-derived CDR sequences
DTLLQPS

SEQ ID NO: 1493 - protein, 7 aa, Artificial Sequence
  Murine-derived CDR sequences
DTLLQTS

SEQ ID NO: 1494 - protein, 7 aa, Artificial Sequence
  Murine-derived CDR sequences
DTLLQDS

SEQ ID NO: 1495 - protein, 9 aa, Artificial Sequence
  Murine-derived CDR sequences
FQGSYYPFT

SEQ ID NO: 1496 - protein, 9 aa, Artificial Sequence
  Murine-derived CDR sequences
FQGSWYPFT

SEQ ID NO: 1497 - protein, 11 aa, Artificial Sequence
  Murine-derived CDR sequences
PKSCDKTHTCP

(19) United States
(12) Patent Application Publication
(10) Pub. No.: US 2010/0278929 A1
(43) Pub. Date: Nov. 4, 2010

(54) ANTIBODY FORMULATIONS HAVING OPTIMIZED AGGREGATION AND FRAGMENTATION PROFILES

(75) Inventors: Ziping Wei, North Potamac, MD (US); Guillermo Tous, East Windsor, NJ (US); Mark Schenerman, Reisterstown, MD (US); Christian B. Allan, Brookeville, MD (US)

Correspondence Address: MEDIMMUNE, LLC;Patrick Scott Alban, ONE MEDIMMUNE WAY, GAITHERSBURG, MD, 20878, US

(73) Assignee: Medlmmune, LLC, Gaithersburg, MD (US)

(21) Appl. No.: 12/689,432

(22) Filed: Jan. 19, 2010

Related U.S. Application Data
(63) Continuation of application No. 11/473,537, filed on Jun. 23, 2006, now abandoned.
(60) Provisional application No. 60/693,603, filed on Jun. 23, 2005.
(60) Provisional application No. 60/699,614, filed on Jul. 15, 2005.

(51) Int. Cl. A61K 9/14; A61K 39/395; A61P 31/14; A61K 39/42; A61P 11/00

(52) U.S. Cl. 424/499; 424/130.1; 424/159.1

(57) ABSTRACT

The present invention provides methods of optimizing the production and purification of antibody formulations that immunospecifically bind to antigens of interest and are suitable for parenteral administration to a subject, which formulations exhibit increased stability due to reduced degradation and aggregation of the antibody component on long term storage. Such methods provide formulations that offer multiple advantages over formulations produced by non-optimized methods including less stringent or more readily available transportation/storage conditions, and less frequent dosing or smaller dosage amounts in the therapeutic, prophylactic and diagnostic use of such formulations. The invention further provides methods of utilizing the formulations of the present invention.

The present application is a Continuation of U.S. Ser. No. 11/473,537, filed Jun. 23, 2006, which claims the benefit of U.S. Provisional Ser. No. 60/693,603, filed Jun. 23, 2005 and U.S. Provisional Ser. No. 60/699,614, filed Jul. 15, 2005, each of which are incorporated by reference in their entireties.

### 1. INTRODUCTION

The present invention provides methods of optimizing the production and purification of antibody formulations that immunospecifically bind to antigens of interest and are suitable for parenteral administration to a subject, which formulations exhibit increased stability due to reduced degradation and aggregation of the antibody component on long term storage. Such methods provide formulations that offer multiple advantages over formulations produced by non-optimized methods including less stringent or more readily available transportation/storage conditions, and less frequent dosing or smaller dosage amounts in the therapeutic, prophylactic and diagnostic use of such formulations. The invention further provides methods of utilizing the formulations of the present invention. In a specific embodiment, the invention provides methods of optimizing the production and purification of antibody formulations that immunospecifically bind to RSV antigens, which formulations exhibit increased stability due to reduced degradation and aggregation of the antibody component on long term storage. Such formulations may be used in the diagnostic, therapeutic or prophylactic treatment of RSV infections.

### 2. BACKGROUND OF THE INVENTION

### Respiratory Syncytial Virus

Respiratory infections are common infections of the upper respiratory tract (e.g., nose, ears, sinuses, and throat) and lower respiratory tract (e.g., trachea, bronchial tubes, and lungs). Symptoms of upper respiratory infection include runny or stuffy nose, irritability, restlessness, poor appetite, decreased activity level, coughing, and fever. Viral upper respiratory infections cause and/or are associated with sore throats, colds, croup, and the flu. Clinical manifestations of a lower respiratory infection include shallow coughing that produces sputum in the lungs, fever, and difficulty breathing.

Respiratory syncytial virus (RSV) is one of the leading causes of respiratory disease worldwide. In the United States, it is responsible for tens of thousands of hospitalizations and thousands of deaths per year (see Black, C. P., Resp. Care 2003 48(3):209-31 for a recent review of the biology and management of RSV). Infants and children are most at risk for serious RSV infections which migrate to the lower respiratory system, resulting in pneumonia or bronchiolitis. In fact, 80% of childhood bronchiolitis cases and 50% of infant pneumonias are attributable to RSV. The virus is so ubiquitous and highly contagious that almost all children have been infected by two years of age. Although infection does not produce lasting immunity, reinfections tend to be less severe so that in older children and healthy adults RSV manifests itself as a cold or flu-like illness affecting the upper and/or lower respiratory system, without progressing to serious lower respiratory tract involvement. However, RSV infections can become serious in elderly or immunocompromised adults. (Evans, A. S., eds., 1989, Viral Infections of Humans. Epidemiology and Control, 3^{rd} ed., Plenum Medical Book, New York at pages 525-544; Falsey, A. R., 1991, Infect. Control Hosp. Epidemiol. 12:602-608; and Garvie et al., 1980, Br. Med. J. 281:1253-1254; Hertz et al., 1989, Medicine 68:269-281).

At present, there is no vaccine against RSV, nor is there any effective treatment. Recent clinical data has failed to support the early promise of the antiviral agent ribavirin, which is the only drug approved for treatment of RSV infection (Black, C. P., Resp. Care 2003 48(3):209-31). Consequently, the American Academy of Pediatrics issued new guidelines suggesting that use of ribavirin be restricted to only the most severe cases (Committee on Infectious Disease, American Academy of Pediatrics. 1996. Pediatrics 97:137-140; Randolph, A. G., and E. E. Wang., 1996, Arch. Pediatr. Adolesc. Med. 150:942-947).

While a vaccine or effective treatment have proven elusive, some success has been achieved in the area of prevention for infants at high risk of serious upper and/or lower respiratory tract RSV infection. In particular, there are two immunoglobulin-based therapies approved to protect high-risk infants from serious lower respiratory tract RSV infection, RSV-IGIV (RSV-immunoglobulin intravenous, also known as RespiGam™) and palivizumab (SYNAGIS®). However, neither RSV-IGIV nor palivizumab has been approved for use other than as a prophylactic agent for lower respiratory tract RSV infections.

RSV is easily spread by physical contact with contaminated secretions. The virus can survive for at least half an hour on hands and for hours on countertops and used tissues. The highly contagious nature of RSV is evident from the risk factors associated with contracting serious infections. One of the greatest risk factors is hospitalization, where in some cases in excess of 50% of the staff on pediatric wards were found to be infected (Black, C. P., Resp. Care 2003 48(3):209-31). Up to 20% of these adult infections are asymptomatic but still produce substantial shedding of the virus. Other risk factors include attendance at day care centers, crowded living conditions, and the presence of school-age siblings in the home. Importantly, an agent that is effective at clearing the virus from the upper and/or lower respiratory tract is likely to be effective in preventing its transmission. Thus, one promising approach to preventing serious RSV infections is the development of therapies to clear or block the virus from the upper and/or lower respiratory tract.

Although RSV-IVIG and palivizumab represent significant advances in the prevention of lower respiratory tract RSV infections, neither has demonstrated efficacy at permissible doses against the virus in the upper respiratory tract. In fact, RSV-IVIG failed to clear nasal RSV when administered as a nasal spray in amounts that were effective to clear pulmonary RSV in every animal of the treatment group (Prince et al., U.S. Pat. No. 4,800,078, issued Jan. 24, 1989). The interperitoneal route of administration also failed to clear RSV from the upper respiratory tract with the same efficacy as the lower respiratory tract. It has recently been noted that the immune response elicited by upper respiratory tract infections differs from that induced by lower respiratory infections (van Benten I. J. et al., J. Med. Virol. 2003 October; 71(2):290-7). Thus, a need exists for the prevention and treatment of upper and/or lower respiratory tract RSV infections.

### Otitis Media

Otitis media is an infection or inflammation of the middle ear. This inflammation often begins when infections that cause sore throats, colds, or other respiratory or breathing problems spread to the middle ear. These can be viral or bacterial infections. RSV is the principal virus that has been correlated with otitis media. Seventy-five percent of children experience at least one episode of otitis media by their third birthday. Almost half of these children will have three or more ear infections during their first 3 years. It is estimated that medical costs and lost wages because of otitis media amount to $5 billion a year in the United States (Gates G A, 1996, Cost-effectiveness considerations in otitis media treatment. Otolaryngol Head Neck Sur. 114 (4): 525-530). Although otitis media is primarily a disease of infants and young children, it can also affect adults.

Otitis media not only causes severe pain but may result in serious complications if it is not treated. An untreated infection can travel from the middle ear to the nearby parts of the head, including the brain. Although the hearing loss caused by otitis media is usually temporary, untreated otitis media may lead to permanent hearing impairment. Persistent fluid in the middle ear and chronic otitis media can reduce a child's hearing at a time that is critical for speech and language development. Children who have early hearing impairment from frequent ear infections are likely to have speech and language disabilities.

Although many physicians recommend the use of antibiotics for the treatment of ear infections, antibiotic resistance has become an important problem in effective treatment of the disease. Further, new therapies are needed to prevent or treat viral infections that are associated with otitis media, particularly RSV.

### Asthma and Reactive Airway Disease (RAD)

About 12 million people in the U.S. have asthma and it is the leading cause of hospitalization for children. The Merck Manual of Diagnosis and Therapy (17th ed., 1999).

Asthma is an inflammatory disease of the lung that is characterized by airway hyperresponsiveness (“AHR”), bronchoconstriction (i.e., wheezing), eosinophilic inflammation, mucus hypersecretion, subepithelial fibrosis, and elevated IgE levels. Asthmatic attacks can be triggered by environmental triggers (e.g. acarids, insects, animals (e.g., cats, dogs, rabbits, mice, rats, hamsters, guinea pigs, mice, rats, and birds), fungi, air pollutants (e.g., tobacco smoke), irritant gases, fumes, vapors, aerosols, chemicals, or pollen), exercise, or cold air. The cause(s) of asthma is unknown. However, it has been speculated that family history of asthma (London et al., 2001, Epidemiology 12(5):577-83), early exposure to allergens, such as dust mites, tobacco smoke, and cockroaches (Melen et al., 2001, 56(7):646-52), and respiratory infections (Wenzel et al., 2002, Am J Med, 112(8):672-33 and Lin et al., 2001, J Microbiol Immuno Infect, 34(4):259-64), such as RSV, may increase the risk of developing asthma. A review of asthma, including risk factors, animal models, and inflammatory markers can be found in O'Byrne and Postma (1999), Am. J. Crit. Care. Med. 159:S41-S66, which is incorporated herein by reference in its entirety.

Current therapies are mainly aimed at managing asthma and include the administration of β-adrenergic drugs (e.g. epinephrine and isoproterenol), theophylline, anticholinergic drugs (e.g., atropine and ipratorpium bromide), corticosteroids, and leukotriene inhibitors. These therapies are associated with side effects such as drug interactions, dry mouth, blurred vision, growth suppression in children, and osteoporosis in menopausal women. Cromolyn and nedocromil are administered prophylatically to inhibit mediator release from inflammatory cells, reduce airway hyperresponsiveness, and block responses to allergens. However, there are no current therapies available that prevent the development of asthma in subjects at increased risk of developing asthma. Thus, new therapies with fewer side effects and better prophylactic and/or therapeutic efficacy are needed for asthma.

Reactive airway disease is a broader (and often times synonymous) characterization for asthma-like symptoms, and is generally characterized by chronic cough, sputum production, wheezing or dyspenea.

### Wheezing

Wheezing (also known as sibilant rhonchi) is generally characterized by a noise made by air flowing through narrowed breathing tubes, especially the smaller, tight airways located deep within the lung. It is a common symptom of RSV infection, and secondary RSV conditions such as asthma and brochiolitis. The clinical importance of wheezing is that it is an indicator of airway narrowing, and it may indicate difficulty breathing.

Wheezing is most obvious when exhaling (breathing out), but may be present during either inspiration (breathing in) or exhalation. Wheezing most often comes from the small bronchial tubes (breathing tubes deep in the chest), but it may originate if larger airways are obstructed.

Citation or discussion of a reference herein shall not be construed as an admission that such is prior art to the present invention.

### 3. SUMMARY OF THE INVENTION

The present invention is based upon the inventors' use of sensitive analytical techniques, such as analytical ultracentrifugation (AUC), size exclusion chromatography (SEC), Liquid Chromatography Mass Spectrometry (LC-MS) or particle counter analysis to analyze the fragmentation and aggregation profiles of formulations of full-length IgG1 monoclonal antibodies, particularly those that have been recombinantly expressed in myeloma cells, such as, but not limited to, NS0 cells. Thus, the present invention provides antibody formulations having fragmentation and aggregation profiles that are improved (i.e., have decreased total fragmentation and/or aggregation or have decreased amounts of certain types of fragments or aggregates or have reduced rates of aggregation or fragmentation) as compared to prior antibody formulations.

In a particular embodiment, the invention provides an antibody formulation comprising a full length IgG₁ antibody, preferably specific for a therapeutic or prophylactic target, wherein no more than 0.5% of the total protein fraction (in certain embodiments, however, at least 0.1% of the total protein fraction or is below detectable levels) of said formulation comprises (or, in other embodiments, consists of as impurities or as fragments to detectable levels) antibody type I fragments. In other embodiments, no more than 0.5% of the total protein fraction (and, in certain embodiments, at least 0.1% of the total protein fraction or is below detectable levels) of said formulation comprises (or, in other embodiments, consists of as impurities or as fragments to detectable levels) antibody type I fragments and antibody type II fragments. Preferably, the antibody type I fragments comprise one or more C-terminal portions of the heavy chain of the antibody, which heavy chain C-terminal portion has a molecular weight of about 25.6 kD, about 25.7 kD, about 25.8 kD, about 26.0 kD, or about 26.1 kD as determined by Liquid Chromatography Mass Spectrometry (LC-MS) analysis of samples of the antibody that have been deglycosylated, reduced and alkylated. Moreover, the antibody type II fragments comprise one or more N-terminal portions of the heavy chain of the antibody, which heavy chain N-terminal portion has a molecular weight of about 24.4 kD, about 24.6 kD, about 24.7 kD, about 24.9 kD, or about 25.1 kD as determined by LC-MS analysis of samples of the antibody that have been deglycosylated, reduced and alkylated. In addition, the antibody type I fragments may comprise one or more C-terminal portions of the heavy chain, which heavy chain C-terminal portion comprises amino acid residues 223-449 of the IgG1 heavy chain (according to Kabat numbering), amino acid residues 224-449 of the IgG1 heavy chain, amino acid residues 225-449 of the IgG1 heavy chain, amino acid residues 226-449 of the IgG1 heavy chain, amino acid residues 227-449 of the IgG1 heavy chain, amino acid residues 228-449 of the IgG1 heavy chain and amino acid residues 229-449 of the IgG1 heavy chain and the antibody type II fragments comprise one or more heavy chain N-terminal portions which comprises amino acid residues 1-222 of the IgG1 heavy chain, amino acid residues 1-223 of the IgG1 heavy chain, amino acid residues 1-224 of the IgG1 heavy chain, amino acid residues 1-225 of the IgG1 heavy chain, amino acid residues 1-226 of the IgG1 heavy chain, amino acid residues 1-227 of the IgG1 heavy chain or amino acid residues 1-228 of the IgG1 heavy chain. In certain embodiments, the antibody formulation does not contain detectable levels of any other types of fragments. In certain embodiments, the antibody formulation contains one, two, three, four, five, six or seven of the type I fragments and/or contains one, two, three, four, five, six or seven of the type II fragments.

In particular embodiments, the formulations of the invention comprise (or consists of as the aggregate fraction) a particle profile of less than about 3.4 E+5 particles/ml of diameter 2-4 μm, less than about 4.0 E+4 particles/ml of diameter 4-10 μm, less than about 4.2 E+3 particles/ml of diameter 10-20 μm, less than about 5.0 E+2 particles/ml of diameter 20-30 μm, less than about 7.5 E+1 particles/ml of diameter 30-40 μm, and less than about 9.4 particles/ml of diameter 40-60 μm as determined by a particle multisizer. In certain embodiments, the formulation contains no detectable particles greater than 40 μm, or greater than 30 μm. In other embodiments, the formulations of the invention have a turbidity value of a degassed sample of said formulation of about 6.4 NTU (in certain embodiments from 4-8 NTU, in other embodiments less than 10 NTU, less than 8 NTU, less than 7 NTU, or less than 6.5 NTU).

The antibody formulations of the invention may likewise have a combination of one or more of the above parameters of fragmentation and aggregation.

The antibody formulations of the invention are preferably at least 10 mg/ml antibody, more preferably, 15 mg/ml, 25 mg/ml, 50 mg/ml, 75 mg/ml, 100 mg/ml, 150 mg/ml or 200 mg/ml. The antibody in the antibody formulations of the invention may be any antibody that has a therapeutic, prophylactic or diagnostic utility. In preferred embodiments, the antibody in the formulations of the invention is specific for RSV and, in a specific embodiment, is not palivizumab. In more specific and preferred embodiments, the anti-RSV antigen binds to the F protein of RSV, and, in particular embodiments, the RSV antigen comprises or even consists of the F protein epitope NSELLSLINDMPITNDQKKLMSNN (SEQ ID NO:337). In other embodiments, the antibody is one of the antibodies listed in Table 2, preferably is the antibody A4B4L1FR-S28R or competes for binding with one of the antibodies listed in Table 2, preferably A4B4L1FR-S28R.

The antibody formulations of the invention preferably maintain improved aggregation and fragmentation profiles upon storage, for example, for extended periods (for example, but not limited to 6 months, 1 year, 2 years, 3 years or 5 years) at room temperature or 4° C. or for periods (such as, but not limited to 1 week, 2 weeks, 3 weeks, 1 month, 2 months, 3 months, 6 months or 1 year) at elevated temperatures such as 38° C.-42° C. Such formulations may be at pH 5-7, preferably at pH 6.0. Thus, in a particular embodiment, an antibody formulation of the invention comprising a full length IgG₁ antibody, upon storage at 38-42° C., pH 6.0 for 1 month, 6 months, 9 months or 14 months, comprises or, alternatively consists (other than the full length IgG1 antibody or as the fragment fraction) one or more antibody type I fragments. In another particular embodiment, an antibody formulation of the invention comprising a full length IgG₁ antibody, upon storage at 38-42° C., pH 6.0 for 1 month, 6 months, 9 months or 14 months, comprises or, alternatively consists (other than the full length IgG1 antibody or as the fragment fraction) one or more antibody type I fragments and one or more antibody type II fragments. Upon storage, the level of fragments as a percentage of the total amount of protein is preferably less than 0.5% and, in certain embodiments is at least 0.1% or is below detectable levels of fragments.

Additionally, during storage, such formulations preferably exhibit constant aggregation and fragmentation rates at temperatures, such as, but not limited to, 0-4° C., 10-15°, 20-24° C. room temperature, or elevated temperatures 38-42° C., and extended periods, such as, but not limited to, two weeks, one month, six months, one year, three years or five years. In certain embodiments, the antibody formulation does not contain detectable levels of any other types of fragments. Thus, in a particular embodiment, an antibody formulation of the invention comprising a full length IgG1 will increase in aggregate percentage relative to total protein, by 0.2%/month-0.35%/month at 20-24° C. and preferably by not more than 0.02%/month at 4° C. In a further embodiment, an antibody formulation of the invention comprising a full length IgG1 will not increase in fragment percentage, relative to total protein, by more than 0.015%/month-0.03%/month at 20-24° C. and preferably by not more than 0.00%/month at 4° C. In certain embodiments, the antibody formulation contains one, two, three, four, five or six or the type I fragments and/or contains one, two, three, four, five, six or seven of the type II fragments.

In particular embodiments, after storage, the formulations of the invention comprise (or consists of as the aggregate fraction) a particle profile of less than about 3.4 E+5 particles/ml of diameter 2-4 μm, less than about 4.0 E+4 particles/ml of diameter 4-10 μm, less than about 4.2 E+3 particles/ml of diameter 10-20 μm, less than about 5.0 E+2 particles/ml of diameter 20-30 μm, less than about 7.5 E+1 particles/ml of diameter 30-40 μm, and less than about 9.4 particles/ml of diameter 40-60 μm as determined by a particle multisizer. In certain embodiments, the formulation contains no detectable particles greater than 40 μm, or greater than 30 μm. In other embodiments, the formulations of the invention, after storage, have a turbidity value of a degassed sample of said formulation of about 6.4 NTU (in certain embodiments from 4-8 NTU, in other embodiments less than 10 NTU, less than 8 NTU, less than 7 NTU, or less than 6.5 NTU).

The antibody formulations of the invention, after storage, may likewise have a combination of one or more of the above parameters of fragmentation and aggregation.

Other aspects of the invention provide for methods of optimizing a particular antibody formulation for the fragmentation and aggregation parameters set forth above. Such methods comprise production, purification and formulation of the antibody and monitoring at one or more steps, or of the final formulation, for the levels of fragmentation and/or aggregation using methods such as, but not limited to AUC, SEC, LC-MS or particle multisizing, and then varying one or more parameters of one or more steps of the production, purification and/or formulation process or the formulation itself and evaluating whether varying the parameter reduces the level of fragmentation and/or aggregation. By such screening and monitoring steps, the method of the invention may be used to optimize an antibody formulation. Such parameters include, the temperature at which one or more steps is carried out, the reduction in or elimination of freeze/thaw cycles of the antibody, introduction of filtration steps, such as ultrafiltration, addition or change in one or more column chromatography steps, change in pH, etc.

The invention provides an antibody comprising a Fab fragment, which immunospecifically binds to an RSV antigen (e.g., the F protein epitope NSELLSLINDMPITNDQKKLMSNN (SEQ ID NO:337)), wherein the Tm of the Fab fragment is at least about 87° C., and wherein said antibody is not any of palivizumab, AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, and A17h4. In a specific embodiment, the Fab in such an antibody is different from the Fab of palivizumab. In another embodiment, such an antibody comprises a VH or VL domain that is different from the VH or VL domain of palivizumab. In preferred embodiment, the Tm of the Fab fragment is at least about 90° C. or at least about 93° C. In another preferred embodiment, the pI of the antibody is between about 8.5 to 9.5 or between about 9.0 to 9.5.

In another specific embodiment, the antibody comprises a VH domain of the antibody A4B4L1FR-S28R (SEQ ID NO:48). In still another embodiment, the antibody comprises a VL domain of the antibody A4B4L1FR-S28R (SEQ ID NO:11). In still another embodiment, said Fab is the Fab of antibody A4B4L1FR-S28R.

The invention also provides an antibody formulation comprising the above described antibody, said formulation having a viscosity of less than 10.00 cP at any temperature in the range of 1 to 26° C.

The invention also provides an antibody formulation comprising the above described antibody, said formulation having an aggregration rate of less than 15% per day at any temperature in the range of 38 to 42° C.

The invention also provides a method of preventing, treating, or ameliorating one or more symptoms associated with a RSV infection in a subject, e.g., otitis media, asthma, and wheezing, said method comprising administering a prophylactically or therapeutically effective amount of an antibody formulation comprising such antibody. In one embodiment, the formulation is administered parenterally, intramuscularly, intravenously, subcutaneously or intranasally.

The invention also provides an antibody formulation comprising a full length IgG₁ antibody, which immunospecifically binds to an RSV antigen, said formulation having a viscosity of less than 10.00 cP at any temperature in the range of 1 to 26° C. The invention also provides an antibody formulation comprising any such antibody, said formulation having an aggregration rate of less than 15% per day at any temperature in the range of 38 to 42° C. In one embodiment, the antibody is not palivizumab. In another embodiment, the antibody is not any of AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, and A17h4.

### 3.1 TERMINOLOGY

In the context of a polypeptide, the term “analog” as used herein refers to a polypeptide that possesses a similar or identical function as a RSV polypeptide, a fragment of a RSV polypeptide, or an antibody but does not necessarily comprise a similar or identical amino acid sequence of a RSV polypeptide, a fragment of a RSV polypeptide, or an antibody, or possess a similar or identical structure of a RSV polypeptide, a fragment of a RSV polypeptide, or an antibody. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a RSV polypeptide, a fragment of a RSV polypeptide, or an antibody described herein; (b) a polypeptide encoded by a nucleotide sequence that hybridizes under stringent conditions to a nucleotide sequence encoding a RSV polypeptide, a fragment of a RSV polypeptide, or an antibody described herein of at least 5 amino acid residues, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a RSV polypeptide, a fragment of a RSV polypeptide, or an antibody described herein. A polypeptide with similar structure to a RSV polypeptide, a fragment of a RSV polypeptide, or an antibody described herein refers to a polypeptide that has a similar secondary, tertiary or quaternary structure of a RSV polypeptide, a fragment of a RSV, or an antibody described herein. The structure of a polypeptide can determined by methods known to those skilled in the art, including but not limited to, X-ray crystallography, nuclear magnetic resonance, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X 100%). In one embodiment, the two sequences are the same length.

The determination of percent identity between two sequences can also be accomplished using a mathematical algorithm. A preferred, non limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score 50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., http://www.ncbi.nlm.nih.gov). Another preferred, non limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The terms “antibodies that immunospecifically bind to a RSV antigen” and analogous terms as used herein refers to antibodies that specifically bind to a RSV polypeptide or a fragment of a RSV polypeptide and do not non-specifically bind to other polypeptides. Antibodies that immunospecifically bind to a RSV polypeptide or fragment thereof may have cross-reactivity with other antigens. Preferably, antibodies that immunospecifically bind to a RSV polypeptide or fragment thereof do not cross-react with other antigens. Antibodies that immunospecifically bind to a RSV polypeptide can be identified, for example, by immunoassays or other techniques known to those of skill in the art.

Antibodies of the invention include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific and bi-specific, etc.), Fab fragments, F(ab′) fragments, disulfide-linked Fvs (sdFv), anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen-binding site that immunospecifically binds to a RSV antigen (preferably, a RSV F antigen) (e.g., one or more complementarity determining regions (CDRs) of an anti-RSV antibody). The antibodies of the invention can be of any type (e.g., IgG, IgE, IgM, IgD, IgA and IgY), class (e.g., IgG₁, IgG₂, IgG₃, IgG₄, IgA₁ and IgA₂) or a subclass of immunoglobulin molecule.

As used herein, the term “analogue” in the context of a non-proteinaceous analog refers to a second organic or inorganic molecule which possess a similar or identical function as a first organic or inorganic molecule and is structurally similar to the first organic or inorganic molecule.

The term “antibody fragment” as used herein refers to a fragment of an antibody that immunospecifically binds to a RSV antigen. Antibody fragments may be generated by any technique known to one of skill in the art and by proteolytic or non-proteolytic cleavage. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the complete light chain, and the variable region, the CH1 region and the hinge region of the heavy chain. Antibody fragments can be also produced by recombinant DNA technologies. Antibody fragments may be one or more complementarity determining regions (CDRs) of antibodies.

The term “antibody type I fragment” as used herein refers to a multimeric protein comprising a full length antibody light chain, a full length antibody heavy chain and a C-terminal portion of an antibody heavy chain that, in human IgG₁ immunoglobulins, has an N-terminus at cysteine 223, aspartic acid 224, lysine 225, threonine 226, histidine 227, threonine 228 or cysteine 229 and a C-terminus at lysine 449. Amino acid numbering for the constant domain is given according to the Kabat EU numbering scheme (Kabat, E. A., T. T. Wu, H. M. Perry, K. S. Gottesman, and Foeller. 1991. Sequences of Proteins of Immunological Interest, U.S. Public Health Service, National Institutes of Health, Washington, D.C., which is incorporated herein by reference), unless otherwise indicated. In a specific embodiment, the full length antibody light chain, full length antibody heavy chain and C-terminal portion of an antibody heavy chain are linked by disulfide bonds as depicted in FIG. 14. In another specific embodiment, the type I fragment is capable of immunospecifically binding to an antigen of interest.

The term “antibody type II fragment” as used herein refers to a peptide, polypeptide, or protein comprising an antibody light chain and an N-terminal portion of an antibody heavy chain that, in human IgG₁ immunoglobulins, has a C-terminus at serine 222, cysteine 223, aspartic acid 224, lysine 225, threonine 226, histidine 227 or threonine 228 and an N-terminus at glycine 1. In a specific embodiment, the full length antibody light chain and N-terminal portion of an antibody heavy chain are linked by disulfide bonds as depicted in FIG. 14. In another specific embodiment, the type II fragment is capable of immunospecifically binding to an antigen of interest.

In the context of a polypeptide, the term “derivative” as used herein refers to a polypeptide that comprises an amino acid sequence of a RSV polypeptide, a fragment of a RSV polypeptide, or an antibody that immunospecifically binds to a RSV polypeptide which has been altered by the introduction of amino acid residue substitutions, deletions or additions. The term “derivative” as used herein also refers to a RSV polypeptide, a fragment of a RSV polypeptide, or an antibody that immunospecifically binds to a RSV polypeptide which has been modified, i.e., by the covalent attachment of any type of molecule to the polypeptide. For example, but not by way of limitation, a RSV polypeptide, a fragment of a RSV polypeptide, or an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a RSV polypeptide, a fragment of a RSV polypeptide, or an antibody may be modified by chemical modifications using techniques known to those of skill in the art, including, but not limited to specific chemical cleavage, acetylation, formylation, metabolic synthesis of tunicamycin, etc. Further, a derivative of a RSV polypeptide, a fragment of a RSV polypeptide, or an antibody may contain one or more non-classical amino acids. A polypeptide derivative possesses a similar or identical function as a RSV polypeptide, a fragment of a RSV polypeptide, or an antibody described herein.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated.

The term “effective amount” as used herein refers to the amount of a therapy (e.g., an antibody of the invention) which is sufficient to reduce and/or ameliorate the severity and/or duration of a disease or disorder. For example, the “effective amount” of an anti RSV antibody is that which is sufficient to reduce and/or ameliorate the severity and/or duration of an upper and/or lower respiratory tract RSV infection, otitis media, and/or a symptom or respiratory condition relating thereto (including, but not limited to, asthma, wheezing, RAD, or a combination thereof), prevent the advancement or progression of the upper and/or lower respiratory tract RSV infection, otitis media and/or a symptom or respiratory condition relating thereto (e.g., prevent the progression of an upper respiratory tract RSV infection to a lower respiratory tract RSV infection), prevent the recurrence, development, or onset of an upper and/or lower respiratory tract RSV infection, otitis media, and/or a symptom or respiratory condition relating thereto (including, but not limited to, asthma, wheezing, RAD, or a combination thereof), and/or enhance/improve the prophylactic or therapeutic effect(s) of another therapy (e.g., a therapy other than an antibody of the invention). Non-limiting examples of effective amounts of an antibody of the invention are provided in Section 5.3, infra. With respect to the treatment of a RSV infection, a therapeutically effective amount refers to the amount of a therapeutic agent sufficient to reduce or inhibit the replication of a virus, inhibit or reduce the infection of cell with the virus, inhibit or reduce the production of the viral particles, inhibit or reduce the release of viral particles, inhibit or reduce the spread of the virus to other tissues or subjects, or ameliorate one or more symptoms associated with the infection. In a specific embodiment, a therapeutically effective amount of a therapeutic agent reduces one or more of the following steps of a RSV life cycle: the docking of the virus particle to a cell, the introduction of viral genetic information into a cell, the expression of viral proteins, the production of new virus particles and the release of virus particles from a cell by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In another specific embodiment, a therapeutically effective amount of a therapeutic agent reduces the replication, multiplication or spread of a virus by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The term “effective neutralizing titer” of an anti-RSV antibody as used herein refers to the amount of antibody which corresponds to the amount present in the serum of animals (human or cotton rat) that has been shown to be either clinically efficacious (in humans) or to reduce virus by 99% in, for example, cotton rats. The 99% reduction is defined by a specific challenge of, e.g., 10³ pfu, 10⁴ pfu, 10⁵ pfu, 10⁶ pfu, 10⁷ pfu, 10⁸ pfu, or 10⁹ pfu of RSV.

The term “elderly” as used herein refers to a human subject who is age 65 or older.

The term “epitopes” as used herein refers to fragments of a polypeptide having antigenic or immunogenic activity in an animal, preferably a mammal, and most preferably in a human. An epitope having immunogenic activity is a fragment of a polypeptide that elicits an antibody response in an animal. An epitope having antigenic activity is a fragment of a polypeptide to which an antibody immunospecifically binds as determined by any method well known in the art, for example, by the immunoassays described herein. Antigenic epitopes need not necessarily be immunogenic.

The term “excipients” as used herein refers to inert substances which are commonly used as a diluent, vehicle, preservatives, binders, or stabilizing agent for drugs and includes, but not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). Also see Remington's Pharmaceutical Sciences (by Joseph P. Remington, 18th ed., Mack Publishing Co., Easton, Pa.), which is hereby incorporated in its entirety.

The term “fragment” as used herein refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a polypeptide or an antibody that immunospecifically binds to a polypeptide. In a specific embodiment, a fragment of a polypeptide or an antibody of that immunospecifically binds to an antigen retains at least 1, at least 2, or at least 3 functions of the polypeptide or antibody.

The term “fusion protein” as used herein refers to a polypeptide that comprises an amino acid sequence of an antibody and an amino acid sequence of a heterologous polypeptide or protein (i.e., a polypeptide or protein not normally a part of the antibody (e.g., a non-anti-RSV antigen antibody)).

The terms “high concentration” and “concentrated antibody” as used herein refer to a concentration of 50 mg/ml or higher, preferably 95 mg/ml or higher of an antibody or antigen-binding fragment thereof in an antibody formulation.

The term “high potency” as used herein refers to antibodies that exhibit high potency as determined in various assays for biological activity (e.g., neutralization of RSV) such as those described herein. For example, high potency antibodies of the invention have an IC₅₀ value less than 5 nM, less than 4 nM, less than 3 nM, less than 2 nM, less than 1.75 nM, less than 1.5 nM, less than 1.25 nM, less than 1 nM, less than 0.75 nM, less than 0.5 nM, less than 0.25 nM, less than 0.1 nM, less than 0.05 nM, less than 0.025 nM, or less than 0.01 nM, as measured by a microneutralization assay described herein. Further, high potency anti-RSV antibodies of the invention result in at least a 75%, preferably at least a 95% and more preferably a 99% lower RSV titer in a cotton rat 5 days after challenge with 10⁵ pfu relative to a cotton rat not administered said antibodies. In certain embodiments of the invention, high potency anti-RSV antibodies of the present invention exhibit a high affinity and/or high avidity for one or more RSV antigens (e.g., antibodies having an affinity of at least 2×10⁸ M⁻¹, preferably at least 2.5×10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, or at least 5×10¹² M⁻¹ for one or more RSV antigens).

The term “host” as used herein refers to an animal, preferably a mammal, and most preferably a human.

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “human infant” as used herein refers to a human less than 24 months, preferably less than 16 months, less than 12 months, less than 6 months, less than 3 months, less than 2 months, or less than 1 month of age.

The term “human infant born prematurely” as used herein refers to a human born at less than 40 weeks gestational age, preferably less than 35 weeks gestational age, who is less than 6 months old, preferably less than 3 months old, more preferably less than 2 months old, and most preferably less than 1 month old.

As used herein, the term “in combination” refers to the use of more than one therapy. The use of the term “in combination” does not restrict the order in which therapies are administered to a subject with an infection. A first therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject which had, has, or is susceptible to a disease or disorder. Any additional therapy can be administered in any order with the other additional therapies. In certain embodiments, the antibodies of the invention can be administered in combination with one or more non-antibody therapies. Non-limiting examples of therapies that can be administered in combination with an antibody of the invention include analgesic agents, anesthetic agents, antibiotics, or immunomodulatory agents.

As used herein, the term “infection” refers to all stages of RSV's life cycle in a host (including, but not limited to the invasion by and replication of RSV in a cell or body tissue), and the pathological state resulting from the invasion by and replication of a RSV. The invasion by and multiplication of a RSV includes, but is not limited to, the following steps: the docking of the RSV particle to a cell, the introduction of viral genetic information into a cell, the expression of RSV proteins, the production of new RSV particles and the release of RSV particles from a cell.

The term “inorganic salt” as used herein refers to any compounds containing no carbon that result from replacement of part or all of the acid hydrogen or an acid by a metal or a group acting like a metal and are often used as a tonicity adjusting compound in pharmaceutical compositions and preparations of biological materials. The most common inorganic salts are NaCl, KCl, NaH₂PO₄, etc.

An “isolated” or “purified” antibody is substantially free of cellular material or other contaminating proteins from the cell or tissue source from which the protein is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). When the antibody is recombinantly produced, it is also preferably substantially free of culture medium, i.e., culture medium represents less than about 20%, 10%, or 5% of the volume of the protein preparation. When the antibody is produced by chemical synthesis, it is preferably substantially free of chemical precursors or other chemicals, i.e., it is separated from chemical precursors or other chemicals which are involved in the synthesis of the protein. Accordingly such preparations of the antibody have less than about 30%, 20%, 10%, 5% (by dry weight) of chemical precursors or compounds other than the antibody of interest. In a preferred embodiment, antibodies of the invention are isolated or purified.

An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, a nucleic acid molecule(s) encoding an antibody of the invention is isolated or purified.

The phrase “low to undetectable levels of aggregation” as used herein refers to samples containing no more than 5%, no more than 4%, no more than 3%, no more than 2%, no more than 1% and most preferably no more than 0.5% aggregation by weight of protein as measured by high performance size exclusion chromatography (HPSEC) or by a multi-sizer.

The term “low to undetectable levels of fragmentation” as used herein refers to samples containing equal to or more than 95%, 98%, 99%, 99.5% or 99.9% of the total protein, for example, as determined by AUC or LC-MS.

The term “lower respiratory” tract refers to the major passages and structures of the lower respiratory tract including the windpipe (trachea) and the lungs, including the bronchi, bronchioles, and alveoli of the lungs.

As used herein, the term “low tolerance” refers to a state in which the patient suffers from side effects from a therapy so that the patient does not benefit from and/or will not continue therapy because of the adverse effects and/or the harm from side effects outweighs the benefit of the therapy.

As used herein, the terms “manage”, “managing” and “management” refer to the beneficial effects that a subject derives from a therapy (e.g., a prophylactic or therapeutic agent), which does not result in a cure of the infection. In certain embodiments, a subject is administered one or more therapies (e.g., prophylactic or therapeutic agents) to “manage” a infection, one or more symptoms thereof, or a respiratory condition associated with, potentiated by, or potentiating a RSV infection, so as to prevent the progression or worsening of the infection.

The terms “non-responsive” and “refractory” as used herein describe patients treated with a currently available therapy (such as but not limited to, a prophylactic or therapeutic agent) for a RSV infection, one or more symptoms thereof, or a respiratory condition associated with, potentiated by, or potentiating a RSV infection, which is not clinically adequate to relieve one or more symptoms associated with the infection. Typically, such patients suffer from severe, persistently active infection and require additional therapy to ameliorate the symptoms associated with their infection or respiratory condition.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogues of DNA or RNA molecules. Such analogues can be generated using, for example, nucleotide analogues, which include, but are not limited to, inosine or tritylated bases. Such analogues can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

The term “pharmaceutically acceptable” as used herein means being approved by a regulatory agency of the Federal or a state government, or listed in the U.S. Pharmacopia, European Pharmacopia or other generally recognized pharmacopia for use in animals, and more particularly in humans.

The term “polyol” as used herein refers to a sugar that contains many —OH groups compared to a normal saccharide.

As used herein, the terms “prevent,” “preventing,” and “prevention” refer to the prevention or inhibition of the development or onset of a disease or disorder, such as an upper and/or lower respiratory tract RSV infection, otitis media or a respiratory condition related thereto in a subject, the prevention or inhibition of the progression of an upper respiratory tract RSV infection to a lower respiratory tract RSV infection, otitis media or a respiratory condition related thereto resulting from the administration of a therapy (e.g., a prophylactic or therapeutic agent), the prevention of a symptom of an upper and/or lower tract RSV infection, otitis media or a respiratory condition related thereto, or the administration of a combination of therapies (e.g., a combination of prophylactic or therapeutic agents).

As used herein, the term “prophylactic agent” refers to any agent that can prevent the recurrence, spread or onset of a disease or disorder, such as an upper and/or lower respiratory tract RSV infection, otitis media, or a symptom or respiratory condition relating thereto (including, but not limited to, asthma, wheezing, RAD, or a combination thereof), and/or prevent the progression of an upper respiratory tract RSV infection to a lower respiratory tract RSV infection or otitis media. In certain embodiments, the term “prophylactic agent” refers to an antibody of the invention. In certain other embodiments, the term “prophylactic agent” refers to an agent other than an antibody of the invention. Preferably, a prophylactic agent is an agent which is known to be useful to or has been or is currently being used to prevent or impede the onset, development, progression and/or severity of a RSV infection (preferably an upper and/or lower respiratory tract RSV infection) otitis media, and/or a symptom or respiratory condition related thereto.

In certain embodiments of the invention, a “prophylactically effective serum titer” is the serum titer in a subject, preferably a human, that reduces the incidence of an upper and/or lower respiratory tract RSV infection, otitis media and/or a symptom or respiratory condition related thereto in said subject. In some embodiments, the prophylactically effective serum titer prevents the progression of an upper respiratory tract RSV infection to a lower respiratory tract RSV infection or otitis media. Preferably, the prophylactically effective serum titer reduces the incidence of RSV infections in humans with the greatest probability of complications resulting from RSV infection (e.g., a human with cystic fibrosis, bronchopulmonary dysplasia, congenital heart disease, congenital immunodeficiency or acquired immunodeficiency, a human who has had a bone marrow transplant, a human infant, or an elderly human). In certain other embodiments of the invention, a “prophylactically effective serum titer” is the serum titer in a cotton rat that results in a RSV titer 5 days after challenge with 10⁵ pfu that is 99% lower than the RSV titer 5 days after challenge with 10⁵ pfu of RSV in a cotton rat not administered an antibody that immunospecifically binds to a RSV antigen.

As used herein, the term “refractory” refers to an upper and/or lower respiratory tract RSV infection, otitis media or a respiratory condition related thereto that is not responsive to one or more therapies (e.g., currently available therapies). In a certain embodiment, an upper and/or lower respiratory tract RSV infection, otitis media or a respiratory condition related thereto is refractory to a therapy means that at least some significant portion of the symptoms associated with said upper and/or lower respiratory tract RSV infection, otitis media or a respiratory condition related thereto are not eliminated or lessened by that therapy. The determination of whether an upper and/or lower respiratory tract RSV infection, otitis media or a respiratory condition related thereto is refractory can be made either in vivo or in vitro by any method known in the art for assaying the effectiveness of therapy for the infection, otitis media or the respiratory condition related thereto.

The term “RSV antigen” refers to a RSV polypeptide to which an antibody immunospecifically binds. A RSV antigen also refers to an analog or derivative of a RSV polypeptide or fragment thereof to which an antibody immunospecifically binds.

The term “serum titer” as used herein refers to an average serum titer in a population of least 10, preferably at least 20, and most preferably at least 40 subjects.

The term “saccharide” as used herein refers to a class of molecules that are derivatives of polyhydric alcohols. Saccharides are commonly referred to as carbohydrates and may contain different amounts of sugar (saccharide) units, e.g., monosaccharides, disaccharides and polysaccharides.

As used herein, the term “side effects” encompasses unwanted and adverse effects of a therapy (e.g., a prophylactic or therapeutic agent). Adverse effects are always unwanted, but unwanted effects are not necessarily adverse. An adverse effect from a therapy (e.g., a prophylactic or therapeutic agent) might be harmful or uncomfortable or risky. Examples of side effects include, but are not limited to, nausea, vomiting, anorexia, abdominal cramping, fever, pain, loss of body weight, dehydration, alopecia, dyspnea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physician's Desk Reference (58^{th} ed., 2004).

The terms “stability” and “stable” as used herein in the context of a formulation comprising an antibody or antigen-binding fragment refer to the resistance of the antibody or antibody fragment in the formulation to thermal and chemical unfolding, aggregation, degradation or fragmentation under given manufacture, preparation, transportation and storage conditions. The “stable” formulations of the invention retain biological activity equal to or more than 80%, 85%, 90%, 95%, 98%, 99%, 99.5%, or 99.9% under given manufacture, preparation, transportation and storage conditions. The stability of the antibody or antibody fragment can be assessed by degrees of aggregation, degradation or fragmentation or levels of particular fragments (e.g., Fragment Type I or Fragment Type II) or types or sizes of aggregates by methods known to those skilled in the art, including but not limited to reduced AUC, SEC, LC-MS, particle multisizer Capillary Gel Electrophoresis (rCGE), Sodium Dodecyl Sulfate Polyacrylamide Gel Electrophoresis (SDS-PAGE) and HPSEC, compared to a reference, for example, a commercially available lyophilized palivizumab reconstituted to 100 mg/ml in 50 mM histidine/3.2 mM glycine buffer with 6% mannitol at pH 6.0. The reference regularly gives a single peak (≧97% area) by HPSEC. The overall stability of a formulation comprising an antibody or fragment thereof that immunospecifically binds to a RSV antigen can be assessed by various immunological assays including, for example, ELISA and radioimmunoassay, using the specific epitope of RSV.

As used herein, the terms “subject” and “patient” are used interchangeably. As used herein, a subject is preferably a mammal such as a non-primate (e.g., cows, pigs, horses, cats, dogs, rats, etc.) and a primate (e.g., monkey and human), most preferably a human. In one embodiment, the subject is a mammal, preferably a human, with an upper and/or lower respiratory tract RSV infection or otitis media. In another embodiment, the subject is a mammal, preferably a human, at risk of developing an upper and/or lower respiratory tract RSV infection or otitis media (e.g., an immunocompromised or immunosuppressed mammal, or a genetically predisposed mammal). In one embodiment, the subject is a human with a respiratory condition (including, but not limited to asthma, wheezing or RAD) that stems from, is caused by or associated with a RSV infection.

As used herein, the term “palivizumab standard reference” or analogous terms refer to commercially available lyophilized palivizumab, as described in the Physicians' Desk Reference, 56^{th} edition, 2002. Reconstituted palivizumab may contain, e.g., the following excipients: 47 mM histidine, 3.0 mM glycine and 5.6% manitol and the active ingredient, the antibody, at a concentration of 100 milligrams per ml solution.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a non-primate (e.g., a monkey such as a cynomolgous monkey and a human), and more preferably a human.

The term “substantially free of surfactant” as used herein refers to a formulation of an antibody or fragment thereof that immunospecifically binds to a RSV antigen, said formulation containing less than 0.0005%, less than 0.0003%, or less than 0.0001% of surfactants and/or less than 0.0005%, less than 0.0003%, or less than 0.0001% of surfactants.

The term “substantially free of salt” as used herein refers to a formulation of an antibody or fragment thereof that immunospecifically binds to a RSV antigen, said formulation containing less than 0.0005%, less than 0.0003%, or less than 0.0001% of inorganic salts.

The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials.

The term “synergistic” as used herein refers to a combination of therapies (e.g., use of prophylactic or therapeutic agents) which is more effective than the additive effects of any two or more single therapy. For example, a synergistic effect of a combination of prophylactic or therapeutic agents permits the use of lower dosages of one or more of the agents and/or less frequent administration of said agents to a subject with a RSV infection. The ability to utilize lower dosages of prophylactic or therapeutic therapies and/or to administer said therapies less frequently reduces the toxicity associated with the administration of said therapies to a subject without reducing the efficacy of said therapies in the prevention, management or treatment of a RSV infection. In addition, a synergistic effect can result in improved efficacy of therapies in the prevention or treatment of a RSV infection. Finally, synergistic effect of a combination of therapies (e.g., prophylactic or therapeutic agents) may avoid or reduce adverse or unwanted side effects associated with the use of any single therapy.

As used herein, the term “therapeutic agent” refers to any agent that can be used in the treatment, management, prevention or amelioration of a disease or disorder, for example, an upper and/or lower respiratory tract RSV infection, otitis media or a respiratory condition related thereto. In certain embodiments, the term “therapeutic agent” refers to an antibody of the invention. In certain other embodiments, the term “therapeutic agent” refers to an agent other than an antibody of the invention. Preferably, a therapeutic agent is an agent which is known to be useful for, or has been or is currently being used for the prevention, treatment, management or amelioration of a RSV infection (i.e., an upper and/or lower respiratory tract RSV infection), otitis media, or one or more symptoms or respiratory conditions related thereto.

In certain embodiments of the invention, a “therapeutically effective serum titer” is the serum titer in a subject, preferably a human, that reduces the severity, the duration and/or the symptoms associated with a RSV infection in said subject. Preferably, the therapeutically effective serum titer reduces the severity, the duration and/or the number symptoms associated with upper and/or lower respiratory tract RSV infections in humans with the greatest probability of complications resulting from the infection (e.g., a human with cystic fibrosis, bronchopulmonary dysplasia, congenital heart disease, congenital immunodeficiency or acquired immunodeficiency, a human who has had a bone marrow transplant, a human infant, or an elderly human). In certain other embodiments of the invention, a “therapeutically effective serum titer” is the serum titer in a cotton rat that results in a RSV titer 5 days after challenge with 10⁵ pfu that is 99% lower than the RSV titer 5 days after challenge with 10⁵ pfu of RSV in a cotton rat not administered an antibody that immunospecifically binds to a RSV antigen.

As used herein, the term “therapy” refers to any protocol, method and/or agent that can be used in the prevention, treatment or management of a disease or disorder, such as an RSV infection (i.e., an upper and/or lower respiratory tract RSV infection), otitis media, or a symptom or respiratory condition relating thereto (including, but not limited to, asthma, wheezing, RAD, or a combination thereof). In certain embodiments, the terms “therapies” and “therapy” refer to a biological therapy, supportive therapy, and/or other therapies useful in the treatment, management, prevention and/or amelioration of a RSV infection (i.e., an upper and/or lower respiratory tract RSV infection), otitis media, or a symptom or respiratory condition relating thereto (including, but not limited to, asthma, wheezing, RAD, or a combination thereof) known to one of skill in the art such as medical personnel.

As used herein, the terms “treat,” “treatment” and “treating” refer to the reduction or amelioration of the progression, severity, and/or duration of a disease or disorder, such as an upper and/or lower respiratory tract RSV infection, otitis media, or a symptom or respiratory condition related thereto (such as asthma, wheezing, RAD, or a combination thereof) resulting from the administration of one or more therapies (including, but not limited to, the administration of one or more prophylactic or therapeutic agents). In specific embodiments, such terms refer to the reduction or inhibition of the replication of RSV, the inhibition or reduction in the spread of RSV to other tissues or subjects (e.g., the spread to the lower respiratory tract), the inhibition or reduction of infection of a cell with a RSV, or the amelioration of one or more symptoms associated with an upper and/or lower respiratory tract RSV infection or otitis media.

The term “upper and/or lower respiratory” tract refers to the major passages and structures of the upper and/or lower respiratory tract including the nose or nostrils, nasal cavity, mouth, throat (pharynx), and voice box (larynx).

### 4. DESCRIPTION OF THE FIGURES

FIG. 1 is a schematic diagram showing an outline for preparing purified antibodies that immunospecifically bind to RSV antigen.

FIG. 2 is a schematic diagram showing an outline for preparing purified antibodies that immunospecifically bind to RSV antigen.

FIG. 3A-3B show the amino acid sequences of the (A) light chain variable region (SEQ ID NO:8) and (B) heavy chain variable region (SEQ ID NO:7) of a monoclonal antibody that binds to a RSV antigen, the potency of which can be increased by methods described herein or in Applicants' copending application Ser. Nos. 60/168,426 and 60/186,252 and U.S. Pat. No. 6,656,467. For reference purposes, this is the amino acid sequence of the palivizumab antibody disclosed in Johnson et al., 1997, J. Infect. Dis. 176:1215-1224 and U.S. Pat. No. 5,824,307. Here, the CDR regions are underlined while non-underlined residues form the framework (FR) regions of the variable regions of the antibody. In this antibody, the CDRs are derived from a mouse antibody while the framework regions are derived from a human antibody. The constant regions (not shown) are also derived from a human antibody.

FIG. 4A-4B show the (A) light chain variable region (SEQ ID NO:54) and (B) heavy light chain variable region (SEQ ID NO:343) for an antibody sequence. CDR regions are underlined, and the non-underlined residues form the framework of the variable regions of the antibody. This sequence differs from the sequence disclosed in FIGS. 1A-1B in the first 4 residues of VH CDR1 of the light chain, residue 103 of the light chain FR4 and residue 112 of the heavy chain FR4. For reference purposes, these VL and VH sequences are identical to the VL and VH domains of IX-493L1FR (see Table 2).

FIG. 5A-5B show the nucleotide and translated amino acid sequence of the A4B4L1FR-S28R (A) VH domain (SEQ ID NO:48) and (B) VL domain (SEQ ID NO:11). CDR sequences are underlined. Where palivizumab differs from A4B4L1FR-S28R, the palivizumab amino acid is shown below the motavizumab sequence. Residues that were introduced on the IX-493L1FR template (see also FIG. 2) are indicated in bold.

FIG. 6A-6C. Quantitation of aggregates, fragments and monomers of A4B4L1FR-S28R during storage at (♦) 2-8° C., (□) 20-24° C. and (▴) 38-42° C.; as determined by SEC with UV detection. (A) Percent Aggregates; (B) Percent Fragments and (C) Percent Purity (monomers).

FIG. 7. Plot of Aggregation and fragmentation rates of A4B4L1FR-S28R based on the SEC data of FIGS. 19A-19C; (♦) rate of aggregation, (▪) rate of fragmentation.

FIG. 8. SEC profile of A4B4L1FR-S28R formulated in 25 mM histidine-HCl, pH 6.0 after storage at 38-42° C. for one month.

FIG. 9. Comparison of AUC and SEC analysis of A4B4L1FR-S28R at initial, 9-month and 14-month time points. All samples were formulated in 25 mM histidine-HCl, pH 6.0 and, for the 9 and 14 moth points, stored at 38-42° C. (A) AUC; (B) SEC.

FIG. 10. Comparison of antibody sample concentration dependence of signal/noise ratio for AUC analysis.

FIG. 11. AUC analysis of A4B4L1FR-S28R formulated in 25 mM histidine-HCl, pH 6.0 and stored at 38-42° C. over the course of 5 days.

FIG. 12. LC-MS analysis of deglycosylated, reduced and alkylated antibody type I fragment. Sample collected from SEC of A4B4L1FR-S28R formulated in 25 mM histidine-HCl, pH 6.0 and stored at 38-42° C. for 1 month.

FIG. 13. LC-MS analysis of deglycosylated, reduced and alkylated antibody type II fragment. Sample collected from SEC of A4B4L1FR-S28R formulated in 25 mM histidine-HCl, pH 6.0 and stored at 38-42° C. for 1 month.

FIG. 14A-14B is a diagram showing the characteristic fragmentation pattern of A4B4L1FR-S28R, forming antibody type I and antibody type II fragments. (A) Cleavage sites within the hinge region of the antibody heavy chain (SEQ ID NO:1497). Bold arrows indicate preferred or predominant cleavage sites. (B) Schematic showing characteristics of antibody type I and antibody type II fragments, an outline for preparing purified antibodies that immunospecifically bind to RSV antigen. The antibody type I fragment comprises a full length antibody light chain, a full length antibody heavy chain and a C-terminal portion of an antibody heavy chain that, in human IgG1 immunoglobulins, has an N-terminus at cysteine 223, aspartic acid 224, lysine 225, threonine 226, histidine 227, threonine 228 or cysteine 229. The antibody type II fragment comprises an antibody light chain and an N-terminal portion of an antibody heavy chain that, in human IgG1 immunoglobulins, has a C-terminus at serine 222, cysteine 223, aspartic acid 224, lysine 225, threonine 226, histidine 227 or threonine 228.

FIG. 15. Chromatograms of Lys-C digested aggregates, monomers and fragments collected from SEC of A4B4L1FR-S28R formulated in 25 mM histidine-HCl, pH 6.0 and stored at 38-42° C. for 1 month. The arrows point to the low level disulfide bond scrambling peaks.

FIG. 16. Chromatograms of Lys-C digested aggregates with and without reduction. Samples were collected from SEC of A4B4L1FR-S28R formulated in 25 mM histidine-HCl, pH 6.0 and stored at 38-42° C. for 1 month. The arrows point to the low level disulfide bond scrambling peaks.

FIG. 17 summarizes the results of a RSV microneutralization assay using the anti-RSV antibodies A4B4L1FR-S28R and palivizumab, comparing the ability of both antibodies to inhibit the in vitro replication of RSV (Long) in the assay.

FIG. 18 summarizes the results of a RSV microneutralization assay demonstrating the ability of A4B4L1FR-S28R to inhibit the in vitro replication of RSV (Long) in the microneutralization assay.

FIG. 19 DSC thermograms of the full length palivizumab (top panel) and an overlay of the thermograms obtained from purified Fab and Fc fragments of palivizumab (bottom panel). Two discrete peaks are seen for the Fc domain at approximately 68° C. and 83° C. A single peak is seen for the Fab fragment at approximately 87° C.

FIG. 20 plot of the Tm and pI values of palivizumab and motavizumab.

FIG. 21 plot of the viscosity of a 100 mg/ml solution of palivizumab and motavizumab at a range of temperatures from about 2 to 25° C.

FIG. 22 plot of the aggregation rates of palivizumab and motavizumab against the Fab Tm for each antibody.

### 5. DETAILED DESCRIPTION OF THE INVENTION

### 5.1 Methods of Preparing Antibody Formulations

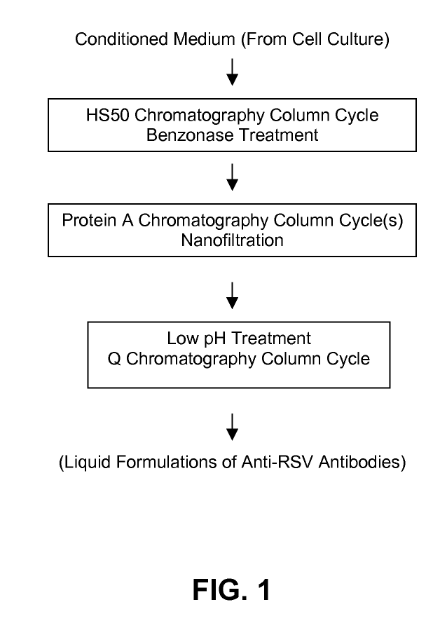

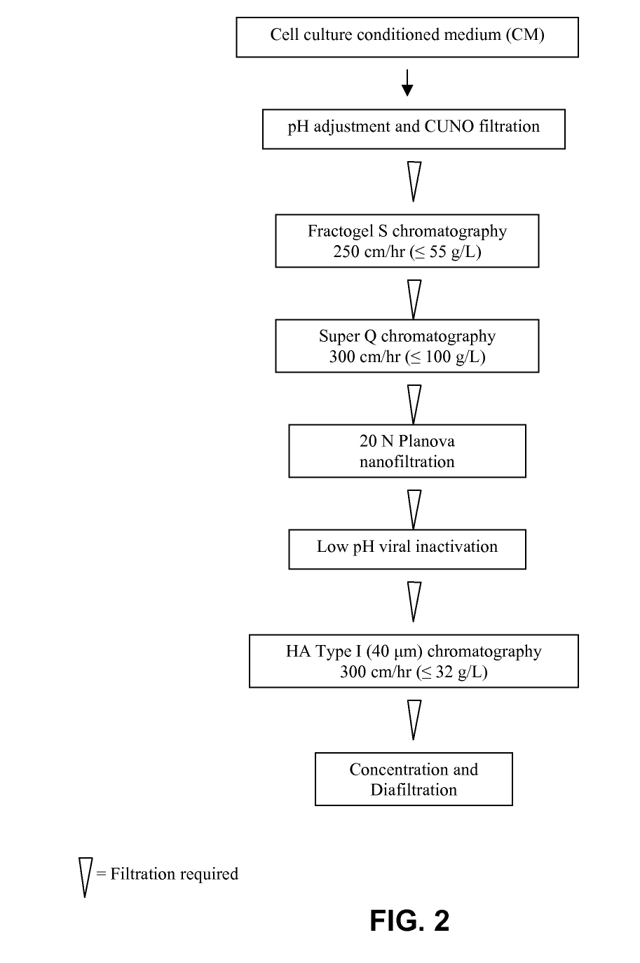

The present invention provides methods for preparing formulations of antibodies, or derivatives, analogues, or fragments thereof that immunospecifically bind to a an antigen of interest. Such antibodies may be purified according to any method known in the art for purification of antibodies. FIGS. 1 and 2 are schematic diagrams showing alternate outlines for preparing purified antibodies. In one embodiment, the methods for preparing liquid formulations of the present invention comprise: concentrating a fraction containing the purified antibody or a fragment to a final antibody or fragment concentration of from about 15 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 110 mg/ml, about 125 mg/ml, about 150 mg/ml, about 200 mg/ml, about 250 mg/ml, or about 300 mg/ml using a semipermeable membrane with an appropriate molecular weight (MW) cutoff (e.g., 30 kD cutoff for whole antibody molecules and F(ab′)₂ fragments; and 10 kD cutoff for antibody fragments, such as Fab fragments) and difiltrating the concentrated antibody fraction into the formulation buffer using the same membrane. Antibodies are preferably expressed in myeloma cells, more preferably murine myeloma cells, most preferably NSO cells.

In the embodiment outlined by FIG. 1, conditioned medium containing antibody or a fragment thereof that immunospecifically binds to an antigen of interest is subjected to CUNO filtration and the filtered antibody is subjected to HS50 cation exchange chromatography. The fraction from the HS50 cation exchange chromatography is then subjected to rProtein A affinity chromatography followed by low pH treatment. Following low pH treatment, the antibody fraction is subject to super Q 650 anion exchange chromatography and then nanofiltration. The fraction of the antibody obtained after nanofiltration is then subjected to diafiltration to concentrate the antibody fraction into the formulation buffer using the same membrane.

Using the embodiment of FIG. 2, conditioned medium containing antibody or a fragment thereof that immunospecifically binds to an antigen of interest is subjected to CUNO filtration and the filtered antibody is subjected to Fractogel® S cation exchange chromatography. The fraction from the cation exchange chromatography is then subjected to super Q anion chromatography, followed by nanofiltration with a Planova® 20 N nanofilter. The antibody fraction recovered after nanofiltration is then subjected to low pH treatment followed by hydroxyapatite (HA) chromatography. The fraction of the antibody obtained after HA chromatography is then subjected to diafiltration to concentrate the antibody fraction into the formulation buffer using the same membrane.

The formulation buffer of the present invention preferably comprises histidine at a concentration ranging from about 1 mM to about 100 mM, about 10 mM to about 50 mM, about 20 mM to about 30 mM, or about 23 mM to about 27 mM. Preferably, the formulation buffer of the present invention comprises histidine at a concentration of about 25 mM. The formulations may further comprise glycine at a concentration of less than 100 mM, less than 50 mM, less than 3.0 mM, less than 2.0 mM, or less than 1.8 mM. Preferably, the formulations comprise glycine at a concentration of 1.6 mM. The amount of glycine in the formulation should not cause a significant buffering in order to avoid antibody precipitation at its isoelectric point. The pH of the formulation may range from about 5.0 to about 7.0, preferably about 5.5 to about 6.5, more preferably about 5.8 to about 6.2, and most preferably about 6.0. To obtain an appropriate pH for a particular antibody, it is preferable that histidine (and glycine, if added) is first dissolved in water to obtain a buffer solution with higher pH than the desired pH and then the pH is brought down to the desired level by adding HCl. This way, the formation of inorganic salts (e.g., formation of NaCl when, for example, histidine hydrochloride is used as histidine and pH is raised to a desired level by adding NaOH) can be avoided.

The formulations of the present invention can be prepared as unit dosage forms by preparing a vial containing an aliquot of the liquid formulation for a one-time use. For example, a unit dosage per vial may contain 1 ml, 2 ml, 3 ml, 4 ml, 5 ml, 6 ml, 7 ml, 8 ml, 9 ml, 10 ml, 15 ml, or 20 ml of different concentrations of an antibody or a fragment thereof that immunospecifically binds to the antigen of interest ranging from about 15 mg/ml to about 300 mg/ml. If necessary, these preparations can be adjusted to a desired concentration by adding a sterile diluent to each vial.

The formulations of the present invention may be sterilized by various sterilization methods, including sterile filtration, radiation, etc. In a most preferred embodiment, the difiltrated antibody formulation is filter-sterilized with a presterilized 0.22-micron filter. In specific embodiments, sterilized liquid formulations of the present invention may be administered to a subject to prevent, treat, manage or ameliorate a RSV infection, one or more symptoms thereof, or a respiratory condition associated with, potentiated by, potentiating a RSV infection.

The formulations of the invention comprise labeled antibodies, derivatives and analogues thereof, that immunospecifically bind to an antigen of interest and can be used for diagnostic purposes to detect, diagnose, or monitor a disorder associated with and/or characterized by the presence or said antigen. In a specific embodiment, the formulations of the invention comprise labeled antibodies, derivatives and analogues thereof, that immunospecifically bind to a RSV antigen and can be used for diagnostic purposes to detect, diagnose, or monitor a RSV infection.

The invention encompasses both liquid and lyophilized forms of the formulations. Methods to produce lyophilized forms of liquid formulations are well-characterized in the art. In one embodiment, the ingredients of formulation of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The formulation of the invention can be further processed into an oral or non-oral dosage form, for immediate or extended release. The formulation can additionally comprise inactive ingredients ordinarily used in pharmaceutical preparation such as diluents, fillers, disintegrants, sweeteners, lubricants and flavors. The formulation may also be processed for intravenous administration, either by bolus injection or sustained drip, or for release from an implanted capsule. A typical formulation for intravenous administration utilizes physiological saline as a diluent.

### 5.2 Formulations of Antibodies

The invention provides formulations comprising antibodies of the invention for use in diagnosing, detecting, or monitoring a disorder, in preventing, treating, managing, or ameliorating of a disorder or one or more symptoms thereof, and/or in research. In a specific embodiment, the formulation of the invention comprises one or more antibodies. In another embodiment, the formulation of the invention comprises one or more antibodies and one or more prophylactic or therapeutic agents other than antibodies. Preferably, the prophylactic or therapeutic agents known to be useful for or having been or currently being used in the prevention, treatment, management, or amelioration of a disorder or one or more symptoms thereof. In accordance with these embodiments, the composition may further comprise of a carrier, diluent or excipient.

The formulations of the present invention provide antibody formulations which are substantially free of surfactant, inorganic salts, and/or other excipients and yet exhibit high stability during long periods of storage. In a specific embodiment, such antibody formulations are homogeneous. The formulations of the present invention comprise histidine at concentrations between 1 and 100 mM and an antibody which immunospecifically binds to a antigen of interest at concentrations of about 15 mg/ml to about 300 mg/ml. In one embodiment, the formulations of the invention do not comprise other ingredients except for water or suitable solvents. In a specific embodiment, the antibody immunospecifically binds to an RSV antigen and in preferred embodiments is not palivizumab or a fragment thereof.

In one embodiment, the antibody of the formulation of the invention is an antibody or antibody fragment conjugated to another moiety, including, but not limited to, a heterologous polypeptide, another antibody or another fragment, a marker sequence, a diagnostic agent, a therapeutic agent, a radioactive metal ion, a polymer, albumin, and a solid support. In another embodiment, formulations of the invention comprise two or more antibodies, or fragments thereof that immunospecifically binds to an antigen of interest. In a specific embodiment, formulations of the invention comprise two or more antibodies, or fragments thereof, that immunospecifically binds to a RSV antigen, wherein at least one of the antibodies or antibody fragments is not palivizumab or a fragment thereof.

The concentration of an antibody or a fragment thereof which is included in the formulations of the invention is at least 15 mg/ml, at least 20 mg/ml, at least 25 mg/ml, at least 30 mg/ml, at least 35 mg/ml, at least 40 mg/ml, at least 45 mg/ml, at least 50 mg/ml, at least 55 mg/ml, at least 60 mg/ml, at least 65 mg/ml, at least 70 mg/ml, at least 75 mg/ml, at least 80 mg/ml, at least 85 mg/ml, at least 90 mg/ml, at least 95 mg/ml, at least 100 mg/ml, at least 105 mg/ml, at least 110 mg/ml, at least 115 mg/ml, at least 120 mg/ml, at least 125 mg/ml, at least 130 mg/ml, at least 135 mg/ml, at least 140 mg/ml, at least 150 mg/ml, at least 200 mg/ml, at least 250 mg/ml, or at least 300 mg/ml.

The concentration of histidine which is included in the formulations of the invention ranges from about 1 mM to about 100 mM, about 10 mM to about 50 mM, about 20 mM to about 30 mM, or about 23 mM to about 27 mM, and is most preferably about 25 mM. Histidine can be in the form of L-histidine, D-histidine, or a mixture thereof, but L-histidine is the most preferable. Histidine can also be in the form of hydrates. Histidine may be used in a form of pharmaceutically acceptable salt, such as hydrochloride (e.g., monohydrochloride and dihydrochloride), hydrobromide, sulfate, acetate, etc. The purity of histidine should be at least 98%, preferably at least 99%, and most preferably at least 99.5%.

The pH of the formulation should not be equal to the isoelectric point of the particular antibody to be used in the formulation and may range from about 5.0 to about 7, preferably about 5.5 to about 6.5, more preferably about 5.8 to about 6.2, and most preferably about 6.0.

In addition to histidine and an antibody or a fragment thereof, the formulations of the present invention may further comprise glycine at a concentration of less than 100 mM, less than 50 mM, less than 3.0 mM, less than 2.0 mM, or less than 1.8 mM, and most preferably 1.6 mM. The amount of glycine in the formulation should not cause a significant buffering effect so that antibody precipitation at its isoelectric point can be avoided. Glycine may be also used in a form of pharmaceutically acceptable salt, such as hydrochloride, hydrobromide, sulfate, acetate, etc. The purity of glycine should be at least 98%, preferably at least 99%, and most preferably 99.5%. In a specific embodiment, glycine is included in the formulations of the present invention.

Optionally, the formulations of the present invention may further comprise other excipients, such as saccharides (e.g., sucrose, mannose, trehalose, etc.) and polyols (e.g., mannitol, sorbitol, etc.). In one embodiment, the other excipient is a saccharide. In a specific embodiment, the saccharide is sucrose, which is at a concentration ranging from between about 1% to about 20%, preferably about 5% to about 15%, and more preferably about 8% to 10%. In another embodiment, the other excipient is a polyol. Preferably, however, the liquid formulations of the present invention do not contain mannitol. In a specific embodiment, the polyol is polysorbate (e.g., Tween 20), which is at a concentration ranging from between about 0.001% to about 1%, preferably, about 0.01 to about 0.1.

The formulations of the present invention exhibit stability at the temperature ranges of 38° C.-42° C. for at least 60 days and, in some embodiments, at least 120 days, of 20° C.-24° C. for at least 1 year, of 2° C.-8° C. (in particular, at 4° C.) for at least 3 years, at least 4 years, or at least 5 years and at −20° C. for at least 3 years, at least 4 years, or at least 5 years, as assessed by AUC, LC-MS, size exclusion chromatography (SEC) or high performance size exclusion chromatography (HPSEC) or particle multisizer. Namely, the formulations of the present invention have low to undetectable levels of aggregation and/or fragmentation, as defined herein, after the storage for the defined periods as set forth above. Preferably, no more than 5%, no more than 4%, no more than 3%, no more than 2%, no more than 1%, and most preferably no more than 0.5% (but in certain embodiments, at least 0.1%) of the antibody or antibody fragment forms an aggregate or fragment (particularly of fragment I or fragment II) as measured by AUC, LC-MS, SEC or HPSEC, after the storage for the defined periods as set forth above. Furthermore, formulations of the present invention exhibit almost no loss in biological activities of the antibody or antibody fragment during the prolonged storage under the condition described above, as assessed by various immunological assays including, but not limited to, enzyme-linked immunosorbent assay (ELISA) and radioimmunoassay to measure the ability of the antibody or antibody fragment to immunospecifically bind to an antigen of interest, and by a C3a/C4a assay to measure the complement activating ability of the antibody. The formulations of the present invention retain after the storage for the above-defined periods more than 80%, more than 85%, more than 90%, more than 95%, more than 98%, more than 99%, or more than 99.5% of the initial biological activities of the formulation prior to the storage.

The formulations of the present invention can be prepared as unit dosage forms. For example, a unit dosage per vial may contain 1 ml, 2 ml, 3 ml, 4 ml, 5 ml, 6 ml, 7 ml, 8 ml, 9 ml, 10 ml, 15 ml, or 20 ml of different concentrations of an antibody or a fragment thereof that immunospecifically binds to a RSV antigen ranging from about 15 mg/ml to about 300 mg/ml. If necessary, these preparations can be adjusted to a desired concentration by adding a sterile diluent to each vial.

The invention encompasses stable liquid formulations comprising a single antibody or fragment thereof that immunospecifically binds to an antigen of interest. In a specific embodiment, the invention encompasses stable liquid formulations comprising a single antibody or fragment thereof that immunospecifically binds to a RSV antigen, with the proviso that said antibody is not palivizumab. The invention also encompasses stable liquid formulations comprising two or more antibodies or fragments thereof that immunospecifically bind to a RSV antigen. In one embodiment, a stable liquid formulation of the invention comprises two or more antibodies or fragments thereof that immunospecifically bind to a RSV antigen, wherein one of the antibodies or antibody fragments is not palivizumab or a fragment thereof.

### 5.3 Antibodies Useful in the Formulations of the Invention

The antibodies useful in the present invention include, but are not limited to, monoclonal antibodies, synthetic antibodies, multispecific antibodies (including bi-specific antibodies), human antibodies, humanized antibodies, chimeric antibodies, single-chain Fvs (scFv) (including bi-specific scFvs), single chain antibodies, Fab fragments, F(ab′) fragments, disulfide-linked Fvs (sdFv), and epitope-binding fragments of any of the above. In particular, antibodies of the present invention include immunoglobulin molecules and immunologically active portions of immunoglobulin molecules, i.e., molecules that contain an antigen binding site that immunospecifically binds to an antigen. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. Preferably, the antibodies of the invention are IgG, more preferably, IgG₁.

The antibodies useful in the present invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice or other animal that express antibodies from human genes.

The antibodies useful in the present invention may be monospecific, bispecific, trispecific or of greater multispecificity. Multispecific antibodies may immunospecifically bind to different epitopes of a polypeptide or may immunospecifically bind to both a polypeptide as well a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

The antibodies useful in the present invention include derivatives of the antibodies. Standard techniques known to those of skill in the art can be used to introduce mutations in the nucleotide sequence encoding an antibody to be used with the methods of the invention, including, for example, site-directed mutagenesis and PCR-mediated mutagenesis which result in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

The antibodies useful in the present invention include derivatives that are modified, i.e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, synthesis in the presence of tunicamycin, etc. Additionally, the derivative may contain one or more non-classical amino acids.

Antibodies useful in the present invention or fragments thereof can also comprise a framework region known to those of skill in the art. In certain embodiments, one or more framework regions, preferably, all of the framework regions, of an antibody to be used in the methods of the invention or fragment thereof are human. In certain other embodiments of the invention, the fragment region of an antibody of the invention or fragment thereof is humanized. In certain embodiments, the antibody to be used with the methods of the invention is a synthetic antibody, a monoclonal antibody, an intrabody, a chimeric antibody, a human antibody, a humanized chimeric antibody, a humanized antibody, a glycosylated antibody, a multispecific antibody, a human antibody, a single-chain antibody, or a bispecific antibody.

In certain embodiments of the invention, the antibodies useful in the present invention have half-lives in a mammal, preferably a human, of greater than 12 hours, greater than 1 day, greater than 3 days, greater than 6 days, greater than 10 days, greater than 15 days, greater than 20 days, greater than 25 days, greater than 30 days, greater than 35 days, greater than 40 days, greater than 45 days, greater than 2 months, greater than 3 months, greater than 4 months, or greater than 5 months. Antibodies or antigen-binding fragments thereof having increased in vivo half-lives can be generated by techniques known to those of skill in the art. For example, antibodies or antigen-binding fragments thereof with increased in vivo half-lives can be generated by modifying (e.g., substituting, deleting or adding) amino acid residues identified as involved in the interaction between the Fc domain and the FcRn receptor (see, e.g., PCT Publication No. WO 97/34631 and U.S. patent application Ser. No. 10/020,354, entitled “Molecules with Extended Half-Lives, Compositions and Uses Thereof”, filed Dec. 12, 2001, by Johnson et al., which are incorporated herein by reference in their entireties). Such antibodies or antigen-binding fragments thereof can be tested for binding activity to RSV antigens as well as for in vivo efficacy using methods known to those skilled in the art, for example, by immunoassays described herein.

Further, antibodies or antigen-binding fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography. PEG-derivatizated antibodies or antigen-binding fragments thereof can be tested for binding activity to RSV antigens as well as for in vivo efficacy using methods known to those skilled in the art, for example, by immunoassays described herein.

The antibodies useful in the present invention can be single-chain antibodies. The design and construction of a single-chain antibody is described in Marasco et al, 1993, Proc Natl Acad Sci 90:7889-7893, which is incorporated herein by reference in its entirety.

In certain embodiments, the antibodies useful in the present invention bind to an intracellular epitope, i.e., are intrabodies. An intrabody comprises at least a portion of an antibody that is capable of immunospecifically binding an antigen and preferably does not contain sequences coding for its secretion. Such antibodies will bind its antigen intracellularly. In one embodiment, the intrabody comprises a single-chain Fv (“sFv”). sFv are antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

In a further embodiment, the intrabody preferably does not encode an operable secretory sequence and thus remains within the cell (see generally Marasco, W A, 1998, “Intrabodies: Basic Research and Clinical Gene Therapy Applications” Springer: New York).

### 5.3.1 Antibody Conjugates

The present invention also encompasses formulations comprising antibodies that are conjugated or fused to one or more moieties, including but not limited to, peptides, polypeptides, proteins, fusion proteins, nucleic acid molecules, small molecules, mimetic agents, synthetic drugs, inorganic molecules, and organic molecules.

The present invention encompasses formulations comprising antibodies that are recombinantly fused or chemically conjugated (including both covalent and non-covalent conjugations) to a heterologous protein or polypeptide (or fragment thereof, preferably to a polypepetide of at least 10, at least 20, at least 30, at least 40, at least 50, at least 60, at least 70, at least 80, at least 90 or at least 100 amino acids) to generate fusion proteins. The fusion does not necessarily need to be direct, but may occur through linker sequences. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., International publication No. WO 93/21232; European Patent No. EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452, which are incorporated by reference in their entireties.

The present invention further includes formulations comprising heterologous proteins, peptides or polypeptides fused or conjugated to antibody fragments. For example, the heterologous polypeptides may be fused or conjugated to a Fab fragment, Fd fragment, Fv fragment, F(ab)₂ fragment, a VH domain, a VL domain, a VH CDR, a VL CDR, or fragment thereof. Methods for fusing or conjugating polypeptides to antibody portions are well-known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references incorporated by reference in their entireties).

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment may be recombined with one or more components, motifs, sections, parts, domains, fragments, etc. of one or more heterologous molecules.

Moreover, the antibodies or fragments thereof can be fused to marker sequences, such as a peptide to facilitate purification. In embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, antibodies useful in the present invention or fragments, analogs or derivatives thereof can be conjugated to a diagnostic or detectable agent. Such antibodies can be useful for monitoring or prognosing the development or progression of a disorder as part of a clinical testing procedure, such as determining the efficacy of a particular therapy. Such diagnosis and detection can be accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I), carbon (¹⁴C), sulfur (³⁵S), tritium (³H), indium (¹¹⁵In, ¹¹³In, ¹¹²In, ¹¹¹In), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), flourine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, noradioactive paramagnetic metal ions, and molecules that are radiolabelled or conjugated to specific radioisotopes.

The present invention further encompasses formulations comprising antibodies that are conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference), hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantaijian et al., Clin Cancer Res. 8(7):2167-76 (2002)), cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, procaine, tetracaine, lidocaine, propranolol, and puromycin and analogs or homologs thereof) and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459), farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305), topoisomerase inhibitors (e.g., camptothecin; irinotecan; SN-38; topotecan; 9-aminocamptothecin; GG-211 (GI 147211); DX-895 If; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin) and pharmaceutically acceptable salts, solvates, clathrates, and prodrugs thereof. See, e.g., Rothenberg, M. L., Annals of Oncology 8:837-855 (1997); and Moreau, P., et al., J. Med. Chem. 41:1631-1640 (1998)), antisense oligonucleotides (e.g., those disclosed in the U.S. Pat. Nos. 6,277,832, 5,998,596, 5,885,834, 5,734,033, and 5,618,709), immunomodulators (e.g., antibodies and cytokines), antibodies, and adenosine deaminase inhibitors (e.g., Fludarabine phosphate and 2-Chlorodeoxyadenosine).

Further, an antibody or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, .alpha.-interferon, .beta.-interferon, nerve growth factor, platelet derived growth factor, tissue plasminogen activator, an apoptotic agent, e.g., TNFα, TNFβ, AIM I (see, International publication No. WO 97/33899), AIM II (see, International Publication No. WO 97/34911), Fas Ligand (Takahashi et al., 1994, J. Immunol., 6:1567-1574), and VEGI (see, International publication No. WO 99/23105), a thrombotic agent or an anti-angiogenic agent, e.g., angiostatin, endostatin or a component of the coagulation pathway (e.g., tissue factor); or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid, fibrinopeptides A and B from the α and β chains of fibrinogen, fibrin monomer).

Moreover, an antibody can be conjugated to therapeutic moieties such as a radioactive metal ion, such as alph-emiters such as ²¹³Bi or macrocyclic chelators useful for conjugating radiometal ions, including but not limited to, ¹³¹In, ¹³¹LU, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody can be conjugated to a second antibody to form an antibody heteroconjugate as described by Segal in U.S. Pat. No. 4,676,980, which is incorporated herein by reference in its entirety.

The therapeutic moiety or drug conjugated to an antibody or fragment thereof should be chosen to achieve the desired prophylactic or therapeutic effect(s) for a particular disorder in a subject. A clinician or other medical personnel should consider the following when deciding on which therapeutic moiety or drug to conjugate to an antibody or fragment thereof: the nature of the disease, the severity of the disease, and the condition of the subject.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### 5.3.2 Formulations Comprising Purified Antibodies that Specifically Bind to a Particular Antigen

In further embodiments, the present invention encompasses formulations comprising isolated antibodies or compositions comprising antibodies, wherein said antibodies specifically bind to one or more particular antigens. In certain embodiments, the antibody of the present invention specifically binds to an antigen of respiratory syncytial virus (RSV). In other embodiments, the antibody of the present invention specifically binds to an antigen of human metapneumovirus (hMPV). In some embodiments, the antibody is a humanized antibody that specifically binds to an antigen of hMPV. In certain embodiments, the antibody of the present invention specifically binds to integrin αᵥβ₃. In some embodiments, the antibody is MEDI-522 (Vitaxin®). In certain embodiments, the antibody of the present invention specifically binds to CD2. In some embodiments, the antibody is siplizumab. In certain embodiments, the antibody of the present invention specifically binds to CD19. In some embodiments, the antibody is MT-103. In further embodiments, the antibody of the present invention specifically binds to EphA2. In some embodiments, the antibody is human or humanized EA2 or EA5. In certain embodiments, the antibody of the present invention specifically binds to EphA4. In some embodiments, the antibody is a humanized antibody that specifically binds to EphA4. In certain embodiments, the antibody of the present invention specifically binds to IL-9. In some embodiments, the antibody is a human or humanized antibody that specifically binds to IL-9. In some embodiments, the antibody is MEDI-528.

In some embodiments, the antibody is not palivizumab. In some embodiments, the antibody is not MEDI-522 (Vitaxin®). In some embodiments, the antibody is not siplizumab. In some embodiments, the antibody is not MT-103. In some embodiments, the antibody is not human or humanized EA2 or EA5. In some embodiments, the antibody is not MEDI-528.

The antibodies useful in the present invention may be high potency antibodies. High potency antibodies can be produced by genetically engineering appropriate antibody gene sequences and expressing the antibody sequences in a suitable host. The antibodies produced can be screened to identify antibodies with, e.g., high kₒₙ values in a BIAcore assay.

In certain embodiments, the antibodies useful in the present invention have a high binding affinity for one or more antigens. In a specific embodiment, the antibodies of the present invention have an association rate constant or kₒₙ rate (antibody (Ab)+antigen (Ag)^{k}^{ₒₙ}→Ab-Ag) of at least 10⁵ M⁻¹s⁻¹, at least 5×10⁵ M⁻¹s⁻¹, at least 10⁶M⁻¹s⁻¹, at least 5×10⁶ M⁻¹s⁻¹, at least 10⁷M⁻¹s⁻¹, at least 5×10⁷M⁻¹s⁻¹, or at least 10⁸M⁻¹s⁻¹. In a preferred embodiment, the antibodies of the present invention have a kₒₙ of at least 2×10⁵ M⁻¹s⁻¹, at least 5×10⁵ M⁻¹s⁻¹, at least 10⁶M⁻¹s⁻¹, at least 5×10⁶ M⁻¹s⁻¹ at least 10⁷ M⁻¹s⁻¹, at least 5×10⁷M⁻¹s⁻¹, or at least 10⁸ M⁻¹s⁻¹.

In another embodiment, the antibodies of the present invention have a k_{off} rate (antibody (Ab)+antigen) of less than 10⁻¹ s⁻¹, less than 5×10⁻¹ s⁻¹, less than 10⁻² s⁻¹, less than 5×10⁻² s⁻¹, less than 10⁻³ s⁻¹, less than 5×10⁻³ s⁻¹, less than 10⁻⁴ s⁻¹, less than 5×10⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹ s⁻¹. In a preferred embodiment, the antibodies of the present invention have a kₒₙ of less than 5×10⁻⁴s⁻¹, less than 10⁻⁵s⁻¹, less than 5×10⁻⁵s⁻¹, less than 10⁻⁶s⁻¹, less than 5×10⁻⁶ s⁻¹, less than 10⁻⁷s⁻¹ less than 5×10⁻⁷s⁻¹ less than 10⁻⁸s⁻¹ less than 5×10⁻⁸s⁻¹ less than 10⁻⁹s⁻¹, less than 5×10⁻⁹s⁻¹, or less than 10⁻¹° s⁻¹.

In certain embodiments, the antibodies of the present invention have an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹ at least 10¹¹ M⁻¹, at least 5×10¹¹ M⁻¹, at least 10¹² M⁻¹, at least 5×10¹² M⁻¹, at least 10¹³M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. The present invention also provides formulations comprising one or more antibodies which immunospecifically bind to an antigen with an affinity constant of at least 2×10⁸ M⁻¹, at least 2.5×10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹.

In yet another embodiment, the antibodies useful in the present invention have a dissociation constant or K_{d} (k_{off}/kₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷ M, less than 5×10⁻⁷M, less than 10⁻⁸ M, less than 5×10⁻⁸M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³M, less than 5×10⁻¹³ M, less than 10⁻¹⁴ M, less than 5×10⁻¹⁴ M, less than 10⁻¹⁵ M, or less than 5×10⁻¹⁵ M.

In certain embodiments, the antibodies useful in the present invention have a median effective concentration (EC₅₀) of less than 0.01 nM, less than 0.025 nM, less than 0.05 nM, less than 0.1 nM, less than 0.25 nM, less than 0.5 nM, less than 0.75 nM, less than 1 nM, less than 1.25 nM, less than 1.5 nM, less than 1.75 nM, or less than 2 nM, in an in vitro microneutralization assay. The median effective concentration is the concentration of antibody or antibody fragments that neutralizes 50% of an antigen in an in vitro microneutralization assay. In a preferred embodiment, the antibodies of the present invention have an EC₅₀ of less than 0.01 nM, less than 0.025 nM, less than 0.05 nM, less than 0.1 nM, less than 0.25 nM, less than 0.5 nM, less than 0.75 nM, less than 1 nM, less than 1.25 nM, less than 1.5 nM, less than 1.75 nM, or less than 2 nM, in an in vitro microneutralization assay.

The present invention also provides antibodies that immunospecifically bind to an antigen of interest, the antibodies comprising derivatives of the VH domains, VH CDRs, VL domains, and VL CDRs described herein that immunospecifically bind to antigens of interest. Standard techniques known to those of skill in the art can be used to introduce mutations in the nucleotide sequence encoding a molecule of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

### 5.3.3 Antibodies that Immunospecifically Bind to RSV Antigen

It should be recognized that antibodies that immunospecifically bind to a RSV antigen are known in the art. For example, palivizumab is a humanized monoclonal antibody presently used for the prevention of RSV infection in pediatric patients. The present invention provides formulations of antibodies that immunospecifically bind to one or more RSV antigens. Preferably, the antibodies useful in the invention immunospecifically bind to one or more RSV antigens regardless of the strain of RSV. The present invention also provides antibodies that differentially or preferentially bind to RSV antigens from one strain of RSV versus another RSV strain. In a specific embodiment, the formulations comprise antibodies that immunospecifically bind to the RSV F glycoprotein, G glycoprotein or SH protein. In a preferred embodiment, the formulations comprise antibodies that immunospecifically bind to the RSV F glycoprotein. In another preferred embodiment, the formulations comprise antibodies that bind to the A, B, or C antigenic sites of the RSV F glycoprotein.

The formulations of the invention comprise antibodies that immunospecifically bind to a RSV antigen and have a dissociation constant (K_{D}) of less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM as assessed using an described herein or known to one of skill in the art (e.g., a BIAcore assay). In a specific embodiment, formulations of the invention comprise antibodies that immunospecifically bind to a RSV antigen and have a dissociation constant (K_{D}) of between 25 to 3400 pM, 25 to 3000 pM, 25 to 2500 pM, 25 to 2000 pM, 25 to 1500 pM, 25 to 1000 pM, 25 to 750 pM, 25 to 500 pM, 25 to 250 pM, 25 to 100 pM, 25 to 75 pM, 25 to 50 pM as assessed using an described herein or known to one of skill in the art (e.g., a BIAcore assay). In another embodiment, formulations of the invention comprise antibodies that immunospecifically bind to a RSV antigen and have a dissociation constant (K_{D}) of 500 pM, preferably 100 pM, more preferably 75 pM and most preferably 50 pM as assessed using an described herein or known to one of skill in the art (e.g., a BIAcore assay).

The present invention provides formulations that comprise antibodies that have a median inhibitory concentration (IC₅₀) of less than 5 nM, less than 4 nM, less than 3 nM, less than 2 nM, less than 1.75 nM, less than 1.5 nM, less than 1.25 nM, less than 1 nM, less than 0.75 nM, less than 0.5 nM, less than 0.25 nM, less than 0.1 nM, less than 0.05 nM, less than 0.025 nM, or less than 0.01 nM, in an in vitro microneutralization assay. The IC₅₀ is the concentration of antibody that neutralizes 50% of the RSV in an in vitro microneutralization assay. In a preferred embodiment, antibody of the invention has an IC₅₀ of less than 5 nM, less than 4 nM, less than 3 nM, less than 2 nM, less than 1.75 nM, less than 1.5 nM, less than 1.25 nM, less than 1 nM, less than 0.75 nM, less than 0.5 nM, less than 0.25 nM, less than 0.1 nM, less than 0.05 nM, less than 0.025 nM, or less than 0.01 nM, in an in vitro microneutralization assay.

In a specific embodiment, the formulations of the invention comprise an antibody that has approximately 20-fold, 25-fold, 30-fold, 35-fold, 40-fold, 45-fold, 50-fold, 55-fold, 60-fold, 65-fold, 70-fold, 75-fold, 80-fold, 90-fold, 100-fold or higher affinity for a RSV F antigen than palivizumab or an antibody-binding fragment thereof as assessed by an assay known in the art or described herein (e.g., a BIAcore assay). In another embodiment, formulations of the invention comprise antibodies that have an approximately 1-fold, 1.5-fold, 2-fold, 3-fold, 4-fold, 5-fold, or more higher Ka than palivizumab or an antigen-binding fragment thereof as assessed by an assay known in the art or described herein. In another embodiment, a formulation of the invention comprises an antibody that is approximately 1-fold, 2-fold, 3-fold, 4-fold, 5-fold, 6-fold, 7-fold, 8-fold, 9-fold, 10-fold, 11-fold 12-fold, 13-fold, 14-fold, 15-fold, 16-fold, 17-fold, 18-fold, 19-fold, or 20-fold or more potent than palivizumab or an antigen-binding fragment thereof in an in vitro microneutralization assay such as described herein. The amino acid sequence of palivizumab is disclosed, e.g., in Johnson et al., 1997, J. Infectious Disease 176:1215-1224, and U.S. Pat. No. 5,824,307, each of which is incorporated herein by reference in its entirety. In a specific embodiment, a formulation of the invention comprise an antibody that is not palivizumab or a fragment of palivizumab or an antigen-binding fragment of palivizumab, e.g., is not an antibody comprising a VH domain of SEQ ID NO:7 and/or a VL domain of SEQ ID NO:8.

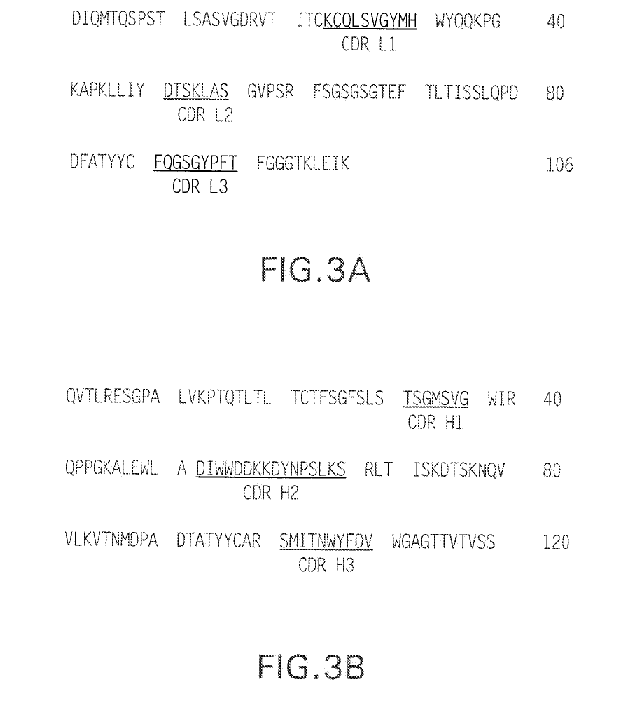

The present invention provides antibodies that immunospecifically bind to one or more RSV antigens, said antibodies comprising the amino acid sequence of palivizumab with one or more amino acid residue substitutions in the variable light (VL) domain and/or variable heavy (VH) domain depicted in FIG. 3. The present invention also provides antibodies that immunospecifically bind to one or more RSV antigens, said antibodies comprising the amino acid sequence of palivizumab with one or more amino acid residue substitutions in one or more VL CDRs and/or one or more VH CDRs. In a specific embodiment, an antibody comprises the amino acid sequence of palivizumab with one or more amino acid residue substitutions of the amino acid residues indicated in bold face and underlining in Table 1. In another embodiment, an antibody comprises the amino sequence of palivizumab with one or more amino acid residue substitutions of the amino acid residues indicated in bold face and underlining in Table 1 and one or more amino acid residue substitutions of the framework regions of the variable domains of palivizumab (e.g., mutations in framework region 4 of the heavy and/or light variable domains). In accordance with these embodiments, the amino acid residue substitutions can be conservative or non-conservative. The antibody generated by introducing substitutions in the VH domain, VH CDRs, VL domain and/or VL CDRs of palivizumab can be tested in vitro and in vivo, for example, for its ability to bind to RSV F antigen, for its ability to neutralize RSV, or for its ability to prevent, treat or ameliorate an upper and/or lower respiratory tract RSV infection, otitis media, or a symptom or respiratory condition relating thereto (including, but not limited to, asthma, wheezing, RAD, or a combination thereof).

TABLE 1
CDR Sequences of palivizumab

| CDR | Sequence | SEQ ID NO: |
|---|---|---|
| VH1 | TSGMSVG | 1 |
| VH2 | DIWWDDKKDYNPSLKS | 2 |
| VH3 | SMITNWYFDV | 3 |
| VL1 | KCQLSVGYMH | 4 |
| VL2 | DTSKLAS | 5 |
| VL3 | FQGSGYPFT | 6 |

* Bold faced & underlined amino acid residues are preferred residues which should be substituted.

The formulations of the present invention also comprise those antibodies and antigen-binding fragments of the antibodies referenced in Table 2 and the Examples Section of the application. In a specific embodiment, a formulation of the present invention comprises antibody AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4. In another embodiment, a formulation of the present invention comprises an antigen-binding fragment (e.g., a Fab fragment of) AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4. In a preferred embodiment, a formulation of the present invention comprises antibody A4B4L1FR-S28R or an antigen-binding fragment thereof.

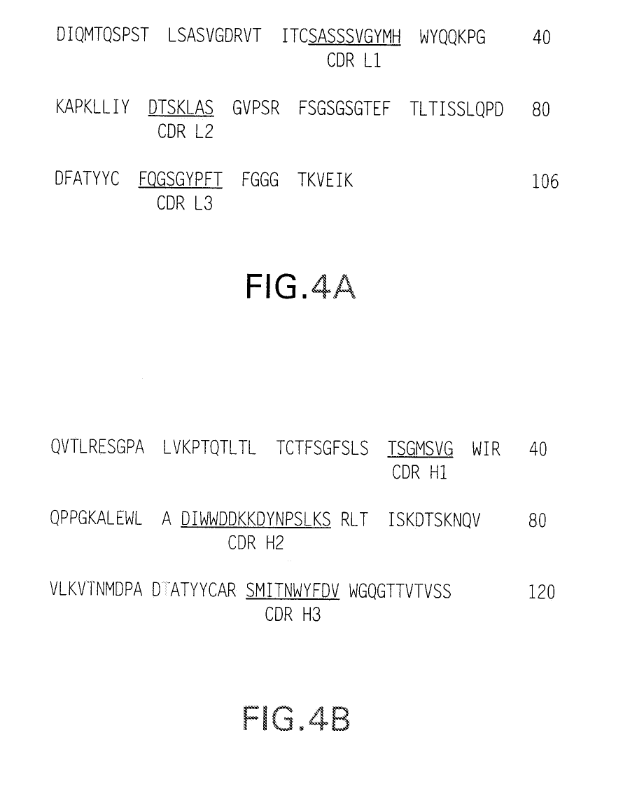

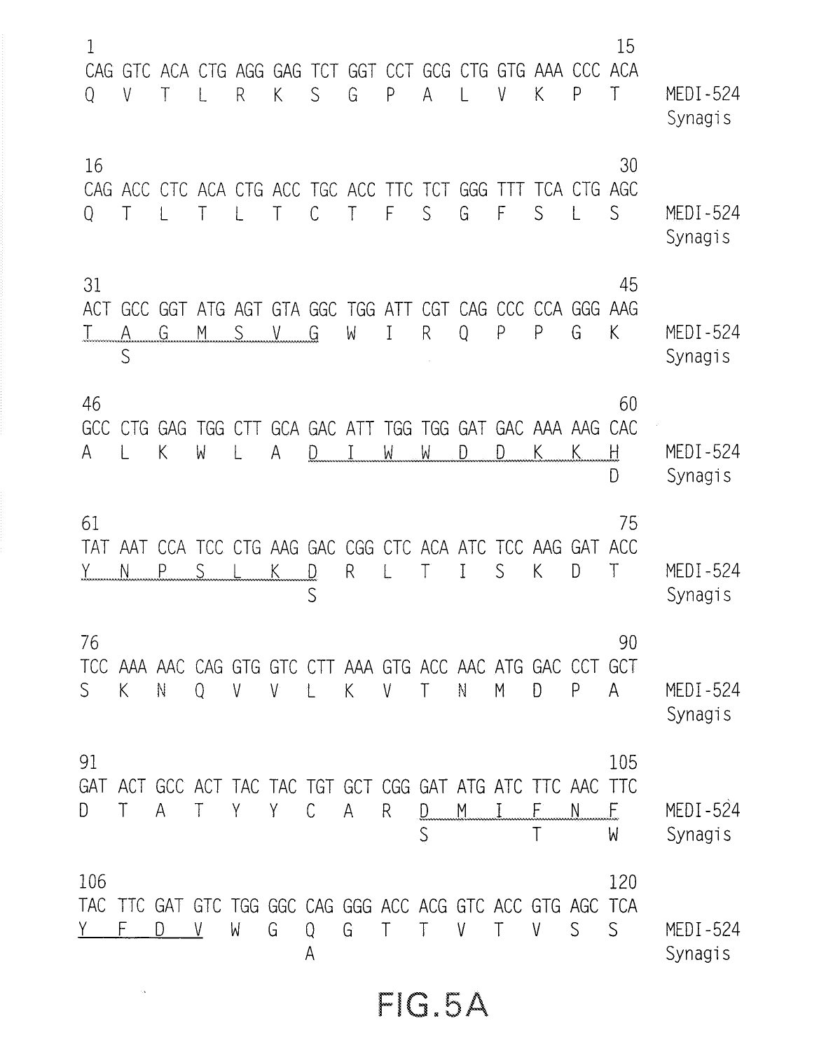

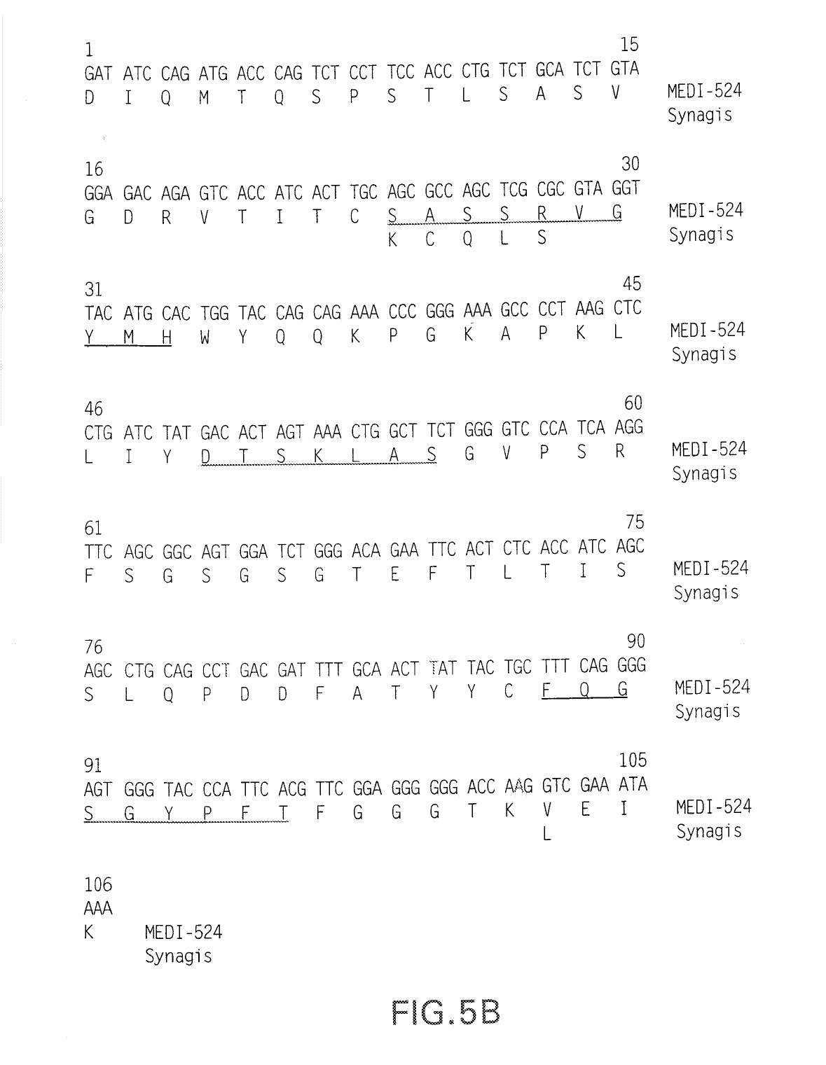

In some embodiments, AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R, A4B4-F52S, A17d4(1), A3e2, A14a4, A16b4, A17b5, A17f5, and/or A17h4 comprise the framework region and constant regions of palivizumab (see FIG. 3). In preferred embodiments, AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, and/or A17h4 comprise the framework region and constant regions of palivizumab with the exception that there is an amino acid substitution of an A105Q in the heavy chain framework 4 (FR4) (numbering used herein according to Kabat et al. (1991). Sequences of proteins of immunological interest. (U.S. Department of Health and Human Services, Washington, D.C.) 5^{th} ed.) (“Kabat numbering”) (i.e., position 112 in SEQ ID NO:7 (palivizumab VH domain)) and an L104V in the light chain FR4 (i.e., position 103 in SEQ ID NO:8 (palivizumab VL domain)). An example of antibodies comprising a framework with these VH and VL single mutations is shown in FIG. 4 (1X-493L1FR) and in FIG. 5 (A4B4L1FR-S28R).

In a specific embodiment, the present invention provides one or more antibodies that immunospecifically bind to one or more RSV F antigens, said antibodies comprising a VH chain and/or VL chain having the amino acid sequence of a VH chain and/or VL chain of AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R, A4B4-F52S, A17d4(1), A3e2, A14a4, A16b4, A17b5, A17f5, or A17h4. In a preferred embodiment, an antibody of the invention immunospecifically binds to a RSV F antigen, and said antibody comprises a VH chain and/or a VL chain having the amino acid sequence of the VH and/or VL chain of A4B4L1FR-S28 (VH chain, SEQ ID NO:254; VL chain SEQ ID NO:255). In another embodiment, the present invention provides one or more antibodies that immunospecifically bind to one or more RSV antigens, said antibodies comprising a VH domain and/or VL domain having the amino acid sequence of a VH domain and/or VL domain of AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R, A4B4-F52S, A17d4(1), A3e2, A14a4, A16b4, A17b5, A17f5, or A17h4. In a preferred embodiment, an antibody of the invention immunospecifically binds to a RSV F antigen, and said antibody comprises a VH domain and/or VL domain having the amino acid sequence of the VH domain and/or VL domain of A4B4L1FR-S28R (VH domain, SEQ ID NO:48; VL domain, SEQ ID NO:11).

In another embodiment, the present invention provides antibodies that immunospecifically bind to one or more RSV antigens, said antibodies comprising one, two, three, or more CDRs having the amino acid sequence of one, two, three, or more CDRs of AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524, motavizumab), A4B4-F52S, A17d4(1), A3e2, A14a4, A16b4, A17b5, A17f5, or A17h4. In a preferred embodiment, a formulation of the present invention comprises an antibody that immunospecifically binds to a RSV antigen, and said antibody comprises one, two, three, or more CDRs having the amino acid sequence of one, two, three, or more CDRs of A4B4L1FR-S28R. In yet another embodiment, the formulation of the present invention comprises an antibody that immunospecifically binds to one or more RSV F antigens, said antibodies comprising a combination of VH CDRs and/or VL CDRs having the amino acid sequence of VH CDRs and/or VL CDRs of AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, and/or A17h4. In a preferred embodiment, a formulation of the present invention comprises an antibody that immunospecifically binds to a RSV F antigen and said antibody comprises a combination of VH CDRs and/or VL CDRs having the amino acid sequence of the VH CDRs and/or VL CDRs of A4B4L1FR-S28R.

The present invention provides antibodies that immunospecifically bind to one or more RSV antigens (e.g., RSV F antigen), said antibodies comprising a variable heavy (“VH”) chain having an amino acid sequence of any one of the VH chains listed in Table 2. In certain embodiments, the antibody is not palivizumab and/or the VH chain is not the VH chain of palivizumab.

The invention also provides antibodies that immunospecifically bind to one or more RSV antigens (e.g., RSV F antigen), said antibodies comprising a VH domain having an amino acid sequence of any one of the VH domains listed in Table 2. In certain embodiments of the invention, the antibody is not palivizumab and/or the VH domain is not the VH domain of palivizumab.

The present invention also provides antibodies that immunospecifically bind to one or more RSV antigens, said antibodies comprising a VH complementarity determining region (“CDR”) (e.g., VH CDR1, VH CDR2, and/or VH CDR3) having an amino acid sequence of any of the VH CDRs listed in Table 2 and/or Tables 3A-3C. In certain embodiments of the invention, an antibody comprising a VH CDR having an amino acid of any of one of the VH CDRs listed in Table 2 and/or Tables 3A-3C is not palivizumab. In some embodiments, the antibody or binding fragment thereof comprises one, two or three of the VH CDRs listed in Table 2 and/or Tables 3A-3C.

TABLE 2
Antibodies & Fragments Thereof

| Antibody | VH | VH | | | | VL | VL | | | |
|---|---|---|---|---|---|---|---|---|---|---|
| Name | Chain | Domain | VH CDR1 | VH CDR2 | VH CDR3 | Chain | Domain | VL CDR1 | VL CDR2 | VL CDR3 |
| **palivizumab | SEQ ID | SEQ ID | TSGMSVG | DIWWDDKKDYN | SMITNWYFDV | SEQ ID | SEQ ID | KCQLSVGYMH | DTSKLAS | FQGSGYPFT |
| | NO: 208 | NO: 7 | (SEQ ID NO: 1) | PSLKS | (SEQ ID NO: 3) | NO: 209 | NO: 8 | (SEQ ID NO: 4) | (SEQ ID NO: 5) | (SEQ ID NO: 6) |
| | | | | (SEQ ID NO: 2) | | | | | | |
| ***AFFF | SEQ ID | SEQ ID | TAGMSVG | DIWWDDKKDYN | SMITNFYFDV | SEQ ID | SEQ ID | SASSSVGYMH | DTFKLAS | FQFSGYPFT |
| | NO: 210 | NO: 9 | (SEQ ID NO: 10) | PSLKS | (SEQ ID NO: 12) | NO: 211 | NO: 13 | (SEQ ID NO: 14) | (SEQ ID | (SEQ ID |
| | | | | (SEQ ID NO: 2) | | | | | NO: 15) | NO: 16) |
| ***P12f2 | SEQ ID | SEQ ID | TPGMSVG | DIWWDDKKHYN | DMIFNFYFDV | SEQ ID | SEQ ID | SLSSRVGYMH | DTFYLSS | FQGSGYPFT |
| | NO: 212 | NO: 17 | (SEQ ID NO: 18) | PSLKD | (SEQ ID NO: 20) | NO: 213 | NO: 21 | (SEQ ID NO: 22) | (SEQ ID | (SEQ ID NO: 6) |
| | | | | (SEQ ID NO: 19) | | | | | NO: 23) | |
| ***P12f4 | SEQ ID | SEQ ID | TPGMSVG | DIWWDGKKHYN | DMIFNFYFDV | SEQ ID | SEQ ID | SLSSRVGYMH | DTRGLPS | FQGSGYPFT |
| | NO: 214 | NO: 24 | (SEQ ID NO: 18) | PSLKD | (SEQ ID NO: 20) | NO: 215 | NO: 26 | (SEQ ID NO: 22) | (SEQ ID | (SEQ ID NO: 6) |
| | | | | (SEQ ID NO: 25) | | | | | NO: 27) | |
| ***P11d4 | SEQ ID | SEQ ID | TPGMSVG | DIWWDGKKHYN | DMIFNWYFDV | SEQ ID | SEQ ID | SPSSRVGYMH | DTMRLAS | FQGSGYPFT |
| | NO: 216 | NO: 28 | (SEQ ID NO: 18) | PSLKD | (SEQ ID NO: 29) | NO: 217 | NO: 30 | (SEQ ID NO: 31) | (SEQ ID | (SEQ ID NO: 6) |
| | | | | (SEQ ID NO: 25) | | | | | NO: 32) | |
| ***Ale9 | SEQ ID | SEQ ID | TAGMSVG | DIWWDGKKHYN | DMIFNWYFDV | SEQ ID | SEQ ID | SLSSRVGYMH | DTFKLSS | FQGSGYPFT |
| | NO: 218 | NO: 33 | (SEQ ID NO: 10) | PSLKD | (SEQ ID NO: 29) | NO: 219 | NO: 34 | (SEQ ID NO: 22) | (SEQ ID | (SEQ ID NO: 6) |
| | | | | (SEQ ID NO: 25) | | | | | NO: 35) | |
| ***A12a6 | SEQ ID | SEQ ID | TAGMSVG | DIWWDGKKDYN | DMIFNFYFDV | SEQ ID | SEQ ID | SASSRVGYMH | DTFKLSS | FQGSGYPFT |
| | NO: 220 | NO: 36 | (SEQ ID NO: 10) | PSLKD | (SEQ ID NO: 20) | NO: 221 | NO: 38 | (SEQ ID NO: 39) | (SEQ ID | (SEQ ID NO: 6) |
| | | | | (SEQ ID NO: 37) | | | | | NO: 35) | |
| ***A13c4 | SEQ ID | SEQ ID | TAGMSVG | DIWWDGKKSYN | DMIFNFYFDV | SEQ ID | SEQ ID | SLSSRVGYMH | DTMYQSS | FQGSGYPFT |
| | NO: 222 | NO: 40 | (SEQ ID NO: 10) | PSLKD | (SEQ ID NO: 20) | NO: 223 | NO: 42 | (SEQ ID NO: 22) | (SEQ ID | (SEQ ID NO: 6) |
| | | | | (SEQ ID NO: 41) | | | | | NO: 43) | |
| ***A17d4 | SEQ ID | SEQ ID | TAGMSVG | DIWWDDKKSYN | DMIFNFYFDV | SEQ ID | SEQ ID | LPSSRVGYMH | DTMYQSS | FQGSGYPFT |
| | NO: 224 | NO: 44 | (SEQ ID NO: 10) | PSLKD | (SEQ ID NO: 20) | NO: 225 | NO: 46 | (SEQ ID NO: 47) | (SEQ ID | (SEQ ID NO: 6) |
| | | | | (SEQ ID NO: 45) | | | | | NO: 43) | |
| ***A4B4 | SEQ ID | SEQ ID | TAGMSVG | DIWWDDKKHYN | DMIFNFYFDV | SEQ ID | SEQ ID | SASSRVGYMH | DTFFLDS | FQGSGYPFT |
| | NO: 226 | NO: 48 | (SEQ ID NO: 10) | PSLKD | (SEQ ID NO: 20) | NO: 227 | NO: 49 | (SEQ ID NO: 39) | (SEQ ID | (SEQ ID NO: 6) |
| | | | | (SEQ ID NO: 19) | | | | | NO: 50) | |
| ****A8c7 | SEQ ID | SEQ ID | TAGMSVG | DIWWDDKKSYN | DMIFNWYFDV | SEQ ID | SEQ ID | SPSSRVGYMH | DTRYQSS | FQGSGYPFT |
| | NO: 228 | NO: 51 | (SEQ ID NO: 10) | PSLKD | (SEQ ID NO: 29) | NO: 229 | NO: 52 | (SEQ ID NO: 31) | (SEQ ID | (SEQ ID NO: 6) |
| | | | | (SEQ ID NO: 45) | | | | | NO: 53) | |
| *1X- | SEQ ID | SEQ ID | TSGMSVG | DIWWDDKKDYN | SMITNWYFDV | SEQ ID | SEQ ID | SASSSVGYMH | DTSKLAS | FQGSGYPFT |
| 493L1FR | NO: 230 | NO: 343 | (SEQ ID NO: 1) | PSLKS | (SEQ ID NO: 3) | NO: 231 | NO: 54 | (SEQ ID NO: 14) | (SEQ ID NO: 5) | (SEQ ID NO: 6) |
| | | | | (SEQ ID NO: 2) | | | | | | |
| *H3-3F4 | SEQ ID | SEQ ID | TAGMSVG | DIWWDDKKDYN | DMIFNWYFDV | SEQ ID | SEQ ID | SASSSVGYMH | DTFKLAS | FQGSGYPFT |
| | NO: 232 | NO: 55 | (SEQ ID NO: 10) | PSLKS | (SEQ ID NO: 29) | NO: 233 | NO: 56 | (SEQ ID NO: 14) | (SEQ ID | (SEQ ID NO: 6) |
| | | | | (SEQ ID NO: 2) | | | | | NO: 15) | |
| *M3H9 | SEQ ID | SEQ ID | TAGMSVG | DIWWDDKKDYN | DMIFNWYFDV | SEQ ID | SEQ ID | SASSSVGYMH | DTYKQTS | FQGSGYPFT |
| | NO: 234 | NO: 55 | (SEQ ID NO: 10) | PSLKS | (SEQ ID NO: 29) | NO: 235 | NO: 70 | (SEQ ID NO: 14) | (SEQ ID | (SEQ ID NO: 6) |
| | | | | (SEQ ID NO: 2) | | | | | NO: 57) | |
| *Y10H6 | SEQ ID | SEQ ID | TAGMSVG | DIWWDDKKDYN | DMIFNWYFDV | SEQ ID | SEQ ID | SASSSVGYMH | DTRYLSS | FQGSGYPFT |
| | NO: 236 | NO: 55 | (SEQ ID NO: 10) | PSLKS | (SEQ ID NO: 29) | NO: 237 | NO: 58 | (SEQ ID NO: 14) | (SEQ ID | (SEQ ID NO: 6) |
| | | | | (SEQ ID NO: 2) | | | | | NO: 59) | |
| *DG | SEQ ID | SEQ ID | TAGMSVG | DIWWDDKKDYN | DMITNFYFDV | SEQ ID | SEQ ID | SASSSVGYMH | DTFKLAS | FQGSGYPFT |
| (aka | NO: 238 | NO: 78 | (SEQ ID NO: 10) | PSLKS | (SEQ ID NO: 79) | NO: 239 | NO: 56 | (SEQ ID NO: 14) | (SEQ ID | (SEQ ID NO: 6) |
| D95/G93) | | | | (SEQ ID NO: 2) | | | | | NO: 15) | |
| AFFF(1) | SEQ ID | SEQ ID | TAGMSVG | DIWWDDKKDYN | SMITNFYFDV | SEQ ID | SEQ ID | SASSSVGYMH | DTFKLAS | FQGSFYPFT |
| | NO: 240 | NO: 9 | (SEQ ID NO: 10) | PSLKS | (SEQ ID NO: 12) | NO: 241 | NO: 60 | (SEQ ID NO: 14) | (SEQ ID | (SEQ ID |
| | | | | (SEQ ID NO: 2) | | | | | NO: 15) | NO: 61) |
| *6H8 | SEQ ID | SEQ ID | TAGMSVG | DIWWDDKKDYN | DMITNFYFDV | SEQ ID | SEQ ID | SASSSVGYMH | DTFKLTS | FQGSGYPFT |
| | NO: 242 | NO: 78 | (SEQ ID NO: 10) | PSLKS | (SEQ ID NO: 79) | NO: 243 | NO: 62 | (SEQ ID NO: 14) | (SEQ ID | (SEQ ID NO: 6) |
| | | | | (SEQ ID NO: 2) | | | | | NO: 63) | |
| *L1-7E5 | SEQ ID | SEQ ID | TAGMSVG | DIWWDDKKDYN | DMITNFYFDV | SEQ ID | SEQ ID | SASSRVGYMH | DTFKLAS | FQGSGYPFT |
| | NO: 244 | NO: 78 | (SEQ ID NO: 10) | PSLKS | (SEQ ID NO: 79) | NO: 245 | NO: 64 | (SEQ ID NO: 39) | (SEQ ID | (SEQ ID NO: 6) |
| | | | | (SEQ ID NO: 2) | | | | | NO: 15) | |
| *L2-15B10 | SEQ ID | SEQ ID | TAGMSVG | DIWWDDKKDYN | DMITNFYFDV | SEQ ID | SEQ ID | SASSSVGYMH | DTFRLAS | FQGSGYPFT |
| | NO: 246 | NO: 78 | (SEQ ID NO: 10) | PSLKS | (SEQ ID NO: 79) | NO: 247 | NO: 65 | (SEQ ID NO: 14) | (SEQ ID | (SEQ ID NO: 6) |
| | | | | (SEQ ID NO: 2) | | | | | NO: 66) | |
| *A13a11 | SEQ ID | SEQ ID | TAGMSVG | DIWWDDKKHYN | DMIFNWYFDV | SEQ ID | SEQ ID | SPSSRVGYMH | DTYRHSS | FQGSGYPFT |
| | NO: 248 | NO: 67 | (SEQ ID NO: 10) | PSLKD | (SEQ ID NO: 29) | NO: 249 | NO: 68 | (SEQ ID NO: 31) | (SEQ ID | (SEQ ID NO: 6) |
| | | | | (SEQ ID NO: 19) | | | | | NO: 69) | |
| *A1h5 | SEQ ID | SEQ ID | TAGMSVG | DIWWDGKKHYN | DMIFNWYFDV | SEQ ID | SEQ ID | SLSSSVGYMH | DTFFHRS | FQGSGYPFT |
| | NO: 250 | NO: 33 | (SEQ ID NO: 10) | PSLKD | (SEQ ID NO: 29) | NO: 251 | NO: 71 | (SEQ ID NO: 72) | (SEQ ID | (SEQ ID NO: 6) |
| | | | | (SEQ ID NO: 25) | | | | | NO: 73) | |
| A4B4(1) | SEQ ID | SEQ ID | TAGMSVG | DIWWDDKKHYN | DMIFNFYFDV | SEQ ID | SEQ ID | SASSRVGYMH | DTLLLDS | FQGSGYPFT |
| | NO: 252 | NO: 48 | (SEQ ID NO: 10) | PSLKD | (SEQ ID NO: 20) | NO: 253 | NO: 74 | (SEQ ID NO: 39) | (SEQ ID | (SEQ ID NO: 6) |
| | | | | (SEQ ID NO: 19) | | | | | NO: 75) | |
| ***A4B4L1FR- | SEQ ID | SEQ ID | TAGMSVG | DIWWDDKKHYN | DMIFNFYFDV | SEQ ID | SEQ ID | SASSRVGYMH | DTSKLAS | FQGSGYPFT |
| S28R | NO: 254 | NO: 48 | (SEQ ID NO: 10) | PSLKD | (SEQ ID NO: 20) | NO: 255 | NO: 11 | (SEQ ID NO: 39) | (SEQ ID NO: 5) | (SEQ ID NO: 6) |
| (aka | | | | (SEQ ID NO: 19) | | | | | | |
| motavizumab) | | | | | | | | | | |
| ***A4B4- | SEQ ID | SEQ ID | TAGMSVG | DIWWDDKKHYN | DMIFNFYFDV | SEQ ID | SEQ ID | SASSRVGYMH | DTSFLDS | FQGSGYPFT |
| F52S | NO: 256 | NO: 48 | (SEQ ID NO: 10) | PSLKD | (SEQ ID NO: 20) | NO: 257 | NO: 76 | (SEQ ID NO: 39) | (SEQ ID | (SEQ ID NO: 6) |
| | | | | (SEQ ID NO: 19) | | | | | NO: 77) | |
| ***A17d4(1) | SEQ ID | SEQ ID | TAGMSVG | DIWWDGKKSYN | DMIFNFYFDV | SEQ ID | SEQ ID | LPSSRVGYMH | DTMYQSS | FQGSGYPFT |
| | NO: 222 | NO: 40 | (SEQ ID NO: 10) | PSLKD | (SEQ ID NO: 20) | NO: 225 | NO: 46 | (SEQ ID NO: 47) | (SEQ ID | (SEQ ID NO: 6) |
| | | | | (SEQ ID NO: 41) | | | | | NO: 43) | |
| ***A3e2 | SEQ ID | SEQ ID | TAGMSVG | DIWWGDKGHYN | DMIFNWYFDV | SEQ ID | SEQ ID | SASSSVGYMH | DTFYLHS | FQGSGYPFT |
| | NO: 303 | NO: 304 | (SEQ ID NO: 10) | PSLKD | (SEQ ID NO: 29) | NO: 306 | NO: 307 | (SEQ ID NO: 14) | (SEQ ID | (SEQ ID NO: 6) |
| | | | | (SEQ ID NO: 305) | | | | | NO: 308) | |
| ***A14a4 | SEQ ID | SEQ ID | TAGMSVG | DIWWDDKKSYN | DMITNWYFDV | SEQ ID | SEQ ID | LLSSRVGYMH | DTYYQTS | FQGSGYPFT |
| | NO: 309 | NO: 310 | (SEQ ID NO: 10) | PSLKD | (SEQ ID NO: 311) | NO: 312 | NO: 313 | (SEQ ID NO: 314) | (SEQ ID | (SEQ ID NO: 6) |
| | | | | (SEQ ID NO: 45) | | | | | NO: 315) | |
| ***A16b4 | SEQ ID | SEQ ID | TAGMSVG | DIWWDDKKHYN | DMIFNWYFDV | SEQ ID | SEQ ID | LLSSRVGYMH | DTMYQAS | FQGSGYPFT |
| | NO: 316 | NO: 317 | (SEQ ID NO: 10) | PSLKD | (SEQ ID NO: 29) | NO: 318 | NO: 319 | (SEQ ID NO: 320) | (SEQ ID | (SEQ ID NO: 6) |
| | | | | (SEQ ID NO: 19) | | | | | NO: 321) | |
| ***A17b5 | SEQ ID | SEQ ID | TAGMSVG | DIWWDDKKHYN | DMIFNWYFDV | SEQ ID | SEQ ID | SLSSRVGYMH | DTYYLPS | FQGSGYPFT |
| | NO: 322 | NO: 323 | (SEQ ID NO: 10) | PSLKD | (SEQ ID NO: 29) | NO: 324 | NO: 325 | (SEQ ID NO: 22) | (SEQ ID | (SEQ ID NO: 6) |
| | | | | (SEQ ID NO: 19) | | | | | NO: 326) | |
| ***A17f5 | SEQ ID | SEQ ID | TAGMSVG | DIWWDDKKDYN | DMIFNWYFDV | SEQ ID | SEQ ID | SLSSRVGYMH | DTFRHTS | FQGSGYPFT |
| | NO: 327 | NO: 328 | (SEQ ID NO: 10) | PSLKD | (SEQ ID NO: 29) | NO: 330 | NO: 331 | (SEQ ID NO: 22) | (SEQ ID | (SEQ ID NO: 6) |
| | | | | (SEQ ID NO: 329) | | | | | NO: 332) | |
| ***A17h4 | SEQ ID | SEQ ID | TAGMSVG | DIWWDGKKHYN | DMIFNWYFDV | SEQ ID | SEQ ID | SPSSSVGYMH | DTYYLAS | FQGSGYPFT |
| | NO: 218 | NO: 33 | (SEQ ID NO: 10) | PSLKD | (SEQ ID NO: 29) | NO: 333 | NO: 334 | (SEQ ID NO: 335) | (SEQ ID | (SEQ ID NO: 6) |
| | | | | (SEQ ID NO: 25) | | | | | NO: 336) | |

Bold faced & underlined amino acid residues are the residues which differ from the amino acid sequence in palivizumab;
Fab fragment produced (*);
Monoclonal antibody produced (**);
Fab fragment & monoclonal antibody produced (***)

TABLE 3A
VH CDR1 Sequences

SVG (SEQ ID NO: 1)
SVG (SEQ ID NO: 10)
SVG (SEQ ID NO: 18)
Bold faced & underlined amino acid residues are the residues which differ from the amino acid sequence in palivizumab

TABLE 3B
VH CDR2 Sequences

| | |
|---|---|
| DDKKDYNPSLKS (SEQ ID NO: 2) | DGKKDYNPSLKS (SEQ ID NO: 100) |
| DDKKDYNPSLKD (SEQ ID NO: 86) | DGKKDYNPSLKD (SEQ ID NO: 103) |
| DDKKHYNPSLKS (SEQ ID NO: 82) | DGKKHYNPSLKS (SEQ ID NO: 106) |
| DDKKHYNPSLKD (SEQ ID NO: 19) | DGKKHYNPSLKD (SEQ ID NO: 25) |
| DDKKSYNPSLKS (SEQ ID NO: 109) | DGKKSYNPSLKS (SEQ ID NO: 114) |
| DDKKSYNPSLKD (SEQ ID NO: 111) | DGKKSYNPSLKD (SEQ ID NO: 41) |
| DDKGDYNPSLKS (SEQ ID NO: 384) | DGKGDYNPSLKS (SEQ ID NO: 390) |
| DDKGDYNPSLKD (SEQ ID NO: 385) | DGKGDYNPSLKD (SEQ ID NO: 391) |
| DDKGHYNPSLKS (SEQ ID NO: 386) | DGKGHYNPSLKS (SEQ ID NO: 392) |
| DDKGHYNPSLKD (SEQ ID NO: 387) | DGKGHYNPSLKD (SEQ ID NO: 393) |
| DDKGSYNPSLKS (SEQ ID NO: 388) | DGKGSYNPSLKS (SEQ ID NO: 394) |
| DDKGSYNPSLKD (SEQ ID NO: 389) | DGKGSYNPSLKD (SEQ ID NO: 395) |

Bold faced & underlined amino acid residues are the residues which differ from the amino acid sequence in palivizumab

TABLE 3C
VH CDR3 Sequences

| | |
|---|---|
| NWYFDV (SEQ ID NO: 3) | NWYFDV (SEQ ID NO: 83) |
| NFYFDV (SEQ ID NO: 12) | NFYFDV (SEQ ID NO: 29) |
| NWYFDV (SEQ ID NO: 94) | NWYFDV (SEQ ID NO: 79) |
| NFYFDV (SEQ ID NO: 97) | NFYFDV (SEQ ID NO: 20) |

Bold faced & underlined amino acid residues are the residues which differ from the amino acid sequence in palivizumab

TABLE 3D
VL CDR1 Sequences

| | | |
|---|---|---|
| KCQLSVGYMH (SEQ ID NO: 4) | SCQLSVGYMH (SEQ ID NO: 127) | LCQLSVGYMH (SEQ ID NO: 204) |
| RVGYMH (SEQ ID NO: 87) | RVGYMH (SEQ ID NO: 132) | RVGYMH (SEQ ID NO: 206) |
| KCQLFVGYMH (SEQ ID NO: 396) | SCQLFVGYMH (SEQ ID NO: 436) | LCQLFVGYMH (SEQ ID NO: 476) |
| KCQSSVGYMH (SEQ ID NO: 80) | SCQSSVGYMH (SEQ ID NO: 129) | LCQSSVGYMH (SEQ ID NO: 205) |
| KCQSRVGYMH (SEQ ID NO: 84) | SCQSRVGYMH (SEQ ID NO: 130) | LCQSRVGYMH (SEQ ID NO: 203) |
| KCQSFVGYMH (SEQ ID NO: 397) | SCQSFVGYMH (SEQ ID NO: 437) | LCQSFVGYMH (SEQ ID NO: 477) |
| KCQVSVGYMH (SEQ ID NO: 398) | SCQVSVGYMH (SEQ ID NO: 438) | LCQVSVGYMH (SEQ ID NO: 478) |
| KCQVRVGYMH (SEQ ID NO: 399) | SCQVRVGYMH (SEQ ID NO: 439) | LCQVRVGYMH (SEQ ID NO: 479) |
| KCQVFVGYMH (SEQ ID NO: 400) | SCQVFVGYMH (SEQ ID NO: 440) | LCQVFVGYMH (SEQ ID NO: 480) |
| KCSLSVGYMH (SEQ ID NO: 112) | SCSLSVGYMH (SEQ ID NO: 142) | LCSLSVGYMH (SEQ ID NO: 196) |
| KCSLRVGYMH (SEQ ID NO: 119) | SCSLRVGYMH (SEQ ID NO: 148) | LCSLRVGYMH (SEQ ID NO: 198) |
| KCSLFVGYMH (SEQ ID NO: 401) | SCSLFVGYMH (SEQ ID NO: 441) | LCSLFVGYMH (SEQ ID NO: 481) |
| KCSSSVGYMH (SEQ ID NO: 115) | SCSSSVGYMH (SEQ ID NO: 144) | LCSSSVGYMH (SEQ ID NO: 197) |
| KCSSRVGYMH (SEQ ID NO: 117) | SCSSRVGYMH (SEQ ID NO: 146) | LCSSRVGYMH (SEQ ID NO: 195) |
| KCSSFVGYMH (SEQ ID NO: 402) | SCSSFVGYMH (SEQ ID NO: 442) | LCSSFVGYMH (SEQ ID NO: 482) |
| KCSVSVGYMH (SEQ ID NO: 403) | SCSVSVGYMH (SEQ ID NO: 443) | LCSVSVGYMH (SEQ ID NO: 483) |
| KCSVRVGYMH (SEQ ID NO: 404) | SCSVRVGYMH (SEQ ID NO: 444) | LCSVRVGYMH (SEQ ID NO: 484) |
| KCSVFVGYMH (SEQ ID NO: 405) | SCSVFVGYMH (SEQ ID NO: 445) | LCSVFVGYMH (SEQ ID NO: 485) |
| KAQLSVGYMH (SEQ ID NO: 182) | SAQLSVGYMH (SEQ ID NO: 207) | LAQLSVGYMH (SEQ ID NO: 486) |
| KAQLRVGYMH (SEQ ID NO: 180) | SAQLRVGYMH (SEQ ID NO: 190) | LAQLRVGYMH (SEQ ID NO: 487) |
| KAQLFVGYMH (SEQ ID NO: 406) | SAQLFVGYMH (SEQ ID NO: 446) | LAQLFVGYMH (SEQ ID NO: 488) |
| KAQSSVGYMH (SEQ ID NO: 181) | SAQSSVGYMH (SEQ ID NO: 191) | LAQSSVGYMH (SEQ ID NO: 489) |
| KAQSRVGYMH (SEQ ID NO: 179) | SAQSRVGYMH (SEQ ID NO: 189) | LAQSRVGYMH (SEQ ID NO: 490) |
| KAQSFVGYMH (SEQ ID NO: 407) | SAQSFVGYMH (SEQ ID NO: 447) | LAQSFVGYMH (SEQ ID NO: 491) |
| KAQVSVGYMH (SEQ ID NO: 408) | SAQVSVGYMH (SEQ ID NO: 448) | LAQVSVGYMH (SEQ ID NO: 492) |
| KAQVRVGYMH (SEQ ID NO: 409) | SAQVRVGYMH (SEQ ID NO: 449) | LAQVRVGYMH (SEQ ID NO: 493) |
| KAQVFVGYMH (SEQ ID NO: 410) | SAQVFVGYMH (SEQ ID NO: 450) | LAQVFVGYMH (SEQ ID NO: 494) |
| KASLSVGYMH (SEQ ID NO: 186) | SASLSVGYMH (SEQ ID NO: 188) | LASLSVGYMH (SEQ ID NO: 495) |
| KASLRVGYMH (SEQ ID NO: 184) | SASLRVGYMH (SEQ ID NO: 187) | LASLRVGYMH (SEQ ID NO: 496) |
| KASLFVGYMH (SEQ ID NO: 411) | SASLFVGYMH (SEQ ID NO: 451) | LASLFVGYMH (SEQ ID NO: 497) |
| KASSSVGYMH (SEQ ID NO: 185) | SASSSVGYMH (SEQ ID NO: 14) | LASSSVGYMH (SEQ ID NO: 498) |
| KASSRVGYMH (SEQ ID NO: 183) | SASSRVGYMH (SEQ ID NO: 39) | LASSRVGYMH (SEQ ID NO: 499) |
| KASSFVGYMH (SEQ ID NO: 412) | SASSFVGYMH (SEQ ID NO: 452) | LASSFVGYMH (SEQ ID NO: 500) |
| KPASVSVGYMH (SEQ ID NO: 413) | SASVSVGYMH (SEQ ID NO: 453) | LASVSVGYMH (SEQ ID NO: 501) |
| KASVRVGYMH (SEQ ID NO: 414) | SASVRVGYMH (SEQ ID NO: 454) | LASVRVGYMH (SEQ ID NO: 502) |
| KASVFVGYMH (SEQ ID NO: 415) | SASVFVGYMH (SEQ ID NO: 455) | LASVFVGYMH (SEQ ID NO: 503) |
| KLQLSVGYMH (SEQ ID NO: 89) | SLQLSVGYMH (SEQ ID NO: 134) | LLQLSVGYMH (SEQ ID NO: 504) |
| KLQLRVGYMH (SEQ ID NO: 98) | SLQLRVGYMH (SEQ ID NO: 140) | LLQLRVGYMH (SEQ ID NO: 505) |
| KLQLFVGYMH (SEQ ID NO: 416) | SLQLFVGYMH (SEQ ID NO: 456) | LLQLFVGYMH (SEQ ID NO: 506) |
| KLQSSVGYMH (SEQ ID NO: 92) | SLQSSVGYMH (SEQ ID NO: 136) | LLQSSVGYMH (SEQ ID NO: 507) |
| KLQSRVGYMH (SEQ ID NO: 95) | SLQSRVGYMH (SEQ ID NO: 138) | LLQSRVGYMH (SEQ ID NO: 508) |
| KLQSFVGYMH (SEQ ID NO: 417) | SLQSFVGYMH (SEQ ID NO: 457) | LLQSFVGYMH (SEQ ID NO: 509) |
| KLQVSVGYMH (SEQ ID NO: 418) | SLQVSVGYMH (SEQ ID NO: 458) | LLQVSVGYMH (SEQ ID NO: 510) |
| KLQVRVGYMH (SEQ ID NO: 419) | SLQVRVGYMH (SEQ ID NO: 459) | LLQVRVGYMH (SEQ ID NO: 511) |
| KLQVFVGYMH (SEQ ID NO: 420) | SLQVFVGYMH (SEQ ID NO: 460) | LLQVFVGYMH (SEQ ID NO: 512) |
| KLSLSVGYMH (SEQ ID NO: 101) | SLSLSVGYMH (SEQ ID NO: 120) | LLSLSVGYMH (SEQ ID NO: 513) |
| KLSLRVGYMH (SEQ ID NO: 110) | SLSLRVGYMH (SEQ ID NO: 125) | LLSLRVGYMH (SEQ ID NO: 514) |
| KLSLFVGYMH (SEQ ID NO: 421) | SLSLFVGYMH (SEQ ID NO: 461) | LLSLFVGYMH (SEQ ID NO: 515) |
| KLSSSVGYMH (SEQ ID NO: 104) | SLSSSVGYMH (SEQ ID NO: 122) | LLSSSVGYMH (SEQ ID NO: 516) |
| KLSSRVGYMH (SEQ ID NO: 107) | SLSSRVGYMH (SEQ ID NO: 22) | LLSSRVGYMH (SEQ ID NO: 517) |
| KLSSFVGYMH (SEQ ID NO: 422) | SLSSFVGYMH (SEQ ID NO: 462) | LLSSFVGYMH (SEQ ID NO: 518) |
| KLSVSVGYMH (SEQ ID NO: 423) | SLSVSVGYMH (SEQ ID NO: 463) | LLSVSVGYMH (SEQ ID NO: 519) |
| KLSVRVGYMH (SEQ ID NO: 424) | SLSVRVGYMH (SEQ ID NO: 464) | LLSVRVGYMH (SEQ ID NO: 520) |
| KLSVFVGYMH (SEQ ID NO: 425) | SLSVFVGYMH (SEQ ID NO: 465) | LLSVFVGYMH (SEQ ID NO: 521) |
| KPQLSVGYMH (SEQ ID NO: 163) | SPQLSVGYMH (SEQ ID NO: 177) | LPQLSVGYMH (SEQ ID NO: 200) |
| KPQLRVGYMH (SEQ ID NO: 159) | SPQLRVGYMH (SEQ ID NO: 173) | LPQLRVGYMH (SEQ ID NO: 202) |
| KPQLFVGYMH (SEQ ID NO: 426) | SPQLFVGYMH (SEQ ID NO: 466) | LPQLFVGYMH (SEQ ID NO: 522) |
| KPQSSVGYMH (SEQ ID NO: 161) | SPQSSVGYMH (SEQ ID NO: 176) | LPQSSVGYMH (SEQ ID NO: 201) |
| KPQSRVGYMH (SEQ ID NO: 157) | SPQSRVGYMH (SEQ ID NO: 171) | LPQSRVGYMH (SEQ ID NO: 199) |
| KPQSFVGYMH (SEQ ID NO: 427) | SPQSFVGYMH (SEQ ID NO: 467) | LPQSFVGYMH (SEQ ID NO: 523) |
| KPQVSVGYMH (SEQ ID NO: 428) | SPQVSVGYMH (SEQ ID NO: 468) | LPQVSVGYMH (SEQ ID NO: 524) |
| KPQVRVGYMH (SEQ ID NO: 429) | SPQVRVGYMH (SEQ ID NO: 469) | LPQVRVGYMH (SEQ ID NO: 525) |
| KPQVFVGYMH (SEQ ID NO: 430) | SPQVFVGYMH (SEQ ID NO: 470) | LPQVFVGYMH (SEQ ID NO: 526) |
| KPSLSVGYMH (SEQ ID NO: 155) | SPSLSVGYMH (SEQ ID NO: 169) | LPSLSVGYMH (SEQ ID NO: 192) |
| KPSLRVGYMH (SEQ ID NO: 152) | SPSLRVGYMH (SEQ ID NO: 166) | LPSLRVGYMH (SEQ ID NO: 194) |
| KPSLFVGYMH (SEQ ID NO: 431) | SPSLFVGYMH (SEQ ID NO: 471) | LPSLFVGYMH (SEQ ID NO: 527) |
| KPPSSSVGYMH (SEQ ID NO: 153) | SPSSSVGYMH (SEQ ID NO: 168) | LPSSSVGYMH (SEQ ID NO: 193) |
| KPSSRVGYMH (SEQ ID NO: 150) | SPSSRVGYMH (SEQ ID NO: 31) | LPSSRVGYMH (SEQ ID NO: 47) |
| KPSSFVGYMH (SEQ ID NO: 432) | SPSSFVGYMH (SEQ ID NO: 472) | LPSSFVGYMH (SEQ ID NO: 528) |
| KPSVSVGYMH (SEQ ID NO: 433) | SPSVSVGYMH (SEQ ID NO: 473) | LPSVSVGYMH (SEQ ID NO: 529) |
| KPSVRVGYMH (SEQ ID NO: 434) | SPSVRVGYMH (SEQ ID NO: 474) | LPSVRVGYMH (SEQ ID NO: 530) |
| KPSVFVGYMH (SEQ ID NO: 435) | SPSVFVGYMH (SEQ ID NO: 475) | LPSVFVGYMH (SEQ ID NO: 531) |

Bold faced & underlined amino acid residues are the residues which differ from the amino acid sequence in palivizumab

TABLE 3E
VL CDR2 Sequences

| | | | | | | |
|---|---|---|---|---|---|---|
| DTSKLAS (SEQ | DTFKLAS (SEQ | DTYKLAS (SEQ | DTRKLAS (SEQ | DTMKLAS (SEQ | DTKKLAS (SEQ | DTLKLAS (SEQ |
| ID NO: 5) | ID NO: 15) | ID NO: 799) | ID | ID | ID NO: 1211) | ID NO: 135) |
| | | | NOS: 113&174) | NOS: 121&162) | | |
| DTSKLSS (SEQ | DTFKLSS (SEQ | DTYKLSS (SEQ | DTRKLSS (SEQ | DTMKLSS (SEQ | DTKKLSS (SEQ | DTLKLSS (SEQ |
| ID NO: 165) | ID NO: 96) | ID NO: 800) | ID NO: 175) | ID NO: 164) | ID NO: 1212) | ID NO: 1355) |
| DTSKLKS (SEQ | DTFKLKS (SEQ | DTYKLKS (SEQ | DTRKLKS (SEQ | DTMKLKS (SEQ | DTKKLKS (SEQ | DTLKLKS (SEQ |
| ID NO: 532) | ID NO: 660) | ID NO: 801) | ID NO: 943) | ID NO: 1076) | ID NO: 1213) | ID NO: 1356) |
| DTSKLRS (SEQ | DTFKLRS (SEQ | DTYKLRS (SEQ | DTRKLRS (SEQ | DTMKLRS (SEQ | DTKKLRS (SEQ | DTLKLRS (SEQ |
| ID NO: 533) | ID NO: 661) | ID NO: 802) | ID NO: 944) | ID NO: 1077) | ID NO: 1214) | ID NO: 1357) |
| DTSKLHS (SEQ | DTFKLHS (SEQ | DTYKLHS (SEQ | DTRKLHS (SEQ | DTMKLHS (SEQ | DTKKLHS (SEQ | DTLKLRS (SEQ |
| ID NO: 534) | ID NO: 662) | ID NO: 803) | ID NO: 945) | ID NO: 1078) | ID NO: 1215) | ID NO: 1358) |
| DTSKLPS (SEQ | DTFKLPS (SEQ | DTYKLPS (SEQ | DTRKLPS (SEQ | DTMKLPS (SEQ | DTKKLPS (SEQ | DTLKLPS (SEQ |
| ID NO: 102) | ID NO: 663) | ID NO: 804) | ID NO: 118) | ID NO: 1079) | ID NO: 1216) | ID NO: 1359) |
| DTSKLTS (SEQ | DTFKLTS (SEQ | DTYKLTS (SEQ | DTRKLTS (SEQ | DTMKLTS (SEQ | DTKKLTS (SEQ | DTLKLTS (SEQ |
| ID NO: 535) | ID NO: 664) | ID NO: 805) | ID NO: 946) | ID NO: 1080) | ID NO: 1217) | ID NO: 1360) |
| DTSKLDS (SEQ | DTFKLDS (SEQ | DTYKLDS (SEQ | DTRKLDS (SEQ | DTMKLDS (SEQ | DTKKLDS (SEQ | DTLKLDS (SEQ |
| ID NO: 128) | ID NO: 665) | ID NO: 806) | ID NO: 947) | ID NO: 1081) | ID NO: 1218) | ID NO: 131) |
| DTSKHAS (SEQ | DTFKHAS (SEQ | DTYKHAS (SEQ | DTRKHAS (SEQ | DTMKHAS (SEQ | DTKKHAS (SEQ | DTLKHAS (SEQ |
| ID NO: 536) | ID NO: 666) | ID NO: 807) | ID NO: 948) | ID NO: 1082) | ID NO: 1219) | ID NO: 1361) |
| DTSKHSS (SEQ | DTFKHSS (SEQ | DTYKHSS (SEQ | DTRKHSS (SEQ | DTMKHSS (SEQ | DTKKHSS (SEQ | DTLKHSS (SEQ |
| ID NO: 537) | ID NO: 667) | ID NO: 808) | ID NO: 949) | ID NO: 1083) | ID NO: 1220) | ID NO: 1362) |
| DTSKHKS (SEQ | DTFKHKS (SEQ | DTYKHKS (SEQ | DTRKHKS (SEQ | DTMKHKS (SEQ | DTKKHKS (SEQ | DTLKHKS (SEQ |
| ID NO: 538) | ID NO: 668) | ID NO: 809) | ID NO: 950) | ID NO: 1084) | ID NO: 1221) | ID NO: 1363) |
| DTSKHRS (SEQ | DTFKHRS (SEQ | DTYKHRS (SEQ | DTRKHRS (SEQ | DTMKHRS (SEQ | DTKKHRS (SEQ | DTLKHRS (SEQ |
| ID NO: 539) | ID NO: 669) | ID NO: 810) | ID NO: 951) | ID NO: 1085) | ID NO: 1222) | ID NO: 1364) |
| DTSKHHS (SEQ | DTFKHHS (SEQ | DTYKHHS (SEQ | DTRKHHS (SEQ | DTMKHHS (SEQ | DTKKHHS (SEQ | DTLKHHS (SEQ |
| ID NO: 540) | ID NO: 670) | ID NO: 811) | ID NO: 952) | ID NO: 1086) | ID NO: 1223) | ID NO: 1365) |
| DTSKHPS (SEQ | DTFKHPS (SEQ | DTYKHPS (SEQ | DTRKHPS (SEQ | DTMKHPS (SEQ | DTKKHPS (SEQ | DTLKHPS (SEQ |
| ID NO: 541) | ID NO: 671) | ID NO: 812) | ID NO: 953) | ID NO: 1087) | ID NO: 1224) | ID NO: 1366) |
| DTSKHTS (SEQ | DTFKHTS (SEQ | DTYKHTS (SEQ | DTRKHTS (SEQ | DTMKHTS (SEQ | DTKKHTS (SEQ | DTLKHTS (SEQ |
| ID NO: 542) | ID NO: 672) | ID NO: 813) | ID NO: 954) | ID NO: 1088) | ID NO: 1225) | ID NO: 1367) |
| DTSKHDS (SEQ | DTFKHDS (SEQ | DTYKHDS (SEQ | DTRKHDS (SEQ | DTMKHDS (SEQ | DTKKHDS (SEQ | DTLKHDS (SEQ |
| ID NO: 543) | ID NO: 673) | ID NO: 814) | ID NO: 955) | ID NO: 1089) | ID NO: 1226) | ID NO: 1368) |
| DTSKQAS (SEQ | DTFKQAS (SEQ | DTYKQAS (SEQ | DTRKQAS (SEQ | DTMKQAS (SEQ | DTKKQAS (SEQ | DTLKQAS (SEQ |
| ID NO: 139) | ID NO: 674) | ID NO: 815) | ID NO: 170) | ID NO: 154) | ID NO: 1227) | ID NO: 1369) |
| DTSKQSS (SEQ | DTFKQSS (SEQ | DTYKQSS (SEQ | DTRKQSS (SEQ | DTMKQSS (SEQ | DTKKQSS (SEQ | DTLKQSS (SEQ |
| ID NO: 141) | ID NO: 675) | ID NO: 816) | ID NO: 172) | ID NO: 156) | ID NO: 1228) | ID NO: 1370) |
| DTSKQKS (SEQ | DTFKQKS (SEQ | DTYKQKS (SEQ | DTRKQKS (SEQ | DTMKQKS (SEQ | DTKKQKS (SEQ | DTLKQKS (SEQ |
| ID NO: 544) | ID NO: 676) | ID NO: 817) | ID NO: 956) | ID NO: 1090) | ID NO: 1229) | ID NO: 1371) |
| DTSKQRS (SEQ | DTFKQRS (SEQ | DTYKQRS (SEQ | DTRKQRS (SEQ | DTMKQRS (SEQ | DTKKQRS (SEQ | DTLKQRS (SEQ |
| ID NO: 545) | ID NO: 677) | ID NO: 818) | ID NO: 957) | ID NO: 1091) | ID NO: 1230) | ID NO: 1372) |
| DTSKQHS (SEQ | DTFKQHS (SEQ | DTYKQHS (SEQ | DTRKQHS (SEQ | DTMKQHS (SEQ | DTKKQHS (SEQ | DTLKQHS (SEQ |
| ID NO: 546) | ID NO: 678) | ID NO: 819) | ID NO: 958) | ID NO: 1092) | ID NO: 1231) | ID NO: 1373) |
| DTSKQPS (SEQ | DTFKQPS (SEQ | DTYKQPS (SEQ | DTRKQPS (SEQ | DTMKQPS (SEQ | DTKKQPS (SEQ | DTLKQPS (SEQ |
| ID NO: 547) | ID NO: 679) | ID NO: 820) | ID NO: 959) | ID NO: 1093) | ID NO: 1232) | ID NO: 1374) |
| DTSKQTS (SEQ | DTFKQTS (SEQ | DTYKQTS (SEQ | DTRKQTS (SEQ | DTMKQTS (SEQ | DTKKQTS (SEQ | DTLKQTS (SEQ |
| ID NO: 548) | ID NO: 680) | ID NO: 821) | ID NO: 960) | ID NO: 1094) | ID NO: 1223) | ID NO: 1375) |
| DTSKQDS (SEQ | DTFKQDS (SEQ | DTYKQDS (SEQ | DTRKQDS (SEQ | DTMKQDS (SEQ | DTKKQDS (SEQ | DTLKQDS (SEQ |
| ID NO: 549) | ID NO: 681) | ID NO: 822) | ID NO: 961) | ID NO: 1095) | ID NO: 1234) | ID NO: 1376) |
| DTSGLAS (SEQ | DTFGLAS (SEQ | DTYGLAS (SEQ | DTRGLAS (SEQ | DTMGLAS (SEQ | DTKGLAS (SEQ | DTLGLAS (SEQ |
| ID NO: 105) | ID NO: 682) | ID NO: 823) | ID NO: 116) | ID NO: 1096) | ID NO: 1235) | ID NO: 1377) |
| DTSGLSS (SEQ | DTFGLSS (SEQ | DTYGLSS (SEQ | DTRGLSS (SEQ | DTMGLSS (SEQ | DTKGLSS (SEQ | DTLGLSS (SEQ |
| ID NO: 550) | ID NO: 683) | ID NO: 824) | ID NO: 962) | ID NO: 1097) | ID NO: 1236) | ID NO: 1378) |
| DTSGLKS (SEQ | DTFGLKS (SEQ | DTYGLKS (SEQ | DTRGLKS (SEQ | DTMGLKS (SEQ | DTKGLKS (SEQ | DTLGLKS (SEQ |
| ID NO: 551) | ID NO: 684) | ID NO: 825) | ID NO: 963) | ID NO: 1098) | ID NO: 1237) | ID NO: 1379) |
| DTSGLRS (SEQ | DTFGLRS (SEQ | DTYGLRS (SEQ | DTRGLRS (SEQ | DTMGLRS (SEQ | DTKGLRS (SEQ | DTLGLRS (SEQ |
| ID NO: 552) | ID NO: 685) | ID NO: 826) | ID NO: 964) | ID NO: 1099) | ID NO: 1238) | ID NO: 1380) |
| DTSGLHS (SEQ | DTFGLHS (SEQ | DTYGLHS (SEQ | DTRGLHS (SEQ | DTMGLHS (SEQ | DTKGLHS (SEQ | DTLGLHS (SEQ |
| ID NO: 553) | ID NO: 686) | ID NO: 827) | ID NO: 965) | ID NO: 1100) | ID NO: 1239) | ID NO: 1381) |
| DTSGLPS (SEQ | DTFGLPS (SEQ | DTYGLPS (SEQ | DTRGLPS(SEQ | DTMGLPS (SEQ | DTKGLPS (SEQ | DTLGLPS (SEQ |
| ID NO: 108) | ID NO: 687) | ID NO: 828) | ID NO: 27) | ID NO: 1101) | ID NO: 1240) | ID NO: 1382) |
| DTSGLTS (SEQ | DTFGLTS (SEQ | DTYGLTS (SEQ | DTRGLTS (SEQ | DTMGLTS (SEQ | DTKGLTS (SEQ | DTLGLTS (SEQ |
| ID NO: 554) | ID NO: 688) | ID NO: 829) | ID NO: 966) | ID NO: 1102) | ID NO: 1241) | ID NO: 1383) |
| DTSGLDS (SEQ | DTFGLDS (SEQ | DTYGLDS (SEQ | DTRGLDS (SEQ | DTMGLDS (SEQ | DTKGLDS (SEQ | DTLGLDS (SEQ |
| ID NO: 555) | ID NO: 689) | ID NO: 830) | ID NO: 967) | ID NO: 1103) | ID NO: 1242) | ID NO: 1384) |
| DTSGHAS (SEQ | DTFGHAS (SEQ | DTYGHAS (SEQ | DTRGHAS (SEQ | DTMGHAS (SEQ | DTKGHAS (SEQ | DTLGHAS (SEQ |
| ID NO: 556) | ID NO: 690) | ID NO: 831) | ID NO: 968) | ID NO: 1104) | ID NO: 1243) | ID NO: 1385) |
| DTSGHSS (SEQ | DTFGHSS (SEQ | DTYGHSS (SEQ | DTRGHSS (SEQ | DTMGHSS (SEQ | DTKGHSS (SEQ | DTLGHSS (SEQ |
| ID NO: 557) | ID NO: 691) | ID NO: 832) | ID NO: 969) | ID NO: 1105) | ID NO: 1244) | ID NO: 1386) |
| DTSGHKS (SEQ | DTFGHKS (SEQ | DTYGHKS (SEQ | DTRGHKS (SEQ | DTMGHKS (SEQ | DTKGHKS (SEQ | DTLGHKS (SEQ |
| ID NO: 558) | ID NO: 692) | ID NO: 833) | ID NO: 970) | ID NO: 1106) | ID NO: 1245) | ID NO: 1387) |
| DTSGHRS (SEQ | DTFGHRS (SEQ | DTYGHRS (SEQ | DTRGHRS (SEQ | DTMGHRS (SEQ | DTKGHRS (SEQ | DTLGHRS (SEQ |
| ID NO: 559) | ID NO: 693) | ID NO: 834) | ID NO: 971) | ID NO: 1107) | ID NO: 1246) | ID NO: 1388) |
| DTSGHHS (SEQ | DTFGHHS (SEQ | DTYGHHS (SEQ | DTRGHHS (SEQ | DTMGHHS (SEQ | DTKGHHS (SEQ | DTLGHHS (SEQ |
| ID NO: 560) | ID NO: 694) | ID NO: 835) | ID NO: 972) | ID NO: 1108) | ID NO: 1247) | ID NO: 1389) |
| DTSGHPS (SEQ | DTFGHPS (SEQ | DTYGHPS (SEQ | DTRGHPS (SEQ | DTMGHPS (SEQ | DTKGHPS (SEQ | DTLGHPS (SEQ |
| ID NO: 561) | ID NO: 695) | ID NO: 836) | ID NO: 973) | ID NO: 1109) | ID NO: 1248) | ID NO: 1390) |
| DTSGHTS (SEQ | DTFGHTS (SEQ | DTYGHTS (SEQ | DTRGHTS (SEQ | DTMGHTS (SEQ | DTKGHTS (SEQ | DTLGHTS (SEQ |
| ID NO: 562) | ID NO: 696) | ID NO: 837) | ID NO: 974) | ID NO: 1110) | ID NO: 1249) | ID NO: 1391) |
| DTSGHDS (SEQ | DTFGHDS (SEQ | DTYGHDS (SEQ | DTRGHDS (SEQ | DTMGHDS (SEQ | DTKGHDS (SEQ | DTLGHDS (SEQ |
| ID NO: 563) | ID NO: 697) | ID NO: 838) | ID NO: 975) | ID NO: 1111) | ID NO: 1250) | ID NO: 1392) |
| DTSGQAS (SEQ | DTFGQAS (SEQ | DTYGQAS (SEQ | DTRGQAS (SEQ | DTMGQAS (SEQ | DTKGQAS (SEQ | DTLGQAS (SEQ |
| ID NO: 564) | ID NO: 698) | ID NO: 839) | ID NO: 976) | ID NO: 1112) | ID NO: 1251) | ID NO: 1393) |
| DTSGQSS (SEQ | DTFGQSS (SEQ | DTYGQSS (SEQ | DTRGQSS (SEQ | DTMGQSS (SEQ | DTKGQSS (SEQ | DTLGQSS (SEQ |
| ID NO: 565) | ID NO: 699) | ID NO: 840) | ID NO: 977) | ID NO: 1113) | ID NO: 1252) | ID NO: 1394) |
| DTSGQKS (SEQ | DTFGQKS (SEQ | DTYGQKS (SEQ | DTRGQKS (SEQ | DTMGQKS (SEQ | DTKGQKS (SEQ | DTLGQKS (SEQ |
| ID NO: 566) | ID NO: 700) | ID NO: 841) | ID NO: 978) | ID NO: 1114) | ID NO: 1253) | ID NO: 1395) |
| DTSGQRS (SEQ | DTFGQRS (SEQ | DTYGQRS (SEQ | DTRGQRS (SEQ | DTMGQRS (SEQ | DTKGQRS (SEQ | DTLGQRS (SEQ |
| ID NO: 567) | ID NO: 701) | ID NO: 842) | ID NO: 979) | ID NO: 1115) | ID NO: 1254) | ID NO: 1396) |
| DTSGQHS (SEQ | DTFGQHS (SEQ | DTYGQHS (SEQ | DTRGQHS (SEQ | DTMGQHS (SEQ | DTKGQHS (SEQ | DTLGQHS (SEQ |
| ID NO: 568) | ID NO: 702) | ID NO: 843) | ID NO: 980) | ID NO: 1116) | ID NO: 1255) | ID NO: 1397) |
| DTSGQPS (SEQ | DTFGQPS (SEQ | DTYGQPS (SEQ | DTRGQPS (SEQ | DTMGQPS (SEQ | DTKGQPS (SEQ | DTLGQPS (SEQ |
| ID NO: 569) | ID NO: 703) | ID NO: 844) | ID NO: 981) | ID NO: 1117) | ID NO: 1256) | ID NO: 1398) |
| DTSGQTS (SEQ | DTFGQTS (SEQ | DTYGQTS (SEQ | DTRGQTS (SEQ | DTMGQTS (SEQ | DTKGQTS (SEQ | DTLGQTS (SEQ |
| ID NO: 570) | ID NO: 704) | ID NO: 845) | ID NO: 982) | ID NO: 1118) | ID NO: 1257) | ID NO: 1399) |
| DTSGQDS (SEQ | DTFGQDS (SEQ | DTYGQDS (SEQ | DTRGQDS (SEQ | DTMGQDS (SEQ | DTKGQDS (SEQ | DTLGQDS (SEQ |
| ID NO: 571) | ID NO: 705) | ID NO: 846) | ID NO: 983) | ID NO: 1119) | ID NO: 1258) | ID NO: 1400) |
| DTSRLAS (SEQ | DTFRLAS (SEQ | DTYRLAS (SEQ | DTRRLAS (SEQ | DTMRLAS (SEQ | DTKRLAS (SEQ | DTLRLAS (SEQ |
| ID NO: 123) | ID NO: 706) | ID NO: 847) | ID NO: 984) | ID NO: 32) | ID NO: 1259) | ID NO: 1401) |
| DTSRLSS (SEQ | DTFRLSS (SEQ | DTYRLSS (SEQ | DTRRLSS (SEQ | DTMRLSS (SEQ | DTKRLSS (SEQ | DTLRLSS (SEQ |
| ID NO: 572) | ID NO: 707) | ID NO: 848) | ID NO: 985) | ID NO: 1120) | ID NO: 1260) | ID NO: 1402) |
| DTSRLKS (SEQ | DTFRLKS (SEQ | DTYRLKS (SEQ | DTRRLKS (SEQ | DTMRLKS (SEQ | DTKRLKS (SEQ | DTLRLKS (SEQ |
| ID NO: 573) | ID NO: 708) | ID NO: 849) | ID NO: 986) | ID NO: 1121) | ID NO: 1261) | ID NO: 1403) |
| DTSRLRS (SEQ | DTFRLRS (SEQ | DTYRLRS (SEQ | DTRRLRS (SEQ | DTMRLRS (SEQ | DTKRLRS (SEQ | DTLRLRS (SEQ |
| ID NO: 574) | ID NO: 709) | ID NO: 850) | ID NO: 987) | ID NO: 1122) | ID NO: 1262) | ID NO: 1404) |
| DTSRLHS (SEQ | DTFRLHS (SEQ | DTYRLHS (SEQ | DTRRLHS (SEQ | DTMRLHS (SEQ | DTKRLHS (SEQ | DTLRLHS (SEQ |
| ID NO: 575) | ID NO: 710) | ID NO: 851) | ID NO: 988) | ID NO: 1123) | ID NO: 1263) | ID NO: 1405) |
| DTSRLPS (SEQ | DTFR1LPS (SEQ | DTYRLPS (SEQ | DTRRLPS (SEQ | DTMRLPS (SEQ | DTKRLPS (SEQ | DTLRLPS (SEQ |
| ID NO: 576) | ID NO: 711) | ID NO: 852) | ID NO: 989) | ID NO: 1124) | ID NO: 1264) | ID NO: 1406) |
| DTSRLTS (SEQ | DTFRLTS (SEQ | DTYRLTS (SEQ | DTRRLTS (SEQ | DTMRLTS (SEQ | DTKRLTS (SEQ | DTLRLTS (SEQ |
| ID NO: 577) | ID NO: 712) | ID NO: 853) | ID NO: 990) | ID NO: 1125) | ID NO: 1265) | ID NO: 1407) |
| DTSRLDS (SEQ | DTFRLDS (SEQ | DTYRLDS (SEQ | DTRRLDS (SEQ | DTMRLDS (SEQ | DTKRLDS (SEQ | DTLRLDS (SEQ |
| ID NO: 578) | ID NO: 713) | ID NO: 854) | ID NO: 991) | ID NO: 1126) | ID NO: 1266) | ID NO: 1408) |
| DTSRHAS (SEQ | DTFRHAS (SEQ | DTYRHAS (SEQ | DTRRHAS (SEQ | DTMRHHS (SEQ | DTKRHAS (SEQ | DTLRHAS (SEQ |
| ID NO: 579) | ID NO: 714) | ID NO: 855) | ID NO: 992) | ID NO: 1127) | ID NO: 1267) | ID NO: 1409) |
| DTSRHSS (SEQ | DTFRHSS (SEQ | DTYRHSS (SEQ | DTRRHSS (SEQ | DTMRHSS (SEQ | DTKRHSS (SEQ | DTLRHSS (SEQ |
| ID NO: 580) | ID NO: 715) | ID NO: 856) | ID NO: 993) | ID NO: 1128) | ID NO: 1268) | ID NO: 1410) |
| DTSRHKS (SEQ | DTFRHKS (SEQ | DTYRHKS (SEQ | DTRRHKS (SEQ | DTMRHKS (SEQ | DTKRHKS (SEQ | DTLRHKS (SEQ |
| ID NO: 581) | ID NO: 716) | ID NO: 857) | ID NO: 994) | ID NO: 1129) | ID NO: 1269) | ID NO: 1411) |
| DTSRHRS (SEQ | DTFRHRS (SEQ | DTYRHRS (SEQ | DTRRHRS (SEQ | DTMRHRS (SEQ | DTKRHRS (SEQ | DTLRHRS (SEQ |
| ID NO: 582) | ID NO: 717) | ID NO: 858) | ID NO: 995) | ID NO: 1130) | ID NO: 1270) | ID NO: 1412) |
| DTSRHHS (SEQ | DTFRHHS (SEQ | DTYRHHS (SEQ | DTRRHHS (SEQ | DTMRHHS (SEQ | DTKRHHS (SEQ | DTLRHHS (SEQ |
| ID NO: 583) | ID NO: 718) | ID NO: 859) | ID NO: 996) | ID NO: 1131) | ID NO: 1271) | ID NO: 1413) |
| DTSRHPS (SEQ | DTFRHPS (SEQ | DTYRHPS (SEQ | DTRRHPS (SEQ | DTMRHPS (SEQ | DTKRHPS (SEQ | DTLRHPS (SEQ |
| ID NO: 584) | ID NO: 719) | ID NO: 860) | ID NO: 997) | ID NO: 1132) | ID NO: 1272) | ID NO: 1414) |
| DTSRHTS (SEQ | DTFRHTS (SEQ | DTYRHTS (SEQ | DTRRHTS (SEQ | DTMRHTS (SEQ | DTKRHTS (SEQ | DTLRHTS (SEQ |
| ID NO: 585) | ID NO: 720) | ID NO: 861) | ID NO: 998) | ID NO: 1133) | ID NO: 1273) | ID NO: 1415) |
| DTSRHDS (SEQ | DTFRHDS (SEQ | DTYRHDS (SEQ | DTRRHDS (SEQ | DTMRHDS (SEQ | DTKRHDS (SEQ | DTLRHDS (SEQ |
| ID NO: 586) | ID NO: 721) | ID NO: 862) | ID NO: 999) | ID NO: 1134) | ID NO: 1274) | ID NO: 1416) |
| DTSRQAS (SEQ | DTFRQAS (SEQ | DTYRQAS (SEQ | DTRRQAS (SEQ | DTMRQAS (SEQ | DTKRQAS (SEQ | DTLRQAS (SEQ |
| ID NO: 587) | ID NO: 722) | ID NO: 863) | ID NO: 1000) | ID NO: 1135) | ID NO: 1275) | ID NO: 1417) |
| DTSRQSS (SEQ | DTFRQSS (SEQ | DTYRQSS (SEQ | DTRRQSS (SEQ | DTMRQSS (SEQ | DTKRQSS (SEQ | DTLRQSS (SEQ |
| ID NO: 588) | ID NO: 723) | ID NO: 864) | ID NO: 1001) | ID NO: 1136) | ID NO: 1276) | ID NO: 1418) |
| DTSRQKS (SEQ | DTFRQKS (SEQ | DTYRQKS (SEQ | DTRRQKS (SEQ | DTMRQKS (SEQ | DTKRQKS (SEQ | DTLRQKS (SEQ |
| ID NO: 589) | ID NO: 724) | ID NO: 865) | ID NO: 1002) | ID NO: 1137) | ID NO: 1277) | ID NO: 1419) |
| DTSRQRS (SEQ | DTFRQRS (SEQ | DTYRQRS (SEQ | DTRRQRS (SEQ | DTMRQRS (SEQ | DTKRQRS (SEQ | DTLRQRS (SEQ |
| ID NO: 590) | ID NO: 725) | ID NO: 866) | ID NO: 1003) | ID NO: 1138) | ID NO: 1278) | ID NO: 1420) |
| DTSRQHS (SEQ | DTFRQHS (SEQ | DTYRQHS (SEQ | DTRRQHS (SEQ | DTMRQHS (SEQ | DTKRQHS (SEQ | DTLRQHS (SEQ |
| ID NO: 591) | ID NO: 726) | ID NO: 867) | ID NO: 1004) | ID NO: 1139) | ID NO: 1279) | ID NO: 1421) |
| DTSRQPS (SEQ | DTFRQPS (SEQ | DTYRQPS (SEQ | DTRRQPS (SEQ | DTMRQPS (SEQ | DTKRQPS (SEQ | DTLRQPS (SEQ |
| ID NO: 592) | ID NO: 727) | ID NO: 868) | ID NO: 1005) | ID NO: 1140) | ID NO: 1280) | ID NO: 1422) |
| DTSRQTS (SEQ | DTFRQTS (SEQ | DTYRQTS (SEQ | DTRRQTS (SEQ | DTMRQTS (SEQ | DTKRQTS (SEQ | DTLRQTS (SEQ |
| ID NO: 593) | ID NO: 728) | ID NO: 869) | ID NO: 1006) | ID NO: 1141) | ID NO: 1281) | ID NO: 1423) |
| DTSRQDS (SEQ | DTFRQDS (SEQ | DTYRQDS (SEQ | DTRRQDS (SEQ | DTMRQDS (SEQ | DTKRQDS (SEQ | DTLRQDS (SEQ |
| ID NO: 594) | ID NO: 729) | ID NO: 870) | ID NO: 1007) | ID NO: 1142) | ID NO: 1282) | ID NO: 1424) |
| DTSYLAS (SEQ | DTFYLAS (SEQ | DTYYLAS (SEQ | DTRYLAS(SEQ | DTMYLAS (SEQ | DTKYLAS (SEQ | DTLYLAS (SEQ |
| ID | ID NO: 99) | ID NO: 871) | ID NO: 178) | ID NO: 158) | ID NO: 1283) | ID NO: 1425) |
| NOS: 81&143) | | | | | | |
| DTSYLSS (SEQ | DTFYLSS (SEQ | DTYYLSS (SEQ | DTRYLSS(SEQ | DTMYLSS (SEQ | DTKYLSS (SEQ | DTLYLSS (SEQ |
| ID | ID NO: 90) | ID NO: 872) | ID NO: 59) | ID NO: 160) | ID NO: 1284) | ID NO: 1426) |
| NOS: 85&145) | | | | | | |
| DTSYLKS (SEQ | DTFYLKS (SEQ | DTYYLKS (SEQ | DTRYLKS (SEQ | DTMYLKS (SEQ | DTKYLKS (SEQ | DTLYLKS (SEQ |
| ID NO: 595) | ID NO: 730) | ID NO: 873) | ID NO: 1008) | ID NO: 1143) | ID NO: 1285) | ID NO: 1427) |
| DTSYLRS (SEQ | DTFYLRS (SEQ | DTYYLRS (SEQ | DTRYLRS (SEQ | DTMYLRS (SEQ | DTKYLRS (SEQ | DTLYLRS (SEQ |
| ID NO: 596) | ID NO: 731) | ID NO: 874) | ID NO: 1009) | ID NO: 1144) | ID NO: 1286) | ID NO: 1428) |
| DTSYLHS (SEQ | DTFYLHS (SEQ | DTYYLHS (SEQ | DTRYLHS (SEQ | DTMYLHS (SEQ | DTKYLHS (SEQ | DTLYLHS (SEQ |
| ID NO: 597) | ID NO: 732) | ID NO: 875) | ID NO: 1010) | ID NO: 1145) | ID NO: 1287) | ID NO: 1429) |
| DTSYLPS (SEQ | DTFYLPS (SEQ | DTYYLPS (SEQ | DTRYLPS (SEQ | DTMYLPS (SEQ | DTKYLPS (SEQ | DTLYLPS (SEQ |
| ID NO: 598) | ID NO: 733) | ID NO: 876) | ID NO: 1011) | ID NO: 1146) | ID NO: 1288) | ID NO: 1430) |
| DTSYLTS (SEQ | DTFYLTS (SEQ | DTYYLTS (SEQ | DTRYLTS (SEQ | DTMYLTS (SEQ | DTKYLTS (SEQ | DTLYLTS (SEQ |
| ID NO: 599) | ID NO: 734) | ID NO: 877) | ID NO: 1012) | ID NO: 1147) | ID NO: 1289) | ID NO: 1431) |
| DTSYLDS (SEQ | DTFYLDS (SEQ | DTYYLDS (SEQ | DTRYLDS (SEQ | DTMYLDS (SEQ | DTKYLDS (SEQ | DTLYLDS (SEQ |
| ID NO: 600) | ID NO: 735) | ID NO: 878) | ID NO: 1013) | ID NO: 1148) | ID NO: 1290) | ID NO: 1432) |
| DTSYHAS (SEQ | DTFYHAS (SEQ | DTYYHAS (SEQ | DTRYHAS (SEQ | DTMYHAS (SEQ | DTKYHAS (SEQ | DTLYHAS (SEQ |
| ID NO: 601) | ID NO: 736) | ID NO: 879) | ID NO: 1014) | ID NO: 1149) | ID NO: 1291) | ID NO: 1433) |
| DTSYHSS (SEQ | DTFYHSS (SEQ | DTYYHSS (SEQ | DTRYHSS (SEQ | DTMYHSS (SEQ | DTKYHSS (SEQ | DTLYHSS (SEQ |
| ID NO: 602) | ID NO: 737) | ID NO: 880) | ID NO: 1015) | ID NO: 1150) | ID NO: 1292) | ID NO: 1434) |
| DTSYHKS (SEQ | DTFYHKS (SEQ | DTYYHKS (SEQ | DTRYHKS (SEQ | DTMYHKS (SEQ | DTKYHKS (SEQ | DTLYHKS (SEQ |
| ID NO: 603) | ID NO: 738) | ID NO: 881) | ID NO: 1016) | ID NO: 1151) | ID NO: 1293) | ID NO: 1435) |
| DTSYHRS (SEQ | DTFYHRS (SEQ | DTYYHRS (SEQ | DTRYHRS (SEQ | DTMYHRS (SEQ | DTKYHRS (SEQ | DTLYHRS (SEQ |
| ID NO: 604) | ID NO: 739) | ID NO: 882) | ID NO: 1017) | ID NO: 1152) | ID NO: 1294) | ID NO: 1436) |
| DTSYHHS (SEQ | DTFYHHS (SEQ | DTYYHHS (SEQ | DTRYHHS (SEQ | DTMYHHS (SEQ | DTKYHHS (SEQ | DTLYHHS (SEQ |
| ID NO: 605) | ID NO: 740) | ID NO: 883) | ID NO: 1018) | ID NO: 1153) | ID NO: 1295) | ID NO: 1437) |
| DTSYHPS (SEQ | DTFYHPS (SEQ | DTYYHPS (SEQ | DTRYHPS (SEQ | DTMYHPS (SEQ | DTKYHPS (SEQ | DTLYHPS (SEQ |
| ID NO: 606) | ID NO: 741) | ID NO: 884) | ID NO: 1019) | ID NO: 1154) | ID NO: 1296) | ID NO: 1438) |
| DTSYHTS (SEQ | DTFYHTS (SEQ | DTYYHTS (SEQ | DTRYHTS (SEQ | DTMYHTS (SEQ | DTKYHTS (SEQ | DTLYHTS (SEQ |
| ID NO: 607) | ID NO: 742) | ID NO: 885) | ID NO: 1020) | ID NO: 1155) | ID NO: 1297) | ID NO: 1439) |
| DTSYHDS (SEQ | DTFYHDS (SEQ | DTYYHDS (SEQ | DTRYHDS (SEQ | DTMYHDS (SEQ | DTKYHDS (SEQ | DTLYHDS (SEQ |
| ID NO: 608) | ID NO: 743) | ID NO: 886) | ID NO: 1021) | ID NO: 1156) | ID NO: 1298) | ID NO: 1440) |
| DTSYQAS (SEQ | DTFYQAS (SEQ | DTYYQAS (SEQ | DTRYQAS(SEQ | DTMYQAS (SEQ | DTKYQAS (SEQ | DTLYQAS (SEQ |
| ID NO: 147) | ID NO: 744) | ID NO: 887) | ID NO: 167) | ID NO: 151) | ID NO: 1299) | ID NO: 1441) |
| DTSYQSS (SEQ | DTFYQSS (SEQ | DTYYQSS (SEQ | DTRYQSS(SEQ | DTMYQSS (SEQ | DTKYQSS (SEQ | DTLYQSS (SEQ |
| ID NO: 149) | ID NO: 745) | ID NO: 888) | ID NO: 53) | ID NO: 43) | ID NO: 1300) | ID NO: 1442) |
| DTSYQKS (SEQ | DTFYQKS (SEQ | DTYYQKS (SEQ | DTRYQKS (SEQ | DTMYQKS (SEQ | DTKYQKS (SEQ | DTLYQKS (SEQ |
| ID NO: 609) | ID NO: 746) | ID NO: 889) | ID NO: 1022) | ID NO: 1157) | ID NO: 1301) | ID NO: 1443) |
| DTSYQRS (SEQ | DTFYQRS (SEQ | DTYYQRS (SEQ | DTRYQRS (SEQ | DTMYQRS (SEQ | DTKYQRS (SEQ | DTLYQRS (SEQ |
| ID NO: 610) | ID NO: 747) | ID NO: 890) | ID NO: 1023) | ID NO: 1158) | ID NO: 1302) | ID NO: 1444) |
| DTSYQHS (SEQ | DTFYQHS (SEQ | DTYYQHS (SEQ | DTRYQHS (SEQ | DTMYQHS (SEQ | DTKYQHS (SEQ | DTLYQHS (SEQ |
| ID NO: 611) | ID NO: 748) | ID NO: 891) | ID NO: 1024) | ID NO: 1159) | ID NO: 1303) | ID NO: 1445) |
| DTSYQPS (SEQ | DTFYQPS (SEQ | DTYYQPS (SEQ | DTRYQPS (SEQ | DTMYQPS (SEQ | DTKYQPS (SEQ | DTLYQPS (SEQ |
| ID NO: 612) | ID NO: 749) | ID NO: 892) | ID NO: 1025) | ID NO: 1160) | ID NO: 1304) | ID NO: 1446) |
| DTSYQTS (SEQ | DTFYQTS (SEQ | DTYYQTS (SEQ | DTRYQTS (SEQ | DTMYQTS (SEQ | DTKYQTS (SEQ | DTLYQTS (SEQ |
| ID NO: 613) | ID NO: 750) | ID NO: 893) | ID NO: 1026) | ID NO: 1161) | ID NO: 1305) | ID NO: 1447) |
| DTSYQDS (SEQ | DTFYQDS (SEQ | DTYYQDS (SEQ | DTRYQDS (SEQ | DTMYQDS (SEQ | DTKYQDS (SEQ | DTLYQDS (SEQ |
| ID NO: 614) | ID NO: 751) | ID NO: 894) | ID NO: 1027) | ID NO: 1162) | ID NO: 1306) | ID NO: 1448) |
| DTSFLAS (SEQ | DTFFLAS (SEQ | DTYFLAS (SEQ | DTRFLAS (SEQ | DTMFLAS (SEQ | DTKFLAS (SEQ | DTLFLAS (SEQ |
| ID NO: 615) | ID NO: 752) | ID NO: 895) | ID NO: 1028) | ID NO: 1163) | ID NO: 1307) | ID NO: 1449) |
| DTSFLSS (SEQ | DTFFLSS (SEQ | DTYFLSS (SEQ | DTRFLSS (SEQ | DTMFLSS (SEQ | DTKFLSS (SEQ | DTLFLSS (SEQ |
| ID NO: 616) | ID NO: 753) | ID NO: 896) | ID NO: 1029) | ID NO: 1164) | ID NO: 1308) | ID NO: 1450) |
| DTSFLKS (SEQ | DTFFLKS (SEQ | DTYFLKS (SEQ | DTRFLKS (SEQ | DTMFLKS (SEQ | DTKFLKS (SEQ | DTLFLKS (SEQ |
| ID NO: 617) | ID NO: 754) | ID NO: 897) | ID NO: 1030) | ID NO: 1165) | ID NO: 1309) | ID NO: 1451) |
| DTSFLRS (SEQ | DTFFLRS (SEQ | DTYFLRS (SEQ | DTRFLRS (SEQ | DTMFLRS (SEQ | DTKFLRS (SEQ | DTLFLRS (SEQ |
| ID NO: 618) | ID NO: 755) | ID NO: 898) | ID NO: 1031) | ID NO: 1166) | ID NO: 1310) | ID NO: 1452) |
| DTSFLHS (SEQ | DTFFLHS (SEQ | DTYFLHS (SEQ | DTRFLHS (SEQ | DTMFLHS (SEQ | DTKFLHS (SEQ | DTLFLHS (SEQ |
| ID NO: 619) | ID NO: 756) | ID NO: 899) | ID NO: 1032) | ID NO: 1167) | ID NO: 1311) | ID NO: 1453) |
| DTSFLPS (SEQ | DTFFLPS (SEQ | DTYFLPS (SEQ | DTRFLPS (SEQ | DTMFLPS (SEQ | DTKFLPS (SEQ | DTLFLPS (SEQ |
| ID NO: 620) | ID NO: 757) | ID NO: 900) | ID NO: 1033) | ID NO: 1168) | ID NO: 1312) | ID NO: 1454) |
| DTSFLTS (SEQ | DTFFLTS (SEQ | DTYFLTS (SEQ | DTRFLTS (SEQ | DTMFLTS (SEQ | DTKFLTS (SEQ | DTLFLTS (SEQ |
| ID NO: 621) | ID NO: 758) | ID NO: 901) | ID NO: 1034) | ID NO: 1169) | ID NO: 1313) | ID NO: 1455) |
| DTSFLDS (SEQ | DTFFLDS (SEQ | DTYFLDS (SEQ | DTRFLDS (SEQ | DTMFLDS (SEQ | DTKFLDS (SEQ | DTLFLDS (SEQ |
| ID NO: 77) | ID NO: 50) | ID NO: 902) | ID NO: 1035) | ID NO: 1170) | ID NO: 1314) | ID NO: 1456) |
| DTSFHAS (SEQ | DTFFHAS (SEQ | DTYFHAS (SEQ | DTRFHAS (SEQ | DTMFHAS (SEQ | DTKFHAS (SEQ | DTLFHAS (SEQ |
| ID NO: 622) | ID NO: 759) | ID NO: 903) | ID NO: 1036) | ID NO: 1171) | ID NO: 1315) | ID NO: 1457) |
| DTSFHSS (SEQ | DTFFHSS (SEQ | DTYFHSS (SEQ | DTRFHSS (SEQ | DTMFHSS (SEQ | DTKFHSS (SEQ | DTLFHSS (SEQ |
| ID NO: 623) | ID NO: 760) | ID NO: 904) | ID NO: 1037) | ID NO: 1172) | ID NO: 1316) | ID NO: 1458) |
| DTSFHKS (SEQ | DTFFHKS (SEQ | DTYFHKS (SEQ | DTRFHKS (SEQ | DTMFHKS (SEQ | DTKFHKS (SEQ | DTLFHKS (SEQ |
| ID NO: 624) | ID NO: 761) | ID NO: 905) | ID NO: 1038) | ID NO: 1173) | ID NO: 1317) | ID NO: 1459) |
| DTSFHRS (SEQ | DTFFHRS (SEQ | DTYFHRS (SEQ | DTRFHRS (SEQ | DTMFHRS (SEQ | DTKFHRS (SEQ | DTLFHRS (SEQ |
| ID NO: 625) | ID NO: 762) | ID NO: 906) | ID NO: 1039) | ID NO: 1174) | ID NO: 1318) | ID NO: 1460) |
| DTSFHHS (SEQ | DTFFHHS (SEQ | DTYFHHS (SEQ | DTRFHHS (SEQ | DTMFHHS (SEQ | DTKFHHS (SEQ | DTLFHHS (SEQ |
| ID NO: 626) | ID NO: 763) | ID NO: 907) | ID NO: 1040) | ID NO: 1175) | ID NO: 1319) | ID NO: 1461) |
| DTSFHPS (SEQ | DTFFHPS (SEQ | DTYFHPS (SEQ | DTRFHPS (SEQ | DTMFHPS (SEQ | DTKFHPS (SEQ | DTLFHPS (SEQ |
| ID NO: 627) | ID NO: 764) | ID NO: 908) | ID NO: 1041) | ID NO: 1176) | ID NO: 1320) | ID NO: 1462) |
| DTSFHTS (SEQ | DTFFHTS (SEQ | DTYFHTS (SEQ | DTRFHTS (SEQ | DTMFHTS (SEQ | DTKFHTS (SEQ | DTLFHTS (SEQ |
| ID NO: 628) | ID NO: 765) | ID NO: 909) | ID NO: 1042) | ID NO: 1177) | ID NO: 1321) | ID NO: 1463) |
| DTSFHDS (SEQ | DTFFHDS (SEQ | DTYFHDS (SEQ | DTRFHDS (SEQ | DTMFHDS (SEQ | DTKFHDS (SEQ | DTLFHDS (SEQ |
| ID NO: 629) | ID NO: 766) | ID NO: 910) | ID NO: 1043) | ID NO: 1178) | ID NO: 1322) | ID NO: 1464) |
| DTSFQAS (SEQ | DTFFQAS (SEQ | DTYFQAS (SEQ | DTRFQAS (SEQ | DTMFQAS (SEQ | DTKFQAS (SEQ | DTLFQAS (SEQ |
| ID NO: 630) | ID NO: 767) | ID NO: 911) | ID NO: 1044) | ID NO: 1179) | ID NO: 1323) | ID NO: 1465) |
| DTSFQSS (SEQ | DTFFQSS (SEQ | DTYFQSS (SEQ | DTRFQSS (SEQ | DTMFQSS (SEQ | DTKFQSS (SEQ | DTLFQSS (SEQ |
| ID NO: 631) | ID NO: 768) | ID NO: 912) | ID NO: 1045) | ID NO: 1180) | ID NO: 1324) | ID NO: 1466) |
| DTSFQKS (SEQ | DTFFQKS (SEQ | DTYFQKS (SEQ | DTRFQKS (SEQ | DTMFQKS (SEQ | DTKFQKS (SEQ | DTLFQKS (SEQ |
| ID NO: 632) | ID NO: 769) | ID NO: 913) | ID NO: 1046) | ID NO: 1181) | ID NO: 1325) | ID NO: 1467) |
| DTSFQRS (SEQ | DTFFQRS (SEQ | DTYFQRS (SEQ | DTRFQRS (SEQ | DTMFQRS (SEQ | DTKFQRS (SEQ | DTLFQRS (SEQ |
| ID NO: 633) | ID NO: 770) | ID NO: 914) | ID NO: 1047) | ID NO: 1182) | ID NO: 1326) | ID NO: 1468) |
| DTSFQHS (SEQ | DTFFQHS (SEQ | DTYFQHS (SEQ | DTRFQHS (SEQ | DTMFQHS (SEQ | DTKFQHS (SEQ | DTLFQHS (SEQ |
| ID NO: 634) | ID NO: 771) | ID NO: 915) | ID NO: 1048) | ID NO: 1183) | ID NO: 1327) | ID NO: 1469) |
| DTSFQPS (SEQ | DTFFQPS (SEQ | DTYFQPS (SEQ | DTRFQPS (SEQ | DTMFQPS (SEQ | DTKFQPS (SEQ | DTLFQPS (SEQ |
| ID NO: 635) | ID NO: 772) | ID NO: 916) | ID NO: 1049) | ID NO: 1184) | ID NO: 1328) | ID NO: 1470) |
| DTSFQTS (SEQ | DTFFQTS (SEQ | DTYFQTS (SEQ | DTRFQTS (SEQ | DTMFQTS (SEQ | DTKFQTS (SEQ | DTLFQTS (SEQ |
| ID NO: 636) | ID NO: 773) | ID NO: 917) | ID NO: 1050) | ID NO: 1185) | ID NO: 1329) | ID NO: 1471) |
| DTSFQDS (SEQ | DTFFQDS (SEQ | DTYFQDS (SEQ | DTRFQDS (SEQ | DTMFQDS (SEQ | DTKFQDS (SEQ | DTLFQDS (SEQ |
| ID NO: 637) | ID NO: 774) | ID NO: 918) | ID NO: 1051) | ID NO: 1186) | ID NO: 1330) | ID NO: 1472) |
| DTSLLAS (SEQ | DTFLLAS (SEQ | DTYLLAS (SEQ | DTRLLAS (SEQ | DTMLLAS (SEQ | DTKLLAS (SEQ | DTLLLAS (SEQ |
| ID NO: 124) | ID NO: 775) | ID NO: 919) | ID NO: 1052) | ID NO: 1187) | ID NO: 1331) | ID NO: 133) |
| DTSLLSS (SEQ | DTFLLSS (SEQ | DTYLLSS (SEQ | DTRLLSS (SEQ | DTMLLSS (SEQ | DTKLLSS (SEQ | DTLLLSS (SEQ |
| ID NO: 638) | ID NO: 776) | ID NO: 920) | ID NO: 1053) | ID NO: 1188) | ID NO: 1332) | ID NO: 1473) |
| DTSLLKS (SEQ | DTFLLKS (SEQ | DTYLLKS (SEQ | DTRLLKS (SEQ | DTMLLKS (SEQ | DTKLLKS (SEQ | DTLLLKS (SEQ |
| ID NO: 639) | ID NO: 777) | ID NO: 921) | ID NO: 1054) | ID NO: 1189) | ID NO: 1333) | ID NO: 1474) |
| DTSLLRS (SEQ | DTFLLRS (SEQ | DTYLLRS (SEQ | DTRLLRS (SEQ | DTMLLRS (SEQ | DTKLLRS (SEQ | DTLLLRS (SEQ |
| ID NO: 640) | ID NO: 778) | ID NO: 922) | ID NO: 1055) | ID NO: 1190) | ID NO: 1334) | ID NO: 1475) |
| DTSLLHS (SEQ | DTFLLHS (SEQ | DTYLLHS (SEQ | DTRLLHS (SEQ | DTMLLHS (SEQ | DTKLLHS (SEQ | DTLLLHS (SEQ |
| ID NO: 641) | ID NO: 779) | ID NO: 923) | ID NO: 1056) | ID NO: 1191) | ID NO: 1335) | ID NO: 1476) |
| DTSLLPS (SEQ | DTFLLPS (SEQ | DTYLLPS (SEQ | DTRLLPS (SEQ | DTMLLPS (SEQ | DTKLLPS (SEQ | DTLLLPS (SEQ |
| ID NO: 642) | ID NO: 780) | ID NO: 924) | ID NO: 1057) | ID NO: 1192) | ID NO: 1336) | ID NO: 1477) |
| DTSLLTS(SEQ | DTFLLTS (SEQ | DTYLLTS (SEQ | DTRLLTS (SEQ | DTMLLTS (SEQ | DTKLLTS (SEQ | DTLLLTS (SEQ |
| ID NO: 643) | ID NO: 781) | ID NO: 925) | ID NO: 1058) | ID NO: 1193) | ID NO: 1337) | ID NO: 1478) |
| DTSLLDS (SEQ | DTFLLDS (SEQ | DTYLLDS (SEQ | DTRLLDS (SEQ | DTMLLDS (SEQ | DTKLLDS (SEQ | DTLLLDS (SEQ |
| ID NO: 126) | ID NO: 782) | ID NO: 926) | ID NO: 1059) | ID NO: 1194) | ID NO: 1338) | ID NO: 75) |
| DTSLHAS (SEQ | DTFLHAS (SEQ | DTYLHAS (SEQ | DTRLHAS (SEQ | DTMLHAS (SEQ | DTKLHAS (SEQ | DTLLHAS (SEQ |
| ID NO: 644) | ID NO: 783) | ID NO: 927) | ID NO: 1060) | ID NO: 1195) | ID NO: 1339) | ID NO: 1479) |
| DTSLHSS (SEQ | DTFLHSS (SEQ | DTYLHSS (SEQ | DTRLHSS (SEQ | DTMLHSS (SEQ | DTKLHSS (SEQ | DTLLHSS (SEQ |
| ID NO: 645) | ID NO: 784) | ID NO: 928) | ID NO: 1061) | ID NO: 1196) | ID NO: 1340) | ID NO: 1480) |
| DTSLHKS (SEQ | DTFLHKS (SEQ | DTYLHKS (SEQ | DTRLHKS (SEQ | DTMLHKS (SEQ | DTKLHKS (SEQ | DTLLHKS (SEQ |
| ID NO: 646) | ID NO: 785) | ID NO: 929) | ID NO: 1062) | ID NO: 1197) | ID NO: 1341) | ID NO: 1481) |
| DTSLHRS (SEQ | DTFLHRS (SEQ | DTYLHRS (SEQ | DTRLHRS (SEQ | DTMLHRS (SEQ | DTKLHRS (SEQ | DTLLHRS (SEQ |
| ID NO: 647) | ID NO: 786) | ID NO: 930) | ID NO: 1063) | ID NO: 1198) | ID NO: 1342) | ID NO: 1482) |
| DTSLHHS (SEQ | DTFLHHS (SEQ | DTYLHHS (SEQ | DTRLHHS (SEQ | DTMLHHS (SEQ | DTKLHHS (SEQ | DTLLHHS (SEQ |
| ID NO: 648) | ID NO: 787) | ID NO: 931) | ID NO: 1064) | ID NO: 1199) | ID NO: 1343) | ID NO: 1483) |
| DTSLHPS (SEQ | DTFLHPS (SEQ | DTYLHPS (SEQ | DTRLHPS (SEQ | DTMLHPS (SEQ | DTKLHPS (SEQ | DTLLHPS (SEQ |
| ID NO: 649) | ID NO: 788) | ID NO: 932) | ID NO: 1065) | ID NO: 1200) | ID NO: 1344) | ID NO: 1484) |
| DTSLHTS (SEQ | DTFLHTS (SEQ | DTYLHTS (SEQ | DTRLHTS (SEQ | DTMLHTS (SEQ | DTKLHTS (SEQ | DTLLHTS (SEQ |
| ID NO: 650) | ID NO: 789) | ID NO: 933) | ID NO: 1066) | ID NO: 1201) | ID NO: 1345) | ID NO: 1485) |
| DTSLHDS (SEQ | DTFLHDS (SEQ | DTYLHDS (SEQ | DTRLHDS (SEQ | DTMLHDS (SEQ | DTKLHDS (SEQ | DTLLHDS (SEQ |
| ID NO: 651) | ID NO: 790) | ID NO: 934) | ID NO: 1067) | ID NO: 1202) | ID NO: 1346) | ID NO: 1486) |
| DTSLQAS (SEQ | DTFLQAS (SEQ | DTYLQAS (SEQ | DTRLQAS (SEQ | DTMLQAS (SEQ | DTKLQAS (SEQ | DTLLQAS (SEQ |
| ID NO: 652) | ID NO: 791) | ID NO: 935) | ID NO: 1068) | ID NO: 1203) | ID NO: 1347) | ID NO: 1487) |
| DTSLQSS (SEQ | DTFLQSS (SEQ | DTYLQSS (SEQ | DTRLQSS (SEQ | DTMLQSS (SEQ | DTKLQSS (SEQ | DTLLQSS (SEQ |
| ID NO: 653) | ID NO: 792) | ID NO: 936) | ID NO: 1069) | ID NO: 1204) | ID NO: 1348) | ID NO: 1488) |
| DTSLQKS (SEQ | DTFLQKS (SEQ | DTYLQKS (SEQ | DTRLQKS (SEQ | DTMLQKS (SEQ | DTKLQKS (SEQ | DTLLQKS (SEQ |
| ID NO: 654) | ID NO: 793) | ID NO: 937) | ID NO: 1070) | ID NO: 1205) | ID NO: 1349) | ID NO: 1489) |
| DTSLQRS (SEQ | DTFLQRS (SEQ | DTYLQRS (SEQ | DTRLQRS (SEQ | DTMLQRS (SEQ | DTKLQRS (SEQ | DTLLQRS (SEQ |
| ID NO: 655) | ID NO: 794) | ID NO: 938) | ID NO: 1071) | ID NO: 1206) | ID NO: 1350) | ID NO: 1490) |
| DTSLQHS (SEQ | DTFLQHS (SEQ | DTYLQHS (SEQ | DTRLQHS (SEQ | DTMLQHS (SEQ | DTKLQHS (SEQ | DTLLQHS (SEQ |
| ID NO: 656) | ID NO: 795) | ID NO: 939) | ID NO: 1072) | ID NO: 1207) | ID NO: 1351) | ID NO: 1491) |
| DTSLQPS (SEQ | DTFLQPS (SEQ | DTYLQPS (SEQ | DTRLQPS (SEQ | DTMLQPS (SEQ | DTKLQPS (SEQ | DTLLQPS (SEQ |
| ID NO: 657) | ID NO: 796) | ID NO: 940) | ID NO: 1073) | ID NO: 1208) | ID NO: 1352) | ID NO: 1492) |
| DTSLQTS (SEQ | DTFLQTS (SEQ | DTYLQTS (SEQ | DTRLQTS (SEQ | DTMLQTS (SEQ | DTKLQTS (SEQ | DTLLQTS (SEQ |
| ID NO: 658) | ID NO: 797) | ID NO: 941) | ID NO: 1074) | ID NO: 1209) | ID NO: 1353) | ID NO: 1493) |
| DTSLQDS (SEQ | DTFLQDS (SEQ | DTYLQDS (SEQ | DTRLQDS (SEQ | DTMLQDS (SEQ | DTKLQDS (SEQ | DTLLQDS (SEQ |
| ID NO: 659) | ID NO: 798) | ID NO: 942) | ID NO: 1075) | ID NO: 1210) | ID NO: 1354) | ID NO: 1494) |

Bold faced & underlined amino acid residues are the residues which differ from the amino acid sequence in palivizumab

TABLE 3F
VL CDR3 Sequences

FQGSGYPFT (SEQ ID NO: 6)
FQGSFYPFT (SEQ ID NO: 61)
FQGSYYPFT (SEQ ID NO: 1495)
FQGSWYPFT (SEQ ID NO: 1496)
Bold faced and underlined amino acid residues are the residues which differ from the amino acid sequence in palivizumab

In one embodiment, formulations of the present invention comprise antibodies that comprise a VH CDR1 having the amino acid sequence of SEQ ID NO:1, SEQ ID NO:10 or SEQ ID NO:18. In another embodiment, formulations of the present invention comprise antibodies that comprise a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329. In another embodiment, formulations of the present invention comprise antibodies that comprise a VH CDR3 having the amino acid sequence of SEQ ID NO:3, SEQ ID NO:12, SEQ ID NO:20, SEQ ID NO:29, SEQ ID NO:79, or SEQ ID NO:311. In another embodiment, formulations of the present invention comprise antibodies that comprise a VH CDR1 having the amino acid sequence of SEQ ID NO:1, SEQ ID NO:10 or SEQ ID NO:18, a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329, and a VH CDR3 having the amino acid sequence of SEQ ID NO:3, SEQ ID NO:12, SEQ ID NO:20, SEQ ID NO:29, SEQ ID NO:79, or SEQ ID NO:311. In a preferred embodiment, formulations of the present invention comprise antibodies that comprise a VH CDR1 having the amino acid sequence of SEQ ID NO:10, a VH CDR2 having the amino acid sequence of SEQ ID NO:19, and a VH CDR3 having the amino acid sequence of SEQ ID NO:20. In accordance with these embodiments, the antibodies immunospecifically bind to a RSV F antigen. In specific embodiments, the antibodies are not palivizumab, a Fab fragment of palivizumab, or an antigen-binding fragment thereof. In specific embodiments, the antibodies have a high affinity for a RSV antigen (e.g., RSV F antigen).

In one embodiment, the amino acid sequence of the VH domain is

(SEQ ID NO: 48)

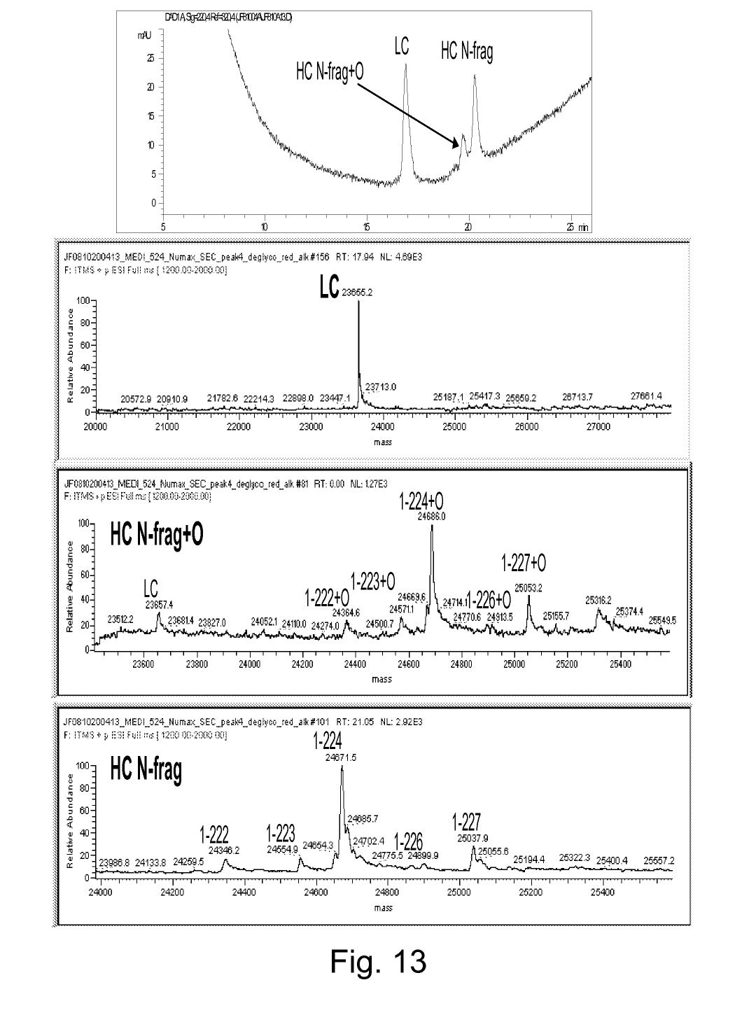

Q V T L R E S G P A L V K P T
Q T L T L T C T F S G F S L S
T A G M S V G W I R Q P P G K
A L E W L A D I W W D D K K H
Y N P S L K D R L T I S K D T
S K N Q V V L K V T N M D P A
D T A T Y Y C A R D M I F N F
Y F D V W G Q* G T T V T V S S,

wherein the three underlined regions indicate the VH CDR1, CDR2, and CDR3 regions, respectively; the four non-underlined regions correlate with the VL FR1, FR2, FR3, FR4, respectively; and the asterisk indicates the position of an A→Q mutation in VH FR4 as compared to the VH FR4 of palivizumab shown in FIG. 1B (SEQ ID NO:7). This VH domain (SEQ ID NO:48) is identical to that of the motavizumab antibody described elsewhere herein and shown in FIG. 13A. In some embodiments, this VH FR can be used in combination with any of the VH CDRs identified in Table 1 and/or Tables 3A-C. In one embodiment, the motavizumab antibody comprises the VH domain of FIG. 13A (SEQ ID NO:208) and the C-gamma-1 (nG1m) constant domain described in Johnson et al. (1997) J. Infect. Dis. 176, 1215-1224 and U.S. Pat. No. 5,824,307. In one embodiment, an antibody of the invention comprises a VH chain having the amino acid sequence of SEQ ID NO:208.

The present invention provides antibodies that immunospecifically bind to one or more RSV antigens (e.g., RSV F antigen), said antibodies comprising a VL chain having an amino acid sequence of any one of the VL chain listed in Table 2. In certain embodiments, the antibody is not palivizumab and/or the VL chain is not the VL chain of palivizumab.

The present invention also provides antibodies that immunospecifically bind to one or more RSV antigens (e.g., RSV F antigens), said antibodies comprising a variable light (“VL”) domain having an amino acid sequence of any one of the VL domains listed in Table 2. In certain embodiments, the antibody is not palivizumab and/or the VH domain is not the VH domain of palivizumab. The present invention also provides antibodies that immunospecifically bind to one or more RSV antigens (e.g., RSV F antigens), said antibodies comprising a VL CDR having an amino acid sequence of any one of the VL CDRs listed in Table 2 and/or Tables 3D-3F. In certain embodiments, the antibody is not palivizumab. In some embodiments, the antibody comprises one, two or three of the VL CDRs listed in Table 2 and/or Tables 3D-3F.

In one embodiment of the present invention, antibodies comprise a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:72, SEQ ID NO:314, SEQ ID NO:320, or SEQ ID NO:335. In another embodiment, formulations of the invention comprise antibodies that comprise a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336. In another embodiment, formulations of the invention comprise antibodies that comprise a VL CDR3 having the amino acid sequence of SEQ ID NO:6, SEQ ID NO:16 or SEQ ID NO:61. In another embodiment, formulations of the invention comprise antibodies that comprise a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:72, SEQ ID NO:314, SEQ ID NO:320, or SEQ ID NO:335, a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336, and a VL CDR3 having the amino acid sequence of SEQ ID NO:6, SEQ ID NO:16 or SEQ ID NO:61. In a preferred embodiment, formulations of the invention comprise antibodies that comprise a VL CDR1 having the amino acid sequence of SEQ ID NO:39, a VLCDR2 having the amino acid sequence of SEQ ID NO:5, and a VLCDR3 having the amino acid sequence of SEQ ID NO:6. In accordance with these embodiments, the antibodies immunospecifically bind to a RSV F antigen. In specific embodiments, the antibodies are not palivizumab or an antigen-binding fragment thereof (e.g., a Fab fragment of palivizumab). In another specific embodiment, the antibodies have a high affinity for RSV antigen (e.g., RSV F antigen).

In one embodiment the amino acid sequence of the VL domain is

(SEQ ID NO: 8)

D I Q M T Q S P S T L S A S V
G D R V T I T C S A S S R V G
Y M H W Y Q Q K P G K A P K L
L I Y D T S K L A S G V P S R
F S G S G S G T E F T L T I S
S L Q P D D F A T Y Y C F Q G
S G Y P F T F G G G T K V* E I
K,

wherein the three underlined regions indicate the VL CDR1, CDR2, and CDR3 regions, respectively; the four non-underlined regions correlate with the VL FR1, FR2, FR3, FR4, respectively; and the asterisk indicates the position of an L→V mutation in VL FR4 as compared to the VL FR4 of palivizumab shown in FIG. 1A. This VL domain (SEQ ID NO:8) is identical to that of the motavizumab antibody described elsewhere herein and shown in FIG. 13B. In some embodiments, this VL framework can be used in combination with any of the VL CDRs identified in Table 1 and/or Tables 3D-3F. In one embodiment, the motavizumab antibody comprises the VL domain of FIG. 13B (SEQ ID NO:209) and the C-kappa constant domain described in Johnson et al. (1997) J. Infect. Dis. 176, 1215-1224 and U.S. Pat. No. 5,824,307. In one embodiment, an antibody of the invention comprises a VL chain having the amino acid sequence of SEQ ID NO:209.

The present invention further provides antibodies that immunospecifically bind to one or more RSV antigens (e.g., RSV F antigen), wherein the antibody comprises a VH chain disclosed herein combined with a VL chain disclosed herein, or other VL chain. The present invention also provides antibodies that immunospecifically bind to one or more RSV antigens (e.g., RSV F antigen), wherein the antibody comprises a VL chain disclosed herein combined with a VH chain disclosed herein, or other VH chain.

The present invention also provides antibodies that immunospecifically bind to one or more RSV antigens (e.g., RSV F antigens), said antibodies comprising a VH domain disclosed herein combined with a VL domain disclosed herein, or other VL domain. The present invention further provides antibodies that immunospecifically bind to one or more RSV antigens (e.g., RSV F antigens), said antibodies comprising a VL domain disclosed herein combined with a VH domain disclosed herein, or other VH domain.

In a specific embodiment, antibodies that immunospecifically bind to a RSV antigen (e.g., RSV F antigens) comprise a VH domain having the amino acid sequence of SEQ ID NO:7, SEQ ID NO:9, SEQ ID NO:17, SEQ ID NO:24, SEQ ID NO:28, SEQ ID NO:33, SEQ ID NO:36, SEQ ID NO:40, SEQ ID NO:44, SEQ ID NO:48, SEQ ID NO:51, SEQ ID NO:55, SEQ ID NO:67, SEQ ID NO:78, SEQ ID NO:304, SEQ ID NO:310, SEQ ID NO:317, SEQ ID NO:323, or SEQ ID NO:328, and a VL domain having the amino acid sequence of SEQ ID NO:8, SEQ ID NO:13, SEQ ID NO:21, SEQ ID NO:26, SEQ ID NO:30, SEQ ID NO:34, SEQ ID NO:38, SEQ ID NO:42, SEQ ID NO:46, SEQ ID NO:49, SEQ ID NO:52, SEQ ID NO:54, SEQ ID NO:56, SEQ ID NO:58, SEQ ID NO:60, SEQ ID NO:62, SEQ ID NO:64, SEQ ID NO:65, SEQ ID NO:68, SEQ ID NO:70, SEQ ID NO:71, SEQ ID NO:74, SEQ ID NO:76, SEQ ID NO:307, SEQ ID NO:313, SEQ ID NO:319, SEQ ID NO:325, SEQ ID NO:331, or SEQ ID NO:334. In a preferred embodiment, antibodies that immunospecifically bind to a RSV F antigen comprise a VH domain having the amino acid sequence of SEQ ID NO:48 and a VL domain comprising the amino acid sequence of SEQ ID NO:11. In specific embodiments, the antibodies are not palivizumab or an antigen-binding fragment thereof (e.g., a Fab fragment). In another specific embodiment, the antibodies of the invention have a high affinity for a RSV antigen (e.g., RSV F antigen).

The present invention further provides antibodies that specifically bind to an RSV antigen (e.g., RSV F antigen), wherein the antibody comprises any VH CDR1 disclosed herein, optionally in combination with any VH CDR2 disclosed herein (or other VH CDR2), and/or optionally in combination with any VH CDR3 disclosed herein (or other VH CDR3)), and/or optionally in combination with any VL CDR1 disclosed herein (or other VL CDR1), and/or optionally in combination with any VL CDR2 disclosed herein (or other VL CDR2), and/or optionally in combination with any VL CDR3 disclosed herein (or other VL CDR3). The present invention also provides antibodies that specifically bind to an RSV antigen (e.g., RSV F antigen), wherein the antibody comprises any VH CDR2 disclosed herein, optionally in combination with any VH CDR1 disclosed herein (or other VH CDR1), and/or optionally in combination with any VH CDR3 disclosed herein (or other VH CDR3)), and/or optionally in combination with any VL CDR1 disclosed herein (or other VL CDR1), and/or optionally in combination with any VL CDR2 disclosed herein (or other VL CDR2), and/or optionally in combination with any VL CDR3 disclosed herein (or other VL CDR3). The present invention also provides antibodies that specifically bind to an RSV antigen (e.g., RSV F antigen), wherein the antibody comprises any VH CDR3 disclosed herein, optionally in combination with any VH CDR1 disclosed herein (or other VH CDR1), and/or optionally in combination with any VH CDR2 disclosed herein (or other VH CDR3)), and/or optionally in combination with any VL CDR1 disclosed herein (or other VL CDR1), and/or optionally in combination with any VL CDR2 disclosed herein (or other VL CDR2), and/or optionally in combination with any VL CDR3 disclosed herein (or other VL CDR3). The present invention also provides antibodies that specifically bind to an RSV antigen (e.g., RSV F antigen), wherein the antibody comprises any VL CDR1 disclosed herein, optionally in combination with any VH CDR1 disclosed herein (or other VH CDR1), and/or optionally in combination with any VH CDR2 disclosed herein (or other VH CDR2)), and/or optionally in combination with any VH CDR3 disclosed herein (or other VH CDR3), and/or optionally in combination with any VL CDR2 disclosed herein (or other VL CDR2), and/or optionally in combination with any VL CDR3 disclosed herein (or other VL CDR3). The present invention further provides antibodies that specifically bind to an RSV antigen (e.g., RSV F antigen), wherein the antibody comprises any VL CDR2 disclosed herein, optionally in combination with any VH CDR1 disclosed herein (or other VH CDR1), and/or optionally in combination with any VH CDR2 disclosed herein (or other VH CDR2)), and/or optionally in combination with any VH CDR3 disclosed herein (or other VH CDR3), and/or optionally in combination with any VL CDR1 disclosed herein (or other VL CDR1), and/or optionally in combination with any VL CDR3 disclosed herein (or other VL CDR3). The present invention also provides antibodies that specifically bind to an RSV antigen (e.g., RSV F antigen), wherein the antibody comprises any VL CDR3 disclosed herein, optionally in combination with any VH CDR1 disclosed herein (or other VH CDR1), and/or optionally in combination with any VH CDR2 disclosed herein (or other VH CDR2)), and/or optionally in combination with any VH CDR3 disclosed herein (or other VH CDR3), and/or optionally in combination with any VL CDR1 disclosed herein (or other VL CDR1), and/or optionally in combination with any VL CDR2 disclosed herein (or other VL CDR2).

The present invention also provides antibodies comprising one or more VH CDRs and one or more VL CDRs listed in Table 2 and/or Tables 3A-3F. In particular, the invention provides for an antibody comprising a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 2 and/or Tables 3A-3F. In a specific embodiment, the formulations of the invention comprise antibodies that have a high affinity for a RSV antigen (e.g., RSV F antigen).

The invention also provides an antibody that immunospecifically binds to a RSV F antigen, comprising a VH CDR1 and a VL CDR1, a VH CDR1 and a VL CDR2, a VH CDR1 and a VL CDR3, a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 2 and/or Tables 3A-3F, supra. In another specific embodiment, the formulations of the invention comprise antibodies that have a high affinity for a RSV antigen (e.g., RSV F antigen).

In one embodiment, a formulation of the invention comprises an antibody that comprises a VH CDR1 having the amino acid sequence of SEQ ID NO:1, SEQ ID NO:10 or SEQ ID NO:18 and a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:314, SEQ ID NO:320, or SEQ ID NO:335. In another embodiment, a formulation of the invention comprises an antibody that comprises a VH CDR1 having the amino acid sequence of SEQ ID NO:1, SEQ ID NO:10 or SEQ ID NO:18 and a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336. In another embodiment, a formulation of the invention comprises an antibody that comprises a VH CDR1 having the amino acid sequence of SEQ ID NO:1, SEQ ID NO:10 or SEQ ID NO:18 and a VL CDR3 having the amino acid sequence of SEQ ID NO:6, SEQ ID NO:16 or SEQ ID NO:61. In accordance with these embodiments, the antibody immunospecifically binds to a RSV F antigen.

In another embodiment, a formulation of the invention comprises an antibody that comprises a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329, and a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:314, SEQ ID NO:320, or SEQ ID NO:335. In another embodiment, an antibody of the invention comprises a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329, and a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336. In another embodiment, an antibody of the invention comprises a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329, and a VL CDR3 having the amino acid sequence of SEQ ID NO:6, SEQ ID NO:16, or SEQ ID NO:61. In accordance with these embodiments, the antibody immunospecifically binds to a RSV F antigen.

In another embodiment, a formulation of the invention comprises an antibody that comprises a VH CDR3 having the amino acid sequence of SEQ ID NO:3, SEQ ID NO:12, SEQ ID NO:20, SEQ ID NO:29, SEQ ID NO:79, or SEQ ID NO:311, and a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:314, SEQ ID NO:320, or SEQ ID NO:335. In another embodiment, a formulation of the invention comprises an antibody that comprises a VH CDR3 having the amino acid sequence of SEQ ID NO:3, SEQ ID NO:12, SEQ ID NO:20, SEQ ID NO:29, SEQ ID NO:79, or SEQ ID NO:311, and a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336. In a preferred embodiment, an antibody of the invention comprises a VH CDR3 having the amino acid sequence of SEQ ID NO:3, SEQ ID NO:12, SEQ ID NO:20, SEQ ID NO:29, SEQ ID NO:79, or SEQ ID NO:311, and a VL CDR3 having the amino acid sequence of SEQ ID NO:6, SEQ ID NO:16, or SEQ ID NO:61. In accordance with these embodiments, the antibody immunospecifically binds to a RSV F antigen.

The present invention provides antibodies that immunospecifically bind to a RSV F antigen, said antibodies comprising the amino acid sequence of the variable heavy domain and/or variable light domain or an antigen-binding fragment thereof of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4 with one or more amino acid residue substitutions in the variable heavy domain and/or variable light domain or antigen-binding fragment. The present invention also provides antibodies that immunospecifically bind to a RSV antigen, said antibodies comprising the amino acid sequence of the variable heavy domain and/or variable light domain or an antigen-binding fragment thereof of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4 with one or more amino acid residue substitutions in one or more VH CDRs and/or one or more VL CDRs. Non-limiting examples of amino acid residues in the VH CDRs and VL CDRs of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4, which may be substituted, are shown in bold in Table 2. The present invention also provides antibodies that immunospecifically bind to a RSV antigen, said antibodies comprising the amino acid sequence of the variable heavy domain and/or variable light domain or an antigen-binding fragment thereof of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4 with one or more amino acid residue substitutions in one or more VH frameworks and/or one or more VL frameworks. The antibody generated by introducing substitutions in the VH domain, VH CDRs, VL domain, VL CDRs and/or frameworks of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4 can be tested in vitro and/or in vivo, for example, for its ability to bind to a RSV antigen, or for its ability to prevent, treat and/or ameliorate a an upper and/or lower respiratory tract RSV infection, otitis media, or one or more symptoms thereof.

In a specific embodiment, an antibody that immunospecifically binds to a RSV F antigen comprises an amino acid sequence encoded by a nucleotide sequence that hybridizes to the nucleotide sequence(s) encoding palivizumab, AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524, motavizumab), A4B4-F52S, A17d4(1), A3e2, A14a4, A16b4, A17b5, A17f5, A17h4, or an antigen-binding fragment thereof under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3).

In another embodiment, an antibody that immunospecifically binds to a RSV Fantigen comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4, or an antigen-binding fragment thereof. In preferred embodiment, an antibody that immunospecifically binds to a RSV F antigen comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence of A4B4L1FR-S28R (motavizumab), or an antigen-binding fragment thereof.

In a specific embodiment, an antibody that immunospecifically binds to a RSV F antigen comprises an amino acid sequence of a VH domain and/or an amino acid sequence a VL domain encoded by a nucleotide sequence that hybridizes to the nucleotide sequence encoding any one of the VH and/or VL domains listed in Table 2 under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3). In another embodiment, an antibody that immunospecifically binds to a RSV antigen comprises an amino acid sequence of a VH CDR or an amino acid sequence of a VL CDRs encoded by a nucleotide sequence that hybridizes to the nucleotide sequence encoding any one of the VH CDRs or VL CDRs listed in Table 2 and/or Tables 3A-3F under stringent conditions e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art. In yet another embodiment, an antibody that immunospecifically binds to a RSV F antigen comprises an amino acid sequence of a VH CDR and an amino acid sequence of a VL CDR encoded by nucleotide sequences that hybridizes to the nucleotide sequences encoding any one of the VH CDRs and VL CDRs, respectively, listed in Table 2 and/or Tables 3A-3F under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In another embodiment, an antibody that immunospecifically binds to a RSV F antigen comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to any one of the VH domains listed in Table 2. In another embodiment, an antibody that immunospecifically binds to a RSV antigen comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VH CDRs listed in Table 2 and/or Tables 3A-3C. In another embodiment, an antibody that immunospecifically binds to a RSV F antigen comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to any one of the VL domains listed in Table 2. In another embodiment, an antibody that immunospecifically binds to a RSV F antigen comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs listed in Table 2 and/or Tables 3D-3F.

The present invention also provides antibodies that compete with an antibody or Fab fragment listed in Table 2 for binding to a RSV F antigen. The present invention also encompasses polypeptides, proteins and peptides comprising VL domains and/or VH domains that compete with a polypeptide, protein or peptide comprising a VL domain and/or a VH domain listed in Table 2 for binding to a RSV F antigen. Further, the present invention encompasses polypeptides, proteins and peptides comprising VL CDRs and/or VH CDRs that compete with a polypeptide, protein or peptide comprising a VL CDR and/or VH CDR listed in Table 2 and/or Tables 3A-3F for binding to a RSV F antigen.

The formulations of the present invention comprise antibodies that include derivatives that are modified, i.e., by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The present invention also provides antibodies that immunospecifically bind to a RSV antigen (e.g., RSV F antigen) which comprise a framework region known to those of skill in the art (e.g., a human or non-human fragment). The framework region may be naturally occurring or consensus framework regions. Preferably, the framework region of an antibody of the invention is human (see, e.g., Chothia et al., 1998, J. Mol. Biol. 278:457-479 for a listing of human framework regions, which is incorporated by reference herein in its entirety). In a specific embodiment, an antibody of the invention comprises the framework region of A4B4L1FR-S28R (motavizumab).

In a specific embodiment, the present invention provides antibodies that immunospecifically bind to a RSV F antigen, said antibodies comprising the amino acid sequence of one or more of the CDRs of an antibody listed in Table 2 (i.e., AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4) and/or one or more of the CDRs in Table 3A-3F, and human framework regions with one or more amino acid substitutions at one, two, three or more of the following residues: (a) rare framework residues that differ between the murine antibody framework (i.e., donor antibody framework) and the human antibody framework (i.e., acceptor antibody framework); (b) Venier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain.

The present invention encompasses formulations that comprise antibodies that immunospecifically bind to a RSV F antigen, said antibodies comprising the amino acid sequence of the variable heavy domain and/or variable light domain or an antigen-binding fragment thereof of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4 with mutations (e.g., one or more amino acid substitutions) in the framework regions. In certain embodiments, antibodies that immunospecifically bind to a RSV antigen comprise the amino acid sequence of the variable heavy domain and/or variable light domain or an antigen-binding fragment thereof of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4 with one or more amino acid residue substitutions in the framework regions of the VH and/or VL domains.

The present invention also encompasses formulations that comprise antibodies which immunospecifically bind to one or more RSV F antigens, said antibodies comprising the amino acid sequence of A4B4L1FR-S28R (motavizumab) with mutations (e.g., one or more amino acid substitutions) in the framework regions. In certain embodiments, antibodies which immunospecifically bind to one or more RSV F antigens comprise the amino acid sequence of A4B4L1FR-S28R (motavizumab) with one or more amino acid residue substitutions in the framework regions of the VH and/or VL domains. In a specific embodiment, antibodies which immunospecifically bind to one or more RSV F antigens comprise the framework regions depicted in FIG. 2 or FIG. 13.

The present invention also provides antibodies that immunospecifically bind to a RSV antigen, said antibodies comprising the amino acid sequence of the variable heavy domain and/or variable light domain of an antibody in Table 2 (i.e., AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4) with mutations (e.g., one or more amino acid residue substitutions) in the hypervariable and framework regions. Preferably, the amino acid substitutions in the hypervariable and framework regions improve binding of the antibody to a RSV antigen.

The present invention also provides antibodies which immunospecifically bind to one or more RSV F antigens, said antibodies comprising the amino acid sequence of A4B4L1FR-S28R (motavizumab) with mutations (e.g., one or more amino acid residue substitutions) in the variable and framework regions.

The present invention also provides antibodies that immunospecifically bind to a RSV antigen (e.g., RSV F antigen) which comprise constant regions known to those of skill in the art. Preferably, the constant regions of an antibody of the invention are human. In a specific embodiment, an antibody of the invention comprises the constant regions of A4B4L1FR-S28R (motavizumab).

The present invention also provides fusion proteins comprising an antibody that immunospecifically binds to a RSV antigen and a heterologous polypeptide. Preferably, the heterologous polypeptide that the antibody is fused to is useful for targeting the antibody to respiratory epithelial cells.

The present invention also encompasses formulations that comprise panels of antibodies that immunospecifically bind to a RSV antigen. In specific embodiments, the invention provides panels of antibodies having different association rate constants different dissociation rate constants, different affinities for a RSV antigen, and/or different specificities for a RSV antigen. The invention provides panels of at least 10, preferably at least 25, at least 50, at least 75, at least 100, at least 125, at least 150, at least 175, at least 200, at least 250, at least 300, at least 350, at least 400, at least 450, at least 500, at least 550, at least 600, at least 650, at least 700, at least 750, at least 800, at least 850, at least 900, at least 950, or at least 1000 antibodies. Panels of antibodies can be used, for example, in 96 well plates for assays such as ELISAs.

The present invention further provides one or more antibodies for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof). In a specific embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, and/or A17h4. In another specific embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises an antigen-binding fragment of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), or A4B4-F52S, A17d4(1), A3e2, A14a4, A16b4, A17b5, A17f5, or A17h4.

In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH domains having an amino acid sequence of any one of the VH domains listed in Table 2. In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH CDR1s having an amino acid sequence of any one of the VH CDR1s listed in Table 2 and/or Table 3A. In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH CDR2s having an amino acid sequence of any one of the VH CDR2s listed in Table 2 and/or Table 3B. In a preferred embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH CDR3s having an amino acid sequence of any one of the VH CDR3s listed in Table 2 and/or Table 3C.

In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VL domains having an amino acid sequence of any one of the VL domains listed in Table 2. In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VL CDR1s having an amino acid sequence of any one of the VL CDR1s listed in Table 2 or Table 3D. In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VL CDR2s having an amino acid sequence of any one of the VL CDR2s listed in Table 2 and/or Table 3E. In a preferred embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VL CDR3s having an amino acid sequence of any one of the VL CDR3s listed in Table 2 and/or Table 3F.

In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH domains having an amino acid sequence of any one of the VH domains listed in Table 2 and one or more VL domains having an amino acid sequence of any one of the VL domains listed in Table 2. In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH CDR1s having an amino acid sequence of any one of the VH CDR1s listed in Table 2 and/or Table 3A and one or more VL CDR1s having an amino acid sequence of any one of the VL CDR1s listed in Table 2 and/or Table 3D. In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH CDR1s having an amino acid sequence of any one of the VH CDR1s listed in Table 2 and/or Table 3A and one or more VL CDR2s having an amino acid sequence of any one of the VL CDR2s listed in Table 2 and/or Table 3E. In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH CDR1s having an amino acid sequence of any one of the VH CDR1s listed in Table 2 and/or Table 3A and one or more VL CDR3s having an amino acid sequence of any one of the VL CDR3s listed in Table 2 and/or Table 3F.

In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH CDR2s having an amino acid sequence of any one of the VH CDR2s listed in Table 2 and/or Table 3B and one or more VL CDR1s having an amino acid sequence of any one of the VL CDR1s listed in Table 2 and/or Table 3D. In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH CDR2s having an amino acid sequence of any one of the VH CDR2s listed in Table 2 and/or Table 3B and one or more VL CDR2s having an amino acid sequence of any one of the VL CDR2s listed in Table 2 and/or Table 3E. In another embodiment, a formulation of the present invention comprises one or more antibodies comprising one or more VH CDR2s having an amino acid sequence of any one of the VH CDR2s listed in Table 2 and/or Table 3B and one or more VL CDR3s having an amino acid sequence of any one of the VL CDR3s listed in Table 2 and/or Table 3F.

In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH CDR3s having an amino acid sequence of any one of the VH CDR3s listed in Table 2 and/or Table 3C and one or more VL CDR1s having an amino acid sequence of any one of the VL CDR1s listed in Table 2 and/or Table 3D. In another embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH CDR3s having an amino acid sequence of any one of the VH CDR3s listed in Table 2 and/or Table 3C and one or more VL CDR2s having an amino acid sequence of any one of the VL CDR2s listed in Table 2 and/or Table 3E. In a preferred embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises one or more antibodies comprising one or more VH CDR3s having an amino acid sequence of any one of the VH CDR3s listed in Table 2 and/or Table 3C and one or more VL CDR3s having an amino acid sequence of any one of the VL CDR3s listed in Table 2 and/or Table 3F. In a preferred embodiment, a formulation for use in the prevention, treatment, and/or amelioration of an upper and/or lower respiratory tract RSV infection, otitis media, or a symptom or respiratory condition relating thereto (including, but not limited to, asthma, wheezing, RAD, or a combination thereof) comprises A4B4L1FR-S28R (motavizumab) or an antigen-binding fragment thereof. In yet another embodiment, a formulation of the present invention comprises one or more fusion proteins of the invention.

As discussed in more detail below, a formulation of the invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionucleotides, or toxins. See, e.g., PCT publications WO 92/08495; WO 91/14438; WO 89/12624; U.S. Pat. No. 5,314,995; and EP 396,387.

Antibodies of the present invention may be used, for example, to purify, detect, and target RSV antigens, in both in vitro and in vivo diagnostic and therapeutic methods. For example, the antibodies have use in immunoassays for qualitatively and quantitatively measuring levels of the RSV in biological samples such as sputum. See, e.g., Harlow et al., Antibodies: A Laboratory Manual, (Cold Spring Harbor Laboratory Press, 2nd ed. 1988) (incorporated by reference herein in its entirety).

The invention provides an antibody comprising a Fab fragment, which immunospecifically binds to an RSV antigen (e.g., the F protein epitope NSELLSLINDMPITNDQKKLMSNN (SEQ ID NO: 337)), wherein the Tm of the Fab fragment is at least about 87° C., and wherein said antibody is not any one of palivizumab, AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, and A17h4. In a specific embodiment, the Fab in such an antibody is different from the Fab of palivizumab. In another embodiment, such an antibody comprises a VH or VL domain that is different from the VH or VL domain of palivizumab. In preferred embodiment, the Tm of the Fab fragment is at least about 90° C. or at least about 93° C. In another preferred embodiment, the pI of the antibody is between about 8.5 to 9.5 or between about 9.0 to 9.5.

In another specific embodiment, the antibody comprises a VH domain of the antibody A4B4L1FR-S28R (SEQ ID NO:48). In still another embodiment, the antibody comprises a VL domain of the antibody A4B4L1FR-S28R (SEQ ID NO:11). In still another embodiment, the Fab of the antibody is the Fab of antibody A4B4L1FR-S28R, preferably having one or more amino acid modifications in this constant domain.

The invention also provides an antibody formulation comprising the above described antibody, said formulation having a viscosity of less than 10.00 cP or less than 5.00cP at any temperature in the range of 1 to 26° C., or in the range of 5 to 25° C., or in the range of 10 to 25° C.

The invention also provides an antibody formulation comprising the above described antibody, said formulation having an aggregration rate of less than about 5%, 10%, or 15% per day at any temperature in the range of 38 to 42° C.

The above described antibodies can be generated by a method described in U.S. Provisional Patent Application No. 60/696,113, by Christian B. Allan, filed on Jul. 1, 2005, which is incorporated by reference herein in its entirety. In a specific embodiment, such an antibody is generated by a method comprising screening a plurality of candidate antibody domains (e.g., Fab, Fc and Fv) that have high binding affinity to a target (e.g., RSV antigen) for their solubility and thermal stability. Any method known in the art for screening protein domains for their solubility and thermal stability can be used. One or more antibody domains having high solubility and/or thermal stability are then selected and used for constructing the full antibodies by combining them with the appropriate domain(s) to generate a full antibody. In one embodiment, one or more candidate Fab domains that have a Tm value higher than at least 87° C., 90° C., 95° C., 100° C., 105° C., 110° C., 115° C., or 120° C. are selected for construction of the full antibody. In another embodiment, one or more candidate domains that have a pI value higher than about 6.5, 7.0, 7.5, 8.0, 8.5 or 9.0 are selected for construction of the full antibody. In a specific embodiment, the plurality of candidate Fab domains comprises Fab domains containing one or more amino acid residue substitutions to the Fab domain of the following antibodies palivizumab, AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, and/or A17h4.

A plurality of antigen binding domains (e.g., Fab, scFv, etc.) that bind a RSV antigen with an affinity above a chosen threshold may be obtained, e.g., by affinity screening of a phage display library. One or more metrics characterizing the antigen binding domains' formulation properties are then evaluated for each of the antigen binding domains. The plurality of antigen binding domains are ranked according to the one or more metrics. In one embodiment, the plurality of antigen binding domains are ranked according to their Tm values, and one or more antigen binding domains are selected from the top of the ranked list. In another embodiment, the plurality of antigen binding domains are ranked according to their pI values, and one or more antigen binding domains are selected from the top of the ranked list. In still another embodiment, the plurality of antigen binding domains are ranked according to a combined Tm and pI rank, and one or more antigen binding domains are selected from the top of the ranked list. The selected antigen binding domains are then used for construction of the full anti-RSV antibody molecule (e.g., antibodies, diabodies, etc.).

In another embodiment, a plurality of antibody constant region domains (e.g., Fc, CH2, CH3, etc) is screened for solubility and thermal stability. In one embodiment, one or more candidate antibody constant region domains that have a Tm value higher than at least 50° C., 55° C., 60° C., 65° C., 70° C., 75° C., 80° C., 85° C., 90° C., 95° C., 100° C., 105° C., 110° C., 115° C., or 120° C. are selected for construction of the full antibody. In another embodiment, one or more candidate antibody constant region domains that have a pI value higher than about 6.5, 7.0, 7.5, 8.0, 8.5 or 9.0 are selected for construction of the full antibody (e.g., antibody, Fc-fusion protein, etc.).

Such an antibody can also be generated by a method for engineering a protein for preferred formulation characteristics and/or properties including but not limited to, Tm, pI, solubility, stability. In one embodiment, the method comprises engineering one or more domains to improve the antibody's formulation characteristics. In a preferred embodiment, the engineered domain exhibits improved formulation characteristics without reducing significantly the antibody's pharmacological characteristics including but not limited to, the antibody's binding specificity, binding affinity and/or avidity to its target, or the antibody's Fc effector functions, e.g., Fc-receptor (FcR) binding, antibody dependent cellular cytotoxicity (ADCC), complement dependent cytotoxicity (CDC), and/or serum half life. In a more preferred embodiment, the engineered domain exhibits improved formulation characteristics without substantially affecting the antibody's pharmacological characteristics.

In a preferred embodiment, a domain is engineered by substituting one or more amino acid residues in the domain such that the stability of the domain is increased. In one embodiment, a domain is engineered such that its Tm value is increased. In one embodiment, a domain is engineered such that it has a Tm greater than a predetermined threshold value. In some preferred embodiments, the predetermined Tm threshold value is at least 50° C., 55° C., 60° C., 65° C., 70° C., 75° C., 80° C., 85° C., 90° C., 95° C., 100° C., 105° C., 110° C., 115° C., or 120° C. In a specific embodiment, such an engineered Fab domain is generated by substituting one or more amino acid residues in the Fab domain of palivizumab, AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (motavizumab), A4B4-F52S, A17d4(1), A3e2, A14a4, A16b4, A17b5, A17f5, or A17h4.

In another preferred embodiment, a domain is engineered by substituting one or more amino acid residues in the domain such that the solubility of the domain is increased. In one embodiment, a domain is engineered such that its pI value is increased. In one embodiment, a domain is engineered such that it has a pI greater than a predetermined threshold value. In some preferred embodiments, the predetermined pI threshold value is about 6.5, 7.0, 7.5, 8.0, 8.5, or 9.0.

In one embodiment, the antigen binding (e.g., Fab) and/or constant region (e.g., Fc) domains are engineered to improve the protein's formulation characteristics, e.g., Tm, pI, or stability. In preferred embodiments, the engineered antibody exhibits improved formulation characteristics without reducing significantly the antibody's pharmacological characteristics, e.g., the antibody's binding specificity, binding affinity and/or avidity to its target, or the antibody's Fc effector functions, e.g., Fc-receptor (FcR) binding, antibody dependent cellular cytotoxicity (ADCC), complement dependent cytotoxicity (CDC), and/or serum half life. In another embodiment, the engineered antibody exhibits improved formulation characteristics and improved pharmacological characteristics, e.g., the antibody's binding specificity, binding affinity and/or avidity to its target, or the antibody's Fc effector functions, e.g., FcR binding, ADCC, CDC, and/or serum half life.

The solubility of a protein may be optimized by altering the number and location of ionizable residues in the protein to adjust the pI. For example the pI of a polypeptide can be manipulated by making the appropriate amino acid substitutions (e.g., by substituting a charged amino acid such as a lysine, for an uncharged residue such as alanine). Without wishing to be bound by any particular theory, amino acid substitutions of a protein that result in changes of the pI of said protein may improve solubility and/or the stability of the protein. One skilled in the art would be able to determine amino acid substitutions that is most appropriate for a particular protein to achieve a desired pI. The pI of a protein may be determined by a variety of methods including but not limited to isoelectric focusing. It can also be estimated using any one of the various computer algorithms (see for example Bjellqvist et al., 1993, Electrophoresis 14:1023, which is incorporated herein by reference in its entirety).

In one embodiment, the pI of an engineered antibody binding domain is between pH 6.2 and pH 10.0. In one embodiment, substitutions resulting in alterations in the pI of the antigen binding domain will not significantly diminish its binding affinity for an antigen. In one embodiment, the pI of an engineered antibody constant region domain is between pH 6.2 and pH 10.0. In still another embodiment, substitutions resulting in alterations in the pI of the constant region domain will not significantly diminish its effector binding and/or function. It is also contemplated that substitutions resulting in alterations in the pI in an antibody domain may be selected such that both the pI and other pharmacological characteristics of the antibody domain, e.g., the antibody's binding specificity, binding affinity and/or avidity to its target, or the antibody's Fc effector functions are improved. The inventors have found that certain modifications of the hinge region do not change the pI and Tm of the antibody significantly. Thus, in one embodiment, the invention provides a method for engineering an antibody to improve the antibody's biological activity without reducing the antibody's formulation properties.

In one embodiment, the modifications of an antibody domain as described herein may be combined with known modifications of the Fc domain such as those disclosed in Duncan et al, 1988, Nature 332:563-564; Lund et al., 1991, J. Immunol 147:2657-2662; Lund et al, 1992, Mol Immunol 29:53-59; Alegre et al, 1994, Transplantation 57:1537-1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490); U.S. Pat. Nos. 5,624,821; 5,885,573; 6,194,551; U.S. Patent Application Nos. 60/601,634 and 60/608,852; PCT Publication Nos. WO 00/42072 and WO 99/58572; each of which is incorporated herein by reference in its entirety.

In one embodiment, the antibodies may be engineered to include modifications in the Fc region, typically to alter one or more functional properties of the antibody, such as serum half-life, complement fixation, Fc receptor binding, and/or antigen-dependent cellular cytotoxicity, without reducing the antibodies' pI and Tm. Furthermore, an antibody may be chemically modified (e.g., one or more chemical moieties can be attached to the antibody) or be modified to alter its glycosylation, again to alter one or more functional properties of the antibody.

In one embodiment, the amino acid sequence of the Fc region is modified by deleting, adding and/or substituting at least amino acid residue to alter one or more of the functional properties of the antibody described above. This approach is described further in Duncan et al, 1988, Nature 332:563-564; Lund et al., 1991, J Immunol 147:2657-2662; Lund et al, 1992, Mol Immunol 29:53-59; Alegre et al, 1994, Transplantation 57:1537-1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, J Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,194,551; 6,737,056 U.S. patent application Ser. Nos. 10/370,749 and PCT Publications WO 94/2935; WO 99/58572; WO 00/42072; WO 04/029207, each of which is incorporated herein by reference in its entirety.

In still another embodiment, the glycosylation of antibodies is modified. For example, an aglycoslated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861, each of which is incorporated herein by reference in its entirety.

Additionally or alternatively, an antibody can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342, each of which is incorporated herein by reference in its entirety.

In another embodiment, the antibodies may be engineered to include modifications in the antigen binding domain to alter the formulation characteristics of the antibody, without reducing the binding characteristics. One skilled in the art will understand that amino acid substitutions and other modifications of an antibody may alter its antigen binding characteristics (examples of binding characteristics include but are not limited to, binding specificity, equilibrium dissociation constant (K_{D}), dissociation and association rates (K_{off} and Kₒₙ respectively), binding affinity and/or avidity) and that certain alterations are more or less desirable. For example a modification that preserves or enhances antigen binding would be more preferable then one that diminished or altered antigen binding. The binding characteristics of an antibody for a target antigen, may be determined by a variety of methods including but not limited it, equilibrium methods (e.g., enzyme-linked immunoabsorbent assay (ELISA) or radioimmunoassay (RIA)), or kinetics (e.g., BIACORE® analysis; see Example 2), for example. Other commonly used methods to examine the binding characteristics of antibodies are described in Using Antibodies: A Laboratory Manual, Cold Spring Harbor Laboratory Press, NY, Harrow et al., 1999 and Antibodies: A Laboratory Manual, Cold Spring Harbor Laboratory Press, NY; Harlow et al., 1989.

It is well known in the art that the equilibrium dissociation constant (K_{D}) is defined as k_{off}/kₒₙ. It is generally understood that an antibody with a low K_{D} is preferable to an antibody with a high K_{D}. However, in some instances the value of the kₒₙ or k_{off} may be more relevant than the value of the K_{D}. One skilled in the art can determine which kinetic parameter is most important for a given antigen binding domain and application. In a preferred embodiment, the method of the invention will result in antigen binding domains with improved formulation characteristics and one or more antigen binding characteristics (e.g., binding specificity, K_{D}, K_{off}, Kₒₙ, binding affinity and/or avidity) that are improved by at least 2%, or by at least 5%, or by at least 10%, or by at least 20%, or by at least 30%, or by at least 40%, or by at least 50%, or by at least 60%, or by at least 70%, or by at least 80% when compared to kinetic parameters of the antigen binding domain without said modification.

In another embodiment, the method of the invention will result in modified antigen binding domains that have improved formulation characteristics, but do not have substantially diminished antigen binding. For example, the method of the invention will generate antigen binding domains that exhibit improved formulation characteristics, but preferably have no reduction in any antigen binding characteristic (e.g., binding specificity, K_{D}, K_{off}, Kₒₙ, binding affinity and/or avidity), or have one or more antigen binding characteristics that are reduced by less than 1%, or by less than 5%, or by less than 10%, or by less than 20%, or by less than 30%, or by less than 40%, or by less than 50%, or by less than 60%, or by less than 70%, or by less than 80% when compared to antigen binding of the antibody without said substitution.

In one embodiment, selected or engineered antigen binding and antibody constant domains are then used to construct a full anti-RSV antibody using methods known in the art. Such antibodies can then be submitted to formulation development to determine the optimal formulations.

### 5.3.4 Antibodies that Immunospecifically Bind to Human Metapneumovirus (hMPV)

The formulations of the present invention comprise an isolated antibody that specifically binds to an antigen of human metapneumovirus (hMPV) and compositions comprising this antibody. The term “anti-hMPV-antigen antibody” refers to an antibody or antibody fragment thereof that binds immunospecifically to a hMPV antigen. A hMPV antigen refers to a hMPV polypeptide or fragment thereof such as of hMPV nucleoprotein, hMPV phosphoprotein, hMPV matrix protein, hMPV small hydrophobic protein, hMPV RNA-dependent hMPV polymerase, hMPV F protein, and hMPV G protein. A hMPV antigen also refers to a polypeptide that has a similar amino acid sequence compared to a hMPV polypeptide or fragment thereof such as of hMPV nucleoprotein, hMPV phosphoprotein, hMPV matrix protein, hMPV small hydrophobic protein, hMPV RNA-dependent hMPV polymerase, hMPV F protein, and hMPV G protein.

The anti-hMPV-antigen antibodies used in this invention can be monoclonal antibodies, human antibodies, humanized antibodies or chimeric antibodies. In some preferred embodiments, the anti-hMPV antibody of the invention is the antibody disclosed in U.S. patent application Ser. No. 10/628,088, filed Jul. 25, 2003 and published May 20, 2004, as U.S. Pat. Pub. No. US 2004/0096451 A1.

The anti-hMPV-antigen antibodies of this section can be made, formulated, administered, used therapeutically or used prophylactically as described in U.S. patent application Ser. No. 10/628,088, filed Jul. 25, 2003 and published May 20, 2004, as U.S. Pat. Pub. No. US 2004/0096451 A1, the contents of which are hereby incorporated by reference in their entirety.

### 5.3.5 Antibodies that Immunospecifically Bind to Integrin αᵥβ₃

The formulations of the present invention also comprise an isolated antibody that specifically binds to integrin αᵥβ₃ and compositions comprising this antibody. The antibodies can be monoclonal antibodies, human antibodies, humanized antibodies or chimeric antibodies. In some preferred embodiments, the anti-integrin αᵥβ₃ antibody of the invention is MEDI-522 (Vitaxin®). Vitaxin® and compositions or formulations comprising Vitaxin® are disclosed, e.g., in International Publication Nos. WO 98/33919, WO 00/78815, and WO 02/070007; U.S. application Ser. No. 09/339,222; U.S. patent application Ser. No. 10/091,236, filed Mar. 4, 2002 and published Nov. 12, 2002, as U.S. Pat. Pub. No. US 2002/0168360, each of which is incorporated herein by reference in its entirety.

In further embodiments, the antibody that immunospecifically binds to integrin αᵥβ₃ is not Vitaxin® or an antigen-binding fragment of Vitaxin®. Examples of known antibodies that immunospecifically bind to integrin αᵥβ₃ include, but are not limited to, 11D2 (Searle), the murine monoclonal LM609 (Scripps, International Publication Nos. WO 89/05155 and U.S. Pat. No. 5,753,230, which is incorporated herein by reference in its entirety), International Publication Nos WO 98/33919 and WO 00/78815, each of which is incorporated herein by reference in its entirety), 17661-37E and 17661-37E 1-5 (USBiological), MON 2032 and 2033 (CalTag), ab7166 (BV3) and ab 7167 (BV4) (Abcam), and WOW-1 (Kiosses et al., Nature Cell Biology, 3:316-320).

αᵥβ₃, an integrin has been found on new blood vessels as well as surface of many solid tumors, activated macrophages, monocytes, and osteoclasts. As the such, the anti-integrin αᵥβ₃ antibodies of this section can be used, for example, as an investigational antibody, or in the prevention or treatment of several destructive diseases.

The anti-integrin αᵥβ₃ antibodies of this section can be made, formulated, administered, used therapeutically or used prophylactically as described in U.S. patent application Ser. No. 10/091,236, filed Mar. 4, 2002 and published Nov. 12, 2002, as U.S. Pat. Pub. No. US 2002/0168360; U.S. patent application Ser. No. 10/769,712, filed Jan. 30, 2004; U.S. patent application Ser. No. 10/769,720, filed Jan. 30, 2004 and published Sep. 9, 2004, as U.S. Pat. Pub. No. US 2004/0176272; U.S. patent application Ser. No. 10/379,145, filed Mar. 4, 2003; U.S. patent application Ser. No. 10/379,189, filed Mar. 4, 2003 and published as U.S. Pat. Pub. No. US 2004/0001835; PCT Application No. PCT/US04/02701, filed Jan. 30, 2004; International Application Publication No.: WO 00/78815 A1, entitled “Anti-αᵥβ₃ recombinant human antibodies, nucleic acids encoding same and methods”, by Huse et al.; and International Application Publication No.: WO 98/33919 A1, entitled “Anti-alpha-V-veta-3 recombinant humanized antibodies, nucleic acids encoding same and methods of use”, by Huse et al.; International Publication No. WO 89/05155, the contents of which are hereby incorporated by reference in their entirety.

### 5.3.6 Antibodies that Immunospecifically Bind to CD2

The formulations of the present invention comprise an isolated antibody that immunospecifically binds to CD2 and compositions comprising this antibody. The antibodies can be monoclonal antibodies, human antibodies, humanized antibodies or chimeric antibodies. In some preferred embodiments, the anti-CD2 antibody of the invention is siplizumab (MEDI-507). Siplizumab can selectively binds to cells expressing the CD2 antigen (specifically T cells, natural killer cells and thymocytes) and can be used, for example, in the prophylaxis and treatment of T cell lymphoma or other related conditions. MEDI-507 is disclosed, e.g., in International Publication No. WO 99/03502, International Application Nos. PCT/US02/22273 and PCT/US02/06761, and U.S. application Ser. Nos. 09/462,140, 10/091,268, and 10/091,313, each of which is incorporated herein by reference in its entirety. MEDI-507 is a humanized IgG1κ class monoclonal antibody that immunospecifically binds to human CD2 polypeptide. MEDI-507 was constructed using molecular techniques to insert the CDRs from the rat monoclonal antibody LO-CD2a/BTI-322 into a human IgG1 framework. LO-CD2a/BTI-322 has the amino acid sequence disclosed, e.g., in U.S. Pat. Nos. 5,730,979, 5,817,311, and 5,951,983; and U.S. application Ser. Nos. 09/056,072 and 09/462,140 (each of which is incorporated herein by reference in its entirety), or the amino acid sequence of the monoclonal antibody produced by the cell line deposited with the American Type Culture Collection (ATCC®), 10801 University Boulevard, Manassas, Va. 20110-2209 on Jul. 28, 1993 as Accession Number HB 11423.

The anti-CD2 antibodies of this section can be made, formulated, administered, used therapeutically or prophylactically, or in other context as described in U.S. patent application Ser. No. 10/091,268, filed Mar. 4, 2002, and published Apr. 15, 2003, as U.S. Pat. Pub. No. US 2003/0068320; U.S. patent application Ser. No. 10/091,313, filed Mar. 4, 2002, and published Mar. 6, 2003, as U.S. Pat. Pub. No. US 2003/0044406; and U.S. patent application Ser. No. 10/657,006, filed Sep. 5, 2003, and published Dec. 30, 2004, as U.S. Pat. Pub. No. US 2004/0265315, the contents of which are hereby incorporated by reference in their entirety.

### 5.3.7 Antibodies that Immunospecifically Bind to CD19

The formulations of the present invention comprise an isolated antibody that immunospecifically binds to CD19 and a composition comprising this antibody. The antibodies can be monoclonal antibodies, human antibodies, humanized antibodies or chimeric antibodies. In some preferred embodiments, the anti-CD19 antibody of the invention is MT-103. MT-103 is the most-advanced clinical representative of a novel class of antibody derivatives called Bi-Specific T Cell Engagers (BiTE™). The BiTE compound MT-103 directs and activates the patient's own immune system against the cancer cells, stimulating T cells (a very potent type of white blood cell) to destroy B tumor cells (cancerous white blood cells). MT-103 specifically targets a particular protein (the CD19 antigen), which is present on cancerous B cells but not on other types of blood cells or healthy tissues, therefore avoiding the side effects of traditional chemotherapy

The anti-CD19 antibodies of this section can be made, formulated, administered, used therapeutically or prophylactically, or in other context as described in U.S. Pat. No. 6,723,538, and U.S. Pat. Pub. No. 2004/0162411.

The human CD19 molecule is a structurally distinct cell surface receptor that is expressed on the surface of human B cells. The invention relates to immunotherapeutic compositions and methods for the prophylaxis and treatment of GVHD, humoral rejection, and post-transplantation lymphoproliferative disorder in human subjects; autoimmune diseases and disorders; and cancers, using therapeutic antibodies that bind to the human CD19 antigen.

Hybridomas producing HB12a and HB12b anti-CD19 antibodies have been deposited under ATCC deposit nos. PTA-6580 and PTA-6581. See, also, U.S. application No. to be assigned (Attorney Docket No.: 11605-006-999) and U.S. application Ser. No. 11/355,905, filed Feb. 15, 2006, each of which is incorporated herein by reference in its entirety.

### 5.3.8 Antibodies that Immunospecifically Bind to EphA2

The formulations of the present invention comprise an isolated antibody that immunospecifically binds to EphA2 and a compositions comprising this antibody. The antibodies of the invention can be monoclonal antibodies, human antibodies, humanized antibodies or chimeric antibodies. In some embodiments, the anti-EphA2 antibody of the invention is EA2. In some preferred embodiments, the EA2 antibody is human or humanized. In other embodiments, the is EA5. In some preferred embodiments, the EA5 antibody is human or humanized. Hybridomas producing the anti-EphA2 antibodies of the invention have been deposited with the American Type Culture Collection (ATCC, P.O. Box 1549, Manassas, Va. 20108) under the provisions of the Budapest Treaty on the International Recognition of the Deposit of Microorganisms for the Purposes of Patent Procedures, and assigned accession numbers, which are incorporated by reference, as shown in TABLE 4.

TABLE 4

| EphA2 Antibodies | Deposit No. | Date of Deposit |
|---|---|---|
| EA2.31 | PTA-4380 | May 22, 2002 |
| EA5.12 | PTA-4381 | May 22, 2002 |
| Eph099B-102.147 | PTA-4572 | Aug. 7, 2002 |
| Eph099B-208.261 | PTA-4573 | Aug. 7, 2002 |
| Eph099B-210.248 | PTA-4574 | Aug. 7, 2002 |
| Eph099B-233.152 | PTA-5194 | May 12, 2003 |
| Eph101.530.241 | PTA-4724 | Sep. 26, 2002 |

EphA2 is a 130 kDa receptor tyrosine kinase that is expressed in adult epithelia, where it is found at low levels and is enriched within sites of cell-cell adhesion (Zantek, et al, Cell Growth & Differentiation 10:629, 1999; Lindberg, et al., Molecular & Cellular Biology 10: 6316, 1990). EphA2 is upregulated on a large number of aggressive carcinoma cells. The anti-EphA2 antibodies of this invention can be used, for example, in the treatment of a variety of tumors, including breast, colon, prostate, lung and skin cancers, as well as to prevent metastasis.

The anti-EphA2 antibodies of this section can be made, formulated, administered, used therapeutically or used prophylactically as described in U.S. patent application Ser. No. 10/823,259, filed Apr. 12, 2004; U.S. patent application Ser. No. 10/823,254, filed on Apr. 12, 2004; U.S. patent application Ser. No. 10/436,782, filed on May 12, 2003 and published Feb. 12, 2004 as U.S. Pat. Pub. No. 2004/0028685; U.S. patent application Ser. No. 10/436,783, filed on May 12, 2003 and published May 13, 2004 as U.S. Pat. Pub. No. 2004/0091486; U.S. patent application Ser. No. 11/004,794, filed on Dec. 3, 2004; U.S. patent application Ser. No. 10/994,129, filed on Nov. 19, 2004; U.S. patent application Ser. No. 11/004,795, filed on Dec. 3, 2004; and U.S. Provisional Application Nos. 60/662,517,60/622,711, 60/622,489, filed Oct. 27, 2004, the contents of which are hereby incorporated by reference in their entirety.

### 5.3.9 Antibodies that Immunospecifically Bind to EphA4

The formulations of the present invention comprise an isolated antibody that immunospecifically binds to an antigen of EphA4 and a composition comprising this antibody. The antibodies of the invention can be monoclonal antibodies, human antibodies, humanized antibodies or chimeric antibodies. Hybridomas producing the anti-EphA4 antibodies of the invention have been deposited with the American Type Culture Collection (ATCC, P.O. Box 1549, Manassas, Va. 20108) on Jun. 4, 2004 under the provisions of the Budapest Treaty on the International Recognition of the Deposit of Microorganisms for the Purposes of Patent Procedures, and assigned accession number PTA-6044 and PTA-4381 and incorporated by reference.

EphA4 is a receptor tyrosine kinase that is expressed in brain, heart, lung, muscle, kidney, placenta, pancreas (Fox, et al, Oncogene 10:897, 1995) and melanocytes (Easty, et al., Int. J. Cancer 71:1061, 1997). EphA4 is overexpressed in a number of cancers. The anti-EphA4 antibodies of this section can be used, for example, to decrease the expression of EphA4 in the treatment of pancreatic cancers etc.

The anti-EphA4 antibodies of this section can be made, formulated, administered, used therapeutically or used prophylactically as described in U.S. patent application Ser. No. 10/863,729, filed Jun. 7, 2004; U.S. patent application Ser. No. 11/004,794, filed on Dec. 3, 2004; U.S. patent application Ser. Nos. 11/004,794 and 11/004,795, filed on Dec. 3, 2004, the contents of which are hereby incorporated by reference in their entirety.

### 5.3.10 Antibodies that Immunospecifically Bind to IL-9

The formulations of the present invention comprise an antibody that immunospecifically binds to IL-9 and a composition comprising this antibody. The antibodies of the invention can be monoclonal antibodies, human antibodies, humanized antibodies or chimeric antibodies. In some preferred embodiments, the anti-IL-9 antibodies is MEDI-528.

It has been shown that IL-9 may be a key mediator of asthma and may also contribute to other respiratory disorders including chronic obstructive pulmonary disease (COPD) and cystic fibrosis. The anti-IL-9 antibodies of this section may be used in the prophylaxis or treatment of asthma.

The anti-IL-9 antibodies of this section can be made, formulated, administered, used therapeutically or used prophylactically as described in U.S. patent application Ser. No. 10/823,253, filed Apr. 12, 2004 and published Jan. 6, 2005, as U.S. Pat. Pub. No. US 2005/0002934 A1; U.S. patent application Ser. No. 10/823,810, filed on Apr. 12, 2004; U.S. Provisional Application Nos. 60/371,728 and 60,371,683, filed Apr. 12, 2002; and U.S. Provisional Application No. 60/561,845, filed Apr. 12, 2004, the contents of which are hereby incorporated by reference in their entirety.

### 5.3.11. Antibodies that Immunospecifically Bind to HMG1

The formulations of the present invention can comprise an antibody that immunospecifically binds to HMG1 and a composition comprising this antibody. The antibodies of the invention can be monoclonal antibodies, human antibodies, humanized antibodies or chimeric antibodies.

The early proinflammatory cytokines (e.g., TNF, IL-1, etc.) mediate inflammation, and induce the late release of high mobility group protein 1 (HMG1) (also known as HMG-1, HMG1, and HMGB1), a protein that accumulates in serum and mediates delayed lethality and further induction of early proinflammatory cytokines.

It has also been shown that HMG1 can be actively secreted by stimulated macrophages or monocytes in a process requiring acetylation of the molecule, which enables translocation from the nucleus to secretory lysosomes and results in the secretion of an acetylated form of HMG1. See, PCT/IB2003/005718. Thus, HMG1 passively released from necrotic cells and HMGB1 actively secreted by inflammatory cells are molecularly different.

Further, HMG1 has been implicated as a cytokine mediator of delayed lethality in endotoxemia. See, e.g., U.S. Pat. Nos. 6,468,533 and 6,448,223. More specifically, it has been demonstrated that bacterial endotoxin (lipopolysaccharide (LPS)) activates monocytes/macrophages to release HMG1 as a late response to activation, resulting in elevated serum HMG1 levels that are toxic. Antibodies against HMG1 have been shown to prevent lethality of endotoxin even when antibody administration is delayed until after the early cytokine response. Like other proinflammatory cytokines, HMG1 is a potent activator of monocytes. Intratracheal application of HMG1 causes acute lung injury, and anti-HMG1 antibodies protect against endotoxin-induced lung edema. In addition, serum HMG1 levels are elevated in critically ill patients with sepsis or hemorrhagic shock, and levels are significantly higher in non-survivors as compared to survivors.

The anti-HMG1 antibodies of this section can be made, formulated, administered, used therapeutically or used prophylactically as described in U.S. Patent Publication No. 2006-0099207 A1 filed Oct. 21, 2005, which is incorporated herein by reference in its entirety. Three clones, S6, S16 and G4 have been deposited with the American Type Culture Collection (10801 University Boulevard, Manassas, Va. 20110-2209) and assigned ATCC Deposit Nos. PTA-6143 (Deposited Aug. 4, 2004), PTA-6259 (Deposited Oct. 19, 2004) and PTA-6258 (Deposited Oct. 19, 2004) (also referred to herein as “S6”, “S16”, and “G4”, respectively) as described in U.S. Patent Publication No. 2006-0099207 A1 filed Oct. 21, 2005, which is incorporated herein by reference in its entirety.

### 5.3.12. Antibodies that Immunospecifically Bind to ALK

The formulations of the present invention can comprise an antibody that immunospecifically binds to ALK and a composition comprising this antibody. The antibodies of the invention can be monoclonal antibodies, human antibodies, humanized antibodies or chimeric antibodies.

Monoclonal antibodies against ALK as well as hybridoma cell lines producing ALK monoclonal antibodies 8B10, 16G2-3 and 9C10-5 (deposited with the American Type Culture Collection (10801 University Boulevard, Manassas, Va. 20110-2209) and assigned ATCC Deposit Nos. to be assigned, respectively) as described in U.S. patent application Ser. No. 09/880,097, filed Jun. 14, 2001 and published Mar. 21, 2002, as U.S. Pat. Pub. No. 20020034768, which is incorporated herein by reference in its entirety.

Pleiotrophin (PTN) is a 136-amino acid, secreted, heparin-binding cytokine that has diverse functions including a role in angiogenesis. PTN has been shown to specifically bind to a receptor tyrosine kinase, Anaplastic Lymphoma Kinase (ALK), and such binding leads to auto-phosphorylation of the receptor and subsequent phosphorylation of a number of signal transduction molecules such as IRS-1, PLC-gamma, PI3 kinase, and Shc, and activates a cell survival pathway. See PCT Pat. App. Pub. No. WO 01/96364. Accordingly, agents and therapeutic treatments that regulate ALK-mediated signal transduction pathways can affect one or more ALK-regulated functions, including, for example, angiogenesis. ALK participates in various disease states, including cancers and diseases related to unwanted or excessive angiogenesis. Additionally, ALK participates in a desirable way in certain processes, such as wound healing. ALK and/or PTN are expressed, often at high levels, in a variety of tumors. Therefore, agents that downregulate ALK and/or PTN function may affect tumors by a direct effect on the tumor cells, an indirect effect on the angiogenic processes recruited by the tumor, or a combination of direct and indirect effects.

### 5.3.13. Antibodies that Immunospecifically Bind to CD20

The formulations of the present invention can comprise an antibody that immunospecifically binds to CD20 and a composition comprising this antibody. The antibodies of the invention can be monoclonal antibodies, human antibodies, humanized antibodies or chimeric antibodies.

CD20 is only expressed by B lymphocytes (Stashenko et al. (1980) J Immunol 125:1678-1685; Tedder et al., 1988a). CD20 forms a homo- or hetero-tetrameric complex that is functionally important for regulating cell cycle progression and signal transduction in B lymphocytes (Tedder and Engel, 1994). CD20 additionally regulates transmembrane Ca⁺⁺ conductance, possibly as a functional component of a Ca⁺⁺-permeable cation channel (Bubien et al. J Cell Biol 121:1121-1132; Kanzaki et al. (1997a) J Biol Chem 272:14733-14739; Kanzaki et al. (1997b) J Biol Chem 272:4964-4969; Kanzaki et al. (1995) J Biol Chem 270:13099-13104). Antibodies against CD20 are effective in treating non-Hodgkin's lymphoma (McLaughlin et al. (1998) Oncology 12:1763-1769; Onrust et al. (1989) J Biol Chem 264:15323-15327; Weiner (1999) Semin Oncol 26:43-51).

See, also, U.S. patent application Ser. No. 10/433,287, filed Sep. 30, 2003, published as US 20040137566 on Jul. 15, 2004, which is incorporated herein by reference in its entirety.

### 5.3.14. Antibodies that Immunospecifically Bind to CD22

The formulations of the present invention can comprise an antibody that immunospecifically binds to CD22 and a composition comprising this antibody. The antibodies of the invention can be monoclonal antibodies, human antibodies, humanized antibodies or chimeric antibodies.

Anti-CD22 antibodies have been described, for example, in U.S. Pat. Nos. 5,484,892; 6,183,744; 6,187,287; 6,254,868; 6,306,393, and in Tuscano et al., Blood 94(4):1382-92 (1999) (each of which is incorporated herein in its entirety by reference). The use of monoclonal antibodies, including anti-CD22 antibodies, in the treatment of non-Hodgkin's lymphoma is reviewed, for example, by Renner et al., Leukemia 11(Suppl. 2):S5509 (1997).

The use of humanized CD22 antibodies has been described for the treatment of autoimmune disorders (see, Tedder U.S. Patent Application Publication No. US2003/0202975) and for the treatment of B cell malignancies, such as lymphomas and leukemias (see, Tuscano U.S. Patent Application Publication No. U.S. 2004/0001828). Humanized CD22 antibodies that target specific epitopes on CD22 have been described for use in immunoconjugates for therapeutic uses in cancer (see U.S. Pat. Nos. 5,789,554 and 6,187,287 to Leung).

Exemplary VH and VK antibody regions of the invention were deposited with the American Type Culture Collection (ATCC). In particular, a plasmid encoding the humanized anti-CD22 VH sequence of the invention designated RHOv2 was deposited under ATCC deposit no. PTA-7372, on Feb. 9, 2006. A plasmid encoding the humanized anti-CD22 VH sequence of the invention designated RHOv2ACD was deposited under ATCC deposit no. PTA-7373, on Feb. 9, 2006. A plasmid encoding the humanized anti-CD22 VK sequence of the invention, RKA was deposited under ATCC deposit no. PTA-7370, on Feb. 9, 2006. A plasmid encoding the humanized anti-CD22 VK sequence of the invention, RKC, was deposited under ATCC deposit no. PTA-7371, on Feb. 9, 2006.

See, also, U.S. Provisional Application No. TBA, filed Mar. 6, 2006, attorney docket no. BC320P1, which is incorporated herein by reference in its entirety.

### 5.3.15. Antibodies that Immunospecifically Bind to Chitinase

The formulations of the present invention can comprise an antibody that immunospecifically binds to Chitinase and a composition comprising this antibody. The antibodies of the invention can be monoclonal antibodies, human antibodies, humanized antibodies or chimeric antibodies.

It is described that blocking a chitinase/chitinase-like protein, in vivo results in protection of bone and cartilage as well as a reduction in weight loss in a mouse RA model. These results support the role of chitinase/chitinase-like proteins in chronic inflammatory diseases and more specifically the role of chitinase/chitinase-like proteins in OCL-related diseases including bone metabolism and connective tissue disorders and diseases. Furthermore, these results validate human chitinase/chitinase-like proteins as potential therapeutic targets for the prevention and treatment of OCL-related diseases.

See, also, U.S. application Ser. No. 10/202,436, filed Jul. 23, 2002, published as US 20030049261 on Mar. 13, 2003, which is incorporated herein by reference in its entirety.

### 5.3.16. Antibodies that Immunospecifically Bind to Interferon Alpha

The formulations of the present invention can comprise an antibody that immunospecifically binds to interferon alpha and a composition comprising this antibody. The antibodies of the invention can be monoclonal antibodies, human antibodies, humanized antibodies or chimeric antibodies.

The invention provides a method of treating an interferon alpha-mediated disease or disorder in a subject, comprising administering to the subject an anti-IFN alpha antibody of the invention, such that the interferon-alpha mediated disease in the subject is treated. Examples of diseases that can be treated include autoimmune diseases (e.g., systemic lupus erythematosus, multiple sclerosis, insulin dependent diabetes mellitus, inflammatory bowel disease, psoriasis, autoimmune thyroiditis, rheumatoid arthritis and glomerulonephritis), transplant rejection and graft versus host disease.

Anti-interferon alpha monoclonal antibody has also been described in U.S. Ser. No. 11/009,410 filed Dec. 10, 2004, which is incorporated herein by reference in its entirety.

### 5.3.17. Antibodies that Immunospecifically Bind to Interferon Alpha Receptor

The formulations of the present invention can comprise an antibody that immunospecifically binds to interferon alpha receptor and a composition comprising this antibody. The antibodies of the invention can be monoclonal antibodies, human antibodies, humanized antibodies or chimeric antibodies.

The invention also provides a method for inhibiting biological activity of a type I interferon on a cell expressing interferon alpha receptor 1 comprising contacting the cell with the antibody of the invention, such that the biological activity of the type I interferon is inhibited. The invention also provides a method of treating a type I interferon-mediated disease or disorder in a subject in need of treatment comprising administering to the subject the antibody, or antigen-binding portion thereof, of the invention, such that the type-I interferon mediated disease in the subject is treated. The type I interferon-mediated disease can be, for example, an interferon alpha-mediated disease.

Examples of disease or disorders that can be treated using the methods of the invention include systemic lupus erythematosus, insulin dependent diabetes mellitus, inflammatory bowel disease, multiple sclerosis, psoriasis, autoimmune thyroiditis, rheumatoid arthritis, glomerulonephritis, HIV infection, AIDS, transplant rejection and graft versus host disease.

Anti-interferon receptor monoclonal antibody has been described in U.S. Patent Publication No. 2006-0029601 A1, published Feb. 9, 2006, filed Jun. 20, 2005, which is incorporated herein by reference in its entirety.

### 5.3.18 Antibodies that have Therapeutic Utility

The formulations of the present invention comprise antibodies that have therapeutic utility, including but not limited to antibodies listed in Table 5.

TABLE 5
THERAPEUTIC ANTIBODIES THAT CAN BE USED IN
CONNECTION WITH THE PRESENT INVENTION

| Company | Product | Disease | Target |
|---|---|---|---|
| Abgenix | ABX-EGF | Cancer | EGF receptor |
| AltaRex | OvaRex | ovarian cancer | tumor antigen CA125 |
| | BravaRex | metastatic | tumor antigen MUC1 |
| | | cancers | |
| Antisoma | Theragyn | ovarian cancer | PEM antigen |
| | (pemtumomabytrrium- | | |
| | 90) | | |
| | Therex | breast cancer | PEM antigen |
| Boehringer | Blvatuzumab | head & neck | CD44 |
| Ingelheim | | cancer | |
| Centocor/J&J | Panorex | Colorectal | 17-1A |
| | | cancer | |
| | ReoPro | PTCA | Gp IIIb/IIIa |
| | ReoPro | Acute MI | Gp IIIb/IIIa |
| | ReoPro | Ischemic stroke | Gp IIIb/IIIa |
| Corixa | Bexocar | NHL | CD20 |
| CRC Technology | MAb, idiotypic 105AD7 | colorectal cancer | Gp72 |
| | | vaccine | |
| Crucell | Anti-EpCAM | cancer | Ep-CAM |
| Cytoclonal | MAb, lung cancer | non-small cell | NA |
| | | lung cancer | |
| Genentech | Herceptin | metastatic breast | HER-2 |
| | | cancer | |
| | Herceptin | early stage | HER-2 |
| | | breast cancer | |
| | Rituxan | Relapsed/refractory | CD20 |
| | | low-grade or | |
| | | follicular NHL | |
| | Rituxan | intermediate & | CD20 |
| | | high-grade NHL | |
| | MAb-VEGF | NSCLC, | VEGF |
| | | metastatic | |
| | MAb-VEGF | Colorectal | VEGF |
| | | cancer, | |
| | | metastatic | |
| | AMD Fab | age-related | CD18 |
| | | macular | |
| | | degeneration | |
| | E-26 (2^{nd} gen. IgE) | allergic asthma | IgE |
| | | & rhinitis | |
| IDEC | Zevalin (Rituxan + | low grade of | CD20 |
| | yttrium-90) | follicular, | |
| | | relapsed or | |
| | | refractory, | |
| | | CD20-positive, | |
| | | B-cell NHL and | |
| | | Rituximab- | |
| | | refractory NHL | |
| ImClone | Cetuximab + innotecan | refractory | EGF receptor |
| | | colorectal | |
| | | carcinoma | |
| | Cetuximab + cisplatin & | newly diagnosed | EGF receptor |
| | radiation | or recurrent head | |
| | | & neck cancer | |
| | Cetuximab + | newly diagnosed | EGF receptor |
| | gemcitabine | metastatic | |
| | | pancreatic | |
| | | carcinoma | |
| | Cetuximab + cisplatin + | recurrent or | EGF receptor |
| | 5FU or Taxol | metastatic head | |
| | | & neck cancer | |
| | Cetuximab + | newly diagnosed | EGF receptor |
| | carboplatin + paclitaxel | non-small cell | |
| | | lung carcinoma | |
| | Cetuximab + cisplatin | head & neck | EGF receptor |
| | | cancer | |
| | | (extensive | |
| | | incurable local- | |
| | | regional disease | |
| | | & distant | |
| | | metasteses) | |
| | Cetuximab + radiation | locally advanced | EGF receptor |
| | | head & neck | |
| | | carcinoma | |
| | BEC2 + Bacillus | small cell lung | mimics ganglioside |
| | Calmette Guerin | carcinoma | GD3 |
| | BEC2 + Bacillus | melanoma | mimics ganglioside |
| | Calmette Guerin | | GD3 |
| | IMC-1C11 | colorectal cancer | VEGF-receptor |
| | | with liver | |
| | | metasteses | |
| ImmonoGen | nuC242-DM1 | Colorectal, | nuC242 |
| | | gastric, and | |
| | | pancreatic | |
| | | cancer | |
| ImmunoMedics | LymphoCide | Non-Hodgkins | CD22 |
| | | lymphoma | |
| | LymphoCide Y-90 | Non-Hodgkins | CD22 |
| | | lymphoma | |
| | CEA-Cide | metastatic solid | CEA |
| | | tumors | |
| | CEA-Cide Y-90 | metastatic solid | CEA |
| | | tumors | |
| | CEA-Scan (Tc-99m- | colorectal cancer | CEA |
| | labeled arcitumomab) | (radioimaging) | |
| | CEA-Scan (Tc-99m- | Breast cancer | CEA |
| | labeled arcitumomab) | (radioimaging) | |
| | CEA-Scan (Tc-99m- | lung cancer | CEA |
| | labeled arcitumomab) | (radioimaging) | |
| | CEA-Scan (Tc-99m- | intraoperative | CEA |
| | labeled arcitumomab) | tumors (radio | |
| | | imaging) | |
| | LeukoScan (Tc-99m- | soft tissue | CEA |
| | labeled sulesomab) | infection | |
| | | (radioimaging) | |
| | LymphoScan (Tc-99m- | lymphomas | CD22 |
| | labeled) | (radioimaging) | |
| | AFP-Scan (Tc-99m- | liver 7 gem-cell | AFP |
| | labeled) | cancers | |
| | | (radioimaging) | |
| Intracel | HumaRAD-HN | head & neck | NA |
| | (+yttrium-90) | cancer | |
| | HumaSPECT | colorectal | NA |
| | | imaging | |
| Medarex | MDX-101 (CTLA-4) | Prostate and | CTLA-4 |
| | | other cancers | |
| | MDX-210 (her-2 | Prostate cancer | HER-2 |
| | overexpression) | | |
| | MDX-210/MAK | Cancer | HER-2 |
| MedImmune | Vitaxin | Cancer | αvβ₃ |
| Merck KGaA | MAb 425 | Various cancers | EGF receptor |
| | IS-IL-2 | Various cancers | Ep-CAM |
| Millennium | Campath | chronic | CD52 |
| | (alemtuzumab) | lymphocytic | |
| | | leukemia | |
| NeoRx | CD20-streptavidin | Non-Hodgkins | CD20 |
| | (+biotin-yttrium 90) | lymphoma | |
| | Avidicin (albumin + | metastatic | NA |
| | NRLU13) | cancer | |
| Peregrine | Oncolym (+iodine-131) | Non-Hodgkins | HLA-DR 10 beta |
| | | lymphoma | |
| | Cotara (+iodine-131) | unresectable | DNA-associated |
| | | malignant | proteins |
| | | glioma | |
| Pharmacia | C215 (+staphylococcal | pancreatic | NA |
| Corporation | enterotoxin) | cancer | |
| | MAb, lung/kidney | lung & kidney | NA |
| | cancer | cancer | |
| | nacolomab tafenatox | colon & | NA |
| | (C242 + staphylococcal | pancreatic | |
| | enterotoxin) | cancer | |
| Protein Design | Nuvion | T cell | CD3 |
| Labs | | malignancies | |
| | SMART M195 | AML | CD33 |
| | SMART 1D10 | NHL | HLA-DR antigen |
| Titan | CEAVac | colorectal | CEA |
| | | cancer, | |
| | | advanced | |
| | TriGem | metastatic | GD2-ganglioside |
| | | melanoma & | |
| | | small cell lung | |
| | | cancer | |
| | TriAb | metastatic breast | MUC-1 |
| | | cancer | |
| Trilex | CEA Vac | colorectal | CEA |
| | | cancer, | |
| | | advanced | |
| | TriGem | metastatic | GD2-ganglioside |
| | | melanoma & | |
| | | small cell lung | |
| | | cancer | |
| | TriAb | metastatic breast | MUC-1 |
| | | cancer | |
| Viventia Biotech | NovoMAb-G2 | Non-Hodgkins | NA |
| | radiolabeled | lymphoma | |
| | Monopharm C | colorectal & | SK-1 antigen |
| | | pancreatic | |
| | | carcinoma | |
| | GlioMAb-H (+ gelonin | gliorna, | NA |
| | toxin) | melanoma & | |
| | | neuroblastoma | |
| Xoma | Rituxan | Relapsed/refractory | CD20 |
| | | low-grade or | |
| | | follicular NHL | |
| | Rituxan | intermediate & | CD20 |
| | | high-grade NHL | |
| | ING-1 | adenomcarcinoma | Ep-CAM |

### 5.3.19. Antibodies that can be Used for Inflammatory Disorders or Autoimmune Diseases

The formulations of the present invention further comprises any of the antibodies known in the art for the treatment and/or prevention of autoimmune disease or inflammatory disease. A non-limiting example of the antibodies that are used for the treatment or prevention of inflammatory disorders which can be engineered according to the invention is presented in Table 6A, and a non-limiting example of the antibodies that are used for the treatment or prevention of autoimmune disorder is presented in Table 6B.

TABLE 6A
ANTIBODIES FOR INFLAMMATORY DISEASES AND
AUTOIMMUNE DISEASES THAT CAN USED IN ACCORDANCE WITH THE
INVENTION.

| Antibody | Target | | | | |
|---|---|---|---|---|---|
| Name | Antigen | Product Type | Isotype | Sponsors | Indication |
| 5G1.1 | Complement | Humanized | IgG | Alexion | Rheumatoid |
| | (C5) | | | Pharm Inc | Arthritis |
| 5G1.1 | Complement | Humanized | IgG | Alexion | SLE |
| | (C5) | | | Pharm Inc | |
| 5G1.1 | Complement | Humanized | IgG | Alexion | Nephritis |
| | (C5) | | | Pharm Inc | |
| 5G1.1-SC | Complement | Humanized | ScFv | Alexion | Cardiopulmonary |
| | (C5) | | | Pharm Inc | Bypass |
| 5G1.1-SC | Complement | Humanized | ScFv | Alexion | Myocardial |
| | (C5) | | | Pharm Inc | Infarction |
| 5G1.1-SC | Complement | Humanized | ScFv | Alexion | Angioplasty |
| | (C5) | | | Pharm Inc | |
| ABX-CBL | CBL | Human | | Abgenix Inc | GvHD |
| ABX-CBL | CD147 | Murine | IgG | Abgenix Inc | Allograft rejection |
| ABX-IL8 | IL-8 | Human | IgG2 | Abgenix Inc | Psoriasis |
| Antegren | VLA-4 | Humanized | IgG | Athena/Elan | Multiple Sclerosis |
| Anti- | CD11a | Humanized | IgG1 | Genentech | Psoriasis |
| CD11a | | | | Inc/Xoma | |
| Anti-CD18 | CD18 | Humanized | Fab′2 | Genentech Inc | Myocardial |
| | | | | | infarction |
| Anti-LFA1 | CD18 | Murine | Fab′2 | Pasteur- | Allograft rejection |
| | | | | Merieux/ | |
| | | | | Immunotech | |
| Antova | CD40L | Humanized | IgG | Biogen | Allograft rejection |
| Antova | CD40L | Humanized | IgG | Biogen | SLE |
| BTI-322 | CD2 | Rat | IgG | Medimmune | GvHD, Psoriasis |
| | | | | Inc | |
| CDP571 | TNF-alpha | Humanized | IgG4 | Celltech | Crohn's |
| CDP571 | TNF-alpha | Humanized | IgG4 | Celltech | Rheumatoid |
| | | | | | Arthritis |
| CDP850 | E-selectin | Humanized | | Celltech | Psoriasis |
| Corsevin M | Fact VII | Chimeric | | Centocor | Anticoagulant |
| D2E7 | TNF-alpha | Human | | CAT/BASF | Rheumatoid |
| | | | | | Arthritis |
| Hu23F2G | CD11/18 | Humanized | | ICOS Pharm | Multiple Sclerosis |
| | | | | Inc | |
| Hu23F2G | CD11/18 | Humanized | IgG | ICOS Pharm | Stroke |
| | | | | Inc | |
| IC14 | CD14 | | | ICOS Pharm | Toxic shock |
| | | | | Inc | |
| ICM3 | ICAM-3 | Humanized | | ICOS Pharm | Psoriasis |
| | | | | Inc | |
| IDEC-114 | CD80 | Primatised | | IDEC | Psoriasis |
| | | | | Pharm/Mitsubishi | |
| IDEC-131 | CD40L | Humanized | | IDEC | SLE |
| | | | | Pharm/Eisai | |
| IDEC-131 | CD40L | Humanized | | IDEC | Multiple Sclerosis |
| | | | | Pharm/Eisai | |
| IDEC-151 | CD4 | Primatised | IgG1 | IDEC | Rheumatoid |
| | | | | Pharm/Glaxo | Arthritis |
| | | | | SmithKline | |
| IDEC-152 | CD23 | Primatised | | IDEC Pharm | Asthma/Allergy |
| Infliximab | TNF-alpha | Chimeric | IgG1 | Centocor | Rheumatoid |
| | | | | | Arthritis |
| Infliximab | TNF-alpha | Chimeric | IgG1 | Centocor | Crohn's |
| LDP-01 | beta2- | Humanized | IgG | Millennium | Stroke |
| | integrin | | | Inc | |
| | | | | (LeukoSite | |
| | | | | Inc.) | |
| LDP-01 | beta2- | Humanized | IgG | Millennium | Allograft rejection |
| | integrin | | | Inc | |
| | | | | (LeukoSite | |
| | | | | Inc.) | |
| LDP-02 | alpha4beta7 | Humanized | | Millennium | Ulcerative Colitis |
| | | | | Inc | |
| | | | | (LeukoSite | |
| | | | | Inc.) | |
| MAK- | TNF alpha | Murine | Fab′2 | Knoll Pharm, | Toxic shock |
| 195F | | | | BASF | |
| MDX-33 | CD64 (FcR) | Human | | Medarex/Centeon | Autoimmune |
| | | | | | haematogical |
| | | | | | disorders |
| MDX-CD4 | CD4 | Human | IgG | Medarex/Eisai/ | Rheumatoid |
| | | | | Genmab | Arthritis |
| MEDI-507 | CD2 | Humanized | | Medimmune | Psoriasis |
| | | | | Inc | |
| MEDI-507 | CD2 | Humanized | | Medimmune | GvHD |
| | | | | Inc | |
| OKT4A | CD4 | Humanized | IgG | Ortho Biotech | Allograft rejection |
| OrthoClone | CD4 | Humanized | IgG | Ortho Biotech | Autoimmune |
| OKT4A | | | | | disease |
| Orthoclone/ | CD3 | Murine | mIgG2a | Ortho Biotech | Allograft rejection |
| anti-CD3 | | | | | |
| OKT3 | | | | | |
| RepPro/ | gpIIbIIIa | Chimeric | Fab | Centocor/Lilly | Complications of |
| Abciximab | | | | | coronary |
| | | | | | angioplasty |
| rhuMab- | IgE | Humanized | IgG1 | Genentech/No | Asthma/Allergy |
| E25 | | | | vartis/Tanox | |
| | | | | Biosystems | |
| SB-240563 | IL5 | Humanized | | GlaxoSmithKline | Asthma/Allergy |
| SB-240683 | IL-4 | Humanized | | GlaxoSmithKline | Asthma/Allergy |
| SCH55700 | IL-5 | Humanized | | Celltech/Schering | Asthma/Allergy |
| Simulect | CD25 | Chimeric | IgG1 | Novartis | Allograft rejection |
| | | | | Pharm | |
| SMART | CD3 | Humanized | | Protein | Autoimmune |
| a-CD3 | | | | Design Lab | disease |
| SMART | CD3 | Humanized | | Protein | Allograft rejection |
| a-CD3 | | | | Design Lab | |
| SMART | CD3 | Humanized | IgG | Protein | Psoriasis |
| a-CD3 | | | | Design Lab | |
| Zenapax | CD25 | Humanized | IgG1 | Protein | Allograft rejection |
| | | | | Design | |
| | | | | Lab/Hoffman- | |
| | | | | La Roche | |

TABLE 6B
ANTIBODIES FOR AUTOIMMUNE DISORDERS THAT CAN
BE USED IN ACCORDANCE WITH THE INVENTION

| Antibody | Indication | Target Antigen |
|---|---|---|
| ABX-RB2 | | antibody to CBL antigen on T cells, |
| | | B cells and NK cells |
| | | fully human antibody from the |
| | | Xenomouse |
| 5c8 (Anti CD-40 | Phase II trials were halted in Oct. | CD-40 |
| ligand antibody) | 99 examine “adverse events” | |
| IDEC 131 | systemic lupus erythyematous | anti CD40 |
| | (SLE) | humanized |
| IDEC 151 | rheumatoid arthritis | primatized; anti-CD4 |
| IDEC 152 | Asthma | primatized; anti-CD23 |
| IDEC 114 | Psoriasis | primatized anti-CD80 |
| MEDI-507 | rheumatoid arthritis; multiple | anti-CD2 |
| | sclerosis | |
| | Crohn's disease | |
| | Psoriasis | |
| LDP-02 (anti-b7 | inflammatory bowel disease | a4b7 integrin receptor on white |
| mAb) | Chron's disease | blood cells (leukocytes) |
| | ulcerative colitis | |
| SMART Anti- | autoimmune disorders | Anti-Gamma Interferon |
| Gamma Interferon | | |
| antibody | | |
| Verteportin | rheumatoid arthritis | |
| MDX-33 | blood disorders caused by | monoclonal antibody against FcRI |
| | autoimmune reactions | receptors |
| | Idiopathic Thrombocytopenia | |
| | Purpurea (ITP) | |
| | autoimmune hemolytic anemia | |
| MDX-CD4 | treat rheumatoid arthritis and other | monoclonal antibody against CD4 |
| | autoimmunity | receptor molecule |
| VX-497 | autoimmune disorders | inhibitor of inosine monophosphate |
| | multiple sclerosis | dehydrogenase |
| | rheumatoid arthritis | (enzyme needed to make new RNA |
| | inflammatory bowel disease | and DNA |
| | lupus | used in production of nucleotides |
| | psoriasis | needed for lymphocyte |
| | | proliferation) |
| VX-740 | rheumatoid arthritis | inhibitor of ICE |
| | | interleukin-1 beta (converting |
| | | enzyme |
| | | controls pathways leading to |
| | | aggressive immune response) |
| VX-745 | specific to inflammation | inhibitor of P38MAP kinase |
| | involved in chemical signalling of | mitogen activated protein kinase |
| | immune response | |
| | onset and progression of | |
| | inflammation | |
| Enbrel (etanercept) | | targets TNF (tumor necrosis factor) |
| IL-8 | | fully human monoclonal antibody |
| | | against IL-8 (interleukin 8) |
| Apogen MP4 | | recombinant antigen |
| | | selectively destroys disease |
| | | associated T-cells |
| | | induces apoptosis |
| | | T-cells eliminated by programmed |
| | | cell death |
| | | no longer attack body's own cells |
| | | specific apogens target specific T- |
| | | cells |

### 5.4 Methods of Producing Antibodies

The antibodies used in the present invention can be produced by any method known in the art for the synthesis of antibodies, in particular, by chemical synthesis or preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with an antigen (either the full length protein or a domain thereof, e.g., the extracellular or the ligand binding domain) and once an immune response is detected, e.g., antibodies specific for the particular antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody of the invention wherein, preferably, the hybridoma is generated by fusing splenocytes isolated from a mouse immunized with the antigen with myeloma cells and then screening the hybridomas resulting from the fusion for hybridoma clones that secrete an antibody able to bind the antigen.

Antibody fragments used in the present invention may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177; Kettleborough et al., 1994, Eur. J. Immunol. 24:952-958; Persic et al., 1997, Gene 187:9; Burton et al., 1994, Advances in Immunology 57:191-280; International Application No. PCT/GB91/01134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

Phage may be screened for antigen binding activities. As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12:864; Sawai et al., 1995, AJRI 34:26; and Better et al., 1988, Science 240:1041 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Fremont, Calif.) and Medarex (Princeton, N.J.) can be engaged to provide human antibodies directed against a selected antigen using technology similar to that described above.

A chimeric antibody is a molecule in which different portions of the antibody are derived from different immunoglobulin molecules such as antibodies having a variable region derived from a non-human antibody and a human immunoglobulin constant region. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 6,311,415, 5,807,715, 4,816,567, and 4,816,397, which are incorporated herein by reference in their entirety. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, PNAS 91:969), and chain shuffling (U.S. Pat. No. 5,565,332).

Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann et al., 1988, Nature 332:323, which are incorporated herein by reference in their entireties.)

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG₁, IgG₂, IgG₃ and IgG₄. Usually the constant domain is a complement fixing constant domain where it is desired that the humanized antibody exhibit cytotoxic activity, and the class is typically IgG₁. Where such cytotoxic activity is not desirable, the constant domain may be of the IgG₂ class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, and most preferably greater than 95%. Humanized antibodies can be produced using variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, 5,585,089, International Publication No. WO 9317105, Tan et al., 2002, J. Immunol. 169:1119-25, Caldas et al., 2000, Protein Eng. 13:353-60, Morea et al., 2000, Methods 20:267-79, Baca et al., 1997, J. Biol. Chem. 272:10678-84, Roguska et al., 1996, Protein Eng. 9:895-904, Couto et al., 1995, Cancer Res. 55 (23 Supp):5973s-5977s, Couto et al., 1995, Cancer Res. 55:1717-22, Sandhu, 1994, Gene 150:409-10, Pedersen et al., 1994, J. Mol. Biol. 235:959-73, Jones et al., 1986, Nature 321:522-525, Riechmann et al., 1988, Nature 332:323, and Presta, 1992, Curr. Op. Struct. Biol. 2:593-596. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

Further, the antibodies of the invention can, in turn, be utilized to generate anti-idiotype antibodies using techniques well known to those skilled in the art. (See, e.g., Greenspan & Bona, 1989, FASEB J. 7:437-444; and Nissinoff, 1991, J. Immunol. 147:2429-2438). The invention provides methods employing the use of polynucleotides comprising a nucleotide sequence encoding an antibody of the invention or a fragment thereof.

### 5.4.1 Recombinant Expression of an Antibody

Recombinant expression of an antibody used in the invention, a derivative, analog or fragment thereof, (e.g., a heavy or light chain of an antibody of the invention or a portion thereof or a single chain antibody of the invention), requires construction of an expression vector containing a polynucleotide that encodes the antibody. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, BioTechnology 8:2). In a specific embodiment, the expression of nucleotide sequences encoding antibodies or fragments thereof which immunospecifically bind to and agonize is regulated by a constitutive promoter, inducible promoter or tissue specific promoter.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, PNAS 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, BT483, Hs578T, HTB2, BT2O, NS1, T47D, NS0 (a murine myeloma cell line that does not endogenously produce any immunoglobulin chains), CRL7O3O and HsS78Bst cells.

The antibodies comprising at least one zero-order thioether can be recombinantly produced by any cell lines for producing antibodies known to those skilled in the art. It has been found that it is advantageous to produce the antibodies of the invention in melanoma cells. In certain embodiments, the antibodies of the invention are recombinantly produced in melanoma cells. In some embodiments, the antibodies of the invention are not recombinantly produced in CHO cell line. In other embodiments, the antibodies of the invention are not recombinantly produced in NS0 cell line.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), glutamine synthase, hypoxanthine guanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, gs-, hgprt- or aprt-cells, respectively. Also, antimetabolite resistance can be used as the basis of selection for the following genes: dhfr, which confers resistance to methotrexate (Wigler et al., 1980, PNAS 77:357; O'Hare et al., 1981, PNAS 78:1527); gpt, which confers resistance to mycophenolic acid (Mulligan & Berg, 1981, PNAS 78:2072); neo, which confers resistance to the aminoglycoside G-418 (Wu and Wu, 1991, Biotherapy 3:87; Tolstoshev, 1993, Ann. Rev. Pharmacol. Toxicol. 32:573; Mulligan, 1993, Science 260:926; and Morgan and Anderson, 1993, Ann. Rev. Biochem. 62: 191; May, 1993, TIB TECH 11:155-); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, PNAS 77:2197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

### 5.5 Use of the Antibodies and Compositions of the Present Invention

The formulations comprising antibodies and compositions thereof can be used in any context that those of skilled in the art recognize. For example, the formulations of the invention can be used directly against a particular antigen. The formulations of the invention comprising antibodies and compositions can also be used in diagnostic assays either in vivo or in vitro for detection/identification of the expression of an antigen in a subject or a biological sample (e.g., cells or tissues), formulations of the invention comprising antibodies and compositions can be used alone or in combination with other prophylactic or therapeutic agents for treating, managing, preventing or ameliorating a disorder or one or more symptoms thereof.

The present invention provides methods for preventing, managing, treating, or ameliorating a disorder comprising administering to a subject in need thereof one or more antibodies of the invention alone or in combination with one or more therapies (e.g., one or more prophylactic or therapeutic agents) other than an antibody of the invention. The present invention also provides formulations comprising one or more antibodies of the invention and one or more prophylactic or therapeutic agents other than antibodies of the invention and methods of preventing, managing, treating, or ameliorating a disorder or one or more symptoms thereof utilizing said compositions. Therapeutic or prophylactic agents include, but are not limited to, small molecules, synthetic drugs, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides) antibodies, synthetic or natural inorganic molecules, mimetic agents, and synthetic or natural organic molecules.

Any therapy which is known to be useful, or which has been used or is currently being used for the prevention, management, treatment, or amelioration of a disorder or one or more symptoms thereof can be used in combination with an antibody or a composition of the invention in accordance with the invention described herein. See, e.g., Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W. B. Saunders, Philadelphia, 1996 for information regarding therapies (e.g., prophylactic or therapeutic agents) which have been or are currently being used for preventing, treating, managing, or ameliorating a disorder or one or more symptoms thereof. Examples of such agents include, but are not limited to, immunomodulatory agents, anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, non-steriodal anti-inflammatory drugs (e.g., aspirin, ibuprofen, diclofenac, and COX-2 inhibitors), anti-cancer agents, pain relievers, leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythomycin, penicillin, mithramycin, and anthramycin (AMC)).

In a specific embodiment, the present invention provides a method comprising administering a formulation comprising one or more humanized anti-IL-9 antibodies to a subject, preferably a human subject, for preventing, treating, managing, or ameliorating a respiratory condition or one or more symptoms thereof. In one embodiment, the invention encompasses a method of preventing, treating, managing, or ameliorating a respiratory disorder or one or more symptoms thereof (e.g., an allergy, wheezing, and asthma), said method comprising administering to a subject in need thereof a dose of a prophylactically or therapeutically effective amount of a formulation comprising one or more of humanized anti-IL-9 antibodies. In another embodiment, the invention provides a method of preventing, treating, managing, or ameliorating a respiratory infection or one or more symptoms thereof, said method comprising administering a prophylactically or therapeutic effective amount of one or more humanized anti-IL-9 antibodies.

In a specific embodiment, the present invention provides a method comprising administering a formulation of one or more humanized anti-EphA2 antibodies to a subject, preferably a human subject, for preventing, treating, managing, or ameliorating a hyperproliferative cell disease or one or more symptoms thereof. In one embodiment, one or more humanized anti-EphA2 antibodies are administered alone or in combination with other agents to a subject to prevent, treat, manage, or ameliorate cancer or one or more symptoms thereof (see, e.g., U.S. application Ser. No. 10/436,782, which is incorporated herein by reference in its entirety). In another embodiment, one or more humanized anti-EphA2 antibodies are administered alone or in combination with other agents to a subject to prevent, treat, manage, or ameliorate a disorder involving non-neoplastic hyperproliferative cells, in particular hyperproliferative epithlial and endothelial cells, or one or symptoms thereof (see e.g., U.S. Application Ser. No. 60/462,024, which is incorporated herein by reference in its entirety). In yet another embodiment, one or more humanized anti-EphA2 antibodies are used for diagnostic or screening purposes.

The formulations comprising antibodies and compositions of the invention can be used directly against a particular antigen. In some embodiments, the antibodies and compositions of the invention belong to a subclass or isotype that is capable of mediating the lysis of cells to which the antibody binds. In a specific embodiment, the antibodies of the invention belong to a subclass or isotype that, upon complexing with cell surface proteins, activates serum complement and/or mediates antibody dependent cellular cytotoxicity (ADCC) by activating effector cells such as natural killer cells or macrophages.

The biological activities of antibodies are known to be determined, to a large extent, by the constant domains or Fc region of the antibody molecule (Uananue and Benacerraf, Textbook of Immunology, 2nd Edition, Williams & Wilkins, p. 218 (1984)). This includes their ability to activate complement and to mediate antibody-dependent cellular cytotoxicity (ADCC) as effected by leukocytes. Antibodies of different classes and subclasses differ in this respect, as do antibodies from the same subclass but different species; according to the present invention, antibodies of those classes having the desired biological activity are prepared. Preparation of these antibodies involves the selection of antibody constant domains and their incorporation in the humanized antibody by known technique. For example, mouse immunoglobulins of the IgG3 and IgG2a class are capable of activating serum complement upon binding to the target cells which express the cognate antigen, and therefore humanized antibodies which incorporate IgG3 and IgG2a effector functions are desirable for certain therapeutic applications.

In some embodiments, formulations of the invention comprising antibodies and compositions are useful in passively immunizing patients.

The formulations of the invention comprising antibodies and compositions can also be used in diagnostic assays either in vivo or in vitro for detection/identification of the expression of an antigen in a subject or a biological sample (e.g., cells or tissues). Non-limiting examples of using an antibody, or a composition comprising an antibody in a diagnostic assay are given in U.S. Pat. Nos. 6,392,020; 6,156,498; 6,136,526; 6,048,528; 6,015,555; 5,833,988; 5,811,310; 8 5,652,114; 5,604,126; 5,484,704; 5,346,687; 5,318,892; 5,273,743; 5,182,107; 5,122,447; 5,080,883; 5,057,313; 4,910,133; 4,816,402; 4,742,000; 4,724,213; 4,724,212; 4,624,846; 4,623,627; 4,618,486; 4,176,174 (all of which are incorporated herein by reference). Suitable diagnostic assays for the antigen and its antibodies depend on the particular antibody used. Non-limiting examples are an ELISA, sandwich assay, and steric inhibition assays. For in vivo diagnostic assays using the antibodies of the invention, the antibodies may be conjugated to a label that can be detected by imaging techniques, such as X-ray, computed tomography (CT), ultrasound, or magnetic resonance imaging (MRI). The antibodies of the invention can also be used for the affinity purification of the antigen from recombinant cell culture or natural sources.

### 5.5.1 Prophylactic and Therapeutic Use of Formulations of Antibodies Against RSV Infections

The present invention provides antibody-based therapies which involve administering antibodies of the invention to a subject, preferably a human, for preventing, treating, or ameliorating a RSV infection (i.e., an upper and/or lower respiratory tract RSV infection), otitis media (stemming from, caused by, or associated with a RSV infection), or a symptom or respiratory condition relating thereto (including, but not limited to, asthma, wheezing, RAD, or a combination thereof). Prophylactic and therapeutic agents of the invention include, but are not limited to, antibodies (including analogs and derivatives thereof as described herein) and nucleic acids encoding antibodies (including analogs and derivatives thereof and anti-idiotypic antibodies as described herein). Antibodies may be provided in pharmaceutically acceptable compositions as known in the art or as described herein.

Formulations of the present invention comprising antibodies that function as antagonists of a RSV infection can be administered to a subject, preferably a human, to treat, prevent or ameliorate an upper and/or lower respiratory tract RSV infection, otitis media (preferably, stemming from, caused by, or associated with a RSV infection), or a symptom or respiratory condition relating thereto (including, but not limited to, asthma, wheezing, RAD, or a combination thereof). For example, antibodies which disrupt or prevent the interaction between a RSV antigen and its host cell receptor may be administered to subject, preferably a human, to treat, prevent or ameliorate an upper and/or lower respiratory tract RSV infection, otitis media (stemming from, caused by, or associated with a RSV infection), or a symptom or respiratory condition relating thereto (including, but not limited to, asthma, wheezing, RAD, or a combination thereof).

In a specific embodiment, an antibody prevents or inhibits RSV from binding to its host cell receptor by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV binding to its host cell receptor in the absence of said antibodies or in the presence of a negative control in an assay known to one of skill in the art or described herein. In another embodiment, a combination of antibodies prevent or inhibit RSV from binding to its host cell receptor by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV binding to its host cell receptor in the absence of said antibodies or in the presence of a negative control in an assay known to one of skill in the art or described herein.

In a specific embodiment, an antibody prevents or inhibits RSV-induced fusion by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV-induced fusion in the absence of said antibodies or in the presence of a negative control in an assay known to one of skill in the art or described herein. In another embodiment, a combination of antibodies prevent or inhibit RSV-induced fusion by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV-induced fusion in the absence of said antibodies or in the presence of a negative control in an assay known to one of skill in the art or described herein.

In a specific embodiment, an antibody prevents or inhibits RSV-induced fusion after viral attachment to cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV-induced fusion after viral attachment to cells in the absence of said antibodies or in the presence of a negative control in an assay known to one of skill in the art or described herein. In another embodiment, a combination of antibodies prevent or inhibit RSV-induced fusion after viral attachment to cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV-induced fusion after viral attachment to cells in the absence of said antibodies or in the presence of a negative control in an assay known to one of skill in the art or described herein.

Antibodies which do not prevent RSV from binding its host cell receptor but inhibit or downregulate RSV replication can also be administered to a subject to treat, prevent or ameliorate an upper and/or lower respiratory tract RSV infection, otitis media (stemming from, caused by, or associated with a RSV infection), or a symptom or respiratory condition relating thereto (including, but not limited to, asthma, wheezing, RAD, or a combination thereof). The ability of an antibody to inhibit or downregulate RSV replication may be determined by techniques described herein or otherwise known in the art. For example, the inhibition or downregulation of RSV replication can be determined by detecting the RSV titer in the lungs of a subject, preferably a human. In further embodiments, the inhibition or downregulation of RSV replication can be determined by detecting the amount of RSV in the nasal passages or in the middle ear by methods known in the art (e.g., Northern blot analysis, RT-PCR, Western Blot analysis, etc.).

In some embodiments, a formulations of the present invention comprises an antibody that results in reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105 fold, about 110-fold, about 115-fold, about 120 fold, about 125-fold or higher in RSV titer in the lung. The fold-reduction in RSV titer may be as compared to a negative control (such as placebo), as compared to another treatment (including, but not limited to treatment with palivizumab), or as compared to the titer in the patient prior to antibody administration.

In a specific embodiment, formulation of the present invention comprises an antibody that inhibits or downregulates RSV replication by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV replication in absence of said antibodies or in the presence of a negative control in an assay known in the art or described herein. In another embodiment, a combination of antibodies inhibit or downregulate RSV replication by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV replication in absence of said antibodies or in the presence of a negative control in an assay known in the art or described herein.

In some embodiments, formulation of the present invention comprises an antibody that results in reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105 fold, about 110-fold, about 115-fold, about 120 fold, about 125-fold or higher in RSV titer in the upper respiratory tract. The fold-reduction in RSV titer may be as compared to a negative control (such as placebo), as compared to another treatment (including, but not limited to treatment with palivizumab), or as compared to the titer in the patient prior to antibody administration.

In other embodiments, formulation of the present invention comprises an antibody that results in reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105 fold, about 110-fold, about 115-fold, about 120 fold, about 125-fold or higher in RSV titer in the lower respiratory tract. The fold-reduction in RSV titer may be as compared to a negative control (such as placebo), as compared to another treatment (including, but not limited to treatment with palivizumab), or as compared to the titer in the patient prior to antibody administration.

One or more antibodies in connection with the present invention that immunospecifically bind to one or more RSV antigens may be used locally or systemically in the body as a prophylactic or therapeutic agent. The antibodies may also be advantageously utilized in combination with other monoclonal or chimeric antibodies, or with lymphokines or hematopoietic growth factors (such as, e.g., IL-2, IL-3 and IL-7), which, for example, serve to increase the number or activity of effector cells which interact with the antibodies. The antibodies may also be advantageously utilized in combination with other monoclonal or chimeric antibodies, or with lymphokines or hematopoietic growth factors (such as, e.g., IL-2, IL-3 and IL-7), which, for example, serve to increase the immune response. The antibodies may also be advantageously utilized in combination with one or more drugs used to treat RSV infection such as, for example anti-viral agents. Antibodies of the invention may be used in combination with one or more of the following drugs: NIH-351 (Gemini Technologies), recombinant RSV vaccine (Aviron), RSVf-2 (Intracel), F-50042 (Pierre Fabre), T-786 (Trimeris), VP-36676 (ViroPharma), RFI-641 (American Home Products), VP-14637 (ViroPharma), PFP-1 and PFP-2 (American Home Products), RSV vaccine (Avant Immunotherapeutics), and F-50077 (Pierre Fabre). In a specific embodiment, an effective amount of an antibody and an effective amount of another therapy is used.

The formulations of the invention comprising antibodies may be administered alone or in combination with other types of therapies (e.g., hormonal therapy, immunotherapy, and anti-inflammatory agents). Generally, administration of products of a species origin or species reactivity (in the case of antibodies) that is the same species as that of the patient is preferred. Thus, in a preferred embodiment, human or humanized antibodies, derivatives, analogs, or nucleic acids, are administered to a human patient for therapy or prophylaxis.

In specific embodiments, an antibody is administered in combination with one or more anti-IL-9 antibodies (such as those disclosed in U.S. Publication No. 2005/0002934) either alone or in combination with one or more antibodies of the invention and/or other types of therapies or other agents (e.g., hormone therapy, immunotherapy, and anti-inflammatory agents, such as those disclosed in U.S. Publication No. 2005/0002934, which is herein incorporated by reference in its entirety).

It is preferred to use high affinity and/or potent in vivo inhibiting antibodies and/or neutralizing antibodies that immunospecifically bind to a RSV antigen, for both immunoassays directed to RSV, and the prevention, management or treatment of an upper and/or lower respiratory tract RSV infection, otitis media (preferably, stemming from, caused by, or associated with a RSV infection), or a symptom or respiratory condition relating thereto (including, but not limited to, asthma, wheezing, RAD, or a combination thereof). It is also preferred to use polynucleotides encoding high affinity and/or potent in vivo inhibiting antibodies and/or neutralizing antibodies that immunospecifically bind to a RSV antigen, for both immunoassays directed to RSV and therapy for an upper and/or lower respiratory tract RSV infection, otitis media (stemming from, caused by, or associated with a RSV infection), or a symptom or respiratory condition relating thereto (including, but not limited to, asthma, wheezing, RAD, or a combination thereof). Such antibodies will preferably have an affinity for the RSV F glycoprotein and/or fragments of the F glycoprotein.

The methods of the invention comprise the administration of one or more antibodies to patients suffering from or expected to suffer from (e.g., patients with a genetic predisposition for or patients that have previously suffered from) an upper and/or lower respiratory tract RSV infection, otitis media (preferably, stemming from, caused by, or associated with a RSV infection), or a symptom or respiratory condition relating thereto (including, but not limited to, asthma, wheezing, RAD, or a combination thereof). Such patients may have been previously treated or are currently being treated for the infection, otitis media, or a respiratory condition, e.g., with a therapy other than an antibody of the invention. In one embodiment, the methods of the invention comprise the administration of one or more antibodies to patients that are immunocompromised or immunosuppressed. In a certain embodiment, an antibody administered to patients that are immunocompromised or immunosuppressed. In another embodiment, an antibody is administered to a human with cystic fibrosis, bronchopulmonary dysplasia, congenital heart disease, congenital immunodeficiency or acquired immunodeficiency, or to a human who has had a bone marrow transplant to treat, prevent or ameliorate one or more symptoms associated with an upper and/or lower respiratory tract RSV infection or otitis media (preferably, stemming from, caused by, or associated with a RSV infection), or a symptom or respiratory condition relating thereto (including, but not limited to, asthma, wheezing, RAD, or a combination thereof). In another embodiment, a formulation of the invention comprising an antibody is administered to a human infant, preferably a human infant born prematurely or a human infant at risk of hospitalization for RSV infection to treat, prevent or ameliorate one or more symptoms associated with an upper and/or lower respiratory tract RSV infection or otitis media. In yet another embodiment, a formulation of the invention comprising an antibody is administered to the elderly or people in group homes (e.g., nursing homes or rehabilitation centers). In accordance with the invention, a formulation of the invention comprising an antibody may be used as any line of therapy, including, but not limited to, a first, second, third and fourth line of therapy, including, but not limited to, a first, second, third and fourth line of therapy. Further, in accordance with the invention, a formulation of the invention comprising an antibody can be used before any adverse effects or intolerance of the therapies other than an antibody occurs. The invention encompasses methods for administering one or more antibodies to prevent the onset or recurrence of an upper and/or lower respiratory tract RSV infection or otitis media.

In one embodiment, the invention also provides methods of treatment, management, prevention and/or amelioration of an upper and/or lower respiratory tract RSV infection (preferably stemming from, caused by, or associated with a RSV infection), otitis media or a symptom or respiratory condition related thereto (including, but not limited to, asthma, wheezing, RAD, or a combination thereof) as alternatives to current therapies. In a specific embodiment, the current therapy has proven or may prove too toxic (i.e., results in unacceptable or unbearable side effects) for the patient. In another embodiment, an antibody decreases the side effects as compared to the current therapy. In another embodiment, the patient has proven refractory to a current therapy. In such embodiments, the invention provides for the administration of one or more antibodies of the invention without any other anti-infection therapies. In certain embodiments, one or more antibodies can be administered to a patient in need thereof instead of another therapy to treat an upper and/or lower respiratory tract RSV infection, otitis media or a symptom or respiratory condition related thereto (including, but not limited to, asthma, wheezing, RAD, or a combination thereof). In one embodiment, the invention provides methods of treating, managing, preventing and/or ameliorating an active upper and/or lower respiratory tract RSV infection, otitis media or a symptom or respiratory condition related thereto (including, but not limited to, asthma, wheezing, RAD, or a combination thereof).

The present invention also encompasses methods for administering one or more antibodies to treat or ameliorate symptoms of an upper and/or lower respiratory tract RSV infection or otitis media in patients that are or have become refractory to therapies other than the antibodies. The determination whether the infection is refractory can be made either in vivo or in vitro by any method known in the art for assaying the effectiveness of a therapy on affected cells in the infection, particularly epithelial cells, or in patients that are or have become refractory to therapies other than antibodies of the invention.

In certain embodiments, an effective amount of one or more antibodies in the formulation of the invention is administered in combination with one or more supportive measures to a subject in need thereof to prevent, manage, treat, and/or ameliorate an upper and/or lower respiratory tract RSV infection, otitis media (preferably, stemming from, caused by, or associated with a RSV infection), or a symptom or respiratory condition relating thereto (including, but not limited to, asthma, wheezing, RAD, or a combination thereof). Non-limiting examples of supportive measures include humidification of the air by an ultrasonic nebulizer, aerolized recemic epinephrine, oral dexamethasone, intravenous fluids, intubation, fever reducers (e.g., ibuprofen, acetometaphin), and antibiotic and/or anti-fungal therapy (i.e., to prevent or treat secondary bacterial and/or fungal infections).

In a specific embodiment, the invention provides methods for preventing, treating, managing, and/or ameliorating a RSV infection (i.e., an upper and/or lower respiratory tract RSV infection), otitis media (preferably, stemming from, caused by, or associated with a RSV infection), or a symptom or respiratory condition relating thereto (including, but not limited to, asthma, wheezing, RAD, or a combination thereof), said methods comprising administering to a subject in need thereof an effective amount of one or more antibodies of the invention alone or in combination with one or more anti-viral agents such as, but not limited to, amantadine, rimantadine, oseltamivir, znamivir, ribavarin, RSV-IVIG (i.e., intravenous immune globulin infusion) (RESPIGAM™), EphA2/EphrinA1 Modulators, and/or an anti-IL-9 antibody (see, e.g., U.S. Publication No. 2005/0002934).

In a specific embodiment, the invention provides methods for preventing, treating, managing, and/or ameliorating one or more secondary responses to a primary viral infection, said methods comprising administering an effective amount of one or more antibodies alone or in combination with an effective amount of other therapies (e.g., other prophylactic or therapeutic agents). Examples of secondary responses to a primary viral infection include, but are not limited to, asthma-like responsiveness to mucosal stimula, elevated total respiratory resistance, increased susceptibility to secondary viral, bacterial, and fungal infections, and development of conditions such as, but not limited to, bronchiolitis, pneumonia, croup, and febrile bronchitis.

In a specific embodiment, the invention provides methods of preventing, treating, managing, and/or ameliorating a RSV infection (i.e., an upper and/or lower respiratory tract RSV infection), otitis media (preferably, stemming from, caused by or associated with a RSV infection, such as an upper and/or lower respiratory tract RSV infection) or a symptom or respiratory condition relating thereto (including, but not limited to asthma, wheezing, RAD, or a combination thereof), said methods comprising administering to a subject in need thereof an effective amount of one or more antibodies in combination with an effective amount of an EphA2/EphrinA1 Modulator (U.S. Provisional Appn. Ser. No. 60/622,489, filed Oct. 27, 2004, entitled “Use of Modulators of EphA2 and EphrinA1 for the Treatment and Prevention of Infections”, which is incorporated by reference herein in its entirety). In another specific embodiment, the invention provides methods for preventing, treating, managing, and/or ameliorating a RSV infection (i.e., an upper and/or lower respiratory tract RSV infection), otitis media (preferably, stemming from, caused by or associated with a RSV infection, such as an upper and/or lower respiratory tract RSV infection) or a symptom or respiratory condition relating thereto (including, but not limited to asthma, wheezing, RAD, or a combination thereof), said methods comprising administering to a subject in need thereof an effective amount of one or more antibodies in combination with an effective amount of siplizumab (MedImmune, Inc., International Pub. No. WO 02/069904, which is incorporated herein by reference in its entirety). In another embodiment, the invention provides methods of preventing, treating, managing and/or ameliorating a RSV infection (i.e., an upper and/or lower respiratory tract RSV infection), otitis media (preferably, stemming from, caused by or associated with a RSV infection, such as an upper and/or lower respiratory tract RSV infection) or a symptom or respiratory condition relating thereto (including, but not limited to asthma, wheezing, RAD, or a combination thereof), said methods comprising administering to a subject in need thereof an effective amount of one or more antibodies in combination with an effective amount of one or more anti-IL-9 antibodies, such as those disclosed in U.S. Publication No. 2005/0002934, which is incorporated herein by reference in its entirety. In yet another embodiment, the invention provides methods for preventing, treating, managing, and/or ameliorating a RSV infection (i.e., an upper and/or lower respiratory tract RSV infection), otitis media (preferably, stemming from, caused by or associated with a RSV infection, such as an upper and/or lower respiratory tract RSV infection) or a symptom or respiratory condition relating thereto (including, but not limited to asthma, wheezing, RAD, or a combination thereof), said methods comprising administering to a subject in need thereof an effective amount of one or more antibodies of the invention in combination with an effective amount of two or more of the following: EphA2/EphrinA1 Modulators, an anti-IL-9 antibody and/or siplizumab.

The formulations of the invention, comprising antibodies, compositions, or combination therapies of the invention may be used as any line of therapy, including but not limited to, the first, second, third, fourth, or fifth line of therapy, to prevent, treat, and/or ameliorate an upper and/or lower respiratory tract RSV infection, otitis media (preferably, stemming from, caused by or associated with a RSV infection, such as an upper and/or lower respiratory tract RSV infection) or a symptom or respiratory condition relating thereto (including, but not limited to asthma, wheezing, RAD, or a combination thereof). The invention also includes methods of preventing, treating, managing, and/or ameliorating a RSV infection (i.e., an upper and/or lower respiratory tract RSV infection), otitis media (preferably, stemming from, caused by or associated with a RSV infection, such as an upper and/or lower respiratory tract RSV infection), or a symptom or respiratory condition relating thereto (including, but not limited to, asthma, wheezing, RAD, or a combination thereof) in a patient undergoing therapies for other diseases or disorders (e.g., non-RSV infections). The invention encompasses methods of preventing, managing, treating, and/or ameliorating a RSV infection (i.e., an upper and/or lower respiratory tract RSV infection), otitis media (preferably, stemming from, caused by or associated with a RSV infection, such as an upper and/or lower respiratory tract RSV infection) or a symptom or respiratory condition relating thereto (including, but not limited to asthma, wheezing, RAD, or a combination thereof) in a patient before any adverse effects or intolerance to therapies other than antibodies of the invention develops.

The invention also encompasses methods of preventing, treating, managing, and/or ameliorating a RSV infection (i.e., an upper and/or lower respiratory tract RSV infection), otitis media (preferably, stemming from, caused by or associated with a RSV infection, such as an upper and/or lower respiratory tract RSV infection), or a symptom or respiratory condition relating thereto (including, but not limited to, asthma, wheezing, RAD, or a combination thereof) in refractory patients. In certain embodiments, a patient with an upper and/or lower respiratory tract RSV infection, is refractory to a therapy when the infection has not significantly been eradicated and/or the symptoms have not been significantly alleviated. The determination of whether a patient is refractory can be made either in vivo or in vitro by any method known in the art for assaying the effectiveness of a therapy for infections, using art-accepted meanings of “refractory” in such a context. In various embodiments, a patient with an upper and/or lower respiratory tract RSV infection is refractory when viral replication has not decreased or has increased. The invention also encompasses methods of preventing the onset or reoccurrence of an upper and/or lower respiratory tract RSV infection or otitis media (preferably, stemming from, caused by or associated with a RSV infection, such as an upper and/or lower respiratory tract RSV infection) in patients at risk of developing such infections or otitis media.

The invention also encompasses methods of preventing, managing, treating, and/or ameliorating a RSV infection (i.e., an upper and/or lower respiratory tract RSV infection), otitis media (preferably, stemming from, caused by or associated with a RSV infection, such as an upper and/or lower respiratory tract RSV infection), or a symptom or respiratory condition relating thereto (including, but not limited to, asthma, wheezing, RAD, or a combination thereof) in patients who are susceptible to adverse reactions to conventional therapies. The invention further encompasses methods for preventing, treating, managing, and/or ameliorating a RSV infection (i.e., an upper and/or lower respiratory tract RSV infection) or otitis media (preferably, stemming from, caused by or associated with a RSV infection, such as an upper and/or lower respiratory tract RSV infection) or a symptom or respiratory condition relating thereto (including, but not limited to, asthma, wheezing, RAD, or a combination thereof) for which no anti-viral therapy is available.

The invention encompasses methods for preventing, treating, managing, and/or ameliorating a RSV infection (i.e., an upper and/or lower respiratory tract RSV infection), otitis media (preferably, stemming from, caused by or associated with a RSV infection, such as an upper and/or lower respiratory tract RSV infection), or a symptom or respiratory condition relating thereto (including, but not limited to, asthma, wheezing, RAD, or a combination thereof) in a patient who has proven refractory to therapies other than antibodies of the invention but are no longer on these therapies. In certain embodiments, the patients being treated in accordance with the methods of this invention are patients already being treated with antibiotics, anti-virals, anti-fungals, or other biological therapy/immunotherapy. Among these patients are refractory patients, patients who are too young for conventional therapies, and patients with reoccurring upper and/or lower respiratory tract RSV infections or otitis media (preferably, stemming from, caused by or associated with a RSV infection, such as an upper and/or lower respiratory tract RSV infection) or a symptom or respiratory condition relating thereto (including, but not limited to, asthma, wheezing, RAD, or a combination thereof) despite treatment with existing therapies.

The present invention encompasses methods for preventing, treating and/or ameliorating a RSV infection (i.e., an upper and/or lower respiratory tract RSV infection), otitis media (preferably, stemming from, caused by or associated with a RSV infection, such as an upper and/or lower respiratory tract RSV infection), or a symptom or respiratory condition relating thereto (including, but not limited to, asthma, wheezing, RAD, or a combination thereof) as an alternative to other conventional therapies. In specific embodiments, the patient being or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, treat, and/or ameliorate an upper and/or lower respiratory tract RSV infection, otitis media (preferably, stemming from, caused by or associated with a RSV infection, such as an upper and/or lower respiratory tract RSV infection) or a symptom or respiratory condition relating thereto (including, but not limited to, asthma, wheezing, RAD, or a combination thereof).

The dosage amounts and frequencies of administration provided herein are encompassed by the terms “effective amount”, “therapeutically effective” and “prophylactically” effective. The dosage and frequency further will typically vary according to factors specific for each patient depending on the specific therapeutic or prophylactic agents administered, the severity and type of infection, the route of administration, as well as age, body weight, response, and the past medical history of the patient. Suitable regimens can be selected by one skilled in the art by considering such factors and by following, for example, dosages reported in the literature and recommended in the Physician's Desk Reference (58th ed., a 2004). See Section 5.3 for specific dosage amounts and frequencies of administration of the prophylactic and therapeutic agents provided by the invention.

### 5.6 Methods of Administration of Antibodies

The a specific embodiment, the invention provides methods of treatment, prophylaxis, and amelioration of an upper and/or lower respiratory tract RSV infection, otitis media (preferably, stemming from, caused by or associated with a RSV infection, such as an upper and/or lower respiratory tract RSV infection) or a symptom or respiratory condition relating thereto (including, but not limited to, asthma, wheezing, RAD, or a combination thereof) by administrating to a subject of an effective amount of antibody, or pharmaceutical composition comprising the formulation comprising an antibody of the invention. In a preferred aspect, an antibody is substantially purified (i.e., substantially free from substances that limit its effect or produce undesired side-effects). The subject administered a therapy is preferably a mammal such as non-primate (e.g., cows, pigs, horses, cats, dogs, rats etc.) and a primate (e.g., monkey such as a cynomolgous monkey and a human). In a preferred embodiment, the subject is a human. In another preferred embodiment, the subject is a human infant or a human infant born prematurely. In another embodiment, the subject is a human with an upper and/or lower respiratory tract RSV infection, otitis media stemming from, caused by or associated with a RSV infection, cystic fibrosis, bronchopulmonary dysplasia, congenital heart disease, congenital immunodeficiency or acquired immunodeficiency, a human who has had a bone marrow transplant, or an elderly human.

Various delivery systems are known and can be used to administer a prophylactic or therapeutic agent (e.g., an antibody of the invention), including, but not limited to, encapsulation in liposomes, microparticles, microcapsules, recombinant cells capable of expressing the antibody, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent (e.g., an antibody of the invention), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antibody of the present invention), or a pharmaceutical composition is administered intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. In a specific embodiment, an antibody, or formulation of the invention is administered using Alkermes AIR™ pulmonary drug delivery technology (Alkermes, Inc., Cambridge, Mass.).

In a specific embodiment, it may be desirable to administer a prophylactic or therapeutic agent, or a pharmaceutical formulation of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering an antibody of the invention, care must be taken to use materials to which the antibody does not absorb.

In another embodiment, a prophylactic or therapeutic agent, or a formulation of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, a prophylactic or therapeutic agent, or a formulation of the invention can be delivered in a controlled release or sustained release system. In one embodiment, a pump may be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of a prophylactic or therapeutic agent (e.g., an antibodies of the invention) or a formulation of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the nasal passages or lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the invention. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment, a formulation of the invention comprises one, two or more antibodies described, infra. In another embodiment, a formulation of the invention comprises one, two or more antibodies described, infra, and a prophylactic or therapeutic agent other than an said antibody. In a specific embodiment, the agents are known to be useful for or have been or are currently used for the prevention, treatment or ameliorating of a RSV infection (preferably, an upper and/or lower respiratory tract RSV infection), otitis media (preferably stemming from, caused by or associated with a RSV infection, such as an upper and/or lower respiratory tract RSV infection) or a symptom or respiratory condition relating thereto (including, but not limited to, asthma, wheezing, RAD, or a combination thereof). In addition to prophylactic or therapeutic agents, the compositions of the invention may also comprise a carrier.

The formulations of the invention include bulk drug compositions useful in the manufacture of pharmaceutical compositions (e.g., compositions that are suitable for administration to a subject or patient) which can be used in the preparation of unit dosage forms. In a preferred embodiment, a composition of the invention is a pharmaceutical composition. Such compositions comprise a prophylactically or therapeutically effective amount of one or more prophylactic or therapeutic agents (e.g., an antibody of the invention or other prophylactic or therapeutic agent), and a pharmaceutically acceptable carrier. Preferably, the pharmaceutical compositions are formulated to be suitable for the route of administration to a subject.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of the antibody, preferably in purified form, together with a suitable amount of carrier so as to provide the form for proper administration to the patient. The formulation should suit the mode of administration.

In a preferred embodiment, the formulations are manufactured in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides that the formulation is packaged in a hermetically sealed container such as an ampoule or sachette indicating the quantity of antibody. In one embodiment, the formulation of the invention comprising an antibody is supplied as a dry sterilized lyophilized powder or water free concentrate in a hermetically sealed container and can be reconstituted, e.g., with water or saline to the appropriate concentration for administration to a subject. In one embodiment, the formulation of the invention comprising an antibody is supplied as a dry sterile lyophilized powder in a hermetically sealed container at a unit dosage of at least 3 mg, more preferably at least 5 mg, at least 10 mg, at least 15 mg, at least 25 mg, at least 30 mg, at least 35 mg, at least 45 mg, at least 50 mg, at least 60 mg, or at least 75 mg. The lyophilized formulation of the invention comprising an antibody should be stored at between 2 and 8° C. in its original container and the antibody should be administered within 12 hours, preferably within 6 hours, within 5 hours, within 3 hours, or within 1 hour after being reconstituted. In an alternative embodiment, a formulation of the invention comprising an antibody is supplied in liquid form in a hermetically sealed container indicating the quantity and concentration of the antibody. Preferably, the liquid form of the formulation of the invention comprising an antibody is supplied in a hermetically sealed container at least 1 mg/ml, more preferably at least 2.5 mg/ml, at least 3 mg/ml, at least 5 mg/ml, at least 8 mg/ml, at least 10 mg/ml, at least 15 mg/ml, at least 25 mg/ml, at least 30 mg/ml, or at least 60 mg/ml.

The formulation of the invention comprising antibodies can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a prophylactic or therapeutic agent (e.g., an antibody of the invention), or a composition of the invention which will be effective in the treatment, prevention or amelioration of an upper and/or lower respiratory tract RSV infection, otitis media (preferably, stemming from, caused by or associated with a RSV infection, such as an upper and/or lower respiratory tract RSV infection) or a symptom or respiratory condition relating thereto (including, but not limited to, asthma, wheezing, RAD, or a combination thereof) can be determined by standard clinical techniques. For example, the dosage of a prophylactic or therapeutic agent, or a composition which will be effective in the treatment, prevention or amelioration of an upper and/or lower respiratory tract RSV infection or one or more symptoms thereof can be determined by administering the composition to a cotton rat, measuring the RSV titer after challenging the cotton rat with 10⁵ pfu of RSV and comparing the RSV titer to that obtain for a cotton rat not administered the prophylactic or therapeutic agent, or the composition. Accordingly, a dosage that results in a 2 log decrease or a 99% reduction in RSV titer in the cotton rat challenged with 10⁵ pfu of RSV relative to the cotton rat challenged with 10⁵ pfu of RSV but not administered the prophylactic or therapeutic agent, or the composition is the dosage of the composition that can be administered to a human for the treatment, prevention or amelioration of an upper and/or lower respiratory tract RSV infection, otitis media (preferably, stemming from, caused by or associated with a RSV infection, such as an upper and/or lower respiratory tract RSV infection) or one or more symptoms thereof.

The dosage of a composition which will be effective in the treatment, prevention or amelioration of an upper and/or lower respiratory tract RSV infection, otitis media (preferably, stemming from, caused by or associated with a RSV infection, such as an upper and/or lower respiratory tract RSV infection) or one or more symptoms thereof can be determined by administering the composition to an animal model (e.g., a cotton rat or monkey) and measuring the serum titer, lung concentration or nasal turbinate and/or nasal secretion concentration of an antibody that immunospecifically bind to a RSV antigen. Accordingly, a dosage of an antibody or a composition that results in a serum titer of at least 1 μg/ml, preferably 2 μg/ml, 5 μg/ml, 10 μg/ml, 15 μg/ml, 20 μg/ml, 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the treatment, prevention or amelioration of an upper and/or lower respiratory tract RSV infection, otitis media (preferably, stemming from, caused by or associated with a RSV infection, such as an upper and/or lower respiratory tract RSV infection) or one or more symptoms thereof. In addition, in vitro assays may optionally be employed to help identify optimal dosage ranges.

The precise dose to be employed in the formulation will also depend on the route of administration, and the seriousness of the upper and/or lower respiratory tract RSV infection or otitis media, and should be decided according to the judgment of the practitioner and each patient's circumstances. Effective doses may be extrapolated from dose-response curves derived from in vitro or animal model (e.g., the cotton rat or Cynomolgous monkey) test systems.

For antibodies, the dosage administered to a patient is typically 0.1 mg/kg to 100 mg/kg of the patient's body weight. In some embodiments, the dosage administered to the patient is about 3 mg/kg to about 60 mg/kg of the patient's body weight. Preferably, the dosage administered to a patient is between 0.1 mg/kg and 20 mg/kg of the patient's body weight, more preferably 1 mg/kg to 15 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the nasal passages and/or lung) of the antibodies by modifications such as, for example, lipidation. In a preferred embodiment, the dosage of A4B4L1FR-S28R (motavizumab) or antigen-binding fragment thereof to be administered to is 60 mg/kg, 50 mg/kg, 40 mg/kg, 30 mg/kg, 15 mg/kg, 10 mg/kg, 5 mg/kg, 3 mg/kg, or 2 mg/kg of the patient's body weight.

In a specific embodiment, formulations of the invention comprising antibodies or compositions comprising antibodies are administered once a month just prior to or during the RSV season. In another embodiment, formulation of the invention comprising an antibody, or compositions comprising antibodies produced in accordance with the methods of the invention are administered every two months just prior to or during the RSV season. In yet another embodiment, antibodies, or compositions comprising antibodies are administered once just prior to or during the RSV season. The term “RSV season” refers to the season when RSV infection is most likely to occur. Typically, the RSV season in the northern hemisphere commences in November and lasts through April. Preferably, the antibody comprises the VH and VL domain of A4B4L1FR-S28R (motavizumab) (FIG. 13) or an antigen-binding fragment thereof.

In one embodiment, approximately 60 mg/kg or less, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, or approximately 1.5 mg/kg or less of an antibody the invention is administered 5 times, 4 times, 3 times, 2 times or 1 time during a RSV season to a subject, preferably a human. In some embodiments, the antibody is administered about 1-12 times during the RSV season to a subject, wherein the doses may be administered as necessary, e.g., weekly, biweekly, monthly, bimonthly, trimonthly, etc., as determined by a physician. In some embodiments, a lower dose (e.g., 5-15 mg/kg) can be administered more frequently (e.g., 3-6 times) during a RSV season. In other embodiments, a higher dose (e.g., 30-60 mg/kg) can be administered less frequently (e.g., 1-3 times) during a RSV season. However, as will be apparent to those in the art, other dosing amounts and schedules are easily determinable and within the scope of the invention.

In one embodiment, approximately 60 mg/kg or less, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, or approximately 1.5 mg/kg or less of an antibody is administered to monthly five times during a RSV season to a subject, preferably a human, intramuscularly. In another embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, or approximately 1.5 mg/kg or less of an antibody the invention is administered monthly three times during a RSV season to a subject, preferably a human, intramuscularly. In yet another embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, or approximately 1.5 mg/kg or less of an antibody is administered monthly one to two times during a RSV season to a subject, preferably a human, intramuscularly. Preferably, the antibody comprises the VH and VL domain of A4B4L1FR-S28R (motavizumab) (FIG. 13) or an antigen-binding fragment thereof.

In a specific embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, or approximately 1.5 mg/kg or less of an antibody in a sustained release formulation is administered to a subject, preferably a human, to prevent, treat or ameliorate an upper and/or lower respiratory tract RSV infection, otitis media (preferably, stemming from, caused by or associated with a RSV infection, such as an upper and/or lower respiratory tract RSV infection) or one or more symptoms thereof. In another specific embodiment, an approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, or approximately 1.5 mg/kg or less bolus of an antibody the invention not in a sustained release formulation is administered to a subject, preferably a human, to prevent, treat or ameliorate an upper and/or lower respiratory tract RSV infection, otitis media (preferably, stemming from, caused by or associated with a RSV infection, such as an upper and/or lower respiratory tract RSV infection) or one or more symptoms thereof and after a certain period of time approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, or approximately 1.5 mg/kg or less of the invention in a sustained release is administered to said subject intramuscularly two, three or four times during a RSV season. In accordance with this embodiment, a certain period of time can be 1 to 5 days, a week, two weeks, or a month. In another embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, or approximately 1.5 mg/kg or less of an antibody in a sustained release formulation is administered to a subject, preferably a human, intramuscularly two, three or four times during a RSV season to prevent, treat or ameliorate an upper and/or lower respiratory tract RSV infection, otitis media (preferably, stemming from, caused by or associated with a RSV infection, such as an upper and/or lower respiratory tract RSV infection) or one or more symptoms thereof. Preferably, the antibody is A4B4L1FR-S28 or an antigen-binding fragment thereof.

In another embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, or approximately 1.5 mg/kg or less of one or more antibodies of the invention is administered intranasally to a subject to prevent, treat or ameliorate an upper and/or lower respiratory tract RSV infection, otitis media (preferably, stemming from, caused by or associated with a RSV infection, such as an upper and/or lower respiratory tract RSV infection) or one or more symptoms thereof. Preferably, the antibody is A4B4L1FR-S28 or an antigen-binding fragment thereof. Preferably, the antibody is A4B4L1FR-S28 or an antigen-binding fragment thereof.

In one embodiment, a single dose of the formulation of the invention comprising an antibody (preferably motavizumab) is administered to a patient (preferably a human), wherein the dose is selected from the group consisting of about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, or about 75 mg/kg.

In some embodiments, a single dose of a formulation of the invention comprising an antibody (preferably motavizumab) is administered to a patient (preferably a human) two, three, four, five, six, seven, eight, nine, ten, eleven, twelve times, thirteen, fourteen, fifteen, sixteen, seventeen, eighteen, nineteen, twenty, twenty-one, twenty-two, twenty-three, twenty-four, twenty five, or twenty six at bi-weekly (e.g., about 14 day) intervals over the course of a year (or alternatively over the course of a RSV season), wherein the dose is selected from the group consisting of about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or a combination thereof (i.e., each dose monthly dose may or may not be identical).

In another embodiment, a single dose of a formulation of the invention comprising an antibody (preferably motavizumab) is administered to patient (preferably a human) two, three, four, five, six, seven, eight, nine, ten, eleven, or twelve times at about monthly (e.g., about 30 day) intervals over the course of a year (or alternatively over the course of a RSV season), wherein the dose is selected from the group consisting of about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or a combination thereof (i.e., each dose monthly dose may or may not be identical).

In one embodiment, a single dose of a formulation of the invention comprising an antibody (preferably motavizumab) is administered to a patient (preferably a human) two, three, four, five, or six times at about bi-monthly (e.g., about 60 day) intervals over the course of a year (or alternatively over the course of a RSV season), wherein the dose is selected from the group consisting of about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or a combination thereof (i.e., each bi-monthly dose may or may not be identical).

In some embodiments, a single dose of a formulation of the invention comprising an antibody (preferably motavizumab) is administered to a patient (preferably a human) two, three, or four times at about tri-monthly (e.g., about 120 day) intervals over the course of a year (or alternatively over the course of a RSV season), wherein the dose is selected from the group consisting of about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or a combination thereof (i.e., each tri-monthly dose may or may not be identical).

In certain embodiments, the route of administration for a dose of a formulation of the invention comprising an antibody to a patient is intramuscular, intravenous, or a combination thereof (i.e., each dose may or may not be administered by an identical route of administration). In some embodiments, an antibody of the invention may be administered via multiple routes of administration simultaneously or subsequently to other doses of the same or a different antibody of the invention.

### 5.7 Biological Activity

Formulations of the invention comprising antibodies may be characterized in a variety of ways. In particular, antibodies may be assayed for the ability to immunospecifically bind to a RSV antigen. Such an assay may be performed in solution (e.g., Houghten, 1992, Bio/Techniques 13:412-421), on beads (Lam, 1991, Nature 354:82-84), on chips (Fodor, 1993, Nature 364:555-556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310) (each of these references is incorporated herein in its entirety by reference). Antibodies that have been identified to immunospecifically bind to a RSV antigen (e.g., a RSV F antigen) can then be assayed for their specificity and affinity for a RSV antigen.

Formulations of the invention comprising antibodies may be assayed for immunospecific binding to a RSV antigen and cross-reactivity with other antigens by any method known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, incubating the membrane in blocking solution (e.g., PBS with 3% BSA or non-fat milk), washing the membrane in washing buffer (e.g., PBS-Tween 20), incubating the membrane with primary antibody (the antibody of interest) diluted in blocking buffer, washing the membrane in washing buffer, incubating the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of the present invention for a RSV antigen and the binding off-rates can be determined from the data by scatchard plot analysis. Competition with a second antibody can also be determined using radioimmunoassays. In this case, a RSV antigen is incubated with an antibody of the present invention conjugated to a labeled compound (e.g., ³H or ¹²⁵I) in the presence of increasing amounts of an unlabeled second antibody.

In a preferred embodiment, BIAcore kinetic analysis is used to determine the binding on and off rates of antibodies to a RSV antigen. BIAcore kinetic analysis comprises analyzing the binding and dissociation of a RSV antigen from chips with immobilized antibodies on their surface.

Formulations of the invention comprising antibodies can also be assayed for their ability to inhibit the binding of RSV to its host cell receptor using techniques known to those of skill in the art. For example, cells expressing the receptor for RSV can be contacted with RSV in the presence or absence of an antibody and the ability of the antibody to inhibit RSV's binding can measured by, for example, flow cytometry or a scintillation assay. RSV (e.g., a RSV antigen such as F glycoprotein or G glycoprotein) or the antibody can be labeled with a detectable compound such as a radioactive label (e.g., 32P, 35S, and 125I) or a fluorescent label (e.g., fluorescein isothiocyanate, rhodamine, phycoerythrin, phycocyanin, allophycocyanin, o-phthaldehyde and fluorescamine) to enable detection of an interaction between RSV and its host cell receptor. Alternatively, the ability of antibodies to inhibit RSV from binding to its receptor can be determined in cell-free assays. For example, RSV or a RSV antigen such as G glycoprotein can be contacted with an antibody and the ability of the antibody to inhibit RSV or the RSV antigen from binding to its host cell receptor can be determined. Preferably, the antibody is immobilized on a solid support and RSV or a RSV antigen is labeled with a detectable compound. Alternatively, RSV or a RSV antigen is immobilized on a solid support and the antibody is labeled with a detectable compound. RSV or a RSV antigen may be partially or completely purified (e.g., partially or completely free of other polypeptides) or part of a cell lysate. Further, a RSV antigen may be a fusion protein comprising the RSV antigen and a domain such as glutathionine S transferase. Alternatively, a RSV antigen can be biotinylated using techniques well known to those of skill in the art (e.g., biotinylation kit, Pierce Chemicals; Rockford, Ill.).

Formulations of the invention comprising antibodies can also be assayed for their ability to inhibit or downregulate RSV replication using techniques known to those of skill in the art. For example, RSV replication can be assayed by a plaque assay such as described, e.g., by Johnson et al., 1997, Journal of Infectious Diseases 176:1215-1224. The antibodies of the invention can also be assayed for their ability to inhibit or downregulate the expression of RSV polypeptides. Techniques known to those of skill in the art, including, but not limited to, Western blot analysis, Northern blot analysis, and RT-PCR can be used to measure the expression of RSV polypeptides. Further, the antibodies of the invention can be assayed for their ability to prevent the formation of syncytia.

Formulations of the invention comprising antibodies are preferably tested in vitro, and then in vivo for the desired therapeutic or prophylactic activity, prior to use in humans. For example, in vitro assays which can be used to determine whether administration of a specific antibody or composition of the present invention is indicated, include in vitro cell culture assays in which a subject tissue sample is grown in culture, and exposed to or otherwise administered an antibody or composition of the present invention, and the effect of such an antibody or composition of the present invention upon the tissue sample is observed. In various specific embodiments, in vitro assays can be carried out with representative cells of cell types involved in a RSV infection (e.g., respiratory epithelial cells), to determine if an antibody or composition of the present invention has a desired effect upon such cell types. Preferably, the antibodies or compositions of the invention are also tested in in vitro assays and animal model systems prior to administration to humans. In a specific embodiment, cotton rats are administered an antibody the invention, or a composition of the invention, challenged with 10⁵ pfu of RSV, and four or more days later the rats are sacrificed and RSV titer and anti-RSV antibody serum titer is determined. Further, in accordance with this embodiment, the tissues (e.g., the lung tissues) from the sacrificed rats can be examined for histological changes.

In accordance with the invention, clinical trials with human subjects need not be performed in order to demonstrate the prophylactic and/or therapeutic efficacy of antibodies of the invention. In vitro and animal model studies using the antibodies can be extrapolated to humans and are sufficient for demonstrating the prophylactic and/or therapeutic utility of said antibodies.

Formulations of the invention comprising antibodies or compositions of the present invention for use in therapy can be tested for their toxicity in suitable animal model systems, including but not limited to rats, mice, cows, monkeys, and rabbits. For in vivo testing of an antibody or composition's toxicity any animal model system known in the art may be used.

Efficacy in treating or preventing an upper and/or lower respiratory tract RSV infection may be demonstrated by determining the ability of an antibody or composition of the invention to inhibit the replication of the virus, to inhibit transmission or prevent the virus from establishing itself in its host, to reduce the incidence of an upper and/or lower respiratory tract RSV infection, to prevent or reduce the progression of an upper respiratory tract RSV infection to a lower respiratory tract RSV infection, or to prevent, ameliorate or alleviate one or more symptoms associated with an upper and/or lower respiratory tract RSV infection. Efficacy in treating or preventing otitis media may be demonstrated by determining the ability of an antibody or composition of the invention to reduce the incidence or otitis media, to reduce the duration of otitis media, to prevent or reduce the progression of an upper and/or lower respiratory tract RSV infection to otitis media, or to ameliorate one or more symptoms of otitis media. A therapy is considered therapeutic if there is, for example, a reduction is viral load, amelioration of one or more symptoms of an upper and/or lower respiratory tract RSV infection or otitis media, or a respiratory condition relating thereto (including, but not limited to asthma, wheezing, RAD or a combination thereof), a reduction in the duration of an upper and/or lower respiratory tract RSV infection or otitis media, a reduction in lower respiratory tract RSV infections, or a decrease in mortality and/or morbidity following administration of an antibody or composition of the invention. Further, the treatment is considered therapeutic if there is an increase in the immune response following the administration of one or more antibodies which immunospecifically bind to one or more RSV antigens.

Formulations of the invention comprising antibodies or compositions of the invention can be tested in vitro and in vivo for the ability to induce the expression of cytokines such as IFN-α, IFN-β, IFN-γ, IL-⁻², IL-3, IL-4, IL-5, IL-6, IL-7, IL-8, IL-9, IL-10, IL-12 and IL-15. Techniques known to those of skill in the art can be used to measure the level of expression of cytokines. For example, the level of expression of cytokines can be measured by analyzing the level of RNA of cytokines by, for example, RT-PCR and Northern blot analysis, and by analyzing the level of cytokines by, for example, immunoprecipitation followed by western blot analysis and ELISA. In a preferred embodiment, an antibody or composition of the invention is tested for its ability to induce the expression of IFN-γ.

Formulations of the invention comprising antibodies or compositions of the invention can be tested in vitro and in vivo for their ability to modulate the biological activity of immune cells, preferably human immune cells (e.g., T-cells, B-cells, and Natural Killer cells). The ability of an antibody or composition of the invention to modulate the biological activity of immune cells can be assessed by detecting the expression of antigens, detecting the proliferation of immune cells, detecting the activation of signaling molecules, detecting the effector function of immune cells, or detecting the differentiation of immune cells. Techniques known to those of skill in the art can be used for measuring these activities. For example, cellular proliferation can be assayed by ³H thymidine incorporation assays and trypan blue cell counts. Antigen expression can be assayed, for example, by immunoassays including, but are not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and FACS analysis. The activation of signaling molecules can be assayed, for example, by kinase assays and electrophoretic shift assays (EMSAs).

Formulations of the invention comprising antibodies or compositions of the invention can also be tested for their ability to inhibit viral replication or reduce viral load in in vitro, ex vivo and in vivo assays. Antibodies or compositions of the invention can also be tested for their ability to decrease the time course of a RSV infection (i.e., an upper and/or lower respiratory tract RSV infection), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof). Antibodies or compositions of the invention can also be tested for their ability to increase the survival period of humans suffering from a RSV infection (preferably, an upper and/or lower respiratory tract RSV infection) by at least 25%, preferably at least 50%, at least 60%, at least 75%, at least 85%, at least 95%, or at least 99%. Further, antibodies or compositions of the invention can be tested for their ability reduce the hospitalization period of humans suffering from a RSV infection (preferably, an upper and/or lower respiratory tract RSV infection) by at least 60%, preferably at least 75%, at least 85%, at least 95%, or at least 99%. Techniques known to those of skill in the art can be used to analyze the function of the antibodies or compositions of the invention in vivo.

### 5.8 Diagnostic Uses of Antibodies for Detecting RSV Infections

Labeled antibodies and derivatives and analogs thereof, which immunospecifically bind to a RSV antigen can be used for diagnostic purposes to detect, diagnose, or monitor an upper and/or lower respiratory tract RSV infection or otitis media (preferably, stemming from, caused by or associated with a RSV infection, such as an upper and/or lower respiratory tract RSV infection). The invention provides for the detection of a RSV infection (i.e., an upper and/or lower respiratory tract RSV infection), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof) comprising: (a) assaying the expression of a RSV antigen in cells or a tissue sample of a subject using one or more antibodies that immunospecifically bind to the RSV antigen; and (b) comparing the level of the RSV antigen with a control level, e.g., levels in normal tissue samples not infected with RSV, whereby an increase in the assayed level of RSV antigen compared to the control level of the RSV antigen is indicative of a RSV infection (i.e., an upper and/or lower respiratory tract RSV infection), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof).

The invention provides a diagnostic assay for diagnosing a RSV infection (i.e., an upper and/or lower respiratory tract RSV infection), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof) comprising: (a) assaying for the level of a RSV antigen in cells or a tissue sample of an individual using one or more antibodies that immunospecifically bind to a RSV antigen; and (b) comparing the level of the RSV antigen with a control level, e.g., levels in normal tissue samples not infected with RSV, whereby an increase in the assayed RSV antigen level compared to the control level of the RSV antigen is indicative of a RSV infection (i.e., an upper and/or lower respiratory tract RSV infection), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof). A more definitive diagnosis of a RSV infection (i.e., an upper and/or lower respiratory tract RSV infection), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof) may allow health professionals to employ preventative measures or aggressive treatment earlier thereby preventing the development or further progression of the RSV infection or otitis media.

Antibodies of the invention can be used to assay RSV antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin.

One aspect of the invention is the detection and diagnosis of a RSV infection (i.e., an upper and/or lower respiratory tract RSV infection), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof) in a human. In one embodiment, diagnosis comprises: a) administering (for example, parenterally, subcutaneously, or intraperitoneally) to a subject an effective amount of a labeled antibody that immunospecifically binds to a RSV antigen; b) waiting for a time interval following the administering for permitting the labeled antibody to preferentially concentrate at sites in the subject (e.g., the nasal passages, lungs, mouth and ears) where the RSV antigen is expressed (and for unbound labeled molecule to be cleared to background level); c) determining background level; and d) detecting the labeled antibody in the subject, such that detection of labeled antibody above the background level indicates that the subject has a RSV infection (i.e., an upper and/or lower respiratory tract RSV infection), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof). Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled antibody to preferentially concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

In one embodiment, monitoring of an upper and/or lower respiratory tract RSV infection is carried out by repeating the method for diagnosing the upper and/or lower respiratory tract RSV infection, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### 5.9 Kits

The invention also provides a pharmaceutical pack or kit comprising one or more containers filled with one or more of the ingredients of the pharmaceutical formulation of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises an antibody of the invention, preferably a purified antibody, in one or more containers. In a specific embodiment, the kits of the present invention contain a substantially isolated RSV antigen as a control. Preferably, the kits of the present invention further comprise a control antibody which does not react with the RSV antigen. In another specific embodiment, the kits of the present invention contain a means for detecting the binding of an antibody to a RSV antigen (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate). In specific embodiments, the kit may include a recombinantly produced or chemically synthesized RSV antigen. The RSV antigen provided in the kit may also be attached to a solid support. In a more specific embodiment the detecting means of the above described kit includes a solid support to which RSV antigen is attached. Such a kit may also include a non-attached reporter-labeled anti-human antibody. In this embodiment, binding of the antibody to the RSV antigen can be detected by binding of the said reporter-labeled antibody.

### 6. EXAMPLES

The following examples are offered to illustrate this invention and not to be construed in any way as limiting the scope of this invention.

### 6.1 Example

### Characterization of Antibody Formulation for Antibody Fragmentation and Aggregation

This example illustrates the characterization of an antibody formulation for antibody fragmentation and aggregation. Antibody A4B4L1FR-S28R is used in this example. As discussed in the sections above antibody A4B4L1FR-S28R is an IgG1 monoclonal antibody produced by recombinant DNA technology that specifically binds to an epitope in the A antigenic site of the fusion (F) protein of RSV. A4B4L1FR-S28R is a humanized antibody and consists of the CDR regions specific for the targeted antigen and the constant regions of a human γ1 heavy chain and κ light chain. The monoclonal antibody has two inter-chain disulfide bonds to link heavy and light chains, and another two inter-chain disulfide bonds at the hinge region. Unless otherwise indicated, all antibody samples in this example were formulated at a concentration of 100 mg/ml in 25 mM histidine-HCl, pH 6.0. Further storage conditions are reported in the section describing experimental results.

### Materials and Methods

### Size Exclusion Chromotography (SEC)

Size exclusion chromatography was performed to analyze the antibody formulation for the presence of antibody aggregates and fragments. The test samples were injected onto a size exclusion G3000 SW_{XL} column (5 μm, 300 Å, 7.8×300 mm, TosoHaas). The mobile phase was 0.1 M di-sodium phosphate, 0.1 M sodium sulphate and 0.05% sodium azide (pH 6.7), running isocratically at a flow rate of 0.25-1.0 mL/min. Eluted protein was detected by UV absorbance at 280 nm and collected for further characterization. The relative amount of any protein species detected was reported as the area percent of the product peak as compared to the total area of all other detected peaks excluding the initial included volume peak. Peaks eluting earlier than the antibody monomer peak were recorded in the aggregate percentile, while peaks eluting later than the antibody monomer peak, but earlier than the buffer peak, were recorded in the fragment percentile. The hydrodynamic radius and molecular weight of the individual peaks were obtained with a coupled multiangle light scattering detector.

### Analytical Ultracentrifugation (AUC)

Analytical ultracentrifugation (AUC) was also used to characterize the antibody formulation. AUC is an orthogonal technique which determines the sedimentation coefficients (reported in Svedberg, S) of macromolecules in a liquid sample. Like SEC, AUC is capable of separating and detecting antibody fragments/aggregates from monomers and is further able to provide information on molecular mass. Compared to SEC, AUC eliminates the possibility of aggregate loss due to solid-phase interaction and is better able to resolve differing species of a given macromolecule.

Sedimentation velocity experiments were performed using a Beckman Optima™ XL-A analytical ultracentrifuge. Test samples were diluted to an antibody concentration of 0.5 mg/ml with reference buffer (20 mM citric acid, 100 mM NaCl, 1.5% mannitol, 50 μM diethylenetriamine-pentaacetic acid, 0.02% Polysorbate 80, pH 6.0). 415 μl of the diluted antibody sample and 412 μl or the reference buffer were loaded into a 12 mm centrifuge cell in the sample and reference channels, respectively. Loaded cells were placed into an AN-50Ti analytical rotor and equilibrated to 25 oC. Samples were scanned at 280 nm with a rotor speed of 42000 rpm at full vacuum. A total of 80 scans for each cell were collected for analysis. The first scan for each sample was excluded to avoid artifacts caused by meniscus.

The data were analyzed using the c(s) method developed by Peter Shuck at N.I.H. and the SEDFIT (version 8.8) program with implemented c(s). Using the c(s) method, raw data scans are directly fit to a Lamm function of S in order to derive a distribution of sedimentation coefficients. The parameters used for the fitting procedure were resolution, 400; confidence interval, 0.75; grid size, 1000; partial specific volume, 0.7245; buffer density, 1.000; and buffer viscosity, 0.1002. Frictional ratio, meniscus and bottom positions were set as fitted parameters. Time independent noise was also fitted. The detected peaks were integrated and classified as follows: from 0 to 6 S, fragments; from 6 to 9 S, monomer; and from 9 to 20 S, aggregates.

### Turbidity Measurement

Protein aggregation in the antibody formulation was also characterized by turbidity measurement. Turbidity is a measure of the amount by which the particles in a solution scatter light and, thus, may be used as a general indicator of protein aggregation or denaturation.

Approximately 3 to 4 ml of formulation sample was transferred into a glass test tube and degassed for 2 minutes using an in-line vacuum system. The degassed sample was then placed into a turbidimeter (2100AN or 2100N, Hatch) sample compartment at room temperature for analysis. The turbidimeter was calibrated with STABLCAL® Stabilized Formazin Turbidity standard (Hatch) at 40, 200, 1000 and 4000 NTU (nephelometric turbidity unit) and verified by analyzing control suspensions of formazin at 3, 6, 18, 30 and 60 NTU.

### Results

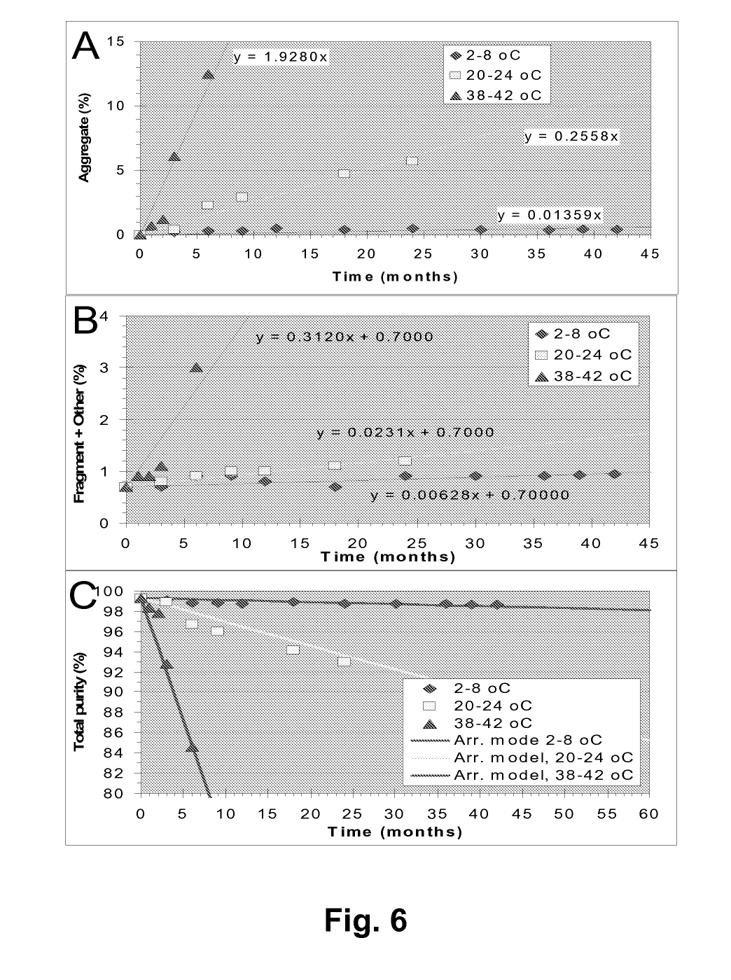

SEC was used to monitor antibody aggregate and fragment formation in formulations of A4B4L1FR-S28R stored at three temperature ranges over the course of 9 months. Temperature ranges above the proposed storage temperature, 2-8° C., were used to stress the formulation and were hoped to simulate the effects of prolonged storage. FIGS. 6 A, B and C present the relative percentage of monomer (purity), aggregates and fragments, respectively, for a single formulation of motavizumab stored at 2-8° C., 20-24° C. and 38-42° C. The relative percentage of fragmentation and aggregation increased with both time and temperature. For a single temperature range, however, both the fragmentation and aggregation rate were constant. This finding proved that a higher storage temperature would accurately simulate an accelerated time scale.

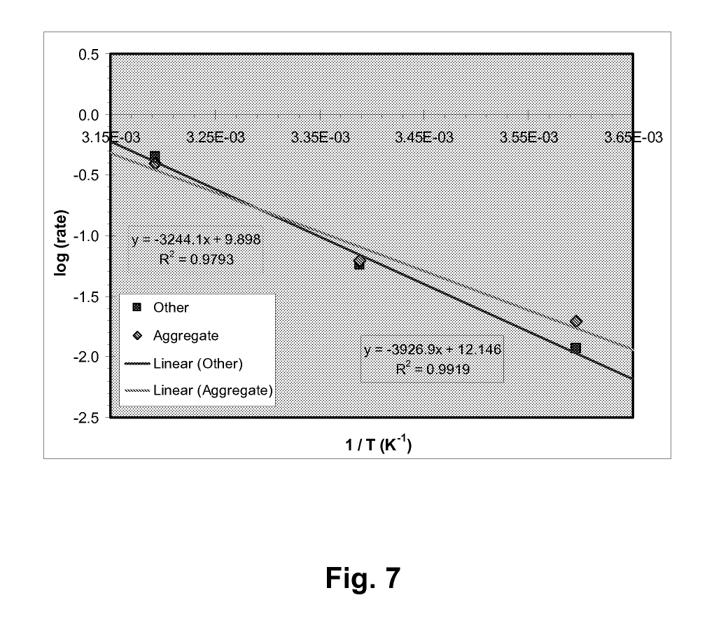

The logarithm of the estimated rates of fragmentation/aggregation also showed a linear dependence to the reciprocal of the storage temperature (FIG. 7). Once this linearity is established, it is then possible to predict the rate of aggregation/fragmentation of a given formulation at any temperature or, more importantly, the formulation characteristics at any time at such temperature.

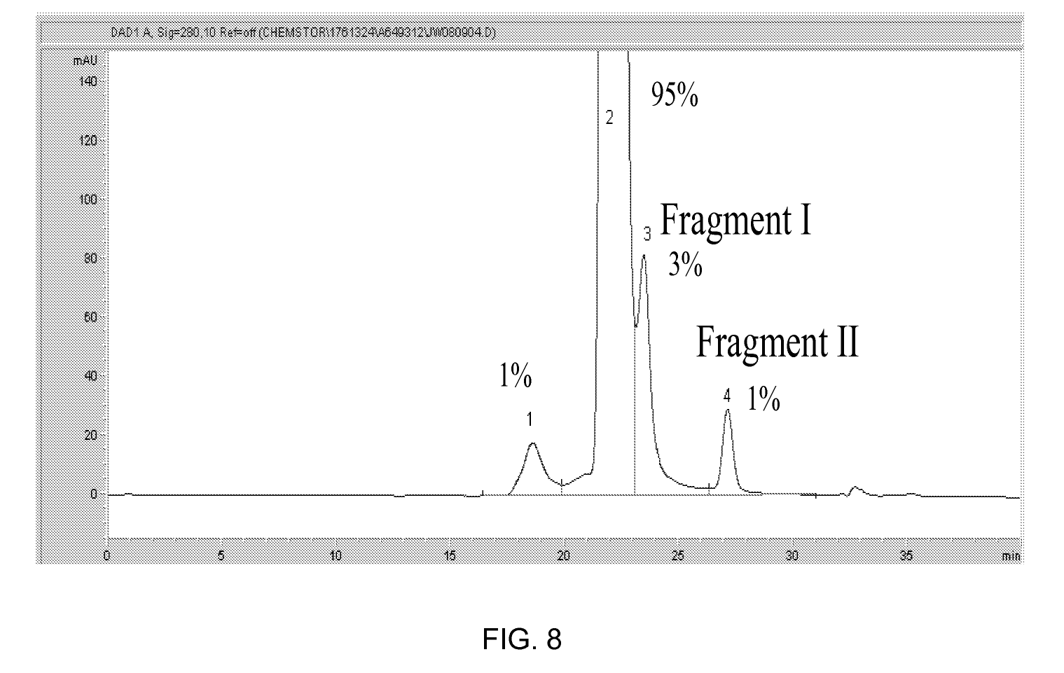

FIG. 8 presents a representative SEC profile of the antibody formulation after storage at 38-42° C. with 70-80% relative humidity for 1 month. Under these conditions, SEC was able to clearly separate antibody aggregates and fragments from monomers. However, at low relative levels of aggregates/fragments, the peaks identified as aggregates and fragment I in FIG. 8 begin to become less distinct and merge into the shoulders of the monomer peak. Such shoulders cannot be accurately analyzed.

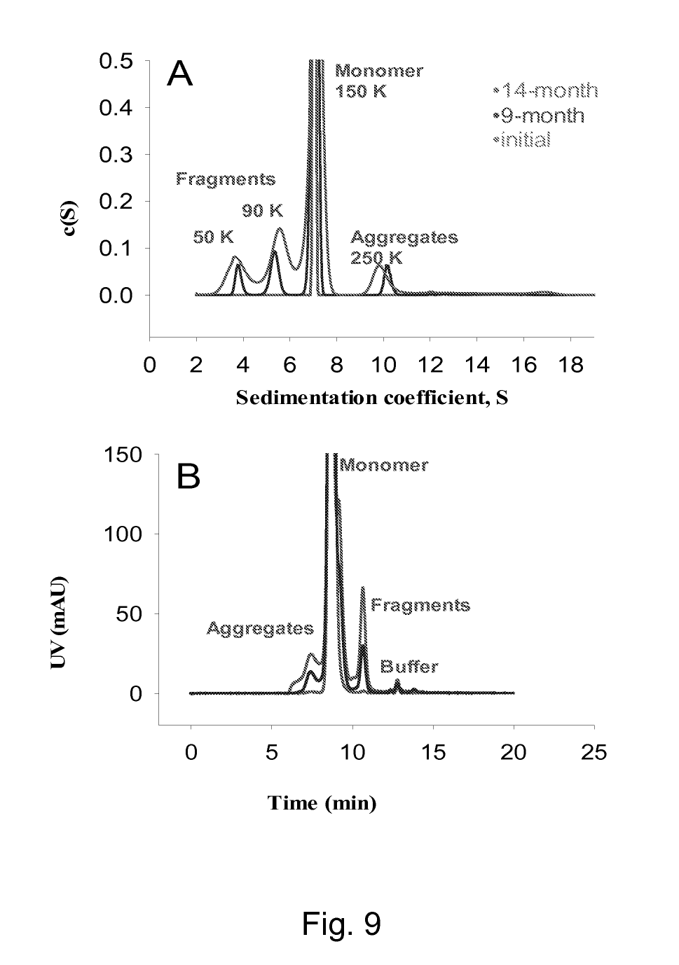

As an alternative, AUC was investigated as a method to characterize low relative levels of aggregation and fragmentation in antibody formulations. FIG. 9 and Table 7 compare the AUC and SEC analysis of formulation samples at initial, 9 month and 14 month time points (the 9 and 14 month samples had been stored at 38-42° C. with 70-80% relative humidity). AUC identified two major fragmentation peaks at about 50 KDa and about 90 KDa. AUC was also able to better resolve the fragmentation and aggregation peaks. For the 9 month sample, SEC did not resolve the large fragment peak, while AUC was clearly capable of resolving it. For the 14 month sample, the large fragment peak in SEC was observed as a shoulder of the monomer peak and, when integrated, resulted in a higher fragment I percent than that determined by AUC. Aggregate values for AUC and SEC were comparable. AUC estimates of the molecular mass of the aggregate peak indicated that the majority of the aggregates were antibody dimers.

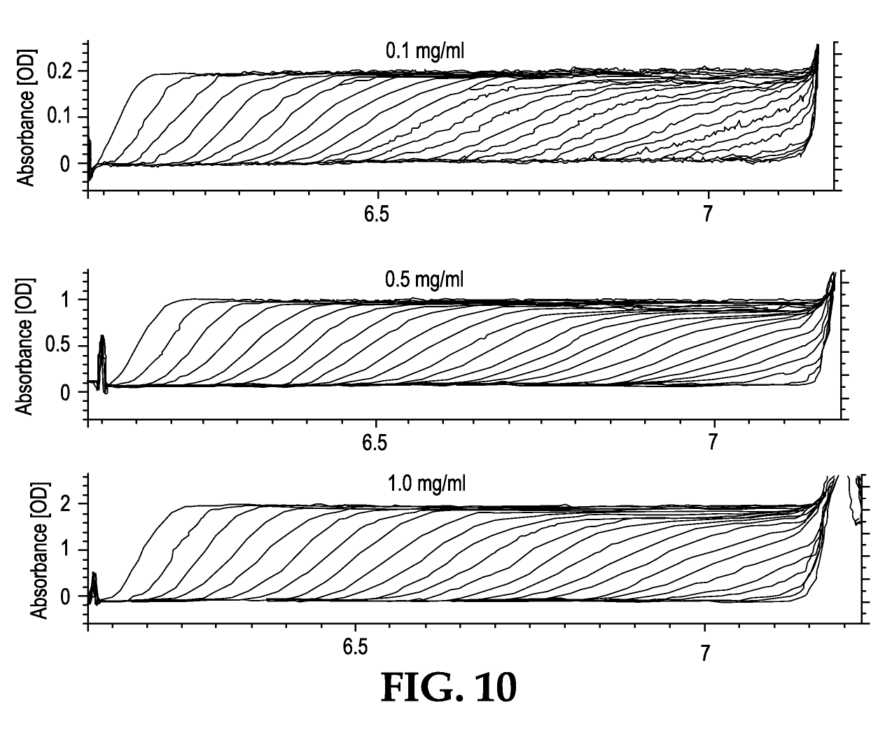

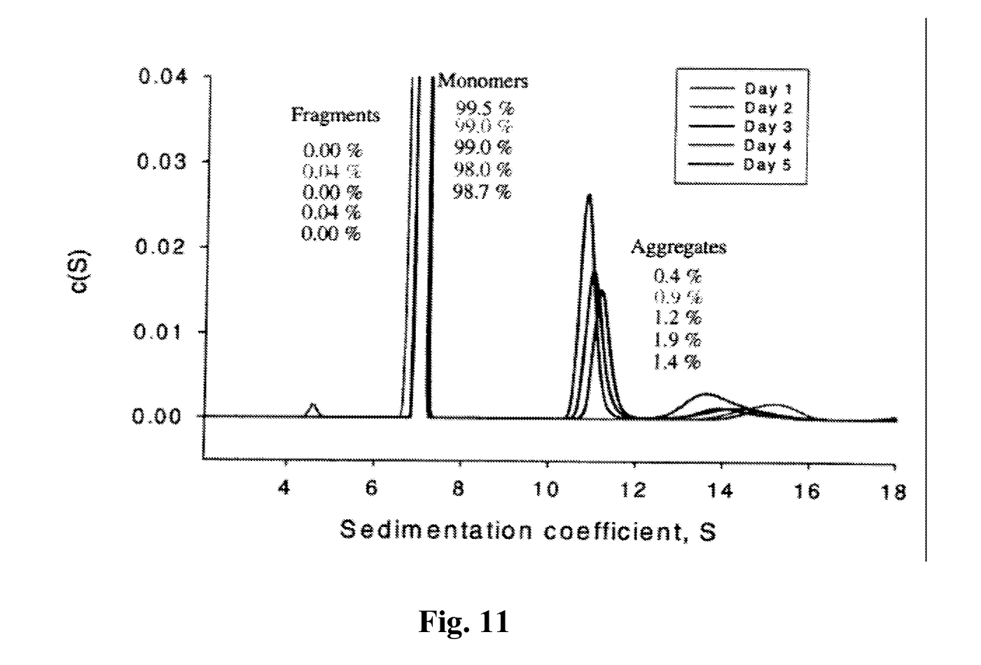

Compared to SEC, AUC is also able to better resolve differing species of a given macromolecule. It is, however, first necessary to establish the proper sample dilution, as the noise/signal ratio of AUC is dependent on the concentration of antibody in the sample (FIG. 10). For the described formulation of A4B4L1FR-S28R (100 mg/ml in 25 mM histidine-HCl, pH 6.0), a 200 fold dilution was used—resulting in a sample antibody concentration of 0.5 mg/ml. Under these conditions, AUC was able to resolve the slight changes in formulation composition observed over 5 days of storage at 38-42° C. with 70-80% relative humidity (FIG. 11).

TABLE 7
COMPARISON OF AUC AND SEC ANALYSIS OF motavizumab
FORMULATIONS AT INITIAL, 9-MONTH AND 14-MONTH TIME
POINTS

| | AUC | | | SEC | | |
|---|---|---|---|---|---|---|
| | Fragments | Monomer | Aggregates | Fragments | Monomer | Aggregates |
| Samples | % | % | % | % | % | % |
| Initial | 0.0 | 99.2 | 0.8 | 0.0 | 99.5 | 0.5 |
| 9-month | 7.5 | 89.3 | 3.2 | 3.3 | 93.7 | 3.0 |
| 14-month | 24.5 | 64.7 | 10.8 | 28.8 | 60.5 | 9.8 |

As a general indicator of protein aggregation, the antibody formulation may also be monitored for changes in turbidity. Four lots of a formulation containing concentrations of antibody at about 100 mg/ml were measured for turbidity using a HACH turbidimeter after storage at 38-42° C. for one month (Table 8). The results indicate that the turbidity levels of the differing lots of the formulation had comparable turbidity measurements, comparable NTU, but that one lot showed an elevated measurement. Elevated turbidity may indicate a higher level of aggregation or an increased number/increased size of particles.

TABLE 8
TURBIDITY VALUES OF FOUR LOTS OF A motavizumab
FORMULATION

| | | Concentration | Turbidity Value |
|---|---|---|---|
| MAb | Lot | (mg/ml) | (NTU) |
| A4B4L1FR-S28R | A | 100 | 5.8 |
| | B | 100 | 7.1 |
| | C | 100 | 6.1 |
| | D | 100 | 5.6 |
| | E | 100 | 5.7 |

### 6.2 Example

### Characterization of Antibody Fragments and Formulation Particle Size Distribution

This example illustrates the characterization of antibody fragments as identified by AUC or SEC. Antibody A4B4L1FR-S28R is used in this example. Unless otherwise indicated, all antibody samples in this example were formulated at a concentration of 100 mg/ml in 25 mM histidine-HCl, pH 6.0. Further storage conditions are reported in the section describing experimental results.

### Materials and Methods

### Liquid Chromatography Mass Spectrometry (LC-MS)

The SEC fragment peaks were collected and digested with N-Glycosidase F, also known as PNGase F, at 37° C. overnight. PNGase F is an amidase that cleaves between the innermost GlcNAc and asparagine residues of high mannose, hybrid and complex oligosaccharides on N-linked glycoproteins. The deglycosylated sample (approximately 7.5 μL) was mixed with approximately 42.5 μL of reducing buffer (2.5 mg/mL DTT, 6.0 M guanindine HCl, pH 8.2) and kept at 56° C. in a water bath for 60 minutes. Neat 4-vinylpyridine (Aldrich Chem. Co., WI) (approximately 0.5 μL) was then added to the sample, and the reaction mixture was held at ambient temperature for 30 minutes. The deglycosylated, reduced and alkylated sample was immediately loaded onto a reversed phase column to separate the modified samples from the reactants, and to analyzed by LC-ESI-MS.

Deglycosylated, reduced, and alkylated samples were fractionated using a reversed phase column (Jupiter 5 μm C4, 300 Å, 250×2.00 mm, Phenomenex®) with a binary gradient HPLC system (Agilent® 1100). Mobile phase A consisted of 30% acetonitrile in water with 0.1% trifluoroacetic acid and mobile phase B consisted of 50% acetonitrile in water with 0.1% trifluoroacetic acid. The samples were separated using a linear gradient of 30-50% acetonitrile in water, over 16 min. with a flow rate of 200 μL/min. The column effluent was directed to a UV detector and then split 1:1, one half going to a switching valve on the Ion Trap mass spectrometer (LTQ, ThermoElectro, San Jose, Calif.), and the remaining half to waste. The switching valve diverted the column effluent flow to the mass spectrometer only between the 15 and 30 minutes portion of the chromatographic run.

A mixture of caffeine, L-methionyl-arginyl-phenylalanyl-alanine acetate H₂O, and Ultramark® 1621 was used to calibrate the ion-trap mass spectrometer according to the manufacturer's instruction. The ESI-MS data were acquired in positive ESI full scan mode. The BioWork deconvolution program (ThermoFinnigan) was used to reconstruct the mass spectra and obtain the molecular masses of the peptides/proteins from their original mass spectra.

### Disulfide Bond Determination

Test samples of antibody were denatured in 10 mM phosphate buffer, 250 mM NaCl, 5 mM NEM, 6 M Guanidine, pH 7.0 at 37° C. for 1 to 3 hr. The denatured samples were then diluted 6 fold with 100 mM phosphate buffer, 0.1 mM EDTA, pH 7.0, to which Lys-C was added at a 1:10 enzyme to protein ratio. The reaction mixtures were incubated at 37° C. for 16 to 24 hours. Half of the reaction mixture was reduced by adding 5-10 μL of 100 mM DTT and incubated at 37° C. for 1 hr.

Lys-C digests were separated by reverse-phase HPLC (Phenomenex Jupiter 5m C18 column; 250×2.1 mm) and analyzed by an UV-detector and an on-line LCQ or LTQ Ion Trap mass spectrometer (ThermoElectron). The RP-HPLC mobile phase A was 0.1% TFA in H₂O and the mobile phase B was 0.1% TFA in acetonitrile. The peptides were eluted at a flow rate of 0.2 mL/min with the following gradient:

0-2 min, 5% Mobile Phase B

2-32 min, 5-20% Mobile Phase B

32-132 min, 20-40% Mobile Phase B

132-152 min, 40-60% Mobile Phase B

152-155 min, 60-95% Mobile Phase B

The column eluant was diverted to waste directly after the UV-detector during the first 15 min to avoid salt contamination of the LCQ source.

### Particle Counting

The number and size of particles in a solution was characterized by a Beckman Coulter Multisizer 3.

### Results

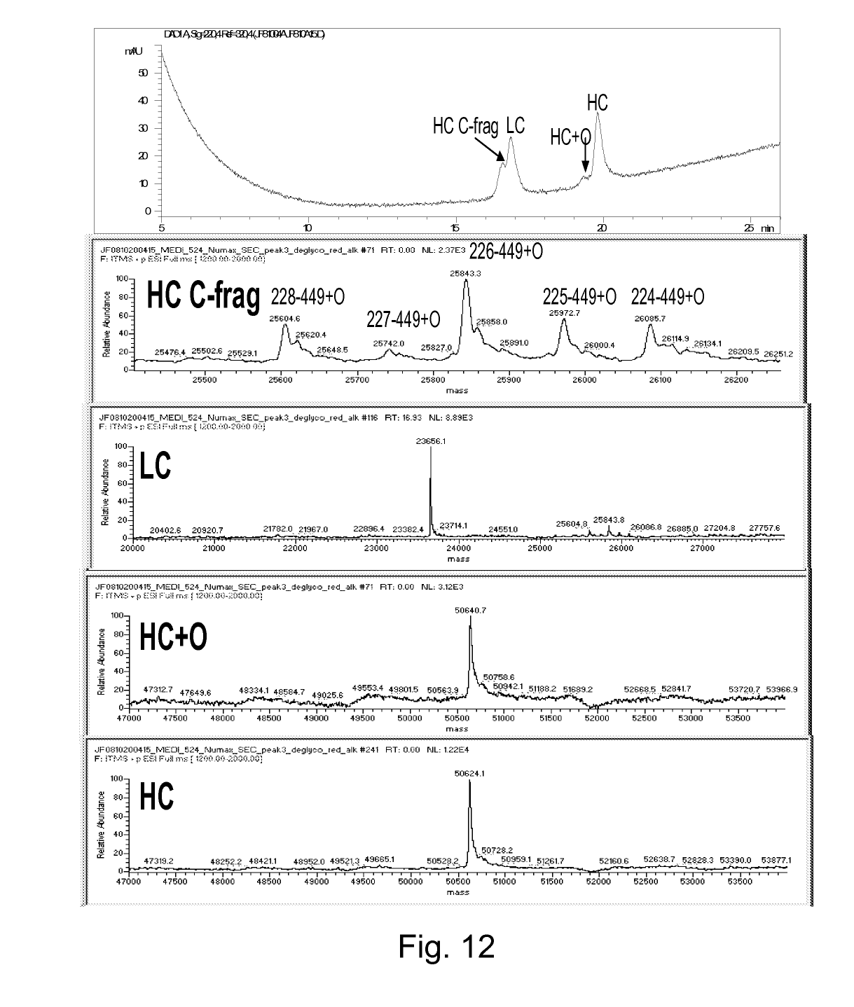

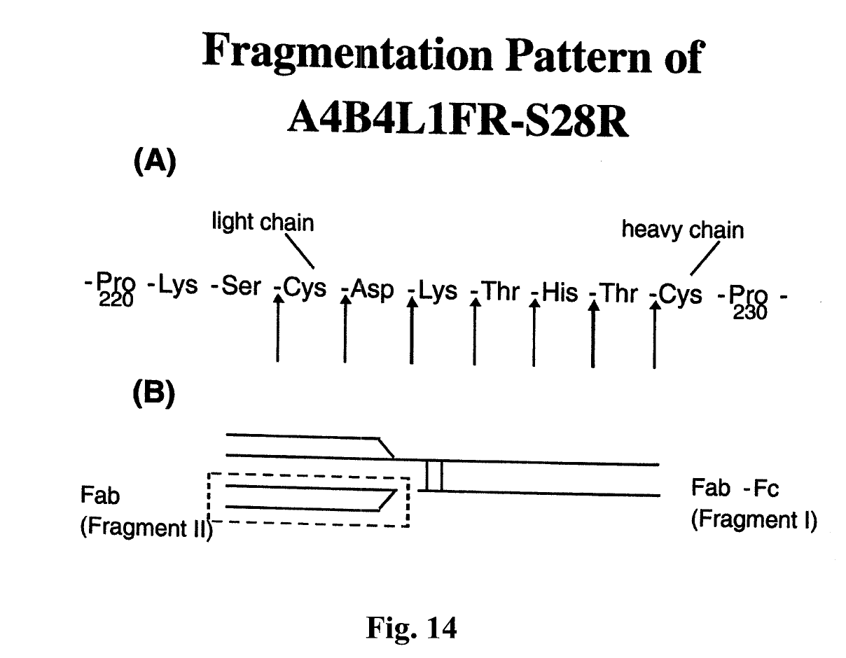

To characterize aggregates and fragments identified by SEC, fragment fractions were collected from the SEC chromatographic system and analyzed by LC-MS (antibody fragment I and antibody fragment II, FIGS. 12 and 13, respectively). The predominant fragments, above the detection limit of LC-MS, were identified for both fragment peaks (antibody type I fragment and antibody type II fragment) (FIG. 14 and Table 9). Antibody Type I and Antibody Type II fragments were generated by cleavage of the heavy chain in one of the hinge regions of the antibody. Observed cleavage sites were between serine 222 and cysteine 223, cysteine 223 and aspartic acid 224, between aspartic acid 224 and lysine 225, between lysine 225 and threonine 226, between threonine 226 and histidine 227, between histidine 227 and threonine 228, and between threonine 228 and cysteine 229.

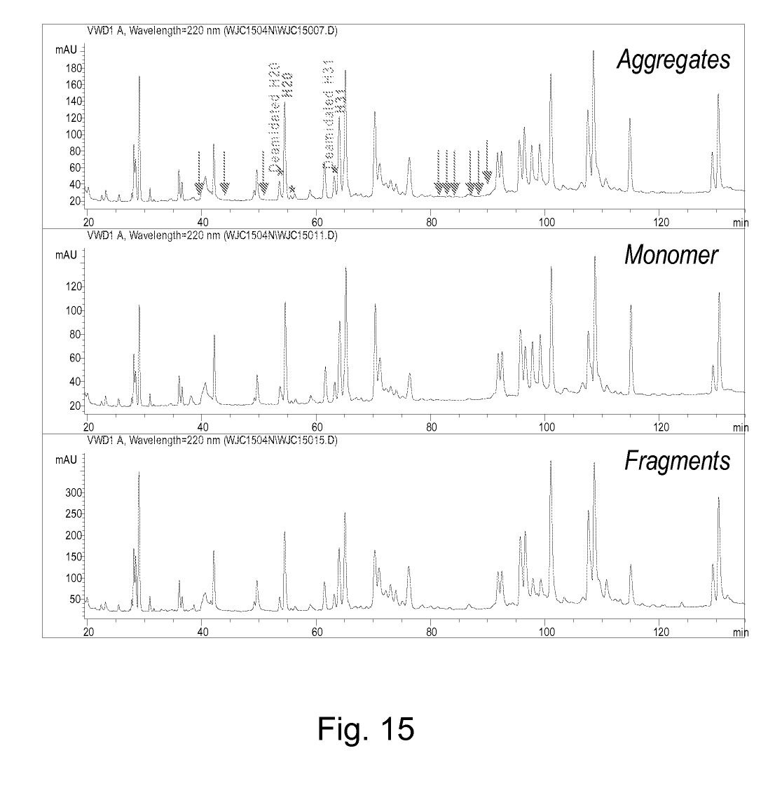

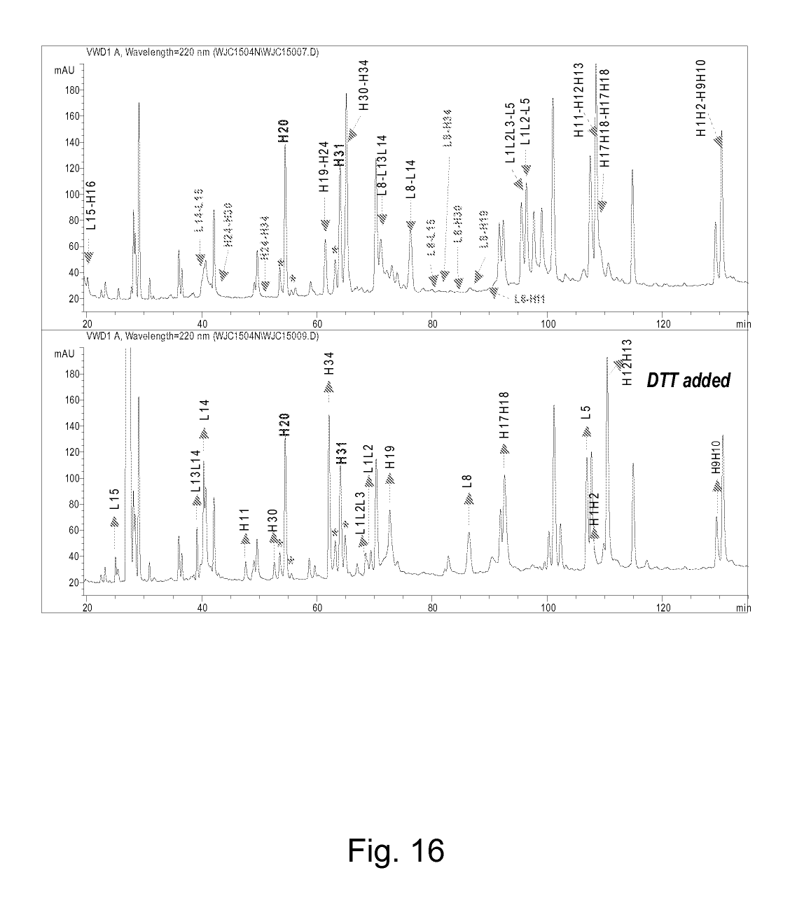

A comparison of peptide maps using reduced and non-reduced conditions of LC-MS/MS was also used to detect disulfide bond scrambling or other covalent modification in the monoclonal antibodies. The profile comparison for aggregates, monomer and fragments indicates that only a low level of disulfide bond scrambling existed in the aggregates (FIGS. 15 and 16). The results also suggest that most of the aggregates were non-covalently linked aggregates, as no significant profile change compared to that of monomer was observed.

TABLE 9
LC MS IDENTIFICATION OF motavizumab FRAGMENTS AFTER
STORAGE OF ANTIBODY FORMULATION AT 38-42° C. FOR 1
MONTH

| | | Calculated | | |
|---|---|---|---|---|
| | Sequence | MW | Measured MW | MW Accuracy |
| Reference | Light Chain | 23654.1 | 23654.9 | 0.0000 |
| Standard | Heavy Chain | 50617.68 | 50619.6 | 0.004% |
| Fragment | Light Chain | 23654.1 | 23655.2 | 0.0000 |
| II | H1-222 + O | 24360.69 | 24364.6 | 0.016% |
| | H1-223 + O | 24568.97 | 24571.1 | 0.009% |
| | H1-224 + O | 24684.06 | 24686 | 0.008% |
| | H1-226 + O | 24913.34 | 24913.5 | 0.001% |
| | H1-227 + O | 25050.48 | 25053.2 | 0.011% |
| | H1-222 | 24344.69 | 24346.2 | 0.006% |
| | H1-223 | 24552.97 | 24554.9 | 0.008% |
| | H1-224 | 24668.06 | 24671.5 | 0.014% |
| | H1-226 | 24897.34 | 24899.9 | 0.010% |
| | H1-227 | 25034.48 | 25037.9 | 0.014% |
| Fragment | Light Chain | 23654.1 | 23655.2 | 0.0000 |
| I | H228-449 + O | 25599.2 | 25604.6 | 0.021% |
| | H227-449 + O | 25736.34 | 25742 | 0.022% |
| | H226-449 + O | 25837.44 | 25843.3 | 0.023% |
| | H225-449 + O | 25965.61 | 25972.7 | 0.027% |
| | H224-449 + O | 26080.7 | 26085.7 | 0.019% |
| | H1-449 + O | 50633.68 | 50640.7 | 0.014% |
| | H1-449 | 50617.68 | 50624.1 | 0.013% |

A multisizer was also used to characterize the particle size distribution of the antibody formulation. A test sample of formulation at 100 mg/ml was analyzed in a Beckman Coulter Multisizer 3 (Table 10).

TABLE 10
PARTICLE ANALYSIS OF motavizumab SAMPLE AFTER STORAGE
OF ANTIBODY FORMULATION AT 38-42° C. FOR 1 MONTH

| | Dilution 1, | | | | | | |
|---|---|---|---|---|---|---|---|
| Size | run 1 | Dilution 1, run 2 | Dilution 1, run 3 | Dilution 2, run 1 | Dilution 2, run 2 | Dilution 2, run 3 | Average |
| μm | Particle/mL | Particle/mL | Particle/mL | Particle/mL | Particle/mL | Particle/mL | Particle/mL |
| 2-4 | 3.08E+05 | 3.11E+05 | 3.09E+05 | 2.81E+05 | 2.83E+05 | 2.82E+05 | 2.96E+05 |
| 4-10 | 3.93E+04 | 3.79E+04 | 3.75E+04 | 3.61E+04 | 3.54E+04 | 3.32E+04 | 3.66E+04 |
| 10-20 | 3.33E+03 | 3.47E+03 | 2.69E+03 | 6.11E+03 | 3.71E+03 | 3.74E+03 | 3.84E+03 |
| 20-30 | 5.97E+02 | 3.06E+02 | 2.55E+02 | 1.01E+03 | 3.06E+02 | 2.40E+02 | 4.52E+02 |
| 30-40 | 1.02E+02 | 5.10E+01 | 0.00E+00 | 1.48E+02 | 5.10E+01 | 5.10E+01 | 6.72E+01 |
| 40-60 | 5.10E+01 | 0.00E+00 | 0.00E+00 | 0.00E+00 | 0.00E+00 | 0.00E+00 | 8.50E+00 |
| 2-60 total | 3.51E+05 | 3.52E+05 | 3.50E+05 | 3.25E+05 | 3.22E+05 | 3.19E+05 | 3.37E+05 |

### 6.3 Kinetic Analysis of Binding of A4B4L1FR-S28R by Biacore™

The kinetics of the interactions of A4B4L1FR-S28R and palivizumab with RSV F-protein were determined by surface plasmon resonance (see, e.g., Jonsson et al., 1991, Biotechniques 11(5):620-627 and Johne, B. (1989). Epitope mapping by surface plasmon resonance in the BIAcore. Molecular Biotechnology 9(1):65-71) using a BIAcore 3000 instrument (BIAcore, Inc., Piscataway, N.J.). A recombinantly produced, C-terminally truncated RSV (A2 strain) F protein (Wathen et al., 1989, J Infect Dis 159(2):255-264) was used as the antigen for these studies. The truncated F protein, lacking the membrane anchor, was produced as a secreted product using a recombinant baculovirus expression system and was purified by successive chromatography steps on concanavalin-A and Q-sepharose columns. Purified F protein was covalently coupled to an N-hydroxysuccinimide-N-ethyl-N′-[3-diethylaminopropyl]-carbodiimide (EDC/NHS) activated CM5 sensor chip at a low protein density according to the manufacturer's protocol; unreacted active ester groups were blocked with 1 M ethanolamine. For reference purposes, a blank surface, containing no antigen, was prepared under identical immobilization conditions.

For kinetic measurements, a serial 2-fold dilution series of each mAb from 100 nm-0.2 nm, made in instrument buffer (HBS/Tween-20, BIAcore, Inc.), was injected over the F-protein and reference cell surfaces, which are connected in series. In each analysis, following the dissociation phase, the remaining bound antibody was removed from the sensor chip by passing a brief pulse of 100 mM HCl over the surface. Once an entire data set was collected, the resulting binding curves were globally fitted to a 1:1 Langmuir binding model using BIAevaluation software (BIAcore, Inc., Piscataway, N.J.). This algorithm calculates both the association rate (kₒₙ) and the dissociation rate (k_{off}), from which the apparent equilibrium binding constant, K_{D}, for each antibody was deduced as the ratio of the two rate constants, k_{off}/kₒₙ. A more detailed explanation of how the individual rate constants are derived can be found in the BIAevaluation Software Handbook (BIAcore, Inc., Piscataway, N.J.).

Kinetic analysis of binding by BIAcore evaluation (Table 11) revealed that, under the conditions of a low-density surface that were employed, A4B4L1FR-S28R (motavizumab) had an approximately 70-fold greater affinity for RSV F protein than palivizumab. The increased affinity of motavizumab for the RSV F protein is attributed to a 4-fold increase in the association rate and an approximately 17-fold decrease in the dissociation rate. Since the rate at which motavizumab dissociates from the F protein surface approaches the detection limits of the BIAcore 3000 instrument, the dissociation rate generated for motavizumab is an estimation.

TABLE 11
Kinetic Analysis of Binding

| mAb | kₒₙ (M⁻¹s⁻¹) | k_{off} (s⁻¹) | K_{D} (pM) |
|---|---|---|---|
| palivizumab | 1.14E+05 | 3.95E−04 | 3460 |
| motavizumab | 4.73E+05 | 2.35E−05 | 50 |

### 6.4 Example

### Microneutralization Assay

Neutralization of the antibodies of the present invention were determined by microneutralization assay. This microneutralization assay is a modification of the procedures described by Anderson et al. (1985, J. Clin. Microbiol. 22:1050-1052, the disclosure of which is hereby incorporated by reference in its entirety). The procedure used here is described in Johnson et al., 1999, J. Infectious Diseases 180:35-40, the disclosure of which is hereby incorporated by reference in its entirety. Antibody dilutions were made in triplicate using a 96-well plate. Ten TCID₅₀ of respiratory syncytial virus (RSV—Long strain) were incubated with serial dilutions of the antibody (or Fabs) to be tested for 2 hours at 37° C. in the wells of a 96-well plate. RSV susceptible HEp-2 cells (2.5×10⁴) were then added to each well and cultured for 5 days at 37° C. in 5% CO₂. After 5 days, the medium was aspirated and cells were washed and fixed to the plates with 80% methanol and 20% PBS. RSV replication was then determined by F protein expression. Fixed cells were incubated with a biotin-conjugated anti-F protein monoclonal antibody (pan F protein, C-site-specific mAb 133-1H) washed and horseradish peroxidase conjugated avidin was added to the wells. The wells were washed again and turnover of substrate TMB (3,3′,5,5′-tetramethylbenzidine) was measured at 450 nm. The neutralizing titer was expressed as the antibody concentration that caused at least 50% reduction in absorbency at 450 nm (the OD₄₅₀) from virus-only control cells. The results from the assay for the monoclonal antibodies and Fab fragments listed in Table 2 are shown in Table 11, supra, and Table 12, infra.

TABLE 12
End Point RSV Microneutralization Titer Of High On Rate Mutant IgG and Fab

| | Mean | | Fold | Mean | | Fold | |
|---|---|---|---|---|---|---|---|
| | IC50 | STDEV | Difference | IC50 | STDEV | Difference | |
| | (Curve) | Curve | (Curve | (Control) | Control | (Control | n (assay |
| Molecule | μg/ml | IC50 | IC50) | μg/ml | IC50 | IC50) | repeat) |
| **palivizumab | 0.4527 | 0.208 | — | 0.5351 | 0.238 | — | 8 |
| **A1e9 | 0.0625 | 0.0268 | 7 | 0.0645 | 0.223 | 8 | 3 |
| **A17d4(1) | 0.0342 | 0.022 | 13 | 0.0354 | 0.0187 | 15 | 4 |
| **P11d4 | 0.0217 | 0.0331 | 21 | 0.0289 | 0.0110 | 19 | 5 |
| **P12f2 | 0.0231 | 0.0141 | 20 | 0.0223 | 0.0083 | 24 | 6 |
| **A8c7 | 0.0337 | 0.0309 | 13 | 0.0383 | 0.0283 | 14 | 5 |
| **A12a6 | 0.0357 | 0.0316 | 13 | 0.0354 | 0.0261 | 15 | 7 |
| **P12f4 | 0.0242 | 0.0163 | 19 | 0.0235 | 0.0076 | 23 | 7 |
| **A13c4 | 0.0376 | 0.0268 | 12 | 0.0375 | 0.0213 | 14 | 6 |
| **A4B4 | 0.0171 | 0.0018 | 27 | 0.0154 | 0.00417 | 35 | 2 |
| *A1e9 | 0.157 | — | 3 | 0.125 | — | 4 | 1 |
| *A17d4(1) | 0.0179 | — | 25 | 0.0171 | — | 31 | 1 |
| *P11d4 | >1.00 | — | — | >1.00 | — | — | 1 |
| *P12f2 | 0.0407 | 0.0112 | 11 | 0.0326 | 0.00905 | 16 | 2 |
| *A8c7 | 0.177 | — | 3 | 0.157 | — | 34 | 1 |
| *A12a6 | 0.0287 | 0.00417 | 16 | 0.0310 | 0.00982 | 17 | 2 |
| *P12f4 | 0.0464 | 0.00791 | 10 | 0.0351 | 0.0126 | 15 | 2 |
| *A13c4 | 0.0264 | 0.00141 | 17 | 0.0258 | 0.00071 | 21 | 2 |
| *A4B4 | 0.0414 | — | 11 | 0.0411 | — | 13 | 1 |
| *A13a11 | 0.120 | 0.0222 | 4 | 0.1022 | 0.0260 | 5 | 2 |
| *A1h5 | 0.194 | 0.462 | 2 | 0.176 | 0.0625 | 3 | 2 |

**Monoclonal Antibody
*Fab Fragment

### 6.5 RSV Microneutralization Assay

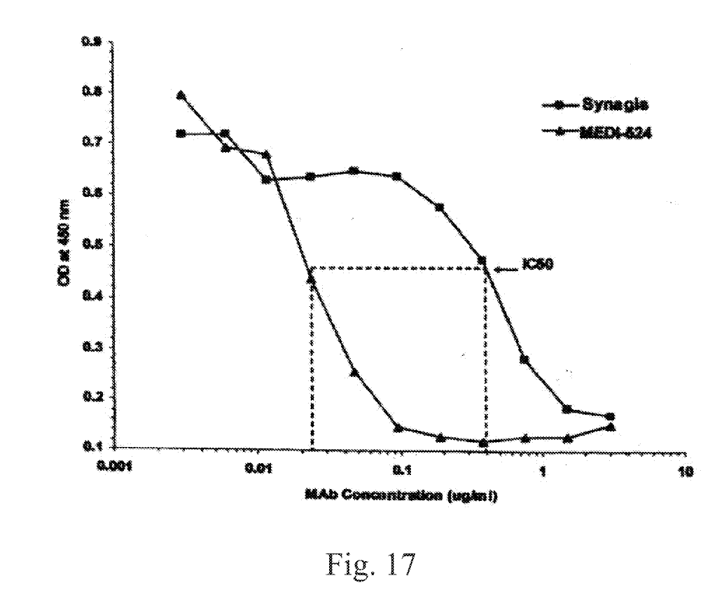

The ability of A4B4L1FR-S28R (motavizumab) and palivizumab to inhibit the in vitro replication of RSV (Long strain) was evaluated using a RSV microneutralization assay. This assay is a modification of the procedure of Anderson et al. (Anderson et al., 1985, J Clin Microbiol 22: 1050-1052) as described by Johnson et al. (Johnson et al., 1997, J Infect Dis 176: 1215-1224). Antibody dilutions were made in duplicate to quadruplicate wells of a 96-well plate. Approximately 100-1000 TCID₅₀ of RSV (Long) were added to each dilution well and incubated for two hours at 37° C. Low passage, RSV susceptible HEp-2 cells (2.5×10⁴) were then added to each well and cultured for five days at 37° C. in a humidified 5% CO₂ incubator. After four or five days the cells were washed with PBS −0.1% Tween 20 and fixed to the plate with 80% acetone with 20% PBS. RSV replication was determined by quantitation of F protein expression using an F protein-specific ELISA. Fixed cells were incubated with the C-site specific, pa RSV F protein mAb 133-1H (Chemicon, Inc.), washed, and then incubated with horseradish peroxidase-conjugated goat anti-mouse IgG and washed again. The peroxidase substrate TMB (3,3′,5,5′-tetramethylbenzidine) was added to each well and the reaction was stopped after twenty minutes by the addition of 2 M H₂S0₄. Substrate turnover was measured at 450 nm (OD450) using a microplate reader. The neutralizing titer is expressed as the antibody concentration resulting in at least a 50% reduction in the OD450 value from control wells with virus only (IC₅₀). The results of this assay, shown in FIG. 17, indicate that motavizumab (average IC₅₀=18 ng/ml) is approximately 18-fold more potent than palivizumab (average IC₅₀=315 ng/ml).

### 6.6 RSV Microneutralization Assay with Cynomolgus BAL Samples

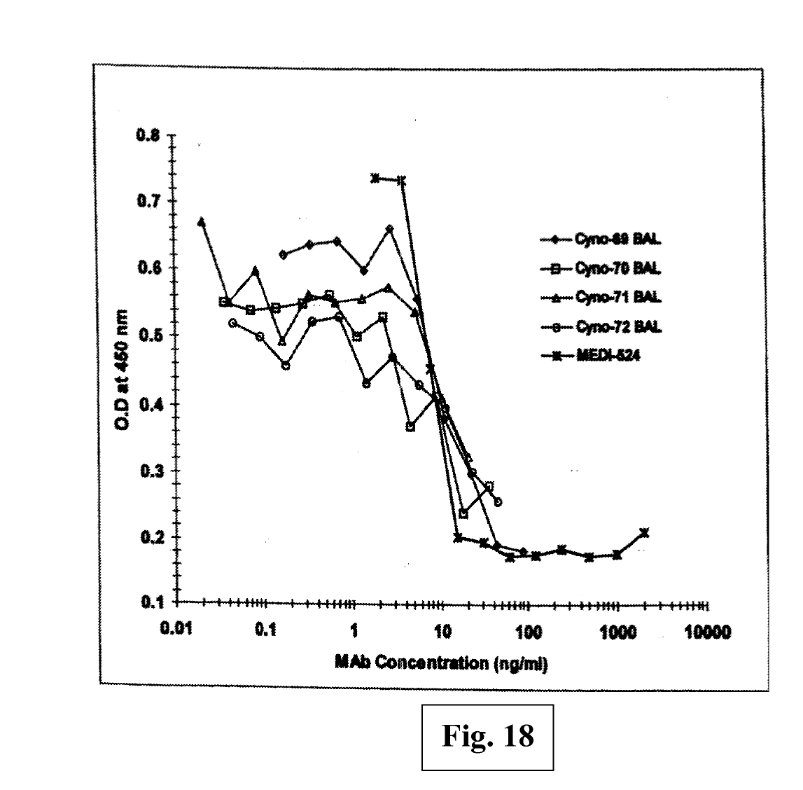

The ability of motavizumab present in the lungs of treated animals to inhibit the in vitro replication of RSV was evaluated using the RSV microneutralization assay. Four juvenile female cynomolgus monkeys (average weight 2.0 kg) were sedated with Telazol and dosed intravenously (i.v.) with motavizumab at 30 mg/kg body weight via the saphenous vein using an external infusion pump. Four days later, the animals were anesthetized with Telazol and a bronchial alveolar lavage (BAL) was performed on one lobe of the right lung with phosphate buffered saline (PBS). Titers of motavizumab in the BAL fluid were determined using a motavizumab-specific ELISA. The BAL samples were tested undiluted and at serial 2-fold dilutions in the RSV microneutralization assay as above with purified motavizumab included as a control. The results of this assay, shown in FIG. 18, show that motavizumab retains full RSV neutralizing activity in the lungs of cynomolgus monkeys four days after infusion.

### 6.7 RSV Fusion Inhibition Assay

The ability of the antibodies of the invention to block RSV-induced fusion after viral attachment to the cells is determined in a fusion inhibition assay. This assay is identical to the microneutralization assay, except that the cells are infected with RSV (Long) for four hours prior to addition of antibody (Taylor et al., 1992, J. Gen. Virol. 73:2217-2223).

### 6.8 Physical Characterization

The example illustrates the physical characteristics of motavizumab and palivizumab. A number of parameters were examined including the Tm and pI. In addition, the aggregation rates and viscosity profiles of motavizumab and palivizumab were determined.

### Materials and Methods

### Generation of Antibody Fragments

Fab and Fc domains were generated from full length palivizumab antibody using papain. A commercial kit from Pierce (ImmunoPure Fab Preparation Kit Pierce Product # 44885: ImmunoPure IgG Binding Buffer, ImmunoPure IgG Elution Buffer, AffinityPak Immobilized Protein A Column, Immobilized Papain, Cysteine monohydrochloride, Phosphate Buffer, and Serum Separators) was used to digest the intact antibodies. The enzymology was optimized to achieve the best cleavage of the Mab in a reasonable time. Fab and Fc domains were generated from palivizumab using the following steps: a) adding antibody to papain and incubating overnight at 37° C., ˜10 mg of IgG per digestion; b) separating crude digest from immobilized enzyme; c) applying digest to Protein A column; d) eluting the Fab fragment in unretained fraction at pH-8.0; e) eluting the Fc fragment at pH-3.0; and f) dialyzing the fragments into a required buffer.

### Differential Scanning Calorimetry

Thermal melting temperatures (Tₘ) were measured with a VP-DSC (MicroCal, LLC) using a scan rate of 1.0° C./min and a temperature range of 25-120° C. A filter period of 8 seconds was used along with a 5 minute pre-scan thermostating. Samples were prepared by dialysis into 10 mM Histidine-HCl, pH 6 using Pierce dialysis cups (3.5 kD). Average Mab concentrations were 50 μg/mL as determined by A₂₈₀. Melting temperatures were determined following manufacturer procedures using Origin software supplied with the system. Briefly, multiple baselines were run with buffer in both the sample and reference cell to establish thermal equilibrium. After the baseline was subtracted from the sample thermogram, the data were concentration normalized and fitted using the deconvolution function.

### Isoelectric Focusing Gel Electrophoresis

Isoelectric points were determined using a Pharmacia Biotech Multiphor 2 electrophoresis system with a multi temp 3 refrigerated bath recirculation unit and an EPS 3501 XL power supply. Pre-cast ampholine gels (Amersham Biosciences, pI range 2.5-10) were loaded with 5 μg of protein. Broad range pI marker standards (Amersham, pI range 3-10, 8 μL) were used to determine relative pI for the Mabs. Electrophoresis was performed at 1500 V, 50 mA for 105 minutes. The gel was fixed using a Sigma fixing solution (5×) diluted with purified water to 1×. Staining was performed overnight at room temperature using Simply Blue stain (Invitrogen). Destaining was carried out with a solution that consisted of 25% ethanol, 8% acetic acid and 67% purified water. Isoelectric points were determined using a Bio-Rad Densitometer relative to calibration curves of the standards.

### Viscosity Profile

Viscosities of mAB solutions were measured using a ViscoLab 4000 Viscometer System (Cambridge Applied Systems) equipped with a ViscoLab Piston (SN:7497, 0.3055″, 1-20 cP) and S6S Reference Standard (Koehler Instrument Company, Inc.). The viscometer was connected to a water bath and equilibrate the system to 20° C. Piston was checked using S6S viscosity reference standard (8.530 cP @ 20.00° C.). Piston was also checked using RODI H₂O (1.00 cP @ 20.0° C.). The piston was cleaned and rinsed thoroughly with soap and water between measurements of each different solution type. Each Mab was in 10 mM Histidine-HCl, pH 6 at a concentration of 100 mg/mL. The system was then cooled to ≦2° C. When the system temperature was at or below 2° C., sample was loaded into the chamber and the piston was lowered into the sample. After sample was equilibrated to the temperature of the chamber, measurement was initiated. The temperature was increased at 1° C. increments every 7-10 minutes to a final temperature of ≧25° C. The temperature was adjusted on the water bath but the recorded temperature was what was displayed on the viscometer. The viscosity result was recorded immediately prior to increasing the temperature. The piston remained in motion during measurements to minimize the need for re-equilibration.

### Aggregation Rate

Aggregation profiles over a range of temperatures were determined by HPSEC. Specifically, approximately 250 μg of, for example, the antibody or antibody fragment that immunospecifically binds to a target antigen (approximately 25 μl of a liquid formulation comprising 10 mg/ml said antibody or antibody fragment) was injected onto a TosoH Biosep TSK G3000SWXL column (7.8 mm×30 cm) fitted with a TSK SW ×1 guard column (6.0 mm CX 4.0 cm). The antibody or antibody fragment was eluted isocratically with 0.1 M disodium phosphate containing 0.1 M sodium sulfate and 0.05% sodium azide, at a flow rate of 0.8 to 1.0 ml/min. Eluted protein was detected using UV absorbance at 280 nm. A suitable reference standard was run in the assay as a control, and the results were reported as the area percent of the product monomer peak compared to all other peaks excluding the included volume peak observed at approximately 12 to 14 minutes. Peaks eluting earlier than the monomer peak were recorded as percent aggregate.

### Results

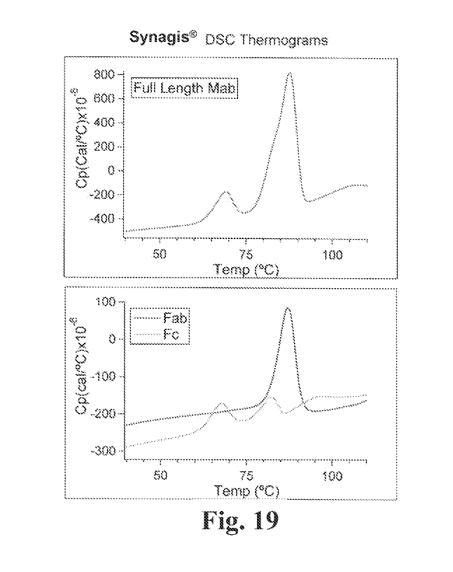

Differential Scanning Calorimetry (DSC) was used to examine the melting curve of the full length palivizumab (FIG. 19, top). Fab and Fc domain fragments were generated from palivizumab and the purified fragments were analyzed individually by DSC (FIG. 19, bottom). The results show that individual Tm peaks in a full antibody may be assigned to individual domains. In particular, the largest peak represents the Tm of the Fab portion of a full length antibody. The Tm of the palivizumab Fab is about 87.6° C.

A similar analysis was performed on motavizumab (data not shown). The Tm of the motavizumab Fab was found to be significantly higher, about 93.1. This finding is unexpected as these two molecules differ by only 13 amino acids.

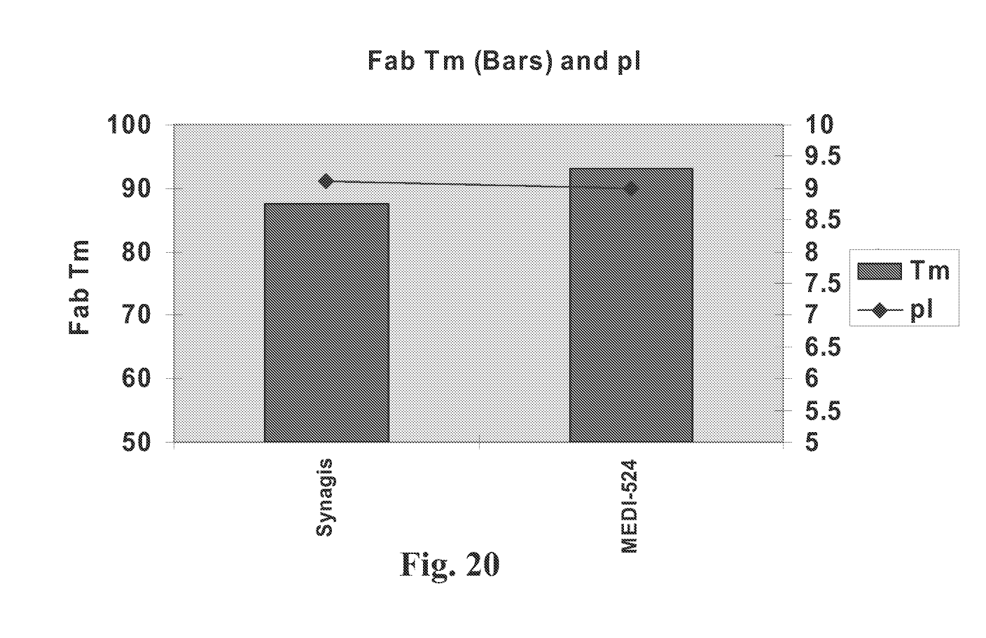

To further characterize these molecules, the pI for each full length mAb was determined by isoelectric focusing gel electrophoresis, motavizumab has a pI of 9.0 and palivizumab was found to have a pI of 9.1. The Fab-Tm and mAb-pI values for each antibody are plotted in FIG. 20 for comparison.

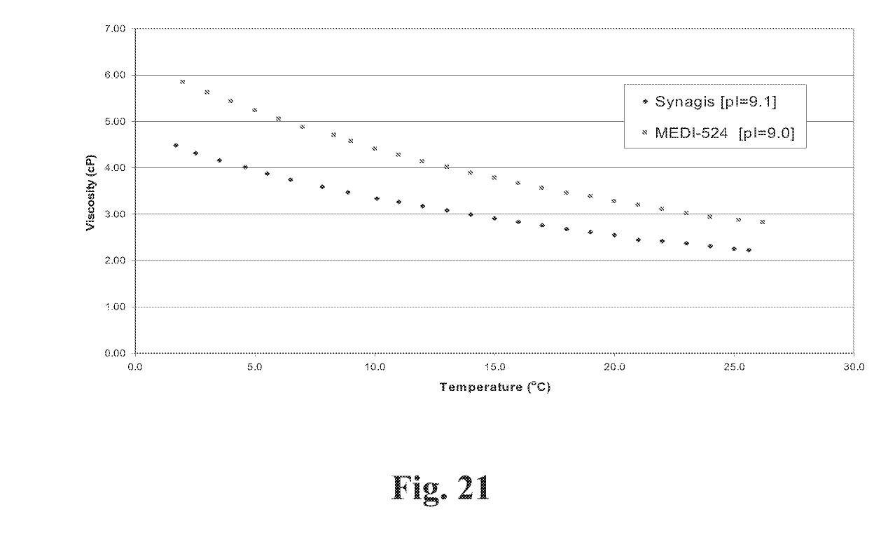

The viscosities of 100 mg/ml solutions of motavizumab and palivizumab were respectively examined over a range of temperatures from about 2 to about 25° C. The viscosity of motavizumab ranged from a high of about 6.0 cP at 2° C. to a low of about 3.0 cP at about 25° C. The viscosity of palivizumab ranged from a high of about 4.5 cP at 2° C. to a low of about 2.0 cP at about 25° C. (FIG. 21).

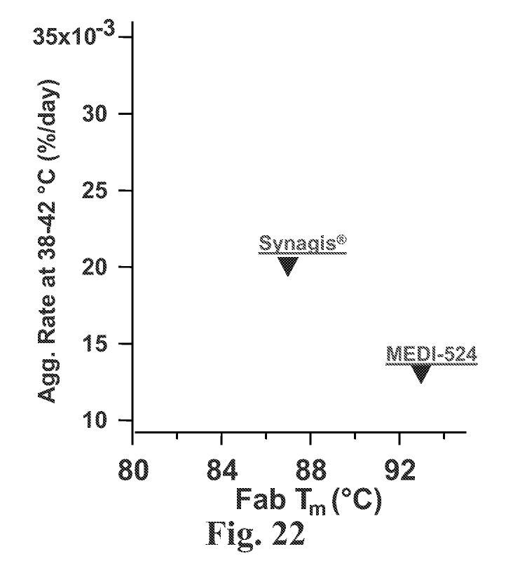

The aggregation rates of palivizumab and motavizumab were plotted against the Fab Tm for each antibody (FIG. 22). A correlation between Fab Tm and reduced aggregation rates is seen. motavizumab, which has a significantly higher Fab Tm, is much less prone to forming aggregates than palivizumab.

### 7. Equivalents

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

### 8. Sequence Listing

This application includes a Sequence Listing submitted on compact disc, recorded on two compact discs (CD-ROM), including one duplicate, containing Filename 10271-170-999 (US as filed).txt of file size 606,000 bytes created Jun. 22, 2006. The sequence listing on the compact discs is incorporated by reference herein in its entirety.

1.-20. (canceled)

21. A liquid antibody formulation comprising a full length IgG₁ antibody comprising a variable heavy (VH) domain of SEQ ID NO:48 and a variable light (VL) domain of SEQ ID NO:11, wherein said liquid antibody formulation is produced by the method steps comprising:
(a) antibody manufacture step;
(b) antibody purification step; and
(c) antibody formulation step,
wherein said antibody manufacture step comprises:
(i) transformation of NS0 host cells with a recombinant vector capable of directing transcription of mRNA encoding said antibody; and
(ii) expression of said full length IgG₁ antibody from said transformed NS0 host cells;
wherein said antibody purification step comprises:
(iii) cation exchange chromatography;
(iv) anion exchange chromatography;
(v) nanofiltration;
(vi) low pH treatment;
(vii) hydroxyapatite chromatography, and
(viii) resulting full length IgG₁ antibody fraction,
wherein said antibody formulation step comprises about 100 mg/ml of said resulting
full length IgG₁ antibody fraction in a histidine buffer,
wherein no more than 0.1% of the total protein of said resulting full length IgG₁ antibody in said pure liquid antibody formulation comprises antibody type I fragments and antibody type II fragments as determined by size exclusion chromatography (SEC) with UV detection or by analytical ultracentrifugation (AUC).

22. The liquid antibody formulation of claim 21, wherein the antibody type I fragments comprise one or more C-terminal portions of the heavy chain of the resulting full length IgG₁ antibody, which heavy chain C-terminal portion has a molecular weight of about 25.6 kD, about 25.7 kD, about 25.8 kD, about 26.0 kD, or about 26.1 kD and wherein the antibody type II fragments comprise one or more N-terminal portions of the heavy chain of the resulting full length IgG₁ antibody, which heavy chain N-terminal portion has a molecular weight of about 24.4 kD, about 24.6 kD, about 24.7 kD, about 24.9 kD, or about 25.1 kD, both as determined by Liquid Chromatography Mass Spectrometry (LC-MS) analysis.

23. The liquid antibody formulation of claim 21, wherein the antibody type I fragments comprise one or more C-terminal portions of the heavy chain of the resulting full length IgG₁ antibody, which heavy chain C-terminal portion comprises amino acid residues 223-449 of the IgG1 heavy chain, amino acid residues 224-449 of the IgG1 heavy chain, amino acid residues 225-449 of the IgG1 heavy chain, amino acid residues 226-449 of the IgG1 heavy chain, amino acid residues 227-449 of the IgG1 heavy chain, amino acid residues 228-449 of the IgG1 heavy chain and amino acid residues 229-449 of the IgG1 heavy chain, and wherein the antibody type II fragments comprise one or more heavy chain N-terminal portions of the heavy chain of the resulting full length IgG₁ antibody which heavy chain N-terminal portion comprises amino acid residues 1-222 of the IgG1 heavy chain, amino acid residues 1-223 of the IgG1 heavy chain, amino acid residues 1-224 of the IgG1 heavy chain, amino acid residues 1-225 of the IgG1 heavy chain, amino acid residues 1-226 of the IgG1 heavy chain, amino acid residues 1-227 of the IgG1 heavy chain and amino acid residues 1-228 of the IgG1 heavy chain, as determined by LC-MS.

24. The formulation of claim 21, wherein a degassed sample of said pure liquid antibody formulation has a turbidity value of less than 10 NTU as measured by a turbidimeter.

25. The formulation of claim 21, wherein a degassed sample of said formulation has a turbidity value of between 4 to 8 NTU as measured by a turbidimeter.

26. The formulation of claim 21, wherein said formulation comprises a particle profile of less than about 3.4 E+5 particles/ml of diameter 2-4 μm, less than about 4.0 E+4 particles/ml of diameter 4-10 μm, less than about 4.2 E+3 particles/ml of diameter 10-20 μm, less than about 5.0 E+2 particles/ml of diameter 20-30 μm, less than about 7.5 E+1 particles/ml of diameter 30-40 μm, and less than about 9.4 particles/ml of diameter 40-60 μm, as determined by a multisizer.

27. The formulation of claim 21, wherein said resulting full length IgG₁ antibody in said stable liquid antibody formulation neutralizes respiratory syncytial virus (RSV) at an EC50 of less than 2.0 nM as measured by an in vitro microneutralization assay.

28. The formulation of claim 21, wherein the formulation is sterile.

29. A sealed container comprising the formulation of claim 21.

30. The formulation of claim 21, wherein the formulation is suitable for intramuscular administration.

31. A method of preventing RSV infection in a patient, the method comprising administering an effective amount of the antibody formulation of claim 28.
